(19) United States
(12) Patent Application Publication
(10) Pub. No.: US 2024/0352123 A1
(43) Pub. Date: Oct. 24, 2024

(54) HIGH SELECTIVE CD229 ANTIGEN BINDING DOMAINS AND METHODS OF USE

(71) Applicants: UNIVERSITY OF UTAH RESEARCH FOUNDATION, Salt Lake City, UT (US); UNIVERSITY OF MARYLAND BALTIMORE, OFFICE OF TECHNOLOGY TRANSFER, Baltimore, MD (US)

(72) Inventors: Tim C. Luetkens, Salt Lake City, UT (US); Erica Vander Mause, Salt Lake City, UT (US); Djordje Atanackovic, Salt Lake City, UT (US)

(21) Appl. No.: 18/715,007

(22) PCT Filed: Dec. 2, 2022
(86) PCT No.: PCT/US2022/051680

§ 371 Date: May 30, 2024

Related U.S. Application Data
(60) Provisional application No. 63/285,843, filed on Dec. 3, 2021.

(51) Int. Cl. C07K 16/28; A61K 39/00; A61K 39/395; A61K 45/06; A61P 35/00; C07K 14/705; C07K 14/725; C07K 14/73

(52) U.S. Cl. CPC C07K 16/2803; A61K 39/3955; A61K 39/4611; A61K 39/4631; A61K 39/464411; A61K 45/06; A61P 35/00; C07K 14/70503; C07K 14/7051; C07K 14/70514; C07K 14/70521; C07K 14/70578; C07K 2317/622; C07K 2319/02; C07K 2319/03; C07K 2319/30; C07K 2319/42

(57) ABSTRACT

Disclosed are chimeric antigen receptor (CAR) polypeptides comprising a CD229 antigen binding domain, a transmembrane domain, and an intracellular signaling domain, wherein the CD229 binding domain is a variant CD229 antigen binding domain. Disclosed are chimeric antigen receptor (CAR) polypeptides comprising a CD229 antigen binding domain, a transmembrane domain, and an intracellular signaling domain, wherein the CD229 antigen binding domain comprises the sequence of SEQ ID NO:134, SEQ ID NO:53, or SEQ ID NO:84. Disclosed are methods of using the CAR polypeptides or antibodies comprising the same CD229 antigen binding domain as the CAR polypeptides.

### CROSS-REFERENCE TO RELATED APPLICATIONS

This application claims the benefit of U.S. Provisional Patent Application No. 63/285,843, filed Dec. 3, 2021, which is incorporated by reference herein in its entirety.

### BACKGROUND

T cells expressing chimeric antigen receptors (CAR) using single-chain variable fragments (scFv) to target cancer-associated surface antigens are highly effective against several hematologic malignancies, including B cell lymphoma(1) and multiple myeloma(2, 3). However, their extraordinary cytotoxic activity poses new challenges, such as the unintended killing of healthy tissues expressing the targeted antigen, despite often at substantially lower levels(4). In the case of the widely used CD19 CAR T cells, this on-target off-tumor toxicity results in the elimination of healthy B cells(5, 6) and various other CAR T cell approaches have resulted in life-threatening toxicities and even patient deaths due to the targeting of healthy tissues(7-9). It has been shown previously that CAR T cells exert potent anti-tumor activity across a wide range of affinities(10-12) and many CAR T cell strategies currently in clinical use likely exceed the required affinity threshold. Consequently, low affinity antibodies have been developed for several cancer targets to increase cancer selectivity as well as CAR T cell persistence and function(13-16). However, none of these binding domains were derived from existing and extensively tested high-affinity antibodies already in clinical use and had to, again, undergo rigorous preclinical evaluation with the risk for substantial liabilities, such as off-target reactivity and unstable epitopes.

### BRIEF SUMMARY

Disclosed is an approach of the systematic optimization of antibody affinity of existing CAR binding domains by way of modifying parental high-affinity antibodies that can be an important tool in the development of more effective and safer CAR T cell approaches.

Disclosed are chimeric antigen receptor (CAR) polypeptides comprising a CD229 antigen binding domain, a transmembrane domain, and an intracellular signaling domain, wherein the CD229 binding domain is a variant CD229 antigen binding domain.

Disclosed are chimeric antigen receptor (CAR) polypeptides comprising a CD229 antigen binding domain, a transmembrane domain, and an intracellular signaling domain, wherein the CD229 binding domain comprises the amino acid sequence of SEQ ID NO:1 having one or more amino acid variations, wherein an amino acid variation can be a deletion, substitution, or modification.

Disclosed are chimeric antigen receptor (CAR) polypeptides comprising a CD229 antigen binding domain, a transmembrane domain, and an intracellular signaling domain, wherein the CD229 antigen binding domain comprises the sequence of SEQ ID NO:134, SEQ ID NO:53, or SEQ ID NO:84.

Disclosed are chimeric antigen receptor (CAR) polypeptides comprising a CD229 antigen binding domain, a transmembrane domain, and an intracellular signaling domain, wherein the CD229 antigen binding domain comprises a HCDR3 comprising the sequence of AKRDNSNSFDYW (SEQ ID NO:253), AKRGNENSFDYW (SEQ ID NO:284, or AKRGNSNSQDYW (SEQ ID NO: 134).

Disclosed are nucleic acid sequences capable of encoding any of the disclosed CAR polypeptides.

Disclosed are vectors comprising the nucleic acid sequence of the disclosed CAR nucleic acid sequences.

Disclosed are cells comprising any of the disclosed CAR polypeptides, CAR nucleic acids, or disclosed vectors.

Disclosed are antibodies or fragments thereof that bind to human CD229, wherein said antibody comprises a CD229 antigen binding domain comprising the sequence of SEQ ID NO:134, SEQ ID NO:53, or SEQ ID NO:84. Disclosed are antibodies or fragments thereof that bind to human CD229, wherein said antibody comprises a CD229 antigen binding domain comprising a HCDR3 comprising the sequence of AKRDNSNSFDYW (SEQ ID NO:??), AKRGNENSFDYW (SEQ ID NO:**), or AKRGNSNSQDYW (SEQ ID NO: ##).

Disclosed herein are compositions comprising any of the disclosed polypeptides, nucleic acids, vectors or cells.

Disclosed are methods of treating multiple myeloma comprising administering an effective amount of a T cell genetically modified to express one or more of the disclosed CAR polypeptides to a subject in need thereof.

Disclosed are methods of detecting CD229 on a cell comprising administering a composition comprising one or more of the disclosed antibodies or fragments thereof to a sample and detecting the binding of the antibody or fragment thereof to CD229.

Disclosed are methods of killing CD229 positive cells comprising administering an effective amount of a cell genetically modified to express one or more of the disclosed CAR polypeptides to a sample comprising CD229 positive cells.

Disclosed are methods of preferentially targeting cancer cells comprising administering a composition comprising one or more of the disclosed antibodies or fragments thereof to a sample and detecting the binding of the antibody or fragment thereof to CD229.

Disclosed are methods of making a cell comprising transducing a cell with one or more of the disclosed vectors.

Disclosed are methods of activating a T cell expressing one of the CAR polypeptides disclosed herein comprising culturing the T cell with a cell expressing CD229 and detecting the presence or absence of IFN-γ after culturing, wherein the presence of IFN-γ indicates the activation of the T cell.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1G show the generation of 2D3-based CDR3 variant library for the generation of low-affinity CD229 antibodies with increased selectivity. (FIG. 1A) Killing of CD229-positive MM cell line U266B1 expressing luciferase by CD229 CAR T cells (2D3) or T cells expressing a CAR without an scFv binding domain (AscFv) as determined by luminescence assay. Data indicate mean±S.D. from technical replicates (N=3). (FIG. 1B) Killing of healthy human T cells by CD229 CAR T cells (2D3) or T cells expressing a CAR without a binding domain (AscFv) as determined by flow cytometry cytotoxicity assay. Data indicate mean±S.D. from technical replicates (N=3). (FIG. 1C) Surface expression of CD229 on MM and T cells from patients with relapsed/refractory MM (N=6). Expression was determined after staining with an anti-CD229 antibody (clone: HLy9.1.25) on a CytoFLEX LX flow cytometer (Beckman Coulter). (FIG. 1D) Schema of relationship between CAR affinity and targeting of cells expressing high antigen levels (Ag^{hi}) and low antigen levels (Ag^{lo}). (FIG. 1E) Sensorgram of 2D3 binding to CD229. Equilibrium and rate constants of the 2D3 scFv were determined by bio-layer interferometry (BLI). Biotinylated 2D3 was immobilized on a streptavidin biosensor and the recombinant extracellular domain of CD229 was added in the following concentrations: 2 μM, 1 μM, 0.5 μM, 0.25 μM. Sensorgram indicates binding curves for descending CD229 concentrations. Plot shows a representative result of three independent experiments. (FIG. 1F) Structure of the 2D3 scFv as predicted by AlphaFold2 with the GS-linker omitted. CDR3 loops of the variable heavy (black) and the variable light (dark grey) chains as well as exposed residues are highlighted. (FIG. 1G) Schema of single amino acid substitution 2D3 CDR3 variant library indicating represented residues. Missing variants (light grey) and wild-type residues (black for HCDR3/dark grey for LCDR3) are highlighted.

FIGS. 2A-2D show single amino acid substitutions result in substantially reduced CD229 binding. (FIG. 2A) Concentration-dependence of 2D3 scFv binding to recombinant CD229 as determined by solid-phase time-resolved fluorescence (TRF) assay. Data indicate mean±S.D. from technical replicates (N=3). (FIG. 2B) Schema of solid-phase assay for the determination of FLAG-tagged scFv concentrations using rat IgG2a λ anti-FLAG antibody, biotinylated Protein L, and streptavidin-Eu. (FIG. 2C) Total yield of 2D3 scFv variants from autoinduction cultures as determined by assay illustrated in 2B. White squares with black outlines indicate amino acids in wildtype 2D3. Plot shows representative result of two independent experiments. (FIG. 2D) Binding of all expressed 2D3 heavy and light chain complementary determining region 3 (HCDR3 and LCD3, respectively) variants at 2 ng/l to recombinant CD229 as determined by solid-phase TRF assay. Line indicates wildtype 2D3 binding to CD229. Circles indicate variants selected for downstream assays based on amino acid position and binding signal. Plot shows representative result of two independent experiments.

FIGS. 3A-3D shows an affinity tuning approach results in predominantly off-rate-driven affinity reductions. (FIG. 3A) Schema of construct used for production of biotinylated 2D3 scFv variants including C-terminal AviTag to facilitate in vivo biotinylation. (FIG. 3B) Schema of biolayer interferometry (BLI) setup used for kinetic characterization of CD229 binding. Biotinylated 2D3 variants were immobilized on streptavidin biosensors and the recombinant extracellular domain of CD229 was added in the following concentrations: 2 μM, 1 μM, 0.5 μM, 0.25 μM. (FIG. 3C) Sensorgrams of 2D3 variants were determined using an Octet K2 (Sartorius). Plots show representative result of two independent experiments. (FIG. 3D) Correlation between rate and equilibrium constants of 2D3 variant scFvs as determined by BLI.

FIGS. 4A-4F show multiple HCDR3 variants maintain anti-tumor activity but exhibit minimal T cell killing. (FIG. 4A) Schema of 4-1BB-based second-generation CAR construct with GFP reporter. (FIG. 4B) Schema of the gammaretrovirus-based CAR T cell production process. (FIG. 4C) Correlation between CAR T cell yield and viability of 26 2D3 variant CARs. (FIG. 4D) Surface expression of 2D3 variant CARs and GFP reporter expression as determined by anti-HA staining using flow cytometry. (FIG. 4E) Cytotoxic activity of 2D3 variant CAR T cells against MM cell line U266B1 expressing luciferase at different effector-target ratios using a luminescence-based cytotoxicity assay. Data indicate mean±S.D. from technical replicates (N=3). (FIG. 4F) Correlation of cytotoxic activity of 2D3 variant CAR T cells against MM cells and T cells overnight at an effector-target ratio of 0.5:1. Data indicate mean killing (fold of wildtype 2D3)±S.D. from technical replicates (N=3). Candidates were selected for downstream assays based on increased selectivity and anti-tumor activity (arrow points to these candidates).

FIGS. 5A-5P show low affinity variants exhibit minimal T cell killing and reduced trogocytosis, while maintaining target specificity and anti-MM activity. (FIG. 5A) Killing of parental luciferase-expressing CD229-negative K562 cells and K562 cells transduced with a CD229 expression construct. Target cell killing was determined by luciferase-based cytotoxicity assay. Data indicate mean±S.D. from technical replicates (N=3). (FIG. 5B) Killing of primary human MM cells after overnight co-culture by CD229 CAR T cells as determined by flow cytometry. Data indicate mean±S.D. from technical replicates (N=3). Statistical differences between conditions were determined by two-sided Student's t-test. (FIG. 5C) Expansion of CD229 CAR T cells during manufacturing as determined by cell counting. Data are representative of 2 independent experiments. (FIG. 5D) NRG mice bearing U266B1 tumors were injected with T cells expressing FH9Q CAR T cells with or without c-Jun. Mice were euthanized between days 7 and 9 and CAR T cell numbers determined by flow cytometry. Data indicate mean±S.D. from 5 animals per group. Statistical differences between conditions were determined by two-sided Student's t-test. (FIG. 5E) Retroviral construct used for the simultaneous expression of CARs and c-Jun. (FIG. 5F) Killing of U266B1-Luc cells by HA^{int}sorted CD229 CAR T cells after an overnight co-culture as determined by luciferase-based cytotoxicity assay. Data indicate mean±S.D. from technical replicates (N=3). (FIG. 5G) Killing of healthy autologous T cells by CD229 CAR T cells in an overnight in vitro co-culture at an effector-target ratio of 1:1. Data indicate mean±S.D. from technical replicates (N=3). Statistical differences between conditions were determined by two-sided Student's t test. (FIG. 5H) Repeated killing of luciferase-expressing U266B1 cells in an in vitro overnight co-culture assay by CD229 CAR T cells after daily rechallenge with tumor cells. Data indicate mean±S.D. from technical replicates (N=3). (FIG. 5I) Membrane transfer from U266B1 cells to CD229 CAR T cells after 4-hour co-culture at an effector-target ratio of 4:1 as determined following Biotracker 555 staining of U266B1 cells using flow cytometry. Data indicate mean±S.D. from technical replicates (N=3). Statistical differences between conditions were determined by two-sided Student's t-test. (FIG. 5J) Schema of in vivo experiment to determine the efficacy of low affinity CD229 CAR T cells. (FIG. 5K) Bioluminescence of mice was determined using an in vivo imaging system (IVIS). Data indicate mean±S.D. from 6 animals per group. (FIG. 5L) Cumulative survival of NSG mice injected intravenously with 3×10⁶ U266B1 cells on day 0 and 5×10⁶ CD229 CAR T cells on day 7. Statistical significance was determined by log-rank test. (FIG. 5M) Overnight cytotoxicity assay to determine relative targeting of MM and healthy T cells by CD229 CAR T cells using flow cytometry. 5×10⁴ T cells and 5×10⁴ U266B1 cells were cocultured with 1×10\hat{ }5 CD229 CAR T cells and relative killing was determined by flow cytometry. Numbers indicate total cell numbers within the respective gates normalized using counting beads. (FIG. 5N) Schema of short-term in vivo experiment to determine targeting of healthy T cells by CD229 CAR T cells. (FIG. 5O) Numbers of CD3^{pos} S HA/CAR^{neg} healthy T cells as determined by flow cytometry per 50,000 events in spleens from mice injected with 5×10⁶ purified PBMCs and subsequently treated with CD229 CAR T cells. Data indicate mean S.D. from 3-5 individual animals. Statistical differences between conditions were determined by two-sided Student's t-test. (FIG. 5P) Surface expression of CD229 on healthy T cells after co-culture with indicated CAR T cells for 8-hour at an effector-target ratio of 5:1 as determined by flow cytometry.

FIG. 6 shows IFN-γ production by CD229 CAR T cells co-cultured with U266B1 cells. CAR T cells were co-cultured overnight with U266B1 cells at an effector-target ratio of 1:1. Supernatants were harvested and analyzed by IFN-γ ELISA. Data indicate mean±S.D. from technical replicates (N=3). Statistical significance was determined by two-tailed Student's t test.

FIG. 7 shows purity of biotinylated 2D3 variants. A fixed volume of each in vivo biotinylated antibodies was subjected to SDS-PAGE immediately after NiNTA purification and dialysis. Gels were stained with GelCode Blue (Thermo) and imaged using an iBright imaging system (Thermo).

FIG. 8 shows a correlation of variant binding by solid phase TRF assay and equilibrium constant. For the solid phase assay 2 ng/μl 2D3 variant scFvs were incubated with immobilized recombinant CD229 and binding was determined using anti-FLAG (clone: L5) and anti-mouse IgG-Eu (Perkin-Elmer). Equilibrium constants were determined by BLI. Significance of correlation was determined by Pearson r test and two-tailed p value.

FIG. 9 shows IFN-γ production by CD229 variant CAR T cells. CAR T cells were co-cultured overnight with U266B1 cells at an effector-target ratio of 1:1. Supernatants were harvested and analyzed by IFN-γ ELISA. Data indicate mean±S.D. from technical replicates (N=3). Statistical significance was determined by two-tailed Student's t test.

FIG. 10 shows purity of target T cells following negative selection. Healthy T cells were purified by negative selection (Stem Cell Technologies), stained with anti-CD3/PE or anti-CD229/PE antibodies, and purity determined by flow cytometry.

FIG. 11 shows CD229 expression on primary MM cells used in cytotoxicity assay. CD229 expression on the surface of primary human MM cells used for in vitro cytotoxicity assay as determined by flow cytometry.

FIG. 12 shows CAR surface expression levels before and after cell sorting. Following CAR T cell production, cells were stained with an anti-HA antibody to determine CAR surface expression levels and subsequently sorted by flow cytometry. CAR and GFP expression were determined in sorted and unsorted cell products following anti-HA staining by flow cytometry. Mean fluorescence intensity for anti-HA-PE of the shown population is indicated in each panel.

FIG. 13 shows the killing of MM cells by CD229 CAR T cells following normalization of CAR surface expression levels. Using a luminescence-based cytotoxicity assay, killing of luciferase-expressing U266B1 cells by CD229 CAR T cells before and after sorting for comparable HA/CAR expression was determined. Data indicate mean±S.D. from technical replicates (N=3). Statistical significance was determined by two-tailed Student's t test.

FIG. 14 shows CD229 loss from U266B1 cells after CD229 variant CAR T cell co-culture. U266B1 cells were labeled with CellTrace Far Red and incubated with CD229 variant CAR T cells for 4 hours. Co-cultures were stained with an anti-CD229/PE antibody and CD229 MFIs on CellTrace-positive U266B1 cells determined by flow cytometry. Data indicate mean S.D. from technical replicates (N=3). Statistical significance was determined by two-tailed Student's t test.

FIG. 15 shows cytokines secreted by CD229 CAR T cells during co-culture with MM cells. FH9Q- and 2D3-based CAR T cells were incubated with U266 cell over night at an effector-target ratio of 0.5:1 and supernatants subjected to Isoplexis CodePlex analysis. Data indicate mean from technical replicates (N=2).

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”, “an”, and “the” include plural reference unless the context clearly dictates otherwise. Thus, for example, reference to “a polypeptide” includes a plurality of such polypeptides, reference to “the composition*” is a reference to one or more compositions and equivalents thereof known to those skilled in the art, and so forth.

By “treat” is meant to administer a polypeptide, composition, nucleic acid, vector or cell of the invention to a subject, such as a human or other mammal (for example, an animal model), that has an increased susceptibility for developing a disease, disorder or infection in order to prevent or delay onset of the disease disorder or infection, prevent or delay a worsening of the effects of the disease, disorder or infection, or to partially or fully reverse the effects of the disease, disorder or infection. In some aspects, treat can mean to ameliorate a symptom of a disease, disorder or infection.

By “prevent” is meant to minimize the chance that a subject who has an increased susceptibility for developing a disease, disorder or infection will actually develop the disease, disorder or infection.

As used herein, the term “subject” or “patient” can be used interchangeably and refer to any organism to which a peptide or composition of the invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates, and the like.

As used herein, the terms “administering” and “administration” refer to any method of providing a disclosed polypeptide, composition, nucleic acid, vector or cell of the invention to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed exosome so as to treat a subject.

The terms “variant” and “mutant” are used interchangeably herein. As used herein, the term “mutant” refers to a modified nucleic acid or protein which displays the same characteristics when compared to a reference nucleic acid or protein sequence. A variant can be at least 65, 70, 75, 80, 85, 90, 95, or 99 percent homologous to a reference sequence. In some aspects, a reference sequence can be a fragment of CD229 antigen binding domain nucleic acid sequence or protein sequence (e.g. SEQ ID NO: 1). A “variant” can mean a difference in some way from the reference sequence other than just a simple deletion of an N- and/or C-terminal nucleotide. Variants can also or alternatively include at least one substitution and/or at least one addition; there may also be at least one deletion. Alternatively or in addition, variants can comprise modifications, such as non-natural residues at one or more positions with respect to a reference nucleic acid or protein.

Substitutions, deletions, insertions or any combination thereof may be used to arrive at a final derivative or variant. Generally, these changes are done on a few nucleotides to minimize the alteration of the molecule. However, larger changes may be tolerated in certain circumstances.

Generally, the amino acid or nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence (e.g. wild-type sequence) of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3 4 nucleotide base changes as compared to the parent or reference sequence of the invention, and shares or improves biological function, specificity and/or activity of the parent sequence. Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence (e.g. wild-type sequence).

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

The term “percent (%) homology” is used interchangeably herein with the term “percent (%) identity” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for anyone of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty ofl 1.0, and an extended gap penalty of 1.0, and utilize the BLOSUM-62matrix. (See, e.g., Altschul, S. F., et al., Nucleic Acids Res.25:3389-3402, 1997.) A preferred alignment of selected sequences in order to determine “% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Chimeric Antigen Receptor (CAR) Polypeptide

Disclosed are chimeric antigen receptor (CAR) polypeptides comprising a CD229 antigen binding domain, a transmembrane domain, and an intracellular signaling domain, wherein the CD229 binding domain is a variant CD229 antigen binding domain. In some aspects, a CD229 antigen binding domain can be any variant of the wild type CD229 antigen binding domain of the 2D3 clone described herein. For example, a CD229 antigen binding domain can be the amino acid sequence of SEQ ID NO:1 having one or more amino acid variations, wherein an amino acid variation can be a deletion, substitution, or modification.

The CD229 antigen binding domain, transmembrane domain, and intracellular signaling domain can be any of those described herein and any combination of those described herein.

In some instances, any of the disclosed CAR polypeptides can further comprise a tag sequence. In some instances, the tag sequence can be located between the variant CD229 antigen binding domain and the transmembrane domain or between the CD229 antigen binding domain and a hinge region. In some instances, the tag sequence can be a hemagglutinin tag, histidine tag, glutathione-S-transferase tag, or fluorescent tag. For example, the tag can be any sequence capable of aiding in the purification of the CAR polypeptide or capable of detecting the CAR polypeptide.

### 1. CD229 Antigen Binding Domain

In some aspects, the CD229 antigen binding domain is a variant of a wild type CD229 antigen binding domain or can be any of the CD229 antigen binding domains described herein. In some aspects, a variant CD229 antigen binding domain is a variant of the CD229 binding domain of the 2D3 clone described herein.

For example, in some aspects, the CD229 antigen binding domain is a variant of SEQ ID NO:1. Thus, a CD229 antigen binding domain can comprise the sequence of SEQ ID NO:1 having at least one or more amino acid substitutions. For example, the amino acid substitution can be present in CDR1, CDR2 or CDR3 of the heavy or light chain. In some aspects, a CD229 antigen binding domain can comprise the sequence of SEQ ID NO:134, SEQ ID NO:53, or SEQ ID NO:84.

In some instances, the CD229 antigen binding domain can be an antibody fragment or an antigen-binding fragment that specifically binds to CD229. In some instances, the CD229 antigen binding domain can be any recombinant or engineered protein domain capable of binding CD229.

In some instances, the CD229 antigen binding domain can be a Fab or a single-chain variable fragment (scFv) of an antibody that specifically binds CD229. In some instances, the scFv, comprising both the heavy chain variable region and the light chain variable region, can comprise the N-terminal region of the heavy chain variable region linked to the C-terminal region of the light chain variable region. In some instances, the scFv comprises the C-terminal region of the heavy chain variable region linked to the N-terminal region of the light chain variable region.

In some aspects, the CD229 antigen binding domain comprises one or more amino acid substitutions in the HCDR3 of SEQ ID NO: 1, which is the wild type 2D3 clone. For example, in some aspects, the CD229 antigen binding domain comprises a HCDR3 comprising the sequence of AKRDNSNSFDYW (SEQ ID NO:??), AKRGNENSFDYW (SEQ ID NO:**), or AKRGNSNSQDYW (SEQ ID NO: ##). In some aspects, the CD229 antigen binding domain comprises a HCDR3 comprising the sequence of AKRGNSDSFDYW.

In some aspects, the CD229 antigen binding domain comprises a heavy chain immunoglobulin variable region comprising a complementarity determining region 1 (CDR1) comprising the sequence of GFTFDDYA; a CDR2 comprising the sequence of ISWNSGSI; and a CDR3 comprising the sequence of AKRDNSNSFDYW, AKRGNENSFDYW, or AKRGNSNSQDYW.

In some aspects, the CD229 binding antigen can be any of those disclosed in Table 1. In some aspects, the CD229 binding antigen can the low affinity binding antigens as represented by GH4D, SH6E and FH9Q. In some aspects, the CD229 binding antigen can the high affinity binding antigens as represented by NH7D. In some aspects, there is a variation in the light chain of the CD229 binding antigen. In some aspects, there is a variation in the heavy chain of the CD229 binding antigen.

TABLE 1
Examples of CD229 antigen binding domains. Variable heavy chain, linker
(underlined), and variable light chain (bold). GH4D, FH9Q, and SH6E are low affinity CD229
antigen binding domains. NH7D is a high affinity CD229 antigen binding domain.

| Old | Pos | New | NAME | Full sequence | SEQ ID NO: |
|---|---|---|---|---|---|
| — | — | — | WT | QVQLVESGGGLVQPGRSLRLSCAASGFTFDDY | 1 |
| | | | (2D3) | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | C | AH1C | QVQLVESGGGLVQPGRSLRLSCAASGFTFDDY | 2 |
| | | | | AMHWVRQAPGKGLEWVSGISWNSGSIGYADS | |
| | | | | AKGRFTISRDNSKNTLYLQMNSLRAEDTAVYY | |
| | | | | CCKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | D | AH1D | QVQLVESGGGLVQPGRSLRLSCAASGFTFDDY | 3 |
| | | | | AMHWVRQAPGKGLEWVSGISWNSGSIGYADS | |
| | | | | AKGRFTISRDNSKNTLYLQMNSLRAEDTAVYY | |
| | | | | CDKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | E | AH1E | QVQLVESGGGLVQPGRSLRLSCAASGFTFDDY | 4 |
| | | | | AMHWVRQAPGKGLEWVSGISWNSGSIGYADS | |
| | | | | AKGRFTISRDNSKNTLYLQMNSLRAEDTAVYY | |
| | | | | CEKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | F | AH1F | QVQLVESGGGLVQPGRSLRLSCAASGFTFDDY | 5 |
| | | | | AMHWVRQAPGKGLEWVSGISWNSGSIGYADS | |
| | | | | AKGRFTISRDNSKNTLYLQMNSLRAEDTAVYY | |
| | | | | CFKRGNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| A | H1 | G | AH1G | QVQLVESGGGLVQPGRSLRLSCAASGFTFDDY | 6 |
| | | | | AMHWVRQAPGKGLEWVSGISWNSGSIGYADS | |
| | | | | AKGRFTISRDNSKNTLYLQMNSLRAEDTAVYY | |
| | | | | CGKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | H | AH1H | QVQLVESGGGLVQPGRSLRLSCAASGFTFDDY | 7 |
| | | | | AMHWVRQAPGKGLEWVSGISWNSGSIGYADS | |
| | | | | AKGRFTISRDNSKNTLYLQMNSLRAEDTAVYY | |
| | | | | CHKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | I | AH1I | QVQLVESGGGLVQPGRSLRLSCAASGFTFDDY | 8 |
| | | | | AMHWVRQAPGKGLEWVSGISWNSGSIGYADS | |
| | | | | AKGRFTISRDNSKNTLYLQMNSLRAEDTAVYY | |
| | | | | CIKRGNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| A | H1 | K | AH1K | QVQLVESGGGLVQPGRSLRLSCAASGFTFDDY | 9 |
| | | | | AMHWVRQAPGKGLEWVSGISWNSGSIGYADS | |
| | | | | AKGRFTISRDNSKNTLYLQMNSLRAEDTAVYY | |
| | | | | CKKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | L | AH1L | QVQLVESGGGLVQPGRSLRLSCAASGFTFDDY | 10 |
| | | | | AMHWVRQAPGKGLEWVSGISWNSGSIGYADS | |
| | | | | AKGRFTISRDNSKNTLYLQMNSLRAEDTAVYY | |
| | | | | CLKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | M | AH1M | QVQLVESGGGLVQPGRSLRLSCAASGFTFDDY | 11 |
| | | | | AMHWVRQAPGKGLEWVSGISWNSGSIGYADS | |
| | | | | AKGRFTISRDNSKNTLYLQMNSLRAEDTAVYY | |
| | | | | CMKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | N | AH1N | QVQLVESGGGLVQPGRSLRLSCAASGFTFDDY | 12 |
| | | | | AMHWVRQAPGKGLEWVSGISWNSGSIGYADS | |
| | | | | AKGRFTISRDNSKNTLYLQMNSLRAEDTAVYY | |
| | | | | CNKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | P | AH1P | QVQLVESGGGLVQPGRSLRLSCAASGFTFDDY | 13 |
| | | | | AMHWVRQAPGKGLEWVSGISWNSGSIGYADS | |
| | | | | AKGRFTISRDNSKNTLYLQMNSLRAEDTAVYY | |
| | | | | CPKRGNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| A | H1 | S | AH1S | QVQLVESGGGLVQPGRSLRLSCAASGFTFDDY | 14 |
| | | | | AMHWVRQAPGKGLEWVSGISWNSGSIGYADS | |
| | | | | AKGRFTISRDNSKNTLYLQMNSLRAEDTAVYY | |
| | | | | CSKRGNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| A | H1 | T | AH1T | QVQLVESGGGLVQPGRSLRLSCAASGFTFDDY | 15 |
| | | | | AMHWVRQAPGKGLEWVSGISWNSGSIGYADS | |
| | | | | AKGRFTISRDNSKNTLYLQMNSLRAEDTAVYY | |
| | | | | CTKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | V | AH1V | QVQLVESGGGLVQPGRSLRLSCAASGFTFDDY | 16 |
| | | | | AMHWVRQAPGKGLEWVSGISWNSGSIGYADS | |
| | | | | AKGRFTISRDNSKNTLYLQMNSLRAEDTAVYY | |
| | | | | CVKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | W | AH1W | QVQLVESGGGLVQPGRSLRLSCAASGFTFDDY | 17 |
| | | | | AMHWVRQAPGKGLEWVSGISWNSGSIGYADS | |
| | | | | AKGRFTISRDNSKNTLYLQMNSLRAEDTAVYY | |
| | | | | CWKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| A | H1 | Y | AH1Y | QVQLVESGGGLVQPGRSLRLSCAASGFTFDDY | 18 |
| | | | | AMHWVRQAPGKGLEWVSGISWNSGSIGYADS | |
| | | | | AKGRFTISRDNSKNTLYLQMNSLRAEDTAVYY | |
| | | | | CYKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | C | KH2C | QVQLVESGGGLVQPGRSLRLSCAASGFTFDDY | 19 |
| | | | | AMHWVRQAPGKGLEWVSGISWNSGSIGYADS | |
| | | | | AKGRFTISRDNSKNTLYLQMNSLRAEDTAVYY | |
| | | | | CACRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | D | KH2D | QVQLVESGGGLVQPGRSLRLSCAASGFTFDDY | 20 |
| | | | | AMHWVRQAPGKGLEWVSGISWNSGSIGYADS | |
| | | | | AKGRFTISRDNSKNTLYLQMNSLRAEDTAVYY | |
| | | | | CADRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | E | KH2E | QVQLVESGGGLVQPGRSLRLSCAASGFTFDDY | 21 |
| | | | | AMHWVRQAPGKGLEWVSGISWNSGSIGYADS | |
| | | | | AKGRFTISRDNSKNTLYLQMNSLRAEDTAVYY | |
| | | | | CAERGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | F | KH2F | QVQLVESGGGLVQPGRSLRLSCAASGFTFDDY | 22 |
| | | | | AMHWVRQAPGKGLEWVSGISWNSGSIGYADS | |
| | | | | AKGRFTISRDNSKNTLYLQMNSLRAEDTAVYY | |
| | | | | CAFRGNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| K | H2 | G | KH2G | QVQLVESGGGLVQPGRSLRLSCAASGFTFDDY | 23 |
| | | | | AMHWVRQAPGKGLEWVSGISWNSGSIGYADS | |
| | | | | AKGRFTISRDNSKNTLYLQMNSLRAEDTAVYY | |
| | | | | CAGRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | H | KH2H | QVQLVESGGGLVQPGRSLRLSCAASGFTFDDY | 24 |
| | | | | AMHWVRQAPGKGLEWVSGISWNSGSIGYADS | |
| | | | | AKGRFTISRDNSKNTLYLQMNSLRAEDTAVYY | |
| | | | | CAHRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | I | KH2I | QVQLVESGGGLVQPGRSLRLSCAASGFTFDDY | 25 |
| | | | | AMHWVRQAPGKGLEWVSGISWNSGSIGYADS | |
| | | | | AKGRFTISRDNSKNTLYLQMNSLRAEDTAVYY | |
| | | | | CAIRGNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| K | H2 | L | KH2L | QVQLVESGGGLVQPGRSLRLSCAASGFTFDDY | 26 |
| | | | | AMHWVRQAPGKGLEWVSGISWNSGSIGYADS | |
| | | | | AKGRFTISRDNSKNTLYLQMNSLRAEDTAVYY | |
| | | | | CALRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | M | KH2M | QVQLVESGGGLVQPGRSLRLSCAASGFTFDDY | 27 |
| | | | | AMHWVRQAPGKGLEWVSGISWNSGSIGYADS | |
| | | | | AKGRFTISRDNSKNTLYLQMNSLRAEDTAVYY | |
| | | | | CAMRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | N | KH2N | QVQLVESGGGLVQPGRSLRLSCAASGFTFDDY | 28 |
| | | | | AMHWVRQAPGKGLEWVSGISWNSGSIGYADS | |
| | | | | AKGRFTISRDNSKNTLYLQMNSLRAEDTAVYY | |
| | | | | CANRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | P | KH2P | QVQLVESGGGLVQPGRSLRLSCAASGFTFDDY | 29 |
| | | | | AMHWVRQAPGKGLEWVSGISWNSGSIGYADS | |
| | | | | AKGRFTISRDNSKNTLYLQMNSLRAEDTAVYY | |
| | | | | CAPRGNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| K | H2 | Q | KH2Q | QVQLVESGGGLVQPGRSLRLSCAASGFTFDDY | 30 |
| | | | | AMHWVRQAPGKGLEWVSGISWNSGSIGYADS | |
| | | | | AKGRFTISRDNSKNTLYLQMNSLRAEDTAVYY | |
| | | | | CAQRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | R | KH2R | QVQLVESGGGLVQPGRSLRLSCAASGFTFDDY | 31 |
| | | | | AMHWVRQAPGKGLEWVSGISWNSGSIGYADS | |
| | | | | AKGRFTISRDNSKNTLYLQMNSLRAEDTAVYY | |
| | | | | CARRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | S | KH2S | QVQLVESGGGLVQPGRSLRLSCAASGFTFDDY | 32 |
| | | | | AMHWVRQAPGKGLEWVSGISWNSGSIGYADS | |
| | | | | AKGRFTISRDNSKNTLYLQMNSLRAEDTAVYY | |
| | | | | CASRGNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| K | H2 | V | KH2V | QVQLVESGGGLVQPGRSLRLSCAASGFTFDDY | 33 |
| | | | | AMHWVRQAPGKGLEWVSGISWNSGSIGYADS | |
| | | | | AKGRFTISRDNSKNTLYLQMNSLRAEDTAVYY | |
| | | | | CAVRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| K | H2 | W | KH2W | QVQLVESGGGLVQPGRSLRLSCAASGFTFDDY | 34 |
| | | | | AMHWVRQAPGKGLEWVSGISWNSGSIGYADS | |
| | | | | AKGRFTISRDNSKNTLYLQMNSLRAEDTAVYY | |
| | | | | CAWRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | A | RH3A | QVQLVESGGGLVQPGRSLRLSCAASGFTFDDY | 35 |
| | | | | AMHWVRQAPGKGLEWVSGISWNSGSIGYADS | |
| | | | | AKGRFTISRDNSKNTLYLQMNSLRAEDTAVYY | |
| | | | | CAKAGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | C | RH3C | QVQLVESGGGLVQPGRSLRLSCAASGFTFDDY | 36 |
| | | | | AMHWVRQAPGKGLEWVSGISWNSGSIGYADS | |
| | | | | AKGRFTISRDNSKNTLYLQMNSLRAEDTAVYY | |
| | | | | CAKCGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | G | RH3G | QVQLVESGGGLVQPGRSLRLSCAASGFTFDDY | 37 |
| | | | | AMHWVRQAPGKGLEWVSGISWNSGSIGYADS | |
| | | | | AKGRFTISRDNSKNTLYLQMNSLRAEDTAVYY | |
| | | | | CAKGGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | H | RH3H | QVQLVESGGGLVQPGRSLRLSCAASGFTFDDY | 38 |
| | | | | AMHWVRQAPGKGLEWVSGISWNSGSIGYADS | |
| | | | | AKGRFTISRDNSKNTLYLQMNSLRAEDTAVYY | |
| | | | | CAKHGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | I | RH3I | QVQLVESGGGLVQPGRSLRLSCAASGFTFDDY | 39 |
| | | | | AMHWVRQAPGKGLEWVSGISWNSGSIGYADS | |
| | | | | AKGRFTISRDNSKNTLYLQMNSLRAEDTAVYY | |
| | | | | CAKIGNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| R | H3 | K | RH3K | QVQLVESGGGLVQPGRSLRLSCAASGFTFDDY | 40 |
| | | | | AMHWVRQAPGKGLEWVSGISWNSGSIGYADS | |
| | | | | AKGRFTISRDNSKNTLYLQMNSLRAEDTAVYY | |
| | | | | CAKKGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | L | RH3L | QVQLVESGGGLVQPGRSLRLSCAASGFTFDDY | 41 |
| | | | | AMHWVRQAPGKGLEWVSGISWNSGSIGYADS | |
| | | | | AKGRFTISRDNSKNTLYLQMNSLRAEDTAVYY | |
| | | | | CAKLGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | M | RH3M | QVQLVESGGGLVQPGRSLRLSCAASGFTFDDY | 42 |
| | | | | AMHWVRQAPGKGLEWVSGISWNSGSIGYADS | |
| | | | | AKGRFTISRDNSKNTLYLQMNSLRAEDTAVYY | |
| | | | | CAKMGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | N | RH3N | QVQLVESGGGLVQPGRSLRLSCAASGFTFDDY | 43 |
| | | | | AMHWVRQAPGKGLEWVSGISWNSGSIGYADS | |
| | | | | AKGRFTISRDNSKNTLYLQMNSLRAEDTAVYY | |
| | | | | CAKNGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | P | RH3P | QVQLVESGGGLVQPGRSLRLSCAASGFTFDDY | 44 |
| | | | | AMHWVRQAPGKGLEWVSGISWNSGSIGYADS | |
| | | | | AKGRFTISRDNSKNTLYLQMNSLRAEDTAVYY | |
| | | | | CAKPGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | Q | RH3Q | QVQLVESGGGLVQPGRSLRLSCAASGFTFDDY | 45 |
| | | | | AMHWVRQAPGKGLEWVSGISWNSGSIGYADS | |
| | | | | AKGRFTISRDNSKNTLYLQMNSLRAEDTAVYY | |
| | | | | CAKQGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | S | RH3S | QVQLVESGGGLVQPGRSLRLSCAASGFTFDDY | 46 |
| | | | | AMHWVRQAPGKGLEWVSGISWNSGSIGYADS | |
| | | | | AKGRFTISRDNSKNTLYLQMNSLRAEDTAVYY | |
| | | | | CAKSGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | T | RH3T | QVQLVESGGGLVQPGRSLRLSCAASGFTFDDY | 47 |
| | | | | AMHWVRQAPGKGLEWVSGISWNSGSIGYADS | |
| | | | | AKGRFTISRDNSKNTLYLQMNSLRAEDTAVYY | |
| | | | | CAKTGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | V | RH3V | QVQLVESGGGLVQPGRSLRLSCAASGFTFDDY | 48 |
| | | | | AMHWVRQAPGKGLEWVSGISWNSGSIGYADS | |
| | | | | AKGRFTISRDNSKNTLYLQMNSLRAEDTAVYY | |
| | | | | CAKVGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | W | RH3W | QVQLVESGGGLVQPGRSLRLSCAASGFTFDDY | 49 |
| | | | | AMHWVRQAPGKGLEWVSGISWNSGSIGYADS | |
| | | | | AKGRFTISRDNSKNTLYLQMNSLRAEDTAVYY | |
| | | | | CAKWGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| R | H3 | Y | RH3Y | QVQLVESGGGLVQPGRSLRLSCAASGFTFDDY | 50 |
| | | | | AMHWVRQAPGKGLEWVSGISWNSGSIGYADS | |
| | | | | AKGRFTISRDNSKNTLYLQMNSLRAEDTAVYY | |
| | | | | CAKYGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | A | GH4A | QVQLVESGGGLVQPGRSLRLSCAASGFTFDDY | 51 |
| | | | | AMHWVRQAPGKGLEWVSGISWNSGSIGYADS | |
| | | | | AKGRFTISRDNSKNTLYLQMNSLRAEDTAVYY | |
| | | | | CAKRANSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | C | GH4C | QVQLVESGGGLVQPGRSLRLSCAASGFTFDDY | 52 |
| | | | | AMHWVRQAPGKGLEWVSGISWNSGSIGYADS | |
| | | | | AKGRFTISRDNSKNTLYLQMNSLRAEDTAVYY | |
| | | | | CAKRCNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | D | GH4D | QVQLVESGGGLVQPGRSLRLSCAASGFTFDDY | 53 |
| | | | | AMHWVRQAPGKGLEWVSGISWNSGSIGYADS | |
| | | | | AKGRFTISRDNSKNTLYLQMNSLRAEDTAVYY | |
| | | | | CAKRDNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | E | GH4E | QVQLVESGGGLVQPGRSLRLSCAASGFTFDDY | 54 |
| | | | | AMHWVRQAPGKGLEWVSGISWNSGSIGYADS | |
| | | | | AKGRFTISRDNSKNTLYLQMNSLRAEDTAVYY | |
| | | | | CAKRENSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | F | GH4F | QVQLVESGGGLVQPGRSLRLSCAASGFTFDDY | 55 |
| | | | | AMHWVRQAPGKGLEWVSGISWNSGSIGYADS | |
| | | | | AKGRFTISRDNSKNTLYLQMNSLRAEDTAVYY | |
| | | | | CAKRFNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| G | H4 | H | GH4H | QVQLVESGGGLVQPGRSLRLSCAASGFTFDDY | 56 |
| | | | | AMHWVRQAPGKGLEWVSGISWNSGSIGYADS | |
| | | | | AKGRFTISRDNSKNTLYLQMNSLRAEDTAVYY | |
| | | | | CAKRHNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | I | GH4I | QVQLVESGGGLVQPGRSLRLSCAASGFTFDDY | 57 |
| | | | | AMHWVRQAPGKGLEWVSGISWNSGSIGYADS | |
| | | | | AKGRFTISRDNSKNTLYLQMNSLRAEDTAVYY | |
| | | | | CAKRINSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| G | H4 | K | GH4K | QVQLVESGGGLVQPGRSLRLSCAASGFTFDDY | 58 |
| | | | | AMHWVRQAPGKGLEWVSGISWNSGSIGYADS | |
| | | | | AKGRFTISRDNSKNTLYLQMNSLRAEDTAVYY | |
| | | | | CAKRKNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | L | GH4L | QVQLVESGGGLVQPGRSLRLSCAASGFTFDDY | 59 |
| | | | | AMHWVRQAPGKGLEWVSGISWNSGSIGYADS | |
| | | | | AKGRFTISRDNSKNTLYLQMNSLRAEDTAVYY | |
| | | | | CAKRLNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | M | GH4M | QVQLVESGGGLVQPGRSLRLSCAASGFTFDDY | 60 |
| | | | | AMHWVRQAPGKGLEWVSGISWNSGSIGYADS | |
| | | | | AKGRFTISRDNSKNTLYLQMNSLRAEDTAVYY | |
| | | | | CAKRMNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | N | GH4N | QVQLVESGGGLVQPGRSLRLSCAASGFTFDDY | 61 |
| | | | | AMHWVRQAPGKGLEWVSGISWNSGSIGYADS | |
| | | | | AKGRFTISRDNSKNTLYLQMNSLRAEDTAVYY | |
| | | | | CAKRNNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | P | GH4P | QVQLVESGGGLVQPGRSLRLSCAASGFTFDDY | 62 |
| | | | | AMHWVRQAPGKGLEWVSGISWNSGSIGYADS | |
| | | | | AKGRFTISRDNSKNTLYLQMNSLRAEDTAVYY | |
| | | | | CAKRPNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| G | H4 | Q | GH4Q | QVQLVESGGGLVQPGRSLRLSCAASGFTFDDY | 63 |
| | | | | AMHWVRQAPGKGLEWVSGISWNSGSIGYADS | |
| | | | | AKGRFTISRDNSKNTLYLQMNSLRAEDTAVYY | |
| | | | | CAKRQNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | R | GH4R | QVQLVESGGGLVQPGRSLRLSCAASGFTFDDY | 64 |
| | | | | AMHWVRQAPGKGLEWVSGISWNSGSIGYADS | |
| | | | | AKGRFTISRDNSKNTLYLQMNSLRAEDTAVYY | |
| | | | | CAKRRNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | S | GH4S | QVQLVESGGGLVQPGRSLRLSCAASGFTFDDY | 65 |
| | | | | AMHWVRQAPGKGLEWVSGISWNSGSIGYADS | |
| | | | | AKGRFTISRDNSKNTLYLQMNSLRAEDTAVYY | |
| | | | | CAKRSNSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| G | H4 | T | GH4T | QVQLVESGGGLVQPGRSLRLSCAASGFTFDDY | 66 |
| | | | | AMHWVRQAPGKGLEWVSGISWNSGSIGYADS | |
| | | | | AKGRFTISRDNSKNTLYLQMNSLRAEDTAVYY | |
| | | | | CAKRTNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | V | GH4V | QVQLVESGGGLVQPGRSLRLSCAASGFTFDDY | 67 |
| | | | | AMHWVRQAPGKGLEWVSGISWNSGSIGYADS | |
| | | | | AKGRFTISRDNSKNTLYLQMNSLRAEDTAVYY | |
| | | | | CAKRVNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| G | H4 | Y | GH4Y | QVQLVESGGGLVQPGRSLRLSCAASGFTFDDY | 68 |
| | | | | AMHWVRQAPGKGLEWVSGISWNSGSIGYADS | |
| | | | | AKGRFTISRDNSKNTLYLQMNSLRAEDTAVYY | |
| | | | | CAKRYNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H5 | A | NH5A | QVQLVESGGGLVQPGRSLRLSCAASGFTFDDY | 69 |
| | | | | AMHWVRQAPGKGLEWVSGISWNSGSIGYADS | |
| | | | | AKGRFTISRDNSKNTLYLQMNSLRAEDTAVYY | |
| | | | | CAKRGASNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H5 | C | NH5C | QVQLVESGGGLVQPGRSLRLSCAASGFTFDDY | 70 |
| | | | | AMHWVRQAPGKGLEWVSGISWNSGSIGYADS | |
| | | | | AKGRFTISRDNSKNTLYLQMNSLRAEDTAVYY | |
| | | | | CAKRGCSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H5 | D | NH5D | QVQLVESGGGLVQPGRSLRLSCAASGFTFDDY | 71 |
| | | | | AMHWVRQAPGKGLEWVSGISWNSGSIGYADS | |
| | | | | AKGRFTISRDNSKNTLYLQMNSLRAEDTAVYY | |
| | | | | CAKRGDSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H5 | E | NH5E | QVQLVESGGGLVQPGRSLRLSCAASGFTFDDY | 72 |
| | | | | AMHWVRQAPGKGLEWVSGISWNSGSIGYADS | |
| | | | | AKGRFTISRDNSKNTLYLQMNSLRAEDTAVYY | |
| | | | | CAKRGESNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H5 | H | NH5H | QVQLVESGGGLVQPGRSLRLSCAASGFTFDDY | 73 |
| | | | | AMHWVRQAPGKGLEWVSGISWNSGSIGYADS | |
| | | | | AKGRFTISRDNSKNTLYLQMNSLRAEDTAVYY | |
| | | | | CAKRGHSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H5 | I | NH5I | QVQLVESGGGLVQPGRSLRLSCAASGFTFDDY | 74 |
| | | | | AMHWVRQAPGKGLEWVSGISWNSGSIGYADS | |
| | | | | AKGRFTISRDNSKNTLYLQMNSLRAEDTAVYY | |
| | | | | CAKRGISNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| N | H5 | L | NH5L | QVQLVESGGGLVQPGRSLRLSCAASGFTFDDY | 75 |
| | | | | AMHWVRQAPGKGLEWVSGISWNSGSIGYADS | |
| | | | | AKGRFTISRDNSKNTLYLQMNSLRAEDTAVYY | |
| | | | | CAKRGLSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H5 | M | NH5M | QVQLVESGGGLVQPGRSLRLSCAASGFTFDDY | 76 |
| | | | | AMHWVRQAPGKGLEWVSGISWNSGSIGYADS | |
| | | | | AKGRFTISRDNSKNTLYLQMNSLRAEDTAVYY | |
| | | | | CAKRGMSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H5 | P | NH5P | QVQLVESGGGLVQPGRSLRLSCAASGFTFDDY | 77 |
| | | | | AMHWVRQAPGKGLEWVSGISWNSGSIGYADS | |
| | | | | AKGRFTISRDNSKNTLYLQMNSLRAEDTAVYY | |
| | | | | CAKRGPSNSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| N | H5 | T | NH5T | QVQLVESGGGLVQPGRSLRLSCAASGFTFDDY | 78 |
| | | | | AMHWVRQAPGKGLEWVSGISWNSGSIGYADS | |
| | | | | AKGRFTISRDNSKNTLYLQMNSLRAEDTAVYY | |
| | | | | CAKRGTSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H5 | V | NH5V | QVQLVESGGGLVQPGRSLRLSCAASGFTFDDY | 79 |
| | | | | AMHWVRQAPGKGLEWVSGISWNSGSIGYADS | |
| | | | | AKGRFTISRDNSKNTLYLQMNSLRAEDTAVYY | |
| | | | | CAKRGVSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H5 | W | NH5W | QVQLVESGGGLVQPGRSLRLSCAASGFTFDDY | 80 |
| | | | | AMHWVRQAPGKGLEWVSGISWNSGSIGYADS | |
| | | | | AKGRFTISRDNSKNTLYLQMNSLRAEDTAVYY | |
| | | | | CAKRGWSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | A | SH6A | QVQLVESGGGLVQPGRSLRLSCAASGFTFDDY | 81 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNANSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | C | SH6C | QVQLVESGGGLVQPGRSLRLSCAASGFTFDDY | 82 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNCNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | D | SH6D | QVQLVESGGGLVQPGRSLRLSCAASGFTFDDY | 83 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNDNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | E | SH6E | QVQLVESGGGLVQPGRSLRLSCAASGFTFDDY | 84 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNENSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | F | SH6F | QVQLVESGGGLVQPGRSLRLSCAASGFTFDDY | 85 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNFNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | H | SH6H | QVQLVESGGGLVQPGRSLRLSCAASGFTFDDY | 86 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNHNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | K | SH6K | QVQLVESGGGLVQPGRSLRLSCAASGFTFDDY | 87 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNKNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | L | SH6L | QVQLVESGGGLVQPGRSLRLSCAASGFTFDDY | 88 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNLNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | N | SH6N | QVQLVESGGGLVQPGRSLRLSCAASGFTFDDY | 89 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNNNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | P | SH6P | QVQLVESGGGLVQPGRSLRLSCAASGFTFDDY | 90 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNPNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | Q | SH6Q | QVQLVESGGGLVQPGRSLRLSCAASGFTFDDY | 91 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNQNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | T | SH6T | QVQLVESGGGLVQPGRSLRLSCAASGFTFDDY | 92 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNTNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | W | SH6W | QVQLVESGGGLVQPGRSLRLSCAASGFTFDDY | 93 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNWNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H6 | Y | SH6Y | QVQLVESGGGLVQPGRSLRLSCAASGFTFDDY | 94 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNYNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | A | NH7A | QVQLVESGGGLVQPGRSLRLSCAASGFTFDDY | 95 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSASFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | C | NH7C | QVQLVESGGGLVQPGRSLRLSCAASGFTFDDY | 96 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSCSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | D | NH7D | QVQLVESGGGLVQPGRSLRLSCAASGFTFDDY | 97 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSDSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | E | NH7E | QVQLVESGGGLVQPGRSLRLSCAASGFTFDDY | 98 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSESFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | F | NH7F | QVQLVESGGGLVQPGRSLRLSCAASGFTFDDY | 99 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSFSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| N | H7 | I | NH7I | QVQLVESGGGLVQPGRSLRLSCAASGFTFDDY | 100 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSISFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| N | H7 | K | NH7K | QVQLVESGGGLVQPGRSLRLSCAASGFTFDDY | 101 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSKSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | L | NH7L | QVQLVESGGGLVQPGRSLRLSCAASGFTFDDY | 102 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSLSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | M | NH7M | QVQLVESGGGLVQPGRSLRLSCAASGFTFDDY | 103 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSMSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | Q | NH7Q | QVQLVESGGGLVQPGRSLRLSCAASGFTFDDY | 104 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSQSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | R | NH7R | QVQLVESGGGLVQPGRSLRLSCAASGFTFDDY | 105 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSRSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | S | NH7S | QVQLVESGGGLVQPGRSLRLSCAASGFTFDDY | 106 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSSSFDYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| N | H7 | T | NH7T | QVQLVESGGGLVQPGRSLRLSCAASGFTFDDY | 107 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSTSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | W | NH7W | QVQLVESGGGLVQPGRSLRLSCAASGFTFDDY | 108 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSWSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| N | H7 | Y | NH7Y | QVQLVESGGGLVQPGRSLRLSCAASGFTFDDY | 109 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSYSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | A | SH8A | QVQLVESGGGLVQPGRSLRLSCAASGFTFDDY | 110 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNAFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | C | SH8C | QVQLVESGGGLVQPGRSLRLSCAASGFTFDDY | 111 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNCFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | D | SH8D | QVQLVESGGGLVQPGRSLRLSCAASGFTFDDY | 112 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNDFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | E | SH8E | QVQLVESGGGLVQPGRSLRLSCAASGFTFDDY | 113 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNEFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | F | SH8F | QVQLVESGGGLVQPGRSLRLSCAASGFTFDDY | 114 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNFFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | G | SH8G | QVQLVESGGGLVQPGRSLRLSCAASGFTFDDY | 115 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNGFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | H | SH8H | QVQLVESGGGLVQPGRSLRLSCAASGFTFDDY | 116 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNHFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | K | SH8K | QVQLVESGGGLVQPGRSLRLSCAASGFTFDDY | 117 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNKFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | N | SH8N | QVQLVESGGGLVQPGRSLRLSCAASGFTFDDY | 118 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNNFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | Q | SH8Q | QVQLVESGGGLVQPGRSLRLSCAASGFTFDDY | 119 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNQFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | R | SH8R | QVQLVESGGGLVQPGRSLRLSCAASGFTFDDY | 120 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNRFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | T | SH8T | QVQLVESGGGLVQPGRSLRLSCAASGFTFDDY | 121 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNTFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | V | SH8V | QVQLVESGGGLVQPGRSLRLSCAASGFTFDDY | 122 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNVFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| S | H8 | W | SH8W | QVQLVESGGGLVQPGRSLRLSCAASGFTFDDY | 123 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNWFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | A | FH9A | QVQLVESGGGLVQPGRSLRLSCAASGFTFDDY | 124 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSADYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | D | FH9D | QVQLVESGGGLVQPGRSLRLSCAASGFTFDDY | 125 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSDDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | E | FH9E | QVQLVESGGGLVQPGRSLRLSCAASGFTFDDY | 126 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSEDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | G | FH9G | QVQLVESGGGLVQPGRSLRLSCAASGFTFDDY | 127 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSGDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | H | FH9H | QVQLVESGGGLVQPGRSLRLSCAASGFTFDDY | 128 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSHDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | K | FH9K | QVQLVESGGGLVQPGRSLRLSCAASGFTFDDY | 129 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSKDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | L | FH9L | QVQLVESGGGLVQPGRSLRLSCAASGFTFDDY | 130 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSLDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | M | FH9M | QVQLVESGGGLVQPGRSLRLSCAASGFTFDDY | 131 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSMDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | N | FH9N | QVQLVESGGGLVQPGRSLRLSCAASGFTFDDY | 132 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSNDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | P | FH9P | QVQLVESGGGLVQPGRSLRLSCAASGFTFDDY | 133 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSPDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | Q | FH9Q | QVQLVESGGGLVQPGRSLRLSCAASGFTFDDY | 134 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSQDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | T | FH9T | QVQLVESGGGLVQPGRSLRLSCAASGFTFDDY | 135 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSTDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | W | FH9W | QVQLVESGGGLVQPGRSLRLSCAASGFTFDDY | 136 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSWDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| F | H9 | Y | FH9Y | QVQLVESGGGLVQPGRSLRLSCAASGFTFDDY | 137 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSYDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | A | DH10A | QVQLVESGGGLVQPGRSLRLSCAASGFTFDDY | 138 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFAYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | C | DH10C | QVQLVESGGGLVQPGRSLRLSCAASGFTFDDY | 139 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFCYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | E | DH10E | QVQLVESGGGLVQPGRSLRLSCAASGFTFDDY | 140 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFEYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | F | DH1OF | QVQLVESGGGLVQPGRSLRLSCAASGFTFDDY | 141 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFFYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| D | H10 | G | DH10G | QVQLVESGGGLVQPGRSLRLSCAASGFTFDDY | 142 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFGYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | K | DH10K | QVQLVESGGGLVQPGRSLRLSCAASGFTFDDY | 143 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFKYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | L | DH10L | QVQLVESGGGLVQPGRSLRLSCAASGFTFDDY | 144 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFLYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | M | DH10M | QVQLVESGGGLVQPGRSLRLSCAASGFTFDDY | 145 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFMYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | N | DH10N | QVQLVESGGGLVQPGRSLRLSCAASGFTFDDY | 146 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFNYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | P | DH10P | QVQLVESGGGLVQPGRSLRLSCAASGFTFDDY | 147 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFPYWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| D | H10 | Q | DH10Q | QVQLVESGGGLVQPGRSLRLSCAASGFTFDDY | 148 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFQYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | R | DH10R | QVQLVESGGGLVQPGRSLRLSCAASGFTFDDY | 149 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFRYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | T | DH10T | QVQLVESGGGLVQPGRSLRLSCAASGFTFDDY | 150 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFTYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | V | DH10V | QVQLVESGGGLVQPGRSLRLSCAASGFTFDDY | 151 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFVYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| D | H10 | Y | DH10Y | QVQLVESGGGLVQPGRSLRLSCAASGFTFDDY | 152 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFYYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | A | YH11A | QVQLVESGGGLVQPGRSLRLSCAASGFTFDDY | 153 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDAWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | C | YH11C | QVQLVESGGGLVQPGRSLRLSCAASGFTFDDY | 154 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDCWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | D | YH11D | QVQLVESGGGLVQPGRSLRLSCAASGFTFDDY | 155 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDDWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | E | YH11E | QVQLVESGGGLVQPGRSLRLSCAASGFTFDDY | 156 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDEWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | F | YH11F | QVQLVESGGGLVQPGRSLRLSCAASGFTFDDY | 157 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDFWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| Y | H11 | G | YH11G | QVQLVESGGGLVQPGRSLRLSCAASGFTFDDY | 158 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDGWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | K | YH11K | QVQLVESGGGLVQPGRSLRLSCAASGFTFDDY | 159 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDKWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | L | YH11L | QVQLVESGGGLVQPGRSLRLSCAASGFTFDDY | 160 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDLWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | M | YH11M | QVQLVESGGGLVQPGRSLRLSCAASGFTFDDY | 161 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDMWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | Q | YH11Q | QVQLVESGGGLVQPGRSLRLSCAASGFTFDDY | 162 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDQWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | R | YH11R | QVQLVESGGGLVQPGRSLRLSCAASGFTFDDY | 163 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDRWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | S | YH11S | QVQLVESGGGLVQPGRSLRLSCAASGFTFDDY | 164 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDSWGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| Y | H11 | T | YH11T | QVQLVESGGGLVQPGRSLRLSCAASGFTFDDY | 165 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDTWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | V | YH11V | QVQLVESGGGLVQPGRSLRLSCAASGFTFDDY | 166 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDVWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| Y | H11 | W | YH11W | QVQLVESGGGLVQPGRSLRLSCAASGFTFDDY | 167 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDWWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| W | H12 | A | WH12A | QVQLVESGGGLVQPGRSLRLSCAASGFTFDDY | 168 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYAGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | C | WH12C | QVQLVESGGGLVQPGRSLRLSCAASGFTFDDY | 169 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYCGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | E | WH12E | QVQLVESGGGLVQPGRSLRLSCAASGFTFDDY | 170 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYEGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | F | WH12F | QVQLVESGGGLVQPGRSLRLSCAASGFTFDDY | 171 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYFGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | G | WH12G | QVQLVESGGGLVQPGRSLRLSCAASGFTFDDY | 172 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYGGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | H | WH12H | QVQLVESGGGLVQPGRSLRLSCAASGFTFDDY | 173 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYHGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | I | WH12I | QVQLVESGGGLVQPGRSLRLSCAASGFTFDDY | 174 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYIGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | K | WH12K | QVQLVESGGGLVQPGRSLRLSCAASGFTFDDY | 175 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYKGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | L | WH12L | QVQLVESGGGLVQPGRSLRLSCAASGFTFDDY | 176 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYLGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | M | WH12M | QVQLVESGGGLVQPGRSLRLSCAASGFTFDDY | 177 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYMGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTFGQGTKLEIK | |
| W | H12 | N | WH12N | QVQLVESGGGLVQPGRSLRLSCAASGFTFDDY | 178 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYNGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | Q | WH12Q | QVQLVESGGGLVQPGRSLRLSCAASGFTFDDY | 179 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYQGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | R | WH12R | QVQLVESGGGLVQPGRSLRLSCAASGFTFDDY | 180 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYRGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | T | WH12T | QVQLVESGGGLVQPGRSLRLSCAASGFTFDDY | 181 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYTGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| W | H12 | Y | WH12Y | QVQLVESGGGLVQPGRSLRLSCAASGFTFDDY | 182 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYYGQGTLVTVSSLEGGGGSGG | |
| | | | | GGSGGGASDIQMTQSPSSVSASVGDRVTITCR | |
| | | | | ASQSISSYLNWYQQKPGKAPKLLIYAASSLQ | |
| | | | | SGVPSRFSGSGSGTDFTLTISSLQPEDFATYY | |
| | | | | CQQSYSTPWTFGQGTKLEIK | |
| Q | L1 | C | QLIC | QVQLVESGGGLVQPGRSLRLSCAASGFTFDDY | 183 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCCQSYSTPWTFGQGTKLEIK | |
| Q | L1 | E | QLIE | QVQLVESGGGLVQPGRSLRLSCAASGFTFDDY | 184 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCEQSYSTPWTFGQGTKLEIK | |
| Q | L1 | L | QLIL | QVQLVESGGGLVQPGRSLRLSCAASGFTFDDY | 185 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCLQSYSTPWTFGQGTKLEIK | |
| Q | L1 | S | QLIS | QVQLVESGGGLVQPGRSLRLSCAASGFTFDDY | 186 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCSQSYSTPWTFGQGTKLEIK | |
| Q | L1 | T | QLIT | QVQLVESGGGLVQPGRSLRLSCAASGFTFDDY | 187 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCTQSYSTPWTFGQGTKLEIK | |
| Q | L1 | Y | QLIY | QVQLVESGGGLVQPGRSLRLSCAASGFTFDDY | 188 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCYQSYSTPWTFGQGTKLEIK | |
| Q | L2 | A | QL2A | QVQLVESGGGLVQPGRSLRLSCAASGFTFDDY | 189 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQASYSTPWTFGQGTKLEIK | |
| Q | L2 | D | QL2D | QVQLVESGGGLVQPGRSLRLSCAASGFTFDDY | 190 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQDSYSTPWTFGQGTKLEIK | |
| Q | L2 | E | QL2E | QVQLVESGGGLVQPGRSLRLSCAASGFTFDDY | 191 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQESYSTPWTFGQGTKLEIK | |
| Q | L2 | G | QL2G | QVQLVESGGGLVQPGRSLRLSCAASGFTFDDY | 192 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQGSYSTPWTFGQGTKLEIK | |
| Q | L2 | H | QL2H | QVQLVESGGGLVQPGRSLRLSCAASGFTFDDY | 193 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQHSYSTPWTFGQGTKLEIK | |
| Q | L2 | I | QL2I | QVQLVESGGGLVQPGRSLRLSCAASGFTFDDY | 194 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQISYSTPWTFGQGTKLEIK | |
| Q | L2 | K | QL2K | QVQLVESGGGLVQPGRSLRLSCAASGFTFDDY | 195 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQKSYSTPWTFGQGTKLEIK | |
| Q | L2 | L | QL2L | QVQLVESGGGLVQPGRSLRLSCAASGFTFDDY | 196 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQLSYSTPWTFGQGTKLEIK | |
| Q | L2 | M | QL2M | QVQLVESGGGLVQPGRSLRLSCAASGFTFDDY | 197 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQMSYSTPWTFGQGTKLEIK | |
| Q | L2 | N | QL2N | QVQLVESGGGLVQPGRSLRLSCAASGFTFDDY | 198 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQNSYSTPWTFGQGTKLEIK | |
| Q | L2 | P | QL2P | QVQLVESGGGLVQPGRSLRLSCAASGFTFDDY | 199 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQPSYSTPWTFGQGTKLEIK | |
| Q | L2 | R | QL2R | QVQLVESGGGLVQPGRSLRLSCAASGFTFDDY | 200 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQRSYSTPWTFGQGTKLEIK | |
| Q | L2 | S | QL2S | QVQLVESGGGLVQPGRSLRLSCAASGFTFDDY | 201 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQSSYSTPWTFGQGTKLEIK | |
| Q | L2 | V | QL2V | QVQLVESGGGLVQPGRSLRLSCAASGFTFDDY | 202 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQVSYSTPWTFGQGTKLEIK | |
| Q | L2 | Y | QL2Y | QVQLVESGGGLVQPGRSLRLSCAASGFTFDDY | 203 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQYSYSTPWTFGQGTKLEIK | |
| S | L3 | A | SL3A | QVQLVESGGGLVQPGRSLRLSCAASGFTFDDY | 204 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQAYSTPWTFGQGTKLEIK | |
| Y | L4 | D | YL4D | QVQLVESGGGLVQPGRSLRLSCAASGFTFDDY | 205 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSDSTPWTFGQGTKLEIK | |
| Y | L4 | G | YL4G | QVQLVESGGGLVQPGRSLRLSCAASGFTFDDY | 206 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSGSTPWTFGQGTKLEIK | |
| Y | L4 | H | YL4H | QVQLVESGGGLVQPGRSLRLSCAASGFTFDDY | 207 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSHSTPWTFGQGTKLEIK | |
| Y | L4 | I | YL4I | QVQLVESGGGLVQPGRSLRLSCAASGFTFDDY | 208 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSISTPWTFGQGTKLEIK | |
| Y | L4 | L | YL4L | QVQLVESGGGLVQPGRSLRLSCAASGFTFDDY | 209 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSLSTPWTFGQGTKLEIK | |
| Y | L4 | M | YL4M | QVQLVESGGGLVQPGRSLRLSCAASGFTFDDY | 210 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSMSTPWTFGQGTKLEIK | |
| Y | L4 | P | YL4P | QVQLVESGGGLVQPGRSLRLSCAASGFTFDDY | 211 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSPSTPWTFGQGTKLEIK | |
| Y | L4 | Q | YL4Q | QVQLVESGGGLVQPGRSLRLSCAASGFTFDDY | 212 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSQSTPWTFGQGTKLEIK | |
| Y | L4 | R | YL4R | QVQLVESGGGLVQPGRSLRLSCAASGFTFDDY | 213 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSRSTPWTFGQGTKLEIK | |
| Y | L4 | S | YL4S | QVQLVESGGGLVQPGRSLRLSCAASGFTFDDY | 214 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSSSTPWTFGQGTKLEIK | |
| Y | L4 | T | YL4T | QVQLVESGGGLVQPGRSLRLSCAASGFTFDDY | 215 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSTSTPWTFGQGTKLEIK | |
| Y | L4 | V | YL4V | QVQLVESGGGLVQPGRSLRLSCAASGFTFDDY | 216 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSVSTPWTFGQGTKLEIK | |
| Y | L4 | W | YL4W | QVQLVESGGGLVQPGRSLRLSCAASGFTFDDY | 217 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSWSTPWTFGQGTKLEIK | |
| T | L6 | Y | TL6Y | QVQLVESGGGLVQPGRSLRLSCAASGFTFDDY | 218 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYYTPWTFGQGTKLEIK | |
| T | L6 | A | TL6A | QVQLVESGGGLVQPGRSLRLSCAASGFTFDDY | 219 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSAPWTFGQGTKLEIK | |
| T | L6 | C | TL6C | QVQLVESGGGLVQPGRSLRLSCAASGFTFDDY | 220 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSCPWTFGQGTKLEIK | |
| T | L6 | D | TL6D | QVQLVESGGGLVQPGRSLRLSCAASGFTFDDY | 221 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSDPWTFGQGTKLEIK | |
| T | L6 | F | TL6F | QVQLVESGGGLVQPGRSLRLSCAASGFTFDDY | 222 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSFPWTFGQGTKLEIK | |
| T | L6 | H | TL6H | QVQLVESGGGLVQPGRSLRLSCAASGFTFDDY | 223 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSHPWTFGQGTKLEIK | |
| T | L6 | I | TL6I | QVQLVESGGGLVQPGRSLRLSCAASGFTFDDY | 224 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSIPWTFGQGTKLEIK | |
| T | L6 | K | TL6K | QVQLVESGGGLVQPGRSLRLSCAASGFTFDDY | 225 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSKPWTFGQGTKLEIK | |
| T | L6 | L | TL6L | QVQLVESGGGLVQPGRSLRLSCAASGFTFDDY | 226 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSLPWTFGQGTKLEIK | |
| T | L6 | M | TL6M | QVQLVESGGGLVQPGRSLRLSCAASGFTFDDY | 227 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSMPWTFGQGTKLEIK | |
| T | L6 | N | TL6N | QVQLVESGGGLVQPGRSLRLSCAASGFTFDDY | 228 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSNPWTFGQGTKLEIK | |
| T | L6 | P | TL6P | QVQLVESGGGLVQPGRSLRLSCAASGFTFDDY | 229 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSPPWTFGQGTKLEIK | |
| T | L6 | Q | TL6Q | QVQLVESGGGLVQPGRSLRLSCAASGFTFDDY | 230 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSQPWTFGQGTKLEIK | |
| T | L6 | R | TL6R | QVQLVESGGGLVQPGRSLRLSCAASGFTFDDY | 231 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSRPWTFGQGTKLEIK | |
| T | L6 | V | TL6V | QVQLVESGGGLVQPGRSLRLSCAASGFTFDDY | 232 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSVPWTFGQGTKLEIK | |
| P | L7 | D | PL7D | QVQLVESGGGLVQPGRSLRLSCAASGFTFDDY | 233 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTDWTFGQGTKLEIK | |
| P | L7 | I | PL7I | QVQLVESGGGLVQPGRSLRLSCAASGFTFDDY | 234 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTIWTFGQGTKLEIK | |
| P | L7 | L | PL7L | QVQLVESGGGLVQPGRSLRLSCAASGFTFDDY | 235 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTLWTFGQGTKLEIK | |
| P | L7 | M | PL7M | QVQLVESGGGLVQPGRSLRLSCAASGFTFDDY | 236 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTMWTFGQGTKLEIK | |
| P | L7 | N | PL7N | QVQLVESGGGLVQPGRSLRLSCAASGFTFDDY | 237 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTNWTFGQGTKLEIK | |
| P | L7 | Q | PL7Q | QVQLVESGGGLVQPGRSLRLSCAASGFTFDDY | 238 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTQWTFGQGTKLEIK | |
| P | L7 | V | PL7V | QVQLVESGGGLVQPGRSLRLSCAASGFTFDDY | 239 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTVWTFGQGTKLEIK | |
| P | L7 | W | PL7W | QVQLVESGGGLVQPGRSLRLSCAASGFTFDDY | 240 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTWWTFGQGTKLEIK | |
| P | L7 | Y | PL7Y | QVQLVESGGGLVQPGRSLRLSCAASGFTFDDY | 241 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTYWTFGQGTKLEIK | |
| W | L8 | D | WL8D | QVQLVESGGGLVQPGRSLRLSCAASGFTFDDY | 242 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPDTFGQGTKLEIK | |
| W | L8 | F | WL8F | QVQLVESGGGLVQPGRSLRLSCAASGFTFDDY | 243 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPFTFGQGTKLEIK | |
| W | L8 | H | WL8H | QVQLVESGGGLVQPGRSLRLSCAASGFTFDDY | 244 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPHTFGQGTKLEIK | |
| W | L8 | I | WL8I | QVQLVESGGGLVQPGRSLRLSCAASGFTFDDY | 245 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPITFGQGTKLEIK | |
| W | L8 | K | WL8K | QVQLVESGGGLVQPGRSLRLSCAASGFTFDDY | 246 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPKTFGQGTKLEIK | |
| W | L8 | L | WL8L | QVQLVESGGGLVQPGRSLRLSCAASGFTFDDY | 247 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPLTFGQGTKLEIK | |
| W | L8 | M | WL8M | QVQLVESGGGLVQPGRSLRLSCAASGFTFDDY | 248 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPMTFGQGTKLEIK | |
| W | L8 | P | WL8P | QVQLVESGGGLVQPGRSLRLSCAASGFTFDDY | 249 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPPTFGQGTKLEIK | |
| W | L8 | Q | WL8Q | QVQLVESGGGLVQPGRSLRLSCAASGFTFDDY | 250 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPQTFGQGTKLEIK | |
| W | L8 | R | WL8R | QVQLVESGGGLVQPGRSLRLSCAASGFTFDDY | 251 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPRTFGQGTKLEIK | |
| W | L8 | S | WL8S | QVQLVESGGGLVQPGRSLRLSCAASGFTFDDY | 252 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPSTFGQGTKLEIK | |
| W | L8 | T | WL8T | QVQLVESGGGLVQPGRSLRLSCAASGFTFDDY | 253 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPTTFGQGTKLEIK | |
| W | L8 | Y | WL8Y | QVQLVESGGGLVQPGRSLRLSCAASGFTFDDY | 254 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPYTFGQGTKLEIK | |
| T | L9 | D | TL9D | QVQLVESGGGLVQPGRSLRLSCAASGFTFDDY | 255 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWDFGQGTKLEIK | |
| T | L9 | E | TL9E | QVQLVESGGGLVQPGRSLRLSCAASGFTFDDY | 256 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWEFGQGTKLEIK | |
| T | L9 | F | TL9F | QVQLVESGGGLVQPGRSLRLSCAASGFTFDDY | 257 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWFFGQGTKLEIK | |
| T | L9 | G | TL9G | QVQLVESGGGLVQPGRSLRLSCAASGFTFDDY | 258 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWGFGQGTKLEIK | |
| T | L9 | H | TL9H | QVQLVESGGGLVQPGRSLRLSCAASGFTFDDY | 259 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWHFGQGTKLEIK | |
| T | L9 | I | TL9I | QVQLVESGGGLVQPGRSLRLSCAASGFTFDDY | 260 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWIFGQGTKLEIK | |
| T | L9 | K | TL9K | QVQLVESGGGLVQPGRSLRLSCAASGFTFDDY | 261 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWKFGQGTKLEIK | |
| T | L9 | M | TL9M | QVQLVESGGGLVQPGRSLRLSCAASGFTFDDY | 262 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWMFGQGTKLEIK | |
| T | L9 | N | TL9N | QVQLVESGGGLVQPGRSLRLSCAASGFTFDDY | 263 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWNFGQGTKLEIK | |
| T | L9 | P | TL9P | QVQLVESGGGLVQPGRSLRLSCAASGFTFDDY | 264 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWPFGQGTKLEIK | |
| T | L9 | Q | TL9Q | QVQLVESGGGLVQPGRSLRLSCAASGFTFDDY | 265 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWQFGQGTKLEIK | |
| T | L9 | R | TL9R | QVQLVESGGGLVQPGRSLRLSCAASGFTFDDY | 266 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWRFGQGTKLEIK | |
| T | L9 | S | TL9S | QVQLVESGGGLVQPGRSLRLSCAASGFTFDDY | 267 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWSFGQGTKLEIK | |
| T | L9 | V | TL9V | QVQLVESGGGLVQPGRSLRLSCAASGFTFDDY | 268 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWVFGQGTKLEIK | |
| T | L9 | W | TL9W | QVQLVESGGGLVQPGRSLRLSCAASGFTFDDY | 269 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWWFGQGTKLEIK | |
| T | L9 | Y | TL9Y | QVQLVESGGGLVQPGRSLRLSCAASGFTFDDY | 270 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWYFGQGTKLEIK | |
| F | L10 | C | FL10C | QVQLVESGGGLVQPGRSLRLSCAASGFTFDDY | 271 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTCGQGTKLEIK | |
| F | L10 | D | FL10D | QVQLVESGGGLVQPGRSLRLSCAASGFTFDDY | 272 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTDGQGTKLEIK | |
| F | L10 | E | FL10E | QVQLVESGGGLVQPGRSLRLSCAASGFTFDDY | 273 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTEGQGTKLEIK | |
| F | L10 | H | FL10H | QVQLVESGGGLVQPGRSLRLSCAASGFTFDDY | 274 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTHGQGTKLEIK | |
| F | L10 | I | FL10I | QVQLVESGGGLVQPGRSLRLSCAASGFTFDDY | 275 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTIGQGTKLEIK | |
| F | L10 | K | FL10K | QVQLVESGGGLVQPGRSLRLSCAASGFTFDDY | 276 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTKGQGTKLEIK | |
| F | L10 | L | FL10L | QVQLVESGGGLVQPGRSLRLSCAASGFTFDDY | 277 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTLGQGTKLEIK | |
| F | L10 | M | FL10M | QVQLVESGGGLVQPGRSLRLSCAASGFTFDDY | 278 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTMGQGTKLEIK | |
| F | L10 | N | FL10N | QVQLVESGGGLVQPGRSLRLSCAASGFTFDDY | 279 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTNGQGTKLEIK | |
| F | L10 | P | FL10P | QVQLVESGGGLVQPGRSLRLSCAASGFTFDDY | 280 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTPGQGTKLEIK | |
| F | L10 | Q | FL10Q | QVQLVESGGGLVQPGRSLRLSCAASGFTFDDY | 281 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTQGQGTKLEIK | |
| F | L10 | R | FL10R | QVQLVESGGGLVQPGRSLRLSCAASGFTFDDY | 282 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTRGQGTKLEIK | |
| F | L10 | S | FL10S | QVQLVESGGGLVQPGRSLRLSCAASGFTFDDY | 283 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTSGQGTKLEIK | |
| F | L10 | T | FL10T | QVQLVESGGGLVQPGRSLRLSCAASGFTFDDY | 284 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTTGQGTKLEIK | |
| F | L10 | V | FL10V | QVQLVESGGGLVQPGRSLRLSCAASGFTFDDY | 285 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTVGQGTKLEIK | |
| F | L10 | W | FL10W | QVQLVESGGGLVQPGRSLRLSCAASGFTFDDY | 286 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTWGQGTKLEIK | |
| F | L10 | Y | FL10Y | QVQLVESGGGLVQPGRSLRLSCAASGFTFDDY | 287 |
| | | | | AMHWVRQAPGKGLEWVSGISWNSGSIGYADS | |
| | | | | AKGRFTISRDNSKNTLYLQMNSLRAEDTAVYY | |
| | | | | CAKRGNSNSFDYWGQGTLVTVSSLEGGGGSG | |
| | | | | GGGSGGGASDIQMTQSPSSVSASVGDRVTITC | |
| | | | | RASQSISSYLNWYQQKPGKAPKLLIYAASSL | |
| | | | | QSGVPSRFSGSGSGTDFTLTISSLQPEDFATY | |
| | | | | YCQQSYSTPWTYGQGTKLEIK | |

Table 2 provides the light chain sequences provided in Table 1. In some aspects, the light chain sequence of the CD229 antigen binding domain is the wild type sequence. In some aspects, the light chain sequence of the CD229 antigen binding domain has a substitution, mutation or deletion.

TABLE 2
CD229 antigen binding domain Light Chain Sequences

| Old | Pos | New | NAME | FR1 | CDR1 | FR2 | CDR2 | FR3 | CDR3 | FR4 |
|---|---|---|---|---|---|---|---|---|---|---|
| — | — | — | WT | DIQMT | QSIS | LNWY | AAS | SLQSGVPS | QQS | GQGT |
| | | | (2D3) | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 289) | NO: 290) | | (SEQ ID | NO: | NO: |
| | | | | NO: 288) | | | | NO: 291) | 292) | 293) |
| A | H1 | C | AH1C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 295) | NO: 296) | | (SEQ ID | NO: | NO: |
| | | | | NO: 294) | | | | NO: 297) | 298) | 299) |
| A | H1 | D | AH1D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 301) | NO: 302) | | (SEQ ID | NO: | NO: |
| | | | | NO: 300) | | | | NO: 303) | 304) | 305) |
| A | H1 | E | AH1E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 307) | NO: 308) | | (SEQ ID | NO: | NO: |
| | | | | NO: 306) | | | | NO: 309) | 310) | 311) |
| A | H1 | F | AH1F | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 313) | NO: 314) | | (SEQ ID | NO: | NO: |
| | | | | NO: 312) | | | | NO: 315) | 316) | 317) |
| A | H1 | G | AH1G | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 319) | NO: 320) | | (SEQ ID | NO: | NO: |
| | | | | NO: 318) | | | | NO: 321) | 322) | 323) |
| A | H1 | H | AH1H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 325) | NO: 326) | | (SEQ ID | NO: | NO: |
| | | | | NO: 324) | | | | NO: 327) | 238) | 329) |
| A | H1 | I | AH1I | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 331) | NO: 332) | | (SEQ ID | NO: | NO: |
| | | | | NO: 330) | | | | NO: 333) | 334) | 335) |
| A | H1 | K | AH1K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 337) | NO: 338) | | (SEQ ID | NO: | NO: |
| | | | | NO: 336) | | | | NO: 339) | 340) | 341) |
| A | H1 | L | AH1L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 343) | NO: 344) | | (SEQ ID | NO: | NO: |
| | | | | NO: 342) | | | | NO: 345) | 346) | 347) |
| A | H1 | M | AH1M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 349) | NO: 350) | | (SEQ ID | NO: | NO: |
| | | | | NO: 348) | | | | NO: 351) | 352) | 353) |
| A | H1 | N | AH1N | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | | KAPKL | | TDFTLTIS | WTF | K |
| | | | | GDRVT | | LIY | | SLQPEDFA | | |
| | | | | ITCRAS | | | | TYYC | | |
| A | H1 | P | AH1P | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 355) | NO: 356) | | (SEQ ID | NO: | NO: |
| | | | | NO: 354) | | | | NO: 357) | 358) | 359) |
| A | H1 | S | AH1S | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 361) | NO: 362) | | (SEQ ID | NO: | NO: |
| | | | | NO: 360) | | | | NO: 363) | 364) | 365) |
| A | H1 | T | AH1T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPE | (SEQ | (SEQ |
| | | | | ITCRAS | NO: | (SEQ ID | | DFATYYC | ID | ID |
| | | | | (SEQ ID | 367) | NO: 368) | | (SEQ ID | NO: | NO: |
| | | | | NO: 366) | | | | NO: 369) | 370) | 371) |
| A | H1 | V | AH1V | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 373) | NO: 374) | | (SEQ ID | NO: | NO: |
| | | | | NO: 372) | | | | NO: 375 | 376) | 377) |
| A | H1 | W | AH1W | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 379) | NO: 380) | | (SEQ ID | NO: | NO: |
| | | | | NO: 378) | | | | NO: 381) | 382) | 383) |
| A | H1 | Y | AH1Y | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 385) | NO: 386) | | (SEQ ID | NO: | NO: |
| | | | | NO: 384) | | | | NO: 387) | 388) | 389) |
| K | H2 | C | KH2C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 391) | NO: 392) | | (SEQ ID | NO: | NO: |
| | | | | NO: 390) | | | | NO: 393) | 394) | 395) |
| K | H2 | D | KH2D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 397) | NO: 398) | | (SEQ ID | NO: | NO: |
| | | | | NO: 396) | | | | NO: 399) | 400) | 401) |
| K | H2 | E | KH2E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 403) | NO: 404) | | (SEQ ID | NO: | NO: |
| | | | | NO: 402) | | | | NO: 405) | 406) | 407) |
| K | H2 | F | KH2F | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 409) | NO: 410) | | (SEQ ID | NO: | NO: |
| | | | | NO: 408) | | | | NO: 411) | 412) | 413) |
| K | H2 | G | KH2G | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 415) | NO: 416) | | (SEQ ID | NO: | NO: |
| | | | | NO: 414) | | | | NO: 417) | 418) | 419) |
| K | H2 | H | KH2H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 421) | NO: 422) | | (SEQ ID | NO: | NO: |
| | | | | NO: 420) | | | | NO: 423) | 424) | 425) |
| K | H2 | I | KH2I | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 427) | NO: 428) | | (SEQ ID | NO: | NO: |
| | | | | NO: 426) | | | | NO: 429) | 430) | 431) |
| K | H2 | L | KH2L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 433) | NO: 434) | | (SEQ ID | NO: | NO: |
| | | | | NO: 432) | | | | NO: 435) | 436) | 437) |
| K | H2 | M | KH2M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 439) | NO: 440) | | (SEQ ID | NO: | NO: |
| | | | | NO: 438) | | | | NO: 441) | 442) | 443) |
| K | H2 | N | KH2N | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 445) | NO: 446) | | (SEQ ID | NO: | NO: |
| | | | | NO: 444) | | | | NO: 447) | 448) | 449) |
| K | H2 | P | KH2P | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 451) | NO: 452) | | (SEQ ID | NO: | NO: |
| | | | | NO: 450) | | | | NO: 453) | 454) | 455) |
| K | H2 | Q | KH2Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 457) | NO: 458) | | (SEQ ID | NO: | NO: |
| | | | | NO: 456) | | | | NO: 459) | 460) | 461) |
| K | H2 | R | KH2R | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 463) | NO: 464) | | (SEQ ID | NO: | NO: |
| | | | | NO: 462) | | | | NO: 465) | 466) | 467) |
| K | H2 | S | KH2S | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 469) | NO: 470) | | (SEQ ID | NO: | NO: |
| | | | | NO: 468) | | | | NO: 471) | 472) | 473) |
| K | H2 | V | KH2V | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 475) | NO: 476) | | (SEQ ID | NO: | NO: |
| | | | | NO: 474) | | | | NO: 477) | 478) | 479) |
| K | H2 | W | KH2W | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 481) | NO: 482) | | (SEQ ID | NO: | NO: |
| | | | | NO: 480) | | | | NO: 483) | 484) | 485) |
| R | H3 | A | RH3A | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 486) | NO: 487) | | (SEQ ID | NO: | NO: |
| | | | | NO: 486) | | | | NO: 488) | 489) | 490) |
| R | H3 | C | RH3C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 492) | NO: 493) | | (SEQ ID | NO: | NO: |
| | | | | NO: 491) | | | | NO: 494) | 495) | 496) |
| R | H3 | G | RH3G | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 498) | NO: 499) | | (SEQ ID | NO: | NO: |
| | | | | NO: 497) | | | | NO: 500) | 501) | 502) |
| R | H3 | H | RH3H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 504) | NO: 505) | | (SEQ ID | NO: | NO: |
| | | | | NO: 503) | | | | NO: 506) | 507) | 508) |
| R | H3 | I | RH3I | DIQMT | QSI | LNWY | AAS | SLQSGVPS | QQS | GQGT |
| | | | | QSPSS | SSY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 510) | NO: 511) | | (SEQ ID | NO: | NO: |
| | | | | NO: 509) | | | | NO: 512) | 513) | 514) |
| R | H3 | K | RH3K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K(SE |
| | | | | GDRVT | ID | LIY | | SLQPEDFA | (SEQ | Q ID |
| | | | | ITCRAS | NO: | (SEQ ID | | TYYC | ID | NO: |
| | | | | (SEQ ID | 516) | NO: 517) | | (SEQ ID | NO: | 520) |
| | | | | NO: 515) | | | | NO: 518) | 519) | |
| R | H3 | L | RH3L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 522) | NO: 523) | | (SEQ ID | NO: | NO: |
| | | | | NO: 521) | | | | NO: 524) | 525) | 526) |
| R | H3 | M | RH3M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 528) | NO: 529) | | (SEQ ID | NO: | NO: |
| | | | | NO: 527) | | | | NO: 530) | 531) | 532) |
| R | H3 | N | RH3N | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 534) | NO: 535) | | (SEQ ID | NO: | NO: |
| | | | | NO: 533) | | | | NO: 536) | 537) | 538) |
| R | H3 | P | RH3P | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 540) | NO: 541) | | (SEQ ID | NO: | NO: |
| | | | | NO: 539) | | | | NO: 542) | 543) | 544) |
| R | H3 | Q | RH3Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 546) | NO: 547) | | (SEQ ID | NO: | NO: |
| | | | | NO: 545) | | | | NO: 548) | 549) | 550) |
| R | H3 | S | RH3S | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 552) | NO: 553) | | (SEQ ID | NO: | NO: |
| | | | | NO: 551) | | | | NO: 554) | 555) | 556) |
| R | H3 | T | RH3T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 558) | NO: 559) | | (SEQ ID | NO: | NO: |
| | | | | NO: 557) | | | | NO: 560) | 561) | 562) |
| R | H3 | V | RH3V | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 564) | NO: 565) | | (SEQ ID | NO: | NO: |
| | | | | NO: 563) | | | | NO: 566) | 567) | 568) |
| R | H3 | W | RH3W | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 570) | NO: 571) | | (SEQ ID | NO: | NO: |
| | | | | NO: 569) | | | | NO: 572) | 573) | 574) |
| R | H3 | Y | RH3Y | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 576) | NO: 577) | | (SEQ ID | NO: | NO: |
| | | | | NO: 575) | | | | NO: 578) | 579) | 580) |
| G | H4 | A | GH4A | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 582) | NO: 583) | | (SEQ ID | NO: | NO: |
| | | | | NO: 581) | | | | NO: 584) | 585) | 586) |
| G | H4 | C | GH4C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 588) | NO: 589) | | (SEQ ID | NO: | NO: |
| | | | | NO: 587) | | | | NO: 590) | 591) | 592) |
| G | H4 | D | GH4D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 594) | NO: 595) | | (SEQ ID | NO: | NO: |
| | | | | NO: 593) | | | | NO: 596) | 597) | 598) |
| G | H4 | E | GH4E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 600) | NO: 601) | | (SEQ ID | NO: | NO: |
| | | | | NO: 599) | | | | NO: 602) | 603) | 604) |
| G | H4 | F | GH4F | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 606) | NO: 607) | | (SEQ ID | NO: | NO: |
| | | | | NO: 605) | | | | NO: 608) | 609) | 610) |
| G | H4 | H | GH4H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 612) | NO: 613) | | (SEQ ID | NO: | NO: |
| | | | | NO: 611) | | | | NO: 614) | 615) | 616) |
| G | H4 | I | GH4I | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 618) | NO: 619) | | (SEQ ID | NO: | NO: |
| | | | | NO: 617) | | | | NO: 620) | 621) | 622) |
| G | H4 | K | GH4K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 624) | NO: 625) | | (SEQ ID | NO: | NO: |
| | | | | NO: 623) | | | | NO: 626) | 627)62 | 8) |
| G | H4 | L | GH4L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 630) | NO: 631) | | (SEQ ID | NO: | NO: |
| | | | | NO: 629) | | | | NO: 632) | 633) | 634) |
| G | H4 | M | GH4M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 636) | NO: 637) | | (SEQ ID | NO: | NO: |
| | | | | NO: 635) | | | | NO: 638) | 639) | 640) |
| G | H4 | N | GH4N | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 642) | NO: 643) | | (SEQ ID | NO: | NO: |
| | | | | NO: 641) | | | | NO: 644) | 645) | 646) |
| G | H4 | P | GH4P | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 648) | NO: 649) | | (SEQ ID | NO: | NO: |
| | | | | NO: 647) | | | | NO: 649) | 650) | 651) |
| G | H4 | Q | GH4Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 653) | NO: 654) | | (SEQ ID | NO: | NO: |
| | | | | NO: 652) | | | | NO: 655) | 656) | 657) |
| G | H4 | R | GH4R | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 659) | NO: 660) | | (SEQ ID | NO: | NO: |
| | | | | NO: 658) | | | | NO: 661) | 662) | 663) |
| G | H4 | S | GH4S | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 665) | NO: 666) | | (SEQ ID | NO: | NO: |
| | | | | NO: 664) | | | | NO: 667) | 668) | 669) |
| G | H4 | T | GH4T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 671) | NO: 672) | | (SEQ ID | NO: | NO: |
| | | | | NO: 670) | | | | NO: 673) | 674) | 675) |
| G | H4 | V | GH4V | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 677) | NO: 678) | | (SEQ ID | NO: | NO: |
| | | | | NO: 676) | | | | NO: 679) | 680) | 681) |
| G | H4 | Y | GH4Y | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 683) | NO: 684) | | (SEQ ID | NO: | NO: |
| | | | | NO: 682) | | | | NO: 685) | 686) | 687) |
| N | H5 | A | NH5A | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 689) | NO: 690) | | (SEQ ID | NO: | NO: |
| | | | | NO: 688) | | | | NO: 691) | 692) | 693) |
| N | H5 | C | NH5C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 695) | NO: 696) | | (SEQ ID | NO: | NO: |
| | | | | NO: 694) | | | | NO: 697) | 698) | 699) |
| N | H5 | D | NH5D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 701) | NO: 702) | | (SEQ ID | NO: | NO: |
| | | | | NO: 700) | | | | NO: 703) | 704) | 705) |
| N | H5 | E | NH5E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 707) | NO: 708) | | (SEQ ID | NO: | NO: |
| | | | | NO: 706) | | | | NO: 709) | 710) | 711) |
| N | H5 | H | NH5H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 713) | NO: 714) | | (SEQ ID | NO: | NO: |
| | | | | NO: 712) | | | | NO: 715) | 716) | 717) |
| N | H5 | I | NH5I | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 719) | NO: 720) | | (SEQ ID | NO: | NO: |
| | | | | NO: 718) | | | | NO: 721) | 722) | 723) |
| N | H5 | L | NH5L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 725) | NO: 726) | | (SEQ ID | NO: | NO: |
| | | | | NO: 724) | | | | NO: 726) | 727) | 728) |
| N | H5 | M | NH5M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 730) | NO: 731) | | (SEQ ID | NO: | NO: |
| | | | | NO: 729) | | | | NO: 732) | 733) | 734) |
| N | H5 | P | NH5P | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 736) | NO: 737) | | (SEQ ID | NO: | NO: |
| | | | | NO: 735) | | | | NO: 738) | 739) | 740) |
| N | H5 | T | NH5T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 742) | NO: 743) | | (SEQ ID | NO: | NO: |
| | | | | NO: 741) | | | | NO: 744) | 745) | 746) |
| N | H5 | V | NH5V | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 748) | NO: 749) | | (SEQ ID | NO: | NO: |
| | | | | NO: 747) | | | | NO: 750) | 751) | 752) |
| N | H5 | W | NH5W | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 754) | NO: 755) | | (SEQ ID | NO: | NO: |
| | | | | NO: 753) | | | | NO:756) | 757) | 758) |
| S | H6 | A | SH6A | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 760) | NO: 761) | | (SEQ ID | NO: | NO: |
| | | | | NO: 759) | | | | NO: 762) | 763) | 764) |
| S | H6 | C | SH6C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 766) | NO: 767) | | (SEQ ID | NO: | NO: |
| | | | | NO: 765) | | | | NO: 768) | 769) | 770) |
| S | H6 | D | SH6D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 772) | NO: 773) | | (SEQ ID | NO: | NO: |
| | | | | NO: 771) | | | | NO: 774) | 775) | 776) |
| S | H6 | E | SH6E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 778) | NO: 779) | | (SEQ ID | NO: | NO: |
| | | | | NO: 777) | | | | NO: 780) | 781) | 782) |
| S | H6 | F | SH6F | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 784) | NO: 785) | | (SEQ ID | NO: | NO: |
| | | | | NO: 783) | | | | NO: 786) | 787) | 788) |
| S | H6 | H | SH6H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 790) | NO: 791) | | (SEQ ID | NO: | NO: |
| | | | | NO: 789) | | | | NO: 792) | 793) | 794) |
| S | H6 | K | SH6K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 796) | NO: 797) | | (SEQ ID | NO: | NO: |
| | | | | NO: 795) | | | | NO: 798) | 799) | 800) |
| S | H6 | L | SH6L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 802) | NO: 803) | | (SEQ ID | NO: | NO: |
| | | | | NO: 801) | | | | NO: 804) | 805) | 806) |
| S | H6 | N | SH6N | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 808) | NO: 809) | | (SEQ ID | NO: | NO: |
| | | | | NO: 807) | | | | NO: 810) | 811) | 812) |
| S | H6 | P | SH6P | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 814) | NO: 815) | | (SEQ ID | NO: | NO: |
| | | | | NO: 813) | | | | NO: 816) | 817) | 818) |
| S | H6 | Q | SH6Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 820) | NO: 821) | | (SEQ ID | NO: | NO: |
| | | | | NO: 819) | | | | NO: 822) | 823) | 824) |
| S | H6 | T | SH6T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 826) | NO: 827) | | (SEQ ID | NO: | NO: |
| | | | | NO: 825) | | | | NO: 828) | 829) | 830) |
| S | H6 | W | SH6W | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 832) | NO: 833) | | (SEQ ID | NO: | NO: |
| | | | | NO: 831) | | | | NO: 834) | 835) | 836) |
| S | H6 | Y | SH6Y | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 838) | NO: 839) | | (SEQ ID | NO: | NO: |
| | | | | NO: 837) | | | | NO: 840) | 841) | 842) |
| N | H7 | A | NH7A | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 844) | NO: 845) | | (SEQ ID | NO: | NO: |
| | | | | NO: 843) | | | | NO: 846) | 847) | 848) |
| N | H7 | C | NH7C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 850) | NO: 851) | | (SEQ ID | NO: | NO: |
| | | | | NO: 849) | | | | NO: 852) | 853) | 854) |
| N | H7 | D | NH7D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 856) | NO: 857) | | (SEQ ID | NO: | NO: |
| | | | | NO: 855) | | | | NO: 858) | 859) | 860) |
| N | H7 | E | NH7E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 862) | NO: 863) | | (SEQ ID | NO: | NO: |
| | | | | NO: 861) | | | | NO: 864) | 865) | 866) |
| N | H7 | F | NH7F | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 868) | NO: 869) | | (SEQ ID | NO: | NO: |
| | | | | NO: 867) | | | | NO: 870) | 871) | 872) |
| N | H7 | I | NH7I | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 874) | NO: 875) | | (SEQ ID | NO: | NO: |
| | | | | NO: 873) | | | | NO: 876) | 877) | 878) |
| N | H7 | K | NH7K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 880) | NO: 881) | | (SEQ ID | NO: | NO: |
| | | | | NO: 879) | | | | NO: 882) | 883) | 884) |
| N | H7 | L | NH7L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 885) | NO: 886) | | (SEQ ID | NO: | NO: |
| | | | | NO: 885) | | | | NO: 887) | 888) | 889) |
| N | H7 | M | NH7M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 891) | NO: 892) | | (SEQ ID | NO: | NO: |
| | | | | NO: 890) | | | | NO: 893) | 894) | 895) |
| N | H7 | Q | NH7Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 897) | NO: 898) | | (SEQ ID | NO: | NO: |
| | | | | NO: 896) | | | | NO: 899) | 900) | 901) |
| N | H7 | R | NH7R | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 903) | NO: 904) | | (SEQ ID | NO: | NO: |
| | | | | NO: 902) | | | | NO: 905) | 906) | 907) |
| N | H7 | S | NH7S | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 909) | NO: 910) | | (SEQ ID | NO: | NO: |
| | | | | NO: 908) | | | | NO: 911) | 912) | 913) |
| N | H7 | T | NH7T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 915) | NO: 916) | | (SEQ ID | NO: | NO: |
| | | | | NO: 914) | | | | NO: 917) | 918) | 919) |
| N | H7 | W | NH7W | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 921) | NO: 922) | | (SEQ ID | NO: | NO: |
| | | | | NO: 920) | | | | NO: 923) | 924) | 925) |
| N | H7 | Y | NH7Y | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 927) | NO: 928) | | (SEQ ID | NO: | NO: |
| | | | | NO: 926) | | | | NO: 929) | 930) | 931) |
| S | H8 | A | SH8A | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 933) | NO: 934) | | (SEQ ID | NO: | NO: |
| | | | | NO: 932) | | | | NO: 935) | 936) | 937) |
| S | H8 | C | SH8C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 939) | NO: 940) | | (SEQ ID | NO: | NO: |
| | | | | NO: 938) | | | | NO: 941) | 942) | 943) |
| S | H8 | D | SH8D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 945) | NO: 946) | | (SEQ ID | NO: | NO: |
| | | | | NO: 944) | | | | NO: 947) | 948) | 949) |
| S | H8 | E | SH8E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 951) | NO: 952) | | (SEQ ID | NO: | NO: |
| | | | | NO: 950) | | | | NO: 953) | 954) | 955) |
| S | H8 | F | SH8F | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 957) | NO: 958) | | (SEQ ID | NO: | NO: |
| | | | | NO: 956) | | | | NO: 959) | 960) | 961) |
| S | H8 | G | SH8G | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 963) | NO: 964) | | (SEQ ID | NO: | NO: |
| | | | | NO: 962) | | | | NO: 965) | 966) | 967) |
| S | H8 | H | SH8H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 969) | NO: 970) | | (SEQ ID | NO: | NO: |
| | | | | NO: 968) | | | | NO: 971) | 972) | 973) |
| S | H8 | K | SH8K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 975) | NO: 976) | | (SEQ ID | NO: | NO: |
| | | | | NO: 974) | | | | NO: 977) | 978) | 979) |
| S | H8 | N | SH8N | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 981) | NO: 982) | | (SEQ ID | NO: | NO: |
| | | | | NO: 980) | | | | NO: 983) | 984) | 985) |
| S | H8 | Q | SH8Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 987) | NO: 988) | | (SEQ ID | NO: | NO: |
| | | | | NO: 986) | | | | NO: 989) | 990) | 991) |
| S | H8 | R | SH8R | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 993) | NO: 994) | | (SEQ ID | NO: | NO: |
| | | | | NO: 992) | | | | NO: 995) | 996) | 997) |
| S | H8 | T | SH8T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 999) | NO: | | (SEQ ID | NO: | NO: |
| | | | | NO: 998) | | 1000) | | NO: 1001) | 1002) | 1003) |
| S | H8 | V | SH8V | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1005) | NO: | | (SEQ ID | NO: | NO: |
| | | | | NO: | | 1006) | | NO: 1007) | 1008) | 1009) |
| | | | | 1004) | | | | | | |
| S | H8 | W | SH8W | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1011) | NO: | | (SEQ ID | NO: | NO: |
| | | | | NO: | | 1012) | | NO: 1013) | 1014) | 1015) |
| | | | | 1010) | | | | | | |
| F | H9 | A | FH9A | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1017) | NO: | | (SEQ ID | NO: | NO: |
| | | | | NO: | | 1018) | | NO: 1019) | 1020) | 1021) |
| | | | | 1016) | | | | | | |
| F | H9 | D | FH9D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1023) | NO: | | (SEQ ID | NO: | NO: |
| | | | | NO: | | 1024) | | NO: 1025) | 1026) | 1027) |
| | | | | 1022) | | | | | | |
| F | H9 | E | FH9E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1029) | NO: | | (SEQ ID | NO: | NO: |
| | | | | NO: | | 1030) | | NO: 1031) | 1032) | 1033) |
| | | | | 1028) | | | | | | |
| F | H9 | G | FH9G | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1035) | NO: | | (SEQ ID | NO: | NO: |
| | | | | NO: | | 1036) | | NO: 1037) | 1038) | 1039) |
| | | | | 1034) | | | | | | |
| F | H9 | H | FH9H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC(SEQ | ID | ID |
| | | | | (SEQ ID | 1041) | NO: | | ID | NO: | NO: |
| | | | | NO: | | 1042) | | NO: 1043) | 1044) | 1045) |
| | | | | 1040) | | | | | | |
| F | H9 | K | FH9K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1047) | NO: | | (SEQ ID | NO: | NO: |
| | | | | NO: | | 1048) | | NO: 1049) | 1050) | 1051) |
| | | | | 1046) | | | | | | |
| F | H9 | L | FH9L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1053) | NO: | | (SEQ ID | NO: | NO: |
| | | | | NO: | | 1054) | | NO: 1055) | 1056) | 1057) |
| | | | | 1052) | | | | | | |
| F | H9 | M | FH9M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1059) | NO: | | (SEQ ID | NO: | NO: |
| | | | | NO: | | 1060) | | NO: 1061) | 1062) | 1063) |
| | | | | 1058) | | | | | | |
| F | H9 | N | FH9N | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1065) | NO: | | (SEQ ID | NO: | NO: |
| | | | | NO: | | 1066) | | NO: 1067) | 1068) | 1069) |
| | | | | 1064) | | | | | | |
| F | H9 | P | FH9P | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1071) | NO: | | (SEQ ID | NO: | NO: |
| | | | | NO: | | 1072) | | NO: 1073) | 1074) | 1075) |
| | | | | 1070) | | | | | | |
| F | H9 | Q | FH9Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1077) | NO: | | (SEQ ID | NO: | NO: |
| | | | | NO: | | 1078) | | NO: 1079) | 1080) | 1081) |
| | | | | 1076) | | | | | | |
| F | H9 | T | FH9T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1083) | NO: | | (SEQ ID | NO: | NO: |
| | | | | NO: | | 1084) | | NO: 1085) | 1086) | 1087) |
| | | | | 1082) | | | | | | |
| F | H9 | W | FH9W | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1089) | NO: | | (SEQ ID | NO: | NO: |
| | | | | NO: | | 1090) | | NO: 1091) | 1092) | 1093) |
| | | | | 1088) | | | | | | |
| F | H9 | Y | FH9Y | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1095) | NO: | | (SEQ ID | NO: | NO: |
| | | | | NO: | | 1096) | | NO: 1097) | 1098) | 1099) |
| | | | | 1094) | | | | | | |
| D | H10 | A | DH10A | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1101) | NO: | | (SEQ ID | NO: | NO: |
| | | | | NO: | | 1102) | | NO: 1103) | 1104) | 1105) |
| | | | | 1100) | | | | | | |
| D | H10 | C | DH10C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1107) | NO: | | (SEQ ID | NO: | NO: |
| | | | | NO: | | 1108) | | NO: 1109) | 1110) | 1111) |
| | | | | 1106) | | | | | | |
| D | H10 | E | DH10E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1113) | NO: | | (SEQ ID | NO: | NO: |
| | | | | NO: | | 1114) | | NO: 1115) | 1116) | 1117) |
| | | | | 1112) | | | | | | |
| D | H10 | F | DH10F | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1119) | NO: | | (SEQ ID | NO: | NO: |
| | | | | NO: | | 1120) | | NO: 1121) | 1122) | 1122) |
| | | | | 1118) | | | | | | |
| D | H10 | G | DH10G | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1124) | NO: | | (SEQ ID | NO: | NO: |
| | | | | NO: | | 1125) | | NO: 1126) | 1127) | 1128) |
| | | | | 1123) | | | | | | |
| D | H10 | K | DH10K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1130) | NO: | | (SEQ ID | NO: | NO: |
| | | | | NO: | | 1131) | | NO: 1132) | 1133) | 1134) |
| | | | | 1129) | | | | | | |
| D | H10 | L | DH10L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1136) | NO | | (SEQ ID | NO: | NO: |
| | | | | NO: | | :1137) | | NO: 1138) | 1139) | 1140) |
| | | | | 1135) | | | | | | |
| D | H10 | M | DH10M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1142) | NO: | | (SEQ ID | NO: | NO: |
| | | | | NO: | | 1143) | | NO: 1144) | 1145) | 1146) |
| | | | | 1141) | | | | | | |
| D | H10 | N | DH10N | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | G | ID | LIY | | SLQPEDFA | (SEQ | (SEQ |
| | | | | DRVTI | NO: | (SEQ ID | | TYYC | ID | ID |
| | | | | TCRAS | 1148) | NO: | | (SEQ ID | NO: | NO: |
| | | | | (SEQ ID | | 1149) | | NO: 1150) | 1151) | 1152) |
| | | | | NO: | | | | | | |
| | | | | 1147) | | | | | | |
| D | H10 | P | DH10P | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1154) | NO: | | (SEQ ID | NO: | NO: |
| | | | | NO: | | 1155) | | NO: 1156) | 1157) | 1158) |
| | | | | 1153) | | | | | | |
| D | H10 | Q | DH10Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1160) | NO: | | (SEQ ID | NO: | NO: |
| | | | | NO: | | 1161) | | NO: 1162) | 1163) | 1164) |
| | | | | 1159) | | | | | | |
| D | H10 | R | DH10R | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1166) | NO: | | (SEQ ID | NO: | NO: |
| | | | | NO: | | 1167) | | NO: 1168) | 1169) | 1170) |
| | | | | 1165) | | | | | | |
| D | H10 | T | DH10T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1172) | NO: | | (SEQ ID | NO: | NO: |
| | | | | NO: | | 1173) | | NO: 1174) | 1175) | 1176) |
| | | | | 1171) | | | | | | |
| D | H10 | V | DH10V | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1178) | NO: | | (SEQ ID | NO: | NO: |
| | | | | NO: | | 1179) | | NO: 1180) | 1181) | 1182) |
| | | | | 1177) | | | | | | |
| D | H10 | Y | DH10Y | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1184) | NO: | | (SEQ ID | NO: | NO: |
| | | | | NO: | | 1185) | | NO: 1186) | 1187) | 1188) |
| | | | | 1183) | | | | | | |
| Y | H11 | A | YH11A | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1190) | NO: | | (SEQ ID | NO: | NO: |
| | | | | NO: | | 1191) | | NO: 1191) | 1192) | 1193) |
| | | | | 1189) | | | | | | |
| Y | H11 | C | YH11C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1195) | NO: | | (SEQ ID | NO: | NO: |
| | | | | NO: | | 1196) | | NO: 1197) | 1198) | 1199) |
| | | | | 1194) | | | | | | |
| Y | H11 | D | YH11D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1201) | NO: | | (SEQ ID | NO: | NO: |
| | | | | NO: | | 1202) | | NO: 1203) | 1204) | 1205) |
| | | | | 1200) | | | | | | |
| Y | H11 | E | YH11E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1207) | NO: | | (SEQ ID | NO: | NO: |
| | | | | NO: | | 1208) | | NO: 1209) | 1210) | 1211) |
| | | | | 1206) | | | | | | |
| Y | H11 | F | YH11F | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1213) | NO: | | (SEQ ID | NO: | NO: |
| | | | | NO: | | 1214) | | NO: 1215) | 1216) | 1217) |
| | | | | 1212) | | | | | | |
| Y | H11 | G | YH11G | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1219) | NO: | | (SEQ ID | NO: | NO: |
| | | | | NO: | | 0122) | | NO: 1221) | 1222) | 1223) |
| | | | | 1218) | | | | | | |
| Y | H11 | K | YH11K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1225) | NO: | | (SEQ ID | NO: | NO: |
| | | | | NO: | | 1226) | | NO: 1227) | 2128) | 1229) |
| | | | | 1224) | | | | | | |
| Y | H11 | L | YH11L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1231) | NO: | | (SEQ ID | NO: | NO: |
| | | | | NO: | | 1232) | | NO: 1233) | 1234) | 1235) |
| | | | | 1230) | | | | | | |
| Y | H11 | M | YH11M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1237) | NO: | | (SEQ ID | NO: | NO: |
| | | | | NO: | | 1238) | | NO: 1239) | 1240) | 1241) |
| | | | | 1236) | | | | | | |
| Y | H11 | Q | YH11Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1243) | NO: | | (SEQ ID | NO: | NO: |
| | | | | NO: | | 1244) | | NO: 1245) | 1246) | 1247) |
| | | | | 1242) | | | | | | |
| Y | H11 | R | YH11R | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1249) | NO: | | (SEQ ID | NO: | NO: |
| | | | | NO: | | 1250) | | NO: 1251) | 1252) | 1253) |
| | | | | 1248) | | | | | | |
| Y | H11 | S | YH11S | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1255) | NO: | | (SEQ ID | NO: | NO: |
| | | | | NO: | | 1256) | | NO: 1257) | 1258) | 1259) |
| | | | | 1254) | | | | | | |
| Y | H11 | T | YH11T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1261) | NO: | | (SEQ ID | NO: | NO: |
| | | | | NO: | | 1262) | | NO: 1263) | 1264) | 1265) |
| | | | | 1260) | | | | | | |
| Y | H11 | V | YH11V | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1267) | NO: | | (SEQ ID | NO: | NO: |
| | | | | NO: | | 1268) | | NO: 1269) | 1270) | 1271) |
| | | | | 1266) | | | | | | |
| Y | H11 | W | YH11W | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1273) | NO: | | (SEQ ID | NO: | NO: |
| | | | | NO: | | 1274) | | NO: 1275) | 1276) | 1277) |
| | | | | 1272) | | | | | | |
| W | H12 | A | WH12A | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1279) | NO: | | (SEQ ID | NO: | NO: |
| | | | | NO: | | 1280) | | NO: 1281) | 1282) | 1283) |
| | | | | 1278) | | | | | | |
| W | H12 | C | WH12C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1285) | NO: | | (SEQ ID | NO: | NO: |
| | | | | NO: | | 1286) | | NO: 1287) | 1288) | 1289) |
| | | | | 1284) | | | | | | |
| W | H12 | E | WH12E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1291) | NO: | | (SEQ ID | NO: | NO: |
| | | | | NO: | | 1292) | | NO: 1293) | 1294) | 1295) |
| | | | | 1290) | | | | | | |
| W | H12 | F | WH12F | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1297) | NO: | | (SEQ ID | NO: | NO: |
| | | | | NO: | | 1298) | | NO: 1299) | 1300) | 1301) |
| | | | | 1296) | | | | | | |
| W | H12 | G | WH12G | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1303) | NO: | | (SEQ ID | NO: | NO: |
| | | | | NO: | | 1304) | | NO: 1305) | 1306) | 1307) |
| | | | | 1302) | | | | | | |
| W | H12 | H | WH12H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC(SEQ | ID | ID |
| | | | | (SEQ ID | 1309) | NO: | | ID | NO: | NO: |
| | | | | NO: | | 0131) | | NO: 1311) | 1311) | 1312) |
| | | | | 1308) | | | | | | |
| W | H12 | I | WH12I | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1314) | NO: | | (SEQ ID | NO: | NO: |
| | | | | NO: | | 1315) | | NO: 1316) | 1317) | 1318) |
| | | | | 1313) | | | | | | |
| W | H12 | K | WH12K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1320) | NO: | | (SEQ ID | NO: | NO: |
| | | | | NO: | | 1321) | | NO: 1322) | 1323) | 1324) |
| | | | | 1319) | | | | | | |
| W | H12 | L | WH12L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1326) | NO: | | (SEQ ID | NO: | NO: |
| | | | | NO: | | 1327) | | NO: 1328) | 1329) | 1330) |
| | | | | 1325) | | | | | | |
| W | H12 | M | WH12M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1332) | NO: | | (SEQ ID | NO: | NO: |
| | | | | NO: | | 1333) | | NO: 1334) | 1335) | 1336) |
| | | | | 1331) | | | | | | |
| W | H12 | N | WH12N | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1337) | NO: | | (SEQ ID | NO: | NO: |
| | | | | NO: | | 1338) | | NO: 1339) | 1340) | 1341 |
| | | | | 1336) | | | | | | |
| W | H12 | Q | WH12Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1343) | NO: | | (SEQ ID | NO: | NO: |
| | | | | NO: | | 1344) | | NO: 1345) | 1346) | 1347) |
| | | | | 1342) | | | | | | |
| W | H12 | R | WH12R | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1349) | NO: | | (SEQ ID | NO: | NO: |
| | | | | NO: | | 1350) | | NO: 1351) | 1352) | 1353) |
| | | | | 1348) | | | | | | |
| W | H12 | T | WH12T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1355) | NO: | | (SEQ ID | NO: | NO: |
| | | | | NO: | | 1356) | | NO: 1357) | 1358) | 1359) |
| | | | | 1354) | | | | | | |
| W | H12 | Y | WH12Y | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1361) | NO: | | (SEQ ID | NO: | NO: |
| | | | | NO: | | 1362) | | NO: 1363) | 1364) | 1365) |
| | | | | 1360) | | | | | | |
| Q | L1 | C | QL1C | DIQMT | QSIS | LNWY | AAS | SLQSGVPS | CQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1367) | NO: | | (SEQ ID | NO: | NO: |
| | | | | NO: | | 1368) | | NO: 1369) | 1370) | 1371) |
| | | | | 1366) | | | | | | |
| Q | L1 | E | QL1E | DIQMT | QSIS | LNWY | AAS | SLQSGVPS | EQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1373) | NO: | | (SEQ ID | NO: | NO: |
| | | | | NO: | | 1374) | | NO: 1375) | 1376) | 1377) |
| | | | | 1372) | | | | | | |
| Q | L1 | L | QL1L | DIQMT | QSIS | LNWY | AAS | SLQSGVPS | LQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1379) | NO: | | (SEQ ID | NO: | NO: |
| | | | | NO: | | 1380) | | NO: 1381) | 1382) | 1383) |
| | | | | 1378) | | | | | | |
| Q | L1 | S | QL1S | DIQMT | QSIS | LNWY | AAS | SLQSGVPS | SQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1385) | NO: | | (SEQ ID | NO: | NO: |
| | | | | NO: | | 1386) | | NO: 1387) | 1388) | 1389) |
| | | | | 1384) | | | | | | |
| Q | L1 | T | QL1T | DIQMT | QSIS | LNWY | AAS | SLQSGVPS | TQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1391) | NO: | | (SEQ ID | NO: | NO: |
| | | | | NO: | | 1392) | | NO: 1393) | 1394) | 1395) |
| | | | | 1390) | | | | | | |
| Q | L1 | Y | QL1Y | DIQMT | QSIS | LNWY | AAS | SLQSGVPS | YQS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1397) | NO: | | (SEQ ID | NO: | NO: |
| | | | | NO: | | 1398) | | NO: 1399) | 1400) | 1401) |
| | | | | 1396) | | | | | | |
| Q | L2 | A | QL2A | DIQMT | QSIS | LNWY | AAS | SLQSGVPS | QAS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1403) | NO: | | (SEQ ID | NO: | NO: |
| | | | | NO: | | 1404) | | NO: 1405) | 1406) | 1407) |
| | | | | 1402) | | | | | | |
| Q | L2 | D | QL2D | DIQMT | QSIS | LNWY | AAS | SLQSGVPS | QDS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1409) | NO: | | (SEQ ID | NO: | NO: |
| | | | | NO: | | 1410) | | NO: 1411) | 1412) | 1413) |
| | | | | 1408) | | | | | | |
| Q | L2 | E | QL2E | DIQMT | QSIS | LNWY | AAS | SLQSGVPS | QES | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | SEQ | (SEQ |
| | | | | ITCRAS | NO: | (SEQ ID | | TYYC | ID | ID |
| | | | | (SEQ ID | 1415) | NO: | | (SEQ ID | NO: | NO: |
| | | | | NO: | | 1416) | | NO: 1417) | 1418) | 1419) |
| | | | | 1414) | | | | | | |
| Q | L2 | G | QL2G | DIQMT | QSIS | LNWY | AAS | SLQSGVPS | QGS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1421) | NO: | | (SEQ ID | NO: | NO: |
| | | | | NO: | | 1422) | | NO: 1423) | 1424) | 1425) |
| | | | | 1420) | | | | | | |
| Q | L2 | H | QL2H | DIQMT | QSIS | LNWY | AAS | SLQSGVPS | QHS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1427) | NO: | | (SEQ ID | NO: | NO: |
| | | | | NO: | | 1428) | | NO: 1429) | 1430) | 1431) |
| | | | | 1426) | | | | | | |
| Q | L2 | I | QL2I | DIQMT | QSIS | LNWY | AAS | SLQSGVPS | QISY | GQGT |
| | | | | QSPSS | SY | QQKPG | | RFSGSGSG | STP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1433) | NO: | | (SEQ ID | NO: | NO: |
| | | | | NO: | | 1434) | | NO: 1435) | 1436) | 1437) |
| | | | | 1432) | | | | | | |
| Q | L2 | K | QL2K | DIQMT | QSIS | LNWY | AAS | SLQSGVPS | QKS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1439) | NO: | | (SEQ ID | NO: | NO: |
| | | | | NO: | | 1440) | | NO: 1441) | 1442) | 1443) |
| | | | | 1438) | | | | | | |
| Q | L2 | L | QL2L | DIQMT | QSIS | LNWY | AAS | SLQSGVPS | QLS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1445) | NO: | | (SEQ ID | NO: | NO: |
| | | | | NO: | | 1445) | | NO: 1446) | 1447) | 1448) |
| | | | | 1444) | | | | | | |
| Q | L2 | M | QL2M | DIQMT | QSIS | LNWY | AAS | SLQSGVPS | QMS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1450) | NO: | | (SEQ ID | NO: | NO: |
| | | | | NO: | | 1451) | | NO: 1452) | 1453) | 1454) |
| | | | | 1449) | | | | | | |
| Q | L2 | N | QL2N | DIQMT | QSIS | LNWY | AAS | SLQSGVPS | QNS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1456) | NO: | | (SEQ ID | NO: | NO: |
| | | | | NO: | | 1457) | | NO: 1458) | 1459) | 1460) |
| | | | | 1455) | | | | | | |
| Q | L2 | P | QL2P | DIQMT | QSIS | LNWY | AAS | SLQSGVPS | QPS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1462) | NO: | | (SEQ ID | NO: | NO: |
| | | | | NO: | | 1463) | | NO: 1464) | 1465) | 1466) |
| | | | | 1461) | | | | | | |
| Q | L2 | R | QL2R | DIQMT | QSIS | LNWY | AAS | SLQSGVPS | QRS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1468) | NO: | | (SEQ ID | NO: | NO: |
| | | | | NO: | | 1469) | | NO: 1470) | 1471) | 1472) |
| | | | | 1467) | | | | | | |
| Q | L2 | S | QL2S | DIQMT | QSIS | LNWY | AAS | SLQSGVPS | QSS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1474) | NO: | | (SEQ ID | NO: | NO: |
| | | | | NO: | | 1475) | | NO: 1476) | 1477) | 1478) |
| | | | | 1473) | | | | | | |
| Q | L2 | V | QL2V | DIQMT | QSIS | LNWY | AAS | SLQSGVPS | QVS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1480) | NO: | | (SEQ ID | NO: | NO: |
| | | | | NO: | | 1481) | | NO: 1482) | 1483) | 1484) |
| | | | | 1479) | | | | | | |
| Q | L2 | Y | QL2Y | DIQMT | QSIS | LNWY | AAS | SLQSGVPS | QYS | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1486) | NO: | | (SEQ ID | NO: | NO: |
| | | | | NO: | | 1487) | | NO: 1488) | 1489) | 1490) |
| | | | | 1485) | | | | | | |
| S | L3 | A | SL3A | DIQMT | QSIS | LNWY | AAS | SLQSGVPS | QQA | GQGT |
| | | | | QSPSS | SY | QQKPG | | RFSGSGSG | YSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1492) | NO: | | (SEQ ID | NO: | NO: |
| | | | | NO: | | 1493) | | NO:1494) | 1495) | 1496) |
| | | | | 1491) | | | | | | |
| Y | L4 | D | YL4D | DIQMT | QSIS | LNWY | AAS | SLQSGVPS | QQS | GQGT |
| | | | | QSPSS | SY | QQKPG | | RFSGSGSG | DSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1498) | NO: | | (SEQ ID | NO: | NO: |
| | | | | NO: | | 1499) | | NO: 1500) | 1501) | 1502) |
| | | | | 1497) | | | | | | |
| Y | L4 | G | YL4G | DIQMT | QSIS | LNWY | AAS | SLQSGVPS | QQS | GQGT |
| | | | | QSPSS | SY | QQKPG | | RFSGSGSG | GSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1504) | NO: | | (SEQ ID | NO: | NO: |
| | | | | NO: | | 1505) | | NO: 1506) | 1507) | 1508) |
| | | | | 1503) | | | | | | |
| Y | L4 | H | YL4H | DIQMT | QSIS | LNWY | AAS | SLQSGVPS | QQS | GQGT |
| | | | | QSPSS | SY | QQKPG | | RFSGSGSG | HSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1510) | NO: | | (SEQ ID | NO: | NO: |
| | | | | NO: | | 1511) | | NO: 1512) | 1513) | 1514) |
| | | | | 1509) | | | | | | |
| Y | L4 | I | YL4I | DIQMT | QSIS | LNWY | AAS | SLQSGVPS | QQSI | GQGT |
| | | | | QSPSS | SY | QQKPG | | RFSGSGSG | STP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1516) | NO | | (SEQ ID | NO: | NO: |
| | | | | NO: | | :1517) | | NO: 1518) | 1519) | 1520 |
| | | | | 1515) | | | | | | |
| Y | L4 | L | YL4L | DIQMT | QSIS | LNWY | AAS | SLQSGVPS | QQS | GQGT |
| | | | | QSPSS | SY | QQKPG | | RFSGSGSG | LSTP | KLEI |
| | | | | VSASV | (SEQ | KAPKL | | TDFTLTIS | WTF | K |
| | | | | GDRVT | ID | LIY(SE | | SLQPEDFA | (SEQ | (SEQ |
| | | | | ITCRAS | NO: | Q ID | | TYYC | ID | ID |
| | | | | (SEQ ID | 1522) | NO: | | (SEQ ID | NO: | NO: |
| | | | | NO: | | 1523) | | NO: 1524) | 1525) | 1526) |
| | | | | 1521) | | | | | | |
| Y | L4 | M | YL4M | DIQMT | QSIS | LNWY | AAS | SLQSGVPS | QQS | GQGT |
| | | | | QSPSS | SY | QQKPG | | RFSGSGSG | MST | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1527) | NO: | | (SEQ ID | ID | NO: |
| | | | | NO: | | 1528) | | NO: 1529) | NO: | 1531) |
| | | | | 1526) | | | | | 1530) | |
| Y | L4 | P | YL4P | DIQMT | QSIS | LNWY | AAS | SLQSGVPS | QQS | GQGT |
| | | | | QSPSS | SY | QQKPG | | RFSGSGSG | PSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1533) | NO: | | (SEQ ID | NO: | NO: |
| | | | | NO: | | 1534) | | NO: 1535) | 1536) | 1537) |
| | | | | 1532) | | | | | | |
| Y | L4 | Q | YL4Q | DIQMT | QSIS | LNWY | AAS | SLQSGVPS | QQS | GQGT |
| | | | | QSPSS | SY | QQKPG | | RFSGSGSG | QSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1539) | NO: | | (SEQ ID | NO: | NO: |
| | | | | NO: | | 1540) | | NO: 1541) | 1542) | 1543) |
| | | | | 1538) | | | | | | |
| Y | L4 | R | YL4R | DIQMT | QSIS | LNWY | AAS | SLQSGVPS | QQS | GQGT |
| | | | | QSPSS | SY | QQKPG | | RFSGSGSG | RSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1545) | NO: | | (SEQ ID | NO: | NO: |
| | | | | NO: | | 6154) | | NO: 1547) | 5148) | 1549) |
| | | | | 1544) | | | | | | |
| Y | L4 | S | YL4S | DIQMT | QSIS | LNWY | AAS | SLQSGVPS | QQS | GQGT |
| | | | | QSPSS | SY | QQKPG | | RFSGSGSG | SSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1551) | NO: | | (SEQ ID | NO: | NO: |
| | | | | NO: | | 1552) | | NO: 1553) | 1554) | 1555) |
| | | | | 1550) | | | | | | |
| Y | L4 | T | YL4T | DIQMT | QSIS | LNWY | AAS | SLQSGVPS | QQS | GQGT |
| | | | | QSPSS | SY | QQKPG | | RFSGSGSG | TSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1557) | NO: | | (SEQ ID | NO: | NO: |
| | | | | NO: | | 1558) | | NO: 1559) | 1560) | 1561) |
| | | | | 1556) | | | | | | |
| Y | L4 | V | YL4V | DIQMT | QSIS | LNWY | AAS | SLQSGVPS | QQS | GQGT |
| | | | | QSPSS | SY | QQKPG | | RFSGSGSG | VSTP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1563) | NO: | | (SEQ ID | NO: | NO: |
| | | | | NO: | | 1564) | | NO: 1565) | 1566) | 1567) |
| | | | | 1562) | | | | | | |
| Y | L4 | W | YL4W | DIQMT | QSIS | LNWY | AAS | SLQSGVPS | QQS | GQGT |
| | | | | QSPSS | SY | QQKPG | | RFSGSGSG | WST | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1569) | NO: | | (SEQ ID | ID | NO: |
| | | | | NO: | | 1570) | | NO: 1571) | NO: | 7153) |
| | | | | 1568) | | | | | 1572) | |
| T | L6 | Y | TL6Y | DIQMT | QSIS | LNWY | AAS | SLQSGVPS | QQS | GQGT |
| | | | | QSPSS | SY | QQKPG | | RFSGSGSG | YYT | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1575) | NO: | | (SEQ ID | ID | NO: |
| | | | | NO: | | 1576) | | NO: 1577) | NO: | 1579) |
| | | | | 1574) | | | | | 1578) | |
| T | L6 | A | TL6A | DIQMT | QSIS | LNWY | AAS | SLQSGVPS | QQS | GQGT |
| | | | | QSPSS | SY | QQKPG | | RFSGSGSG | YSA | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1581) | NO: | | (SEQ ID | ID | NO: |
| | | | | NO: | | 1582) | | NO: 1583) | NO: | 8155) |
| | | | | 1580) | | | | | 1584) | |
| T | L6 | C | TL6C | DIQMT | QSIS | LNWY | AAS | SLQSGVPS | QQS | GQGT |
| | | | | QSPSS | SY | QQKPG | | RFSGSGSG | YSC | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1587) | NO: | | (SEQ ID | ID | NO: |
| | | | | NO: | | 1588) | | NO: 1589) | NO: | 1591) |
| | | | | 1586) | | | | | 1590) | |
| T | L6 | D | TL6D | DIQMT | QSIS | LNWY | AAS | SLQSGVPS | QQS | GQGT |
| | | | | QSPSS | SY | QQKPG | | RFSGSGSG | YSD | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1593) | NO: | | (SEQ ID | ID | NO: |
| | | | | NO: | | 1594) | | NO: 1595) | NO: | 1597) |
| | | | | 1592) | | | | | 1596) | |
| T | L6 | F | TL6F | DIQMT | QSIS | LNWY | AAS | SLQSGVPS | QQS | GQGT |
| | | | | QSPSS | SY | QQKPG | | RFSGSGSG | YSFP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1599) | NO: | | (SEQ ID | NO: | NO: |
| | | | | NO: | | 1600) | | NO: 1601) | 1602) | 1603) |
| | | | | 1598) | | | | | | |
| T | L6 | H | TL6H | DIQMT | QSIS | LNWY | AAS | SLQSGVPS | QQS | GQGT |
| | | | | QSPSS | SY | QQKPG | | RFSGSGSG | YSH | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1605) | NO: | | (SEQ ID | ID | NO: |
| | | | | NO: | | 1606) | | NO: 1607) | NO: | 1609) |
| | | | | 1604) | | | | | 6108) | |
| T | L6 | I | TL6I | DIQMT | QSIS | LNWY | AAS | SLQSGVPS | QQS | GQGT |
| | | | | QSPSS | SY | QQKPG | | RFSGSGSG | YSIP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1611) | NO: | | (SEQ ID | NO: | NO: |
| | | | | NO: | | 1612) | | NO: 1613) | 1614) | 1615) |
| | | | | 1610) | | | | | | |
| T | L6 | K | TL6K | DIQMT | QSIS | LNWY | AAS | SLQSGVPS | QQS | GQGT |
| | | | | QSPSS | SY | QQKPG | | RFSGSGSG | YSK | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1617) | NO: | | (SEQ ID | ID | NO: |
| | | | | NO: | | 1618) | | NO: 1619) | NO: | 1621) |
| | | | | 1616) | | | | | 1620) | |
| T | L6 | L | TL6L | DIQMT | QSIS | LNWY | AAS | SLQSGVPS | QQS | GQGT |
| | | | | QSPSS | SY | QQKPG | | RFSGSGSG | YSLP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1623) | NO: | | (SEQ ID | NO: | NO: |
| | | | | NO: | | 1624) | | NO: 1625) | 6126) | 1627) |
| | | | | 1622) | | | | | | |
| T | L6 | M | TL6M | DIQMT | QSIS | LNWY | AAS | SLQSGVPS | QQS | GQGT |
| | | | | QSPSS | SY | QQKPG | | RFSGSGSG | YSM | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1629) | NO: | | (SEQ ID | ID | NO: |
| | | | | NO: | | 1630) | | NO: 1631) | NO: | 1633) |
| | | | | 1628) | | | | | 1632) | |
| T | L6 | N | TL6N | DIQMT | QSIS | LNWY | AAS | SLQSGVPS | QQS | GQGT |
| | | | | QSPSS | SY | QQKPG | | RFSGSGSG | YSN | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1635) | NO: | | (SEQ ID | ID | NO: |
| | | | | NO: | | 1636) | | NO: 1637) | NO: | 1639) |
| | | | | 1634) | | | | | 1638) | |
| T | L6 | P | TL6P | DIQMT | QSIS | LNWY | AAS | SLQSGVPS | QQS | GQGT |
| | | | | QSPSS | SY | QQKPG | | RFSGSGSG | YSPP | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1641) | NO: | | (SEQ ID | NO: | NO: |
| | | | | NO: | | 1642) | | NO: 1643) | 6144) | 1645) |
| | | | | 1640) | | | | | | |
| T | L6 | Q | TL6Q | DIQMT | QSIS | LNWY | AAS | SLQSGVPS | QQS | GQGT |
| | | | | QSPSS | SY | QQKPG | | RFSGSGSG | YSQ | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1647) | NO: | | (SEQ ID | ID | NO: |
| | | | | NO: | | 1648) | | NO: 1649) | NO: | 1651) |
| | | | | 1646) | | | | | 1650) | |
| T | L6 | R | TL6R | DIQMT | QSIS | LNWY | AAS | SLQSGVPS | QQS | GQGT |
| | | | | QSPSS | SY | QQKPG | | RFSGSGSG | YSR | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1653) | NO: | | (SEQ ID | ID | NO: |
| | | | | NO: | | 1654) | | NO: 1655) | NO: | 1657) |
| | | | | 1652) | | | | | 1656) | |
| T | L6 | V | TL6V | DIQMT | QSIS | LNWY | AAS | SLQSGVPS | QQS | GQGT |
| | | | | QSPSS | SY | QQKPG | | RFSGSGSG | YSV | KLEI |
| | | | | VSASV | (SEQ | KAPKL | | TDFTLTIS | PWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1659) | NO: | | (SEQ ID | ID | NO: |
| | | | | NO: | | 1660) | | NO: 1661) | NO: | 1663) |
| | | | | 1658) | | | | | 1662) | |
| P | L7 | D | PL7D | DIQMT | QSIS | LNWY | AAS | SLQSGVPS | QQS | GQGT |
| | | | | QSPSS | SY | QQKPG | | RFSGSGSG | YST | KLEI |
| | | | | VSASV | (SEQ | KAPKL | | TDFTLTIS | DWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1665) | NO: | | (SEQ ID | ID | NO: |
| | | | | NO: | | 1666) | | NO: 1667) | NO: | 1669) |
| | | | | 1664) | | | | | 1668) | |
| P | L7 | I | PL7I | DIQMT | QSIS | LNWY | AAS | SLQSGVPS | QQS | GQGT |
| | | | | QSPSS | SY | QQKPG | | RFSGSGSG | YSTI | KLEI |
| | | | | VSASV | (SEQ | KAPKL | | TDFTLTIS | WTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1671) | NO: | | (SEQ ID | NO: | NO: |
| | | | | NO: | | 1672) | | NO: 1673) | 1674) | 1675) |
| | | | | 1670) | | | | | | |
| P | L7 | L | PL7L | DIQMT | QSIS | LNWY | AAS | SLQSGVPS | QQS | GQGT |
| | | | | QSPSS | SY | QQKPG | | RFSGSGSG | YST | KLEI |
| | | | | VSASV | (SEQ | KAPKL | | TDFTLTIS | LWT | K(SE |
| | | | | GDRVT | ID | LIY | | SLQPEDFA | F | Q ID |
| | | | | ITCRAS | NO: | (SEQ ID | | TYYC | (SEQ | NO: |
| | | | | (SEQ ID | 1677) | NO: | | (SEQ ID | ID | 1681) |
| | | | | NO: | | 1678) | | NO: 1679) | NO: | |
| | | | | 1676) | | | | | 1680) | |
| P | L7 | M | PL7M | DIQMT | QSIS | LNWY | AAS | SLQSGVPS | QQS | GQGT |
| | | | | QSPSS | SY | QQKPG | | RFSGSGSG | YST | KLEI |
| | | | | VSASV | (SEQ | KAPKL | | TDFTLTIS | MWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1683) | NO: | | (SEQ ID | ID | NO: |
| | | | | NO: | | 1684) | | NO: 1685) | NO: | 1687) |
| | | | | 1682) | | | | | 1686) | |
| P | L7 | N | PL7N | DIQMT | QSIS | LNWY | AAS | SLQSGVPS | QQS | GQGT |
| | | | | QSPSS | SY | QQKPG | | RFSGSGSG | YST | KLEI |
| | | | | VSASV | (SEQ | KAPKL | | TDFTLTIS | NWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 6189) | NO: | | (SEQ ID | ID | NO: |
| | | | | NO: | | 1690) | | NO: 1691) | NO: | 1693) |
| | | | | 1688) | | | | | 1692) | |
| P | L7 | Q | PL7Q | DIQMT | QSIS | LNWY | AAS | SLQSGVPS | QQS | GQGT |
| | | | | QSPSS | SY | QQKPG | | RFSGSGSG | YST | KLEI |
| | | | | VSASV | (SEQ | KAPKL | | TDFTLTIS | QWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1695) | NO: | | (SEQ ID | ID | NO: |
| | | | | NO: | | 1696) | | NO: 1697) | NO: | 1699) |
| | | | | 1694) | | | | | 1698) | |
| P | L7 | V | PL7V | DIQMT | QSIS | LNWY | AAS | SLQSGVPS | QQS | GQGT |
| | | | | QSPSS | SY | QQKPG | | RFSGSGSG | YST | KLEI |
| | | | | VSASV | (SEQ | KAPKL | | TDFTLTIS | VWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1701) | NO: | | (SEQ ID | ID | NO: |
| | | | | NO: | | 1702) | | NO: 1703) | NO: | 1705) |
| | | | | 1700) | | | | | 1704) | |
| P | L7 | W | PL7W | DIQMT | QSIS | LNWY | AAS | SLQSGVPS | QQS | GQGT |
| | | | | QSPSS | SY | QQKPG | | RFSGSGSG | YST | KLEI |
| | | | | VSASV | (SEQ | KAPKL | | TDFTLTIS | WW | K |
| | | | | GDRVT | ID | LIY | | SLQPEDFA | TF | (SEQ |
| | | | | ITCRAS | NO: | (SEQ ID | | TYYC | (SEQ | ID |
| | | | | (SEQ ID | 1707) | NO: | | (SEQ ID | ID | NO: |
| | | | | NO: | | 1708) | | NO: 1709) | NO: | 1711) |
| | | | | 1706) | | | | | 1710) | |
| P | L7 | Y | PL7Y | DIQMT | QSIS | LNWY | AAS | SLQSGVPS | QQS | GQGT |
| | | | | QSPSS | SY | QQKPG | | RFSGSGSG | YST | KLEI |
| | | | | VSASV | (SEQ | KAPKL | | TDFTLTIS | YWT | K |
| | | | | GDRVT | ID | LIY | | SLQPEDFA | F | (SEQ |
| | | | | ITCRAS | NO: | (SEQ ID | | TYYC | (SEQ | ID |
| | | | | (SEQ ID | 1713) | NO: | | (SEQ ID | ID | NO: |
| | | | | NO: | | 1714) | | NO: 1715) | NO: | 1717) |
| | | | | 1712) | | | | | 1716) | |
| W | L8 | D | WL8D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | DTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1719) | NO: | | (SEQ ID | NO: | NO: |
| | | | | NO: | | 1720) | | NO: 1721) | 1722) | 1723) |
| | | | | 1718) | | | | | | |
| W | L8 | F | WL8F | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | FTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1725) | NO: | | (SEQ ID | NO: | NO: |
| | | | | NO: | | 1726) | | NO: 1727) | 1728) | 1729) |
| | | | | 1724) | | | | | | |
| W | L8 | H | WL8H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | HTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1731) | NO: | | (SEQ ID | NO: | NO: |
| | | | | NO: | | 1732) | | NO: 1733) | 1734) | 1735) |
| | | | | 1730) | | | | | | |
| W | L8 | I | WL8I | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | ITF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1737) | NO: | | (SEQ ID | NO: | NO: |
| | | | | NO: | | 1738) | | NO: 1739) | 1740) | 1741) |
| | | | | 1736) | | | | | | |
| W | L8 | K | WL8K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | KTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1743) | NO: | | (SEQ ID | NO: | NO: |
| | | | | NO: | | 1744) | | NO: 1745) | 1746) | 1747) |
| | | | | 1742) | | | | | | |
| W | L8 | L | WL8L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | LTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1749) | NO: | | (SEQ ID | NO: | NO: |
| | | | | NO: | | 1750) | | NO: 1751) | 1752) | 1753) |
| | | | | 1748) | | | | | | |
| W | L8 | M | WL8M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | MTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1755) | NO: | | (SEQ ID | NO: | NO: |
| | | | | NO: | | 1756) | | NO: 1757) | 1758) | 1759) |
| | | | | 1754) | | | | | | |
| W | L8 | P | WL8P | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | PTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1761) | NO: | | (SEQ ID | NO: | NO: |
| | | | | NO: | | 1762) | | NO: 1763) | 1764) | 1765) |
| | | | | 1760) | | | | | | |
| W | L8 | Q | WL8Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | QTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1767) | NO: | | (SEQ ID | NO: | NO: |
| | | | | NO: | | 1768) | | NO: 1769) | 1770) | 1771) |
| | | | | 1766) | | | | | | |
| W | L8 | R | WL8R | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | RTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1773) | NO: | | (SEQ ID | NO: | NO: |
| | | | | NO: | | 1774) | | NO: 1775) | 1776) | 1777) |
| | | | | 1772) | | | | | | |
| W | L8 | S | WL8S | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | STF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1779) | NO: | | (SEQ ID | NO: | NO: |
| | | | | NO: | | 1780) | | NO: 1781) | 1782) | 1783) |
| | | | | 1778) | | | | | | |
| W | L8 | T | WL8T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | TTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1785) | NO: | | (SEQ ID | NO: | NO: |
| | | | | NO: | | 1786) | | NO: 1787) | 1788) | 1789) |
| | | | | 1784) | | | | | | |
| W | L8 | Y | WL8Y | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | YTF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1791) | NO: | | (SEQ ID | NO: | NO: |
| | | | | NO: | | 1792) | | NO: 1793) | 1794) | 1795) |
| | | | | 17901) | | | | | | |
| T | L9 | D | TL9D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WDF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1797) | NO: | | (SEQ ID | NO: | NO: |
| | | | | NO: | | 8179) | | NO: 1799) | 1800) | 1801) |
| | | | | 1796) | | | | | | |
| T | L9 | E | TL9E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WEF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1803) | NO: | | (SEQ ID | NO: | NO: |
| | | | | NO: | | 4180) | | NO: 1805) | 1806) | 1807) |
| | | | | 1802) | | | | | | |
| T | L9 | F | TL9F | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WFF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1809) | NO: | | (SEQ ID | NO: | NO: |
| | | | | NO: | | 1810) | | NO: 1811) | 1812) | 1813) |
| | | | | 1808) | | | | | | |
| T | L9 | G | TL9G | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WGF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1815) | NO: | | (SEQ ID | NO: | NO: |
| | | | | NO: | | 1816) | | NO: 1817) | 1818) | 1819) |
| | | | | 1814) | | | | | | |
| T | L9 | H | TL9H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WHF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1821) | NO: | | (SEQ ID | NO: | NO: |
| | | | | NO: | | 1822) | | NO: 1823) | 1824) | 1825) |
| | | | | 1820) | | | | | | |
| T | L9 | I | TL9I | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WIF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1827) | NO: | | (SEQ ID | NO: | NO: |
| | | | | NO: | | 8182) | | NO: 1829) | 1830) | 1831) |
| | | | | 1826) | | | | | | |
| T | L9 | K | TL9K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WKF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 8133) | NO: | | (SEQ ID | NO: | NO: |
| | | | | NO: | | 1834) | | NO: 1835) | 1836) | 1837) |
| | | | | 1832) | | | | | | |
| T | L9 | M | TL9M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WMF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1839) | NO: | | (SEQ ID | NO: | NO: |
| | | | | NO: | | 1840) | | NO: 1841) | 1842) | 1843) |
| | | | | 1838) | | | | | | |
| T | L9 | N | TL9N | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WNF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1845) | NO: | | (SEQ ID | NO: | NO: |
| | | | | NO: | | 1846) | | NO: 1847) | 1848) | 1849) |
| | | | | 1844) | | | | | | |
| T | L9 | P | TL9P | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WPF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1851) | NO: | | (SEQ ID | NO: | NO: |
| | | | | NO: | | 1852) | | NO: 1853) | 1854) | 1855) |
| | | | | 1850) | | | | | | |
| T | L9 | Q | TL9Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WQF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1857) | NO: | | (SEQ ID | NO: | NO: |
| | | | | NO: | | 1858) | | NO: 1859) | 1860) | 1861) |
| | | | | 1856) | | | | | | |
| T | L9 | R | TL9R | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WRF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1863) | NO: | | (SEQ ID | NO: | NO: |
| | | | | NO: | | 1864) | | NO: 1865) | 1866) | 1867) |
| | | | | 1862) | | | | | | |
| T | L9 | S | TL9S | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WSF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1869) | NO: | | (SEQ ID | NO: | NO: |
| | | | | NO: | | 1870) | | NO: 1871) | 1872) | 1873) |
| | | | | 1868) | | | | | | |
| T | L9 | V | TL9V | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WVF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1875) | NO: | | (SEQ ID | NO: | NO: |
| | | | | NO: | | 1876) | | NO: 1877) | 1878) | 1879) |
| | | | | 1874) | | | | | | |
| T | L9 | W | TL9W | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WWF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1881) | NO: | | (SEQ ID | NO: | NO: |
| | | | | NO: | | 2188) | | NO: 1883) | 1884) | 1885) |
| | | | | 1880) | | | | | | |
| T | L9 | Y | TL9Y | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WYF | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1887) | NO: | | (SEQ ID | NO: | NO: |
| | | | | NO: | | 1888) | | NO: 1889) | 1890) | 1891) |
| | | | | 1886) | | | | | | |
| F | L10 | C | FL10C | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTC | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1893) | NO: | | (SEQ ID | NO: | NO: |
| | | | | NO: | | 1894) | | NO: 1895) | 1896) | 1897) |
| | | | | 1892) | | | | | | |
| F | L10 | D | FL10D | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTD | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1899) | NO: | | (SEQ ID | NO: | NO: |
| | | | | NO: | | 1900) | | NO: 1901) | 1902) | 1903) |
| | | | | 1898) | | | | | | |
| F | L10 | E | FL10E | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTE | K(SE |
| | | | | GDRVT | ID | LIY | | SLQPEDFA | (SEQ | Q ID |
| | | | | ITCRAS | NO: | (SEQ ID | | TYYC | ID | NO: |
| | | | | (SEQ ID | 1905) | NO: | | (SEQ ID | NO: | 1909) |
| | | | | NO: | | 1906) | | NO: 1907) | 1908) | |
| | | | | 1904) | | | | | | |
| F | L10 | H | FL10H | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTH | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1911) | NO: | | (SEQ ID | NO: | NO: |
| | | | | NO: | | 1912) | | NO: 1913) | 1914) | 1915) |
| | | | | 1910) | | | | | | |
| F | L10 | I | FL10I | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTI | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1917) | NO: | | (SEQ ID | NO: | NO: |
| | | | | NO: | | 1918) | | NO: 1919) | 1920) | 1921) |
| | | | | 1916) | | | | | | |
| F | L10 | K | FL10K | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTK | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1923) | NO: | | (SEQ ID | NO: | NO: |
| | | | | NO: | | 1924) | | NO: 1925) | 1926) | 1927) |
| | | | | 1922) | | | | | | |
| F | L10 | L | FL10L | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTL | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1929) | NO: | | (SEQ ID | NO: | NO: |
| | | | | NO: | | 1930) | | NO: 1931) | 1932) | 1933) |
| | | | | 1928) | | | | | | |
| F | L10 | M | FL10M | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTM | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1935) | NO: | | (SEQ ID | NO: | NO: |
| | | | | NO: | | 1936) | | NO: 1937) | 1938) | 1939) |
| | | | | 1934) | | | | | | |
| F | L10 | N | FL10N | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTN | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1941) | NO: | | (SEQ ID | NO: | NO: |
| | | | | NO: | | 1942) | | NO: 1943) | 1944) | 1945) |
| | | | | 1940) | | | | | | |
| F | L10 | P | FL10P | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTP | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1947) | NO: | | (SEQ ID | NO: | NO: |
| | | | | NO: | | 1948) | | NO: 1949) | 1950) | 1951) |
| | | | | 1946) | | | | | | |
| F | L10 | Q | FL10Q | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTQ | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1953) | NO: | | (SEQ ID | NO: | NO: |
| | | | | NO: | | 1954) | | NO: 1955) | 1956) | 1957) |
| | | | | 1952) | | | | | | |
| F | L10 | R | FL10R | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTR | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1959) | NO: | | (SEQ ID | NO: | NO: |
| | | | | NO: | | 1960) | | NO: 1961) | 1962) | 1963) |
| | | | | 1958) | | | | | | |
| F | L10 | S | FL10S | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTS | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1965) | NO: | | (SEQ ID | NO: | NO: |
| | | | | NO: | | 1966) | | NO: 1967) | 1968) | 1969) |
| | | | | 1964) | | | | | | |
| F | L10 | T | FL10T | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTT | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1971) | NO: | | (SEQ ID | NO: | NO: |
| | | | | NO: | | 1972) | | NO: 1973) | 1974) | 1975) |
| | | | | 1970) | | | | | | |
| F | L10 | V | FL10V | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTV | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1977) | NO: | | (SEQ ID | NO: | NO: |
| | | | | NO: | | 1978) | | NO: 1979) | 1980) | 1981) |
| | | | | 1976) | | | | | | |
| F | L10 | W | FL10W | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WT | K |
| | | | | GDRVT | ID | LIY | | SLQPEDFA | W | (SEQ |
| | | | | ITCRAS | NO: | (SEQ ID | | TYYC | (SEQ | ID |
| | | | | (SEQ ID | 1983) | NO: | | (SEQ ID | ID | NO: |
| | | | | NO: | | 4198) | | NO: 1985) | NO: | 1987) |
| | | | | 1982) | | | | | 1986) | |
| F | L10 | Y | FL10Y | DIQMT | QSIS | LNWY | AAS | SLQSGVPS | QQS | GQGT |
| | | | | QSPSS | SY | QQKPG | | RFSGSGSG | YSTP | KLEI |
| | | | | VSASV | (SEQ | KAPKL | | TDFTLTIS | WTY | K |
| | | | | GDRVT | ID | LIY | | SLQPEDFA | (SEQ | (SEQ |
| | | | | ITCRAS | NO: | (SEQ ID | | TYYC | ID | ID |
| | | | | (SEQ ID | 1989) | NO: | | (SEQ ID | NO: | NO: |
| | | | | NO: | | 1990) | | NO: 1991) | 1992) | 1993) |
| | | | | 1988) | | | | | | |

The light chain sequences can be followed by a linker, such as, LEGGGGSGGGGSGGGAS (SEQ ID NO: 1994). The heavy chain sequences can be attached to the C-terminal end of the linker. Heavy chain sequences are shown in Table 3. In some aspects, the heavy chain sequence is on the N-terminal end of the linker and the light chain sequence is on the C-terminal end of the linker.

Table 3 shows heavy chain sequences for the CD229 antigen binding domain sequences of Table 1. In some aspects, the heavy chain sequence of the CD229 antigen binding domain is the wild type sequence. In some aspects, the heavy chain sequence of the CD229 antigen binding domain has a substitution, mutation or deletion compared to wild type.

TABLE 3
CD229 antigen binding domain heavy chain sequences

| | | | | FR1 | CDR1 | FR2 | CDR2 | FR3 | | FR4 |
|---|---|---|---|---|---|---|---|---|---|---|
| | | | | (SEQ ID | (SEQ ID | (SEQ ID | (SEQ ID | (SEQ ID | | (SEQ ID |
| | | | | NO: | NO: | NO: | NO: | NO: | | NO: |
| Old | Pos | New | NAME | 1995) | 1996) | 1997) | 1998) | 1999) | CDR3 | 2000) |
| — | — | — | WT | QVQL | GFTF | MHW | ISW | GYADSAK | AKR | GQGT |
| | | | (2D3) | VESGG | DDY | VRQA | NSG | GRFTISRD | GNS | LVTV |
| | | | | GLVQP | A | PGKG | SI | NSKNTLY | NSFD | SS |
| | | | | GRSLR | (SEQ | LEWV | (SEQ | LQMNSLR | YW | (SEQ |
| | | | | LSCAA | ID | SG | ID | AEDTAVY | (SEQ | ID |
| | | | | S (SEQ | NO: | (SEQ | NO: | YC (SEQ ID | ID | NO: |
| | | | | ID | 1996) | ID | 1998) | NO: 1999) | NO: | 2000) |
| | | | | NO: | | NO: | | | 2001) | |
| | | | | 1995) | | 1997) | | | | |
| A | H1 | C | AH1C | QVQL | GFTF | MHW | ISW | GYADSAK | CKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2002) | |
| | | | | 1995) | | | | | | |
| A | H1 | D | AH1D | QVQL | GFTF | MHW | ISW | GYADSAK | DKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2003) | |
| | | | | 1995) | | | | | | |
| A | H1 | E | AH1E | QVQL | GFTF | MHW | ISW | GYADSAK | EKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2004) | |
| | | | | 1995) | | | | | | |
| A | H1 | F | AH1F | QVQL | GFTF | MHW | ISW | GYADSAK | FKRG | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2005) | |
| | | | | 1995) | | | | | | |
| A | H1 | G | AH1G | QVQL | GFTF | MHW | ISW | GYADSAK | GKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2006) | |
| | | | | 1995) | | | | | | |
| A | H1 | H | AH1H | QVQL | GFTF | MHW | ISW | GYADSAK | HKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2007) | |
| | | | | 1995) | | | | | | |
| A | H1 | I | AH1I | QVQL | GFTF | MHW | ISW | GYADSAK | IKRG | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2008) | |
| | | | | 1995) | | | | | | |
| A | H1 | K | AH1K | QVQL | GFTF | MHW | ISW | GYADSAK | KKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2009) | |
| | | | | 1995) | | | | | | |
| A | H1 | L | AH1L | QVQL | GFTF | MHW | ISW | GYADSAK | LKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2010) | |
| | | | | 1995) | | | | | | |
| A | H1 | M | AH1M | QVQL | GFTF | MHW | ISW | GYADSAK | MKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2011) | |
| | | | | 1995) | | | | | | |
| A | H1 | N | AH1N | QVQL | GFTF | MHW | ISW | GYADSAK | NKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2012) | |
| | | | | 1995) | | | | | | |
| A | H1 | P | AH1P | QVQL | GFTF | MHW | ISW | GYADSAK | PKRG | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2013) | |
| | | | | 1995) | | | | | | |
| A | H1 | S | AH1S | QVQL | GFTF | MHW | ISW | GYADSAK | SKRG | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2014) | |
| | | | | 1995) | | | | | | |
| A | H1 | T | AH1T | QVQL | GFTF | MHW | ISW | GYADSAK | TKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2015) | |
| | | | | 1995) | | | | | | |
| A | H1 | V | AH1V | QVQL | GFTF | MHW | ISW | GYADSAK | VKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2016) | |
| | | | | 1995) | | | | | | |
| A | H1 | W | AH1W | QVQL | GFTF | MHW | ISW | GYADSAK | WKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2017) | |
| | | | | 1995) | | | | | | |
| A | H1 | Y | AH1Y | QVQL | GFTF | MHW | ISW | GYADSAK | YKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2018) | |
| | | | | 1995) | | | | | | |
| K | H2 | C | KH2C | QVQL | GFTF | MHW | ISW | GYADSAK | ACR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2019) | |
| | | | | 1995) | | | | | | |
| K | H2 | D | KH2D | QVQL | GFTF | MHW | ISW | GYADSAK | ADR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2020) | |
| | | | | 1995) | | | | | | |
| K | H2 | E | KH2E | QVQL | GFTF | MHW | ISW | GYADSAK | AER | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2021) | |
| | | | | 1995) | | | | | | |
| K | H2 | F | KH2F | QVQL | GFTF | MHW | ISW | GYADSAK | AFRG | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S(SEQ | | | | YC | ID | |
| | | | | ID | | | | | NO: | |
| | | | | NO: | | | | | 2022) | |
| | | | | 1995) | | | | | | |
| K | H2 | G | KH2G | QVQL | GFTF | MHW | ISW | GYADSAK | AGR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2023) | |
| | | | | 1995) | | | | | | |
| K | H2 | H | KH2H | QVQL | GFTF | MHW | ISW | GYADSAK | AHR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2024) | |
| | | | | 1995) | | | | | | |
| K | H2 | I | KH2I | QVQL | GFTF | MHW | ISW | GYADSAK | AIRG | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2025) | |
| | | | | 1995) | | | | | | |
| K | H2 | L | KH2L | QVQL | GFTF | MHW | ISW | GYADSAK | ALR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2026) | |
| | | | | 1995) | | | | | | |
| K | H2 | M | KH2M | QVQL | GFTF | MHW | ISW | GYADSAK | AMR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2027) | |
| | | | | 1995) | | | | | | |
| K | H2 | N | KH2N | QVQL | GFTF | MHW | ISW | GYADSAK | ANR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2028) | |
| | | | | 1995) | | | | | | |
| K | H2 | P | KH2P | QVQL | GFTF | MHW | ISW | GYADSAK | APRG | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2029) | |
| | | | | 1995) | | | | | | |
| K | H2 | Q | KH2Q | QVQL | GFTF | MHW | ISW | GYADSAK | AQR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2030) | |
| | | | | 1995) | | | | | | |
| K | H2 | R | KH2R | QVQL | GFTF | MHW | ISW | GYADSAK | ARR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2031) | |
| | | | | 1995) | | | | | | |
| K | H2 | S | KH2S | QVQL | GFTF | MHW | ISW | GYADSAK | ASRG | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2032) | |
| | | | | 1995) | | | | | | |
| K | H2 | V | KH2V | QVQL | GFTF | MHW | ISW | GYADSAK | AVR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2033) | |
| | | | | 1995) | | | | | | |
| K | H2 | W | KH2W | QVQL | GFTF | MHW | ISW | GYADSAK | AWR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2034) | |
| | | | | 1995) | | | | | | |
| R | H3 | A | RH3A | QVQL | GFTF | MHW | ISW | GYADSAK | AKA | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2035) | |
| | | | | 1995) | | | | | | |
| R | H3 | C | RH3C | QVQL | GFTF | MHW | ISW | GYADSAK | AKC | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 0236) | |
| | | | | 1995) | | | | | | |
| R | H3 | G | RH3G | QVQL | GFTF | MHW | ISW | GYADSAK | AKG | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2037) | |
| | | | | 1995) | | | | | | |
| R | H3 | H | RH3H | QVQL | GFTF | MHW | ISW | GYADSAK | AKH | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2038) | |
| | | | | 1995) | | | | | | |
| R | H3 | I | RH3I | QVQL | GFTF | MHW | ISW | GYADSAK | AKIG | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2039) | |
| | | | | 1995) | | | | | | |
| R | H3 | K | RH3K | QVQL | GFTF | MHW | ISW | GYADSAK | AKK | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2040) | |
| | | | | 1995) | | | | | | |
| R | H3 | L | RH3L | QVQL | GFTF | MHW | ISW | GYADSAK | AKL | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2041) | |
| | | | | 1995) | | | | | | |
| R | H3 | M | RH3M | QVQL | GFTF | MHW | ISW | GYADSAK | AKM | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2042) | |
| | | | | 1995) | | | | | | |
| R | H3 | N | RH3N | QVQL | GFTF | MHW | ISW | GYADSAK | AKN | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2043) | |
| | | | | 1995) | | | | | | |
| R | H3 | P | RH3P | QVQL | GFTF | MHW | ISW | GYADSAK | AKP | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2044) | |
| | | | | 1995) | | | | | | |
| R | H3 | Q | RH3Q | QVQL | GFTF | MHW | ISW | GYADSAK | AKQ | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 0245) | |
| | | | | 1995) | | | | | | |
| R | H3 | S | RH3S | QVQL | GFTF | MHW | ISW | GYADSAK | AKS | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2046) | |
| | | | | 1995) | | | | | | |
| R | H3 | T | RH3T | QVQL | GFTF | MHW | ISW | GYADSAK | AKT | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2047) | |
| | | | | 1995) | | | | | | |
| R | H3 | V | RH3V | QVQL | GFTF | MHW | ISW | GYADSAK | AKV | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2048) | |
| | | | | 1995) | | | | | | |
| R | H3 | W | RH3W | QVQL | GFTF | MHW | ISW | GYADSAK | AKW | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2049) | |
| | | | | 1995) | | | | | | |
| R | H3 | Y | RH3Y | QVQL | GFTF | MHW | ISW | GYADSAK | AKY | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2050) | |
| | | | | 1995) | | | | | | |
| G | H4 | A | GH4A | QVQL | GFTF | MHW | ISW | GYADSAK | AKR | GQGT |
| | | | | VESGG | DDY | VRQA | NSG | GRFTISRD | ANS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2051) | |
| | | | | 1995) | | | | | | |
| G | H4 | C | GH4C | QVQL | GFTF | MHW | ISW | GYADSAK | AKR | GQGT |
| | | | | VESGG | DDY | VRQA | NSG | GRFTISRD | CNSN | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2052) | |
| | | | | 1995) | | | | | | |
| G | H4 | D | GH4D | QVQL | GFTF | MHW | ISW | GYADSAK | AKR | GQGT |
| | | | | VESGG | DDY | VRQA | NSG | GRFTISRD | DNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2053) | |
| | | | | 1995) | | | | | | |
| G | H4 | E | GH4E | QVQL | GFTF | MHW | ISW | GYADSAK | AKR | GQGT |
| | | | | VESGG | DDY | VRQA | NSG | GRFTISRD | ENSN | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2054) | |
| | | | | 1995) | | | | | | |
| G | H4 | F | GH4F | QVQL | GFTF | MHW | ISW | GYADSAK | AKRF | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2055) | |
| | | | | 1995) | | | | | | |
| G | H4 | H | GH4H | QVQL | GFTF | MHW | ISW | GYADSAK | AKR | GQGT |
| | | | | VESGG | DDY | VRQA | NSG | GRFTISRD | HNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2056) | |
| | | | | 1995) | | | | | | |
| G | H4 | I | GH4I | QVQL | GFTF | MHW | ISW | GYADSAK | AKRI | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2057) | |
| | | | | 1995) | | | | | | |
| G | H4 | K | GH4K | QVQL | GFTF | MHW | ISW | GYADSAK | AKR | GQGT |
| | | | | VESGG | DDY | VRQA | NSG | GRFTISRD | KNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2058) | |
| | | | | 1995) | | | | | | |
| G | H4 | L | GH4L | QVQL | GFTF | MHW | ISW | GYADSAK | AKR | GQGT |
| | | | | VESGG | DDY | VRQA | NSG | GRFTISRD | LNSN | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2059) | |
| | | | | 1995) | | | | | | |
| G | H4 | M | GH4M | QVQL | GFTF | MHW | ISW | GYADSAK | AKR | GQGT |
| | | | | VESGG | DDY | VRQA | NSG | GRFTISRD | MNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2060) | |
| | | | | 1995) | | | | | | |
| G | H4 | N | GH4N | QVQL | GFTF | MHW | ISW | GYADSAK | AKR | GQGT |
| | | | | VESGG | DDY | VRQA | NSG | GRFTISRD | NNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S | | | | YC | ID | |
| | | | | | | | | | NO: | |
| | | | | | | | | | 2061) | |
| G | H4 | P | GH4P | QVQL | GFTF | MHW | ISW | GYADSAK | AKRP | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2062) | |
| | | | | 1995) | | | | | | |
| G | H4 | Q | GH4Q | QVQL | GFTF | MHW | ISW | GYADSAK | AKR | GQGT |
| | | | | VESGG | DDY | VRQA | NSG | GRFTISRD | QNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2063) | |
| | | | | 1995) | | | | | | |
| G | H4 | R | GH4R | QVQL | GFTF | MHW | ISW | GYADSAK | AKR | GQGT |
| | | | | VESGG | DDY | VRQA | NSG | GRFTISRD | RNSN | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2064) | |
| | | | | 1995) | | | | | | |
| G | H4 | S | GH4S | QVQL | GFTF | MHW | ISW | GYADSAK | AKRS | GQGT |
| | | | | VESGG | DDY | VRQA | NSG | GRFTISRD | NSNS | LVTV |
| | | | | GLVQP | A | PGKG | SI | NSKNTLY | FDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2065) | |
| | | | | 1995) | | | | | | |
| G | H4 | T | GH4T | QVQL | GFTF | MHW | ISW | GYADSAK | AKR | GQGT |
| | | | | VESGG | DDY | VRQA | NSG | GRFTISRD | TNSN | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2066) | |
| | | | | 1995) | | | | | | |
| G | H4 | V | GH4V | QVQL | GFTF | MHW | ISW | GYADSAK | AKR | GQGT |
| | | | | VESGG | DDY | VRQA | NSG | GRFTISRD | VNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2067) | |
| | | | | 1995) | | | | | | |
| G | H4 | Y | GH4Y | QVQL | GFTF | MHW | ISW | GYADSAK | AKR | GQGT |
| | | | | VESGG | DDY | VRQA | NSG | GRFTISRD | YNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2068) | |
| | | | | 1995) | | | | | | |
| N | H5 | A | NH5A | QVQL | GFTF | MHW | ISW | GYADSAK | AKR | GQGT |
| | | | | VESGG | DDY | VRQA | NSG | GRFTISRD | GAS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2069) | |
| | | | | 1995) | | | | | | |
| N | H5 | C | NH5C | QVQL | GFTF | MHW | ISW | GYADSAK | AKR | GQGT |
| | | | | VESGG | DDY | VRQA | NSG | GRFTISRD | GCSN | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 0270) | |
| | | | | 1995) | | | | | | |
| N | H5 | D | NH5D | QVQL | GFTF | MHW | ISW | GYADSAK | AKR | GQGT |
| | | | | VESGG | DDY | VRQA | NSG | GRFTISRD | GDS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2071) | |
| | | | | 1995) | | | | | | |
| N | H5 | E | NH5E | QVQL | GFTF | MHW | ISW | GYADSAK | AKR | GQGT |
| | | | | VESGG | DDY | VRQA | NSG | GRFTISRD | GESN | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2072) | |
| | | | | 1995) | | | | | | |
| N | H5 | H | NH5H | QVQL | GFTF | MHW | ISW | GYADSAK | AKR | GQGT |
| | | | | VESGG | DDY | VRQA | NSG | GRFTISRD | GHS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2073) | |
| | | | | 1995) | | | | | | |
| N | H5 | I | NH5I | QVQL | GFTF | MHW | ISW | GYADSAK | AKR | GQGT |
| | | | | VESGG | DDY | VRQA | NSG | GRFTISRD | GISN | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2074) | |
| | | | | 1995) | | | | | | |
| N | H5 | L | NH5L | QVQL | GFTF | MHW | ISW | GYADSAK | AKR | GQGT |
| | | | | VESGG | DDY | VRQA | NSG | GRFTISRD | GLSN | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2075) | |
| | | | | 1995) | | | | | | |
| N | H5 | M | NH5M | QVQL | GFTF | MHW | ISW | GYADSAK | AKR | GQGT |
| | | | | VESGG | DDY | VRQA | NSG | GRFTISRD | GMS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2076) | |
| | | | | 1995) | | | | | | |
| N | H5 | P | NH5P | QVQL | GFTF | MHW | ISW | GYADSAK | AKR | GQGT |
| | | | | VESGG | DDY | VRQA | NSG | GRFTISRD | GPSN | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 0277) | |
| | | | | 1995) | | | | | | |
| N | H5 | T | NH5T | QVQL | GFTF | MHW | ISW | GYADSAK | AKR | GQGT |
| | | | | VESGG | DDY | VRQA | NSG | GRFTISRD | GTSN | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2078) | |
| | | | | 1995) | | | | | | |
| N | H5 | V | NH5V | QVQL | GFTF | MHW | ISW | GYADSAK | AKR | GQGT |
| | | | | VESGG | DDY | VRQA | NSG | GRFTISRD | GVS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2079) | |
| | | | | 1995) | | | | | | |
| N | H5 | W | NH5W | QVQL | GFTF | MHW | ISW | GYADSAK | AKR | GQGT |
| | | | | VESGG | DDY | VRQA | NSG | GRFTISRD | GWS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2080) | |
| | | | | 1995) | | | | | | |
| S | H6 | A | SH6A | QVQL | GFTF | MHW | ISW | GYADSAK | AKR | GQGT |
| | | | | VESGG | DDY | VRQA | NSG | GRFTISRD | GNA | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2081) | |
| | | | | 1995) | | | | | | |
| S | H6 | C | SH6C | QVQL | GFTF | MHW | ISW | GYADSAK | AKR | GQGT |
| | | | | VESGG | DDY | VRQA | NSG | GRFTISRD | GNC | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2082) | |
| | | | | 1995) | | | | | | |
| S | H6 | D | SH6D | QVQL | GFTF | MHW | ISW | GYADSAK | AKR | GQGT |
| | | | | VESGG | DDY | VRQA | NSG | GRFTISRD | GND | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2083) | |
| | | | | 1995) | | | | | | |
| S | H6 | E | SH6E | QVQL | GFTF | MHW | ISW | GYADSAK | AKR | GQGT |
| | | | | VESGG | DDY | VRQA | NSG | GRFTISRD | GNE | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2084) | |
| | | | | 1995) | | | | | | |
| S | H6 | F | SH6F | QVQL | GFTF | MHW | ISW | GYADSAK | AKR | GQGT |
| | | | | VESGG | DDY | VRQA | NSG | GRFTISRD | GNF | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2085) | |
| | | | | 1995) | | | | | | |
| S | H6 | H | SH6H | QVQL | GFTF | MHW | ISW | GYADSAK | AKR | GQGT |
| | | | | VESGG | DDY | VRQA | NSG | GRFTISRD | GNH | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2086) | |
| | | | | 1995) | | | | | | |
| S | H6 | K | SH6K | QVQL | GFTF | MHW | ISW | GYADSAK | AKR | GQGT |
| | | | | VESGG | DDY | VRQA | NSG | GRFTISRD | GNK | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2087) | |
| | | | | 1995) | | | | | | |
| S | H6 | L | SH6L | QVQL | GFTF | MHW | ISW | GYADSAK | AKR | GQGT |
| | | | | VESGG | DDY | VRQA | NSG | GRFTISRD | GNL | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2088) | |
| | | | | 1995) | | | | | | |
| S | H6 | N | SH6N | QVQL | GFTF | MHW | ISW | GYADSAK | AKR | GQGT |
| | | | | VESGG | DDY | VRQA | NSG | GRFTISRD | GNN | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2089) | |
| | | | | 1995) | | | | | | |
| S | H6 | P | SH6P | QVQL | GFTF | MHW | ISW | GYADSAK | AKR | GQGT |
| | | | | VESGG | DDY | VRQA | NSG | GRFTISRD | GNP | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2090) | |
| | | | | 1995) | | | | | | |
| S | H6 | Q | SH6Q | QVQL | GFTF | MHW | ISW | GYADSAK | AKR | GQGT |
| | | | | VESGG | DDY | VRQA | NSG | GRFTISRD | GNQ | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 0291) | |
| | | | | 1995) | | | | | | |
| S | H6 | T | SH6T | QVQL | GFTF | MHW | ISW | GYADSAK | AKR | GQGT |
| | | | | VESGG | DDY | VRQA | NSG | GRFTISRD | GNT | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2092) | |
| | | | | 1995) | | | | | | |
| S | H6 | W | SH6W | QVQL | GFTF | MHW | ISW | GYADSAK | AKR | GQGT |
| | | | | VESGG | DDY | VRQA | NSG | GRFTISRD | GNW | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2093) | |
| | | | | 1995) | | | | | | |
| S | H6 | Y | SH6Y | QVQL | GFTF | MHW | ISW | GYADSAK | AKR | GQGT |
| | | | | VESGG | DDY | VRQA | NSG | GRFTISRD | GNY | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2094) | |
| | | | | 1995) | | | | | | |
| N | H7 | A | NH7A | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | ASFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2095) | |
| | | | | 1995) | | | | | | |
| N | H7 | C | NH7C | QVQL | GFTF | MHW | ISW | GYADSAK | AKR | GQGT |
| | | | | VESGG | DDY | VRQA | NSG | GRFTISRD | GNSC | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2096) | |
| | | | | 1995) | | | | | | |
| N | H7 | D | NH7D | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | DSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2097) | |
| | | | | 1995) | | | | | | |
| N | H7 | E | NH7E | QVQL | GFTF | MHW | ISW | GYADSAK | AKR | GQGT |
| | | | | VESGG | DDY | VRQA | NSG | GRFTISRD | GNSE | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2098) | |
| | | | | 1995) | | | | | | |
| N | H7 | F | NH7F | QVQL | GFTF | MHW | ISW | GYADSAK | AKR | GQGT |
| | | | | VESGG | DDY | VRQA | NSG | GRFTISRD | GNSF | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2099) | |
| | | | | 1995) | | | | | | |
| N | H7 | I | NH7I | QVQL | GFTF | MHW | ISW | GYADSAK | AKR | GQGT |
| | | | | VESGG | DDY | VRQA | NSG | GRFTISRD | GNSI | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2100) | |
| | | | | 1995) | | | | | | |
| N | H7 | K | NH7K | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | KSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2101) | |
| | | | | 1995) | | | | | | |
| N | H7 | L | NH7L | QVQL | GFTF | MHW | ISW | GYADSAK | AKR | GQGT |
| | | | | VESGG | DDY | VRQA | NSG | GRFTISRD | GNSL | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2102) | |
| | | | | 1995) | | | | | | |
| N | H7 | M | NH7M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | MSF | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 1203) | |
| | | | | 1995) | | | | | | |
| N | H7 | Q | NH7Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | QSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2104) | |
| | | | | 1995) | | | | | | |
| N | H7 | R | NH7R | QVQL | GFTF | MHW | ISW | GYADSAK | AKR | GQGT |
| | | | | VESGG | DDY | VRQA | NSG | GRFTISRD | GNSR | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2105) | |
| | | | | 1995) | | | | | | |
| N | H7 | S | NH7S | QVQL | GFTF | MHW | ISW | GYADSAK | AKR | GQGT |
| | | | | VESGG | DDY | VRQA | NSG | GRFTISRD | GNSS | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2106) | |
| | | | | 1995) | | | | | | |
| N | H7 | T | NH7T | QVQL | GFTF | MHW | ISW | GYADSAK | AKR | GQGT |
| | | | | VESGG | DDY | VRQA | NSG | GRFTISRD | GNST | LVTV |
| | | | | GLVQP | A | PGKG | SI | NSKNTLY | SFDY | SS |
| | | | | GRSLR | | LEWV | | LQMNSLR | W | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2107) | |
| | | | | 1995) | | | | | | |
| N | H7 | W | NH7W | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | WSF | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2108) | |
| | | | | 1995) | | | | | | |
| N | H7 | Y | NH7Y | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | YSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2109) | |
| | | | | 1995) | | | | | | |
| S | H8 | A | SH8A | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NAF | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2110) | |
| | | | | 1995) | | | | | | |
| S | H8 | C | SH8C | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NCFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2111) | |
| | | | | 1995) | | | | | | |
| S | H8 | D | SH8D | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NDF | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2112) | |
| | | | | 1995) | | | | | | |
| S | H8 | E | SH8E | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NEFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2113) | |
| | | | | 1995) | | | | | | |
| S | H8 | F | SH8F | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NFFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2114) | |
| | | | | 1995) | | | | | | |
| S | H8 | G | SH8G | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NGF | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2115) | |
| | | | | 1995) | | | | | | |
| S | H8 | H | SH8H | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NHF | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2116) | |
| | | | | 1995) | | | | | | |
| S | H8 | K | SH8K | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NKF | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2117) | |
| | | | | 1995) | | | | | | |
| S | H8 | N | SH8N | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NNF | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2118) | |
| | | | | 1995) | | | | | | |
| S | H8 | Q | SH8Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NQF | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2119) | |
| | | | | 1995) | | | | | | |
| S | H8 | R | SH8R | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NRFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2120) | |
| | | | | 1995) | | | | | | |
| S | H8 | T | SH8T | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NTFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2121) | |
| | | | | 1995) | | | | | | |
| S | H8 | V | SH8V | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NVF | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2122) | |
| | | | | 1995) | | | | | | |
| S | H8 | W | SH8W | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NWF | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2123) | |
| | | | | 1995) | | | | | | |
| F | H9 | A | FH9A | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSA | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2124) | |
| | | | | 1995) | | | | | | |
| F | H9 | D | FH9D | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSD | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2125) | |
| | | | | 1995) | | | | | | |
| F | H9 | E | FH9E | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSED | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2126) | |
| | | | | 1995) | | | | | | |
| F | H9 | G | FH9G | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSG | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2127) | |
| | | | | 1995) | | | | | | |
| F | H9 | H | FH9H | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSH | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2128) | |
| | | | | 1995) | | | | | | |
| F | H9 | K | FH9K | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSK | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2129) | |
| | | | | 1995) | | | | | | |
| F | H9 | L | FH9L | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSLD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2130) | |
| | | | | 1995) | | | | | | |
| F | H9 | M | FH9M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSM | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2131) | |
| | | | | 1995) | | | | | | |
| F | H9 | N | FH9N | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSN | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2132) | |
| | | | | 1995) | | | | | | |
| F | H9 | P | FH9P | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSPD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2133) | |
| | | | | 1995) | | | | | | |
| F | H9 | Q | FH9Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSQ | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2134) | |
| | | | | 1995) | | | | | | |
| F | H9 | T | FH9T | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSTD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2135) | |
| | | | | 1995) | | | | | | |
| F | H9 | W | FH9W | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSW | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2136) | |
| | | | | 1995) | | | | | | |
| F | H9 | Y | FH9Y | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSY | SS |
| | | | | GRSLR | | LEWV | | LQMNSLR | DYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2137) | |
| | | | | 1995) | | | | | | |
| D | H10 | A | DH10A | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFA | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 1238) | |
| | | | | 1995) | | | | | | |
| D | H10 | C | DH10C | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFC | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2139) | |
| | | | | 1995) | | | | | | |
| D | H10 | E | DH10E | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFE | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2140) | |
| | | | | 1995) | | | | | | |
| D | H10 | F | DH10F | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFF | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2141) | |
| | | | | 1995) | | | | | | |
| D | H10 | G | DH10G | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFG | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2142) | |
| | | | | 1995) | | | | | | |
| D | H10 | K | DH10K | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFK | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2143) | |
| | | | | 1995) | | | | | | |
| D | H10 | L | DH10L | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFL | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2144) | |
| | | | | 1995) | | | | | | |
| D | H10 | M | DH10M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSF | SS |
| | | | | GRSLR | | LEWV | | LQMNSLR | MYW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2145) | |
| | | | | 1995) | | | | | | |
| D | H10 | N | DH10N | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFN | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2146) | |
| | | | | 1995) | | | | | | |
| D | H10 | P | DH10P | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFP | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2147) | |
| | | | | 1995) | | | | | | |
| D | H10 | Q | DH10Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFQ | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2148) | |
| | | | | 1995) | | | | | | |
| D | H10 | R | DH10R | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFR | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2149) | |
| | | | | 1995) | | | | | | |
| D | H10 | T | DH10T | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFT | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2150) | |
| | | | | 1995) | | | | | | |
| D | H10 | V | DH10V | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFV | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2151) | |
| | | | | 1995) | | | | | | |
| D | H10 | Y | DH10Y | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFY | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2152) | |
| | | | | 1995) | | | | | | |
| Y | H11 | A | YH11A | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | AW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2153) | |
| | | | | 1995) | | | | | | |
| Y | H11 | C | YH11C | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | CW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2154) | |
| | | | | 1995) | | | | | | |
| Y | H11 | D | YH11D | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | DW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2155) | |
| | | | | 1995) | | | | | | |
| Y | H11 | E | YH11E | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | EW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2156) | |
| | | | | 1995) | | | | | | |
| Y | H11 | F | YH11F | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | FW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2157) | |
| | | | | 1995) | | | | | | |
| Y | H11 | G | YH11G | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | GW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2158) | |
| | | | | 1995) | | | | | | |
| Y | H111 | K | YH11K | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | KW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2159) | |
| | | | | 1995) | | | | | | |
| Y | H11 | L | YH11L | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | LW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2160) | |
| | | | | 1995) | | | | | | |
| Y | H11 | M | YH11M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | MW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2161) | |
| | | | | 1995) | | | | | | |
| Y | H11 | Q | YH11Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | QW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2162) | |
| | | | | 1995) | | | | | | |
| Y | H11 | R | YH11R | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | RW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2163) | |
| | | | | 1995) | | | | | | |
| Y | H11 | S | YH11S | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | SW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2164) | |
| | | | | 1995) | | | | | | |
| Y | H11 | T | YH11T | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | TW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2165) | |
| | | | | 1995) | | | | | | |
| Y | H11 | V | YH11V | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | VW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2166) | |
| | | | | 1995) | | | | | | |
| Y | H11 | W | YH11W | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | WW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2167) | |
| | | | | 1995) | | | | | | |
| W | H12 | A | WH12A | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YA | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2168) | |
| | | | | 1995) | | | | | | |
| W | H12 | C | WH12C | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YC | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2169) | |
| | | | | 1995) | | | | | | |
| W | H12 | E | WH12E | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YE | |
| | | | | LSCAA | | SC | | AEDTAVY | (SEQ | |
| | | | | S (SEQ | | | | YC | ID | |
| | | | | ID | | | | | NO: | |
| | | | | NO: | | | | | 2170) | |
| | | | | 1995) | | | | | | |
| W | H12 | F | WH12F | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YF | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2171) | |
| | | | | 1995) | | | | | | |
| W | H12 | G | WH12G | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YG | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2172) | |
| | | | | 1995) | | | | | | |
| W | H12 | H | WH12H | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YH | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2173) | |
| | | | | 1995) | | | | | | |
| W | H12 | I | WH12I | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YI | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2174) | |
| | | | | 1995) | | | | | | |
| W | H12 | K | WH12K | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YK | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2175) | |
| | | | | 1995) | | | | | | |
| W | H12 | L | WH12L | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YL | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2176) | |
| | | | | 1995) | | | | | | |
| W | H12 | M | WH12M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YM | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2177) | |
| | | | | 1995) | | | | | | |
| W | H12 | N | WH12N | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YN | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2178) | |
| | | | | 1995) | | | | | | |
| W | H12 | Q | WH12Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YQ | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2179) | |
| | | | | 5199) | | | | | | |
| W | H12 | R | WH12R | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YR | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2180) | |
| | | | | 1995) | | | | | | |
| W | H12 | T | WH12T | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YT | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2181) | |
| | | | | 5199) | | | | | | |
| W | H12 | Y | WH12Y | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YY | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2182) | |
| | | | | 5199) | | | | | | |
| Q | L1 | C | QL1C | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2183) | |
| | | | | 1995) | | | | | | |
| Q | L1 | E | QL1E | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 1284) | |
| | | | | 1995) | | | | | | |
| Q | L1 | L | QL1L | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2185) | |
| | | | | 1995) | | | | | | |
| Q | L1 | S | QL1S | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2186) | |
| | | | | 1995) | | | | | | |
| Q | L1 | T | QL1T | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2187) | |
| | | | | 1995) | | | | | | |
| Q | L1 | Y | QL1Y | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2188) | |
| | | | | 1995) | | | | | | |
| Q | L2 | A | QL2A | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2189) | |
| | | | | 1995) | | | | | | |
| Q | L2 | D | QL2D | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2190) | |
| | | | | 1995) | | | | | | |
| Q | L2 | E | QL2E | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 1291) | |
| | | | | 1995) | | | | | | |
| Q | L2 | G | QL2G | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2192) | |
| | | | | 1995) | | | | | | |
| Q | L2 | H | QL2H | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 1293) | |
| | | | | 1995) | | | | | | |
| Q | L2 | I | QL2I | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2194) | |
| | | | | 1995) | | | | | | |
| Q | L2 | K | QL2K | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2195) | |
| | | | | 1995) | | | | | | |
| Q | L2 | L | QL2L | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2196) | |
| | | | | 1995) | | | | | | |
| Q | L2 | M | QL2M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2197) | |
| | | | | 1995) | | | | | | |
| Q | L2 | N | QL2N | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2198) | |
| | | | | 1995) | | | | | | |
| Q | L2 | P | QL2P | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2199) | |
| | | | | 1995) | | | | | | |
| Q | L2 | R | QL2R | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2200) | |
| | | | | 1995) | | | | | | |
| Q | L2 | S | QL2S | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2201) | |
| | | | | 1995) | | | | | | |
| Q | L2 | V | QL2V | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2202) | |
| | | | | 1995) | | | | | | |
| Q | L2 | Y | QL2Y | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2203) | |
| | | | | 1995) | | | | | | |
| S | L3 | A | SL3A | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2204) | |
| | | | | 1995) | | | | | | |
| Y | L4 | D | YL4D | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2205) | |
| | | | | 1995) | | | | | | |
| Y | L4 | G | YL4G | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2206) | |
| | | | | 1995) | | | | | | |
| Y | L4 | H | YL4H | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2207) | |
| | | | | 1995) | | | | | | |
| Y | L4 | I | YL4I | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2208) | |
| | | | | 1995) | | | | | | |
| Y | L4 | L | YL4L | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2209) | |
| | | | | 1995) | | | | | | |
| Y | L4 | M | YL4M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2210) | |
| | | | | 1995) | | | | | | |
| Y | L4 | P | YL4P | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2211) | |
| | | | | 1995) | | | | | | |
| Y | L4 | Q | YL4Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2212) | |
| | | | | 1995) | | | | | | |
| Y | L4 | R | YL4R | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2213) | |
| | | | | 1995) | | | | | | |
| Y | L4 | S | YL4S | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2214) | |
| | | | | 1995) | | | | | | |
| Y | L4 | T | YL4T | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2215) | |
| | | | | 1995) | | | | | | |
| Y | L4 | V | YL4V | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2216) | |
| | | | | 1995) | | | | | | |
| Y | L4 | W | YL4W | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2217) | |
| | | | | 1995) | | | | | | |
| T | L6 | Y | TL6Y | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2218) | |
| | | | | 1995) | | | | | | |
| T | L6 | A | TL6A | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2219) | |
| | | | | 1995) | | | | | | |
| T | L6 | C | TL6C | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2220) | |
| | | | | 1995) | | | | | | |
| T | L6 | D | TL6D | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2221) | |
| | | | | 1995) | | | | | | |
| T | L6 | F | TL6F | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2222) | |
| | | | | 1995) | | | | | | |
| T | L6 | H | TL6H | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2223) | |
| | | | | 1995) | | | | | | |
| T | L6 | I | TL6I | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2224) | |
| | | | | 1995) | | | | | | |
| T | L6 | K | TL6K | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2225) | |
| | | | | 1995) | | | | | | |
| T | L6 | L | TL6L | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2226) | |
| | | | | 1995) | | | | | | |
| T | L6 | M | TL6M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2227) | |
| | | | | 1995) | | | | | | |
| T | L6 | N | TL6N | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2228) | |
| | | | | 1995) | | | | | | |
| T | L6 | P | TL6P | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2229) | |
| | | | | 1995) | | | | | | |
| T | L6 | Q | TL6Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2230) | |
| | | | | 1995) | | | | | | |
| T | L6 | R | TL6R | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2231) | |
| | | | | 1995) | | | | | | |
| T | L6 | V | TL6V | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2232) | |
| | | | | 1995) | | | | | | |
| P | L7 | D | PL7D | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2233) | |
| | | | | 1995) | | | | | | |
| P | L7 | I | PL7I | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2234) | |
| | | | | 1995) | | | | | | |
| P | L7 | L | PL7L | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2235) | |
| | | | | 1995) | | | | | | |
| P | L7 | M | PL7M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | SEQ | |
| | | | | S (SEQ | | | | YC | ID | |
| | | | | ID | | | | | NO: | |
| | | | | NO: | | | | | 2236) | |
| | | | | 1995) | | | | | | |
| P | L7 | N | PL7N | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2237) | |
| | | | | 5199) | | | | | | |
| P | L7 | Q | PL7Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2238) | |
| | | | | 1995) | | | | | | |
| P | L7 | V | PL7V | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2239) | |
| | | | | 1995) | | | | | | |
| P | L7 | W | PL7W | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2240) | |
| | | | | 1995) | | | | | | |
| P | L7 | Y | PL7Y | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2241) | |
| | | | | 1995) | | | | | | |
| W | L8 | D | WL8D | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2242) | |
| | | | | 1995) | | | | | | |
| W | L8 | F | WL8F | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2243) | |
| | | | | 1995) | | | | | | |
| W | L8 | H | WL8H | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S | | | | YC | ID | |
| | | | | | | | | | NO: | |
| | | | | | | | | | 2244) | |
| W | L8 | I | WL8I | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2245) | |
| | | | | 1995) | | | | | | |
| W | L8 | K | WL8K | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2246) | |
| | | | | 1995) | | | | | | |
| W | L8 | L | WL8L | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2247) | |
| | | | | 1995) | | | | | | |
| W | L8 | M | WL8M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2248) | |
| | | | | 1995) | | | | | | |
| W | L8 | P | WL8P | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2249) | |
| | | | | 1995) | | | | | | |
| W | L8 | Q | WL8Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2250) | |
| | | | | 1995) | | | | | | |
| W | L8 | R | WL8R | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2251) | |
| | | | | 1995) | | | | | | |
| W | L8 | S | WL8S | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2252) | |
| | | | | 1995) | | | | | | |
| W | L8 | T | WL8T | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2253) | |
| | | | | 1995) | | | | | | |
| W | L8 | Y | WL8Y | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2254) | |
| | | | | 1995) | | | | | | |
| T | L9 | D | TL9D | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2255) | |
| | | | | 1995) | | | | | | |
| T | L9 | E | TL9E | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2256) | |
| | | | | 1995) | | | | | | |
| T | L9 | F | TL9F | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2257) | |
| | | | | 1995) | | | | | | |
| T | L9 | G | TL9G | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2258) | |
| | | | | 1995) | | | | | | |
| T | L9 | H | TL9H | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2259) | |
| | | | | 1995) | | | | | | |
| T | L9 | I | TL9I | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2260) | |
| | | | | 1995) | | | | | | |
| T | L9 | K | TL9K | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2261) | |
| | | | | 1995) | | | | | | |
| T | L9 | M | TL9M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2262) | |
| | | | | 1995) | | | | | | |
| T | L9 | N | TL9N | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2263) | |
| | | | | 1995) | | | | | | |
| T | L9 | P | TL9P | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2264) | |
| | | | | 1995) | | | | | | |
| T | L9 | Q | TL9Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2265) | |
| | | | | 1995) | | | | | | |
| T | L9 | R | TL9R | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2266) | |
| | | | | 1995) | | | | | | |
| T | L9 | S | TL9S | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2267) | |
| | | | | 1995) | | | | | | |
| T | L9 | V | TL9V | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2268) | |
| | | | | 1995) | | | | | | |
| T | L9 | W | TL9W | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2269) | |
| | | | | 1995) | | | | | | |
| T | L9 | Y | TL9Y | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2270) | |
| | | | | 1995) | | | | | | |
| F | L10 | C | FL10C | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2271) | |
| | | | | 1995) | | | | | | |
| F | L10 | D | FL10D | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2272) | |
| | | | | 1995) | | | | | | |
| F | L10 | E | FL10E | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2273) | |
| | | | | 1995) | | | | | | |
| F | L10 | H | FL10H | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2274) | |
| | | | | 1995) | | | | | | |
| F | L10 | I | FL10I | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2275) | |
| | | | | 1995) | | | | | | |
| F | L10 | K | FL10K | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2276) | |
| | | | | 1995) | | | | | | |
| F | L10 | L | FL10L | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2277) | |
| | | | | 1995) | | | | | | |
| F | L10 | M | FL10M | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2278) | |
| | | | | 1995) | | | | | | |
| F | L10 | N | FL10N | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2279) | |
| | | | | 1995) | | | | | | |
| F | L10 | P | FL10P | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2280) | |
| | | | | 1995) | | | | | | |
| F | L10 | Q | FL10Q | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2281) | |
| | | | | 1995) | | | | | | |
| F | L10 | R | FL10R | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2282) | |
| | | | | 1995) | | | | | | |
| F | L10 | S | FL10S | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2283) | |
| | | | | 1995) | | | | | | |
| F | L10 | T | FL10T | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2284) | |
| | | | | 1995) | | | | | | |
| F | L10 | V | FL10V | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2285) | |
| | | | | 1995) | | | | | | |
| F | L10 | W | FL10W | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2286) | |
| | | | | 1995) | | | | | | |
| F | L10 | Y | FL10Y | QVQL | GFTF | MHW | ISW | GYADSAK | AKR | GQGT |
| | | | | VESGG | DDY | VRQA | NSG | GRFTISRD | GNS | LVTV |
| | | | | GLVQP | A | PGKG | SI | NSKNTLY | NSFD | SS |
| | | | | GRSLR | | LEWV | | LQMNSLR | YW | |
| | | | | LSCAA | | SG | | AEDTAVY | (SEQ | |
| | | | | S (SEQ | | | | YC | ID | |
| | | | | ID | | | | | NO: | |
| | | | | NO: | | | | | 2287) | |
| | | | | 1995) | | | | | | |

In some aspects, the CD229 antigen binding domain comprises a sequence having at least 70%, 75%, 80%, 85% or 90% identity to the sequence set forth in SEQ ID NOs: 53, 84, or 134, wherein the CD229 antigen binding domain comprises 100% identity to SEQ ID NOs: 53, 84, or 134 at the HCDR3. In some aspects, the CD229 antigen binding domain comprises a sequence having at least 70%, 75%, 80%, 85% or 90% identity to the sequence set forth in SEQ ID NOs: 53, 84, or 134, wherein the CD229 antigen binding domain comprises 10000 identity at the G to D substitution, the S to E substitution, or F to Q substitution, of SEQ ID NOs: 53, 84, or 134, respectively.

In some aspects, the CD229 antigen binding domain comprises an altered affinity for CD229. In some aspects, the altered affinity is a lowered affinity. In some aspects, the variation in SEQ ID NO:1 provides a low affinity CD229 antigen binding domain. In some aspects, these low affinity CD229 antigen binding domain sequences show differential binding between healthy cells and target cells (e.g. cancer cells). For example, low affinity CD229 antigen binding domain sequences preferentially target cancer cells and do not bind to healthy cells. As described herein, GH4D, FH9Q, and SH6E have low affinity CD229 antigen binding domains.

In some aspects, the altered affinity is a higher affinity. In some aspects, the variation in SEQ ID NO:1 provides a high affinity CD229 antigen binding domain. In some aspects, these high affinity CD229 antigen binding domain sequences do not necessarily have differential binding between healthy cells and target cells (e.g. cancer cells) but do have an increased binding affinity. As described herein, NH7D has high affinity CD229 antigen binding domains.

### 2. Transmembrane Domain

In some instances, the transmembrane domain comprises an immunoglobulin Fc domain. In some instances, the immunoglobulin Fc domain can be an immunoglobulin G Fc domain.

In some instances, the transmembrane domain comprises a CD8α domain, CD3ζ, FcεR1γ, CD4, CD7, CD28, OX40, or H2-Kb.

In some instances, the transmembrane domain can be located between the CD229 antigen binding domain and the intracellular signaling domain.

### 3. Intracellular Signaling Domain

In some instances, the intracellular signaling domain comprises a co-stimulatory signaling region. In some instances, the co-stimulatory signaling region can comprise the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

In some instances, the intracellular signaling domain can be a T cell signaling domain. For example, the intracellular signaling domain can comprise a CD3ζ signaling domain. In some instances, CD3ζ signaling domain is the intracellular domain of CD3ζ.

In some instances, the intracellular signaling domain comprises a CD3ζ signaling domain and a co-stimulatory signaling region, wherein the co-stimulatory signaling region comprises the cytoplasmic domain of CD28, 4-1BB, CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

### 4. Hinge Region

Any of the disclosed CAR polypeptides can further comprise a hinge region. For example, disclosed are CAR polypeptides comprising a CD229 antigen binding domain, a transmembrane domain, and an intracellular signaling domain and further comprising a hinge region.

In some instances, the hinge region can be located between the CD229 antigen binding domain and the transmembrane domain.

In some instances, the hinge region allows for the CD229 antigen binding domain to bind to the antigen. For example, the hinge region can increase the distance of the binding domain to the cell surface and provide flexibility.

### C. CAR Nucleic Acid Sequence

Disclosed are nucleic acid sequences capable of encoding any of the disclosed CAR polypeptides. For example, disclosed are nucleic acid sequences capable of encoding the disclosed CAR polypeptides comprising a CD229 antigen binding domain, a transmembrane domain, and an intracellular signaling domain.

### 1. CD229 Antigen Binding Domain

Disclosed are nucleic acid sequences that encode any of the CD229 antigen binding domains described herein.

In some aspects, the nucleic acid sequence that encodes the CD229 antigen binding domain is

(SEQ ID NO: 2288)
CAGGTGCAGCTGGTGGAGTCTGGGGGAGGCTTGGTACAGCCTGGCAGGTCCCTGAG
ACTCTCCTGTGCAGCCTCTGGATTCACCTTTGATGATTATGCCATGCACTGGGTCCG
GCAAGCTCCAGGGAAGGGCCTGGAGTGGGTCTCAGGTATTAGTTGGAATAGTGGTA
GCATAGGCTATGCGGACTCCGCGAAGGGCCGGTTCACCATCTCCAGAGACAATTCC
AAGAACACGCTGTATCTGCAAATGAACAGCCTGAGAGCCGAGGACACGGCCGTCTA
TTACTGTGCGAAAAGGGGGAACTCCAACTCTCAAGACTACTGGGGCCAGGGAACCC
TGGTCACCGTCTCCTCACTCGAGGGTGGAGGCGGTTCAGGCGGAGGTGGCTCTGGC
GGTGGCGCTAGCGACATCCAGATGACCCAGTCTCCATCTTCCGTGTCTGCATCTGTA
GGAGACAGAGTCACCATCACTTGCCGGGCAAGTCAGAGCATTAGCAGCTATTTAAA
TTGGTATCAGCAGAAACCAGGGAAAGCCCCTAAGCTCCTGATCTATGCTGCATCCA
GTTTGCAAAGTGGGGTCCCATCAAGGTTCAGTGGCAGTGGATCTGGGACAGATTTC
ACTCTCACCATCAGCAGTCTGCAACCTGAAGATTTTGCAACTTACTACTGTCAACAG
AGTTACAGTACCCCCTGGACGTTCGGCCAAGGGACCAAGCTGGAGATCAAACGT
In some aspects, SEQ ID NO: 2288 is a nucleic acid sequence
that encodes FH9Q.

In some aspects, the nucleic acid sequence that encodes the CD229 antigen binding domain is

(SEQ ID NO: 2289)
CAGGTGCAGCTGGTGGAGTCTGGGGGAGGCTTGGTACAGCCTGGCAGGTCCCTGAG
ACTCTCCTGTGCAGCCTCTGGATTCACCTTTGATGATTATGCCATGCACTGGGTCCG
GCAAGCTCCAGGGAAGGGCCTGGAGTGGGTCTCAGGTATTAGTTGGAATAGTGGTA
GCATAGGCTATGCGGACTCCGCGAAGGGCCGGTTCACCATCTCCAGAGACAATTCC
AAGAACACGCTGTATCTGCAAATGAACAGCCTGAGAGCCGAGGACACGGCCGTCTA
TTACTGTGCGAAAAGGGATAACTCCAACTCTTTTGACTACTGGGGCCAGGGAACCC
TGGTCACCGTCTCCTCACTCGAGGGTGGAGGCGGTTCAGGCGGAGGTGGCTCTGGC
GGTGGCGCTAGCGACATCCAGATGACCCAGTCTCCATCTTCCGTGTCTGCATCTGTA
GGAGACAGAGTCACCATCACTTGCCGGGCAAGTCAGAGCATTAGCAGCTATTTAAA
TTGGTATCAGCAGAAACCAGGGAAAGCCCCTAAGCTCCTGATCTATGCTGCATCCA
GTTTGCAAAGTGGGGTCCCATCAAGGTTCAGTGGCAGTGGATCTGGGACAGATTTC
ACTCTCACCATCAGCAGTCTGCAACCTGAAGATTTTGCAACTTACTACTGTCAACAG
AGTTACAGTACCCCCTGGACGTTCGGCCAAGGGACCAAGCTGGAGATCAAACGT.
In some aspects, SEQ ID NO: 2289 is a nucleic acid sequence
that encodes GH4D.

In some aspects, the nucleic acid sequence that encodes the CD229 antigen binding domain is

(SEQ ID NO: 2290)
CAGGTGCAGCTGGTGGAGTCTGGGGGAGGCTTGGTACAGCCTGGCAGGTCCCTGAG
ACTCTCCTGTGCAGCCTCTGGATTCACCTTTGATGATTATGCCATGCACTGGGTCCG
GCAAGCTCCAGGGAAGGGCCTGGAGTGGGTCTCAGGTATTAGTTGGAATAGTGGTA
GCATAGGCTATGCGGACTCCGCGAAGGGCCGGTTCACCATCTCCAGAGACAATTCC
AAGAACACGCTGTATCTGCAAATGAACAGCCTGAGAGCCGAGGACACGGCCGTCTA
TTACTGTGCGAAAAGGGGGAACGAAAACTCTTTTGACTACTGGGGCCAGGGAACCC
TGGTCACCGTCTCCTCACTCGAGGGTGGAGGCGGTTCAGGCGGAGGTGGCTCTGGC
GGTGGCGCTAGCGACATCCAGATGACCCAGTCTCCATCTTCCGTGTCTGCATCTGTA
GGAGACAGAGTCACCATCACTTGCCGGGCAAGTCAGAGCATTAGCAGCTATTTAAA
TTGGTATCAGCAGAAACCAGGGAAAGCCCCTAAGCTCCTGATCTATGCTGCATCCA
GTTTGCAAAGTGGGGTCCCATCAAGGTTCAGTGGCAGTGGATCTGGGACAGATTTC
ACTCTCACCATCAGCAGTCTGCAACCTGAAGATTTTGCAACTTACTACTGTCAACAG
AGTTACAGTACCCCCTGGACGTTCGGCCAAGGGACCAAGCTGGAGATCAAACGT.
In some aspects, SEQ ID NO: 2290 is a nucleic acid sequence
that encodes SH6E.

### 2. Transmembrane Domain

In some instances, the transmembrane domain comprises a nucleic acid sequence that encodes an immunoglobulin Fc domain. In some instances, the immunoglobulin Fc domain can be an immunoglobulin G Fc domain.

In some instances, the transmembrane domain comprises a nucleic acid sequence that encodes a CD8α domain, CD3ζ, FcεR1γ, CD4, CD7, CD28, OX40, or H2-Kb.

In some instances, the transmembrane domain can be located between the CD229 antigen binding domain and the intracellular signaling domain.

### 3. Intracellular Domain

In some instances, the intracellular signaling domain comprises a nucleic acid that encodes a co-stimulatory signaling region. In some instances, the co-stimulatory signaling region can comprise the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

In some instances, the intracellular signaling domain can be a nucleic acid sequence encoding a T cell signaling domain. For example, the intracellular signaling domain can comprise a nucleic acid sequence that encodes a CD3ζ signaling domain. In some instances, CD3ζ signaling domain is the intracellular domain of CD3ζ.

In some instances, the intracellular signaling domain comprises a nucleic acid sequence encoding a CD3ζ signaling domain and a co-stimulatory signaling region, wherein the co-stimulatory signaling region comprises the cytoplasmic domain of CD28, 4-1BB, CD27, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

### D. Vectors

Disclosed are vectors comprising the nucleic acid sequence of the disclosed CAR nucleic acid sequences. In some instances, the vector can be selected from the group consisting of a DNA, a RNA, a plasmid, and a viral vector. In some instances, the vector can comprise a promoter.

### E. Cells

Disclosed are cells comprising any of the disclosed CAR polypeptides, CAR nucleic acids, or disclosed vectors. These cells can be considered genetically modified.

In some instances, the cell can be a T cell. For example, T cell can be a CD8+ T cell. In some instances, the can be a human cell.

Thus, disclosed are T cells expressing one of the CAR polypeptides disclosed herein. These can also be referred to as CAR T cells. Therefore, disclosed are CAR T cells comprising a CAR polypeptide, wherein the CAR polypeptide comprises a CD229 antigen binding domain, a transmembrane domain, and an intracellular signaling domain, wherein the CD229 binding domain is a variant of SEQ ID NO:1. In some aspects, the CD229 antigen binding domain comprises the sequence of

(SEQ ID NO: 134)
QVQLVESGGGLVQPGRSLRLSCAASGFTFDDYAMHWVRQAPGKGLEWVS
GISWNSGSIGYADSAKGRFTISRDNSKNTLYLQMNSLRAEDTAVYYCAK
RGNSNSQDYWGQGTLVTVSSLEGGGGSGGGGSGGGASDIQMTQSPSSVS
ASVGDRVTITCRASQSISSYLNWYQQKPGKAPKLLIYAASSLQSGVPSR
FSGSGSGTDFTLTISSLQPEDFATYYCQQSYSTPWTFGQGTKLEIK,
or
(SEQ ID NO: 53)
QVQLVESGGGLVQPGRSLRLSCAASGFTFDDYAMHWVRQAPGKGLEWVS
GISWNSGSIGYADSAKGRFTISRDNSKNTLYLQMNSLRAEDTAVYYCAK
RDNSNSFDYWGQGTLVTVSSLEGGGGSGGGGSGGGASDIQMTQSPSSVS
ASVGDRVTITCRASQSISSYLNWYQQKPGKAPKLLIYAASSLQSGVPSR
FSGSGSGTDFTLTISSLQPEDFATYYCQQSYSTPWTFGQGTKLEIKR.

### F. Antibodies

Disclosed are antibodies or fragments thereof that bind to human CD229, wherein said antibody comprises a variable heavy chain comprising a sequence having at least 90% identity to one of the variable heavy chain amino acid sequences provided in Table 1 or Table 3. Disclosed are antibodies or fragments thereof that bind to human CD229, wherein said antibody comprises a variable heavy chain comprising a sequence having at least 70%, 75%, 80%, 85% or 90% identity to a sequence set forth in SEQ ID NO:134, SEQ ID NO:53, or SEQ ID NO:84, wherein the CD229 antigen binding domain comprises 100% identity to SEQ ID NOs: 53, 84, or 134 at the HCDR3. In some aspects, the CD229 antigen binding domain comprises a sequence having at least 70%, 75%, 80%, 85% or 90% identity to the sequence set forth in SEQ ID NOs: 53, 84, or 134, wherein the CD229 antigen binding domain comprises 100% identity at the G to D substitution, the S to E substitution, or F to Q substitution, of SEQ ID NOs:53, 84, or 134, respectively.

Disclosed are antibodies or fragments thereof that bind to human CD229, wherein said antibody comprises a variable heavy chain comprising a HCDR3 comprising the sequence of AKRDNSNSFDYW (SEQ ID NO:253), AKRGNENSFDYW (SEQ ID NO:284, or AKRGNSNSQDYW (SEQ ID NO: 134).

Disclosed are antibodies or fragments thereof that bind to human CD229, wherein said antibody comprises a variable light chain comprising a sequence having at least 90% identity to one of the variable heavy chain amino acid sequences provided in Table 1 or Table 2. Disclosed are antibodies or fragments thereof that bind to human CD229, wherein said antibody comprises a variable light chain comprising a sequence having at least 90% identity to a sequence set forth in Table 2.

Disclosed are antibodies or fragments thereof that bind to human CD229, wherein said antibody comprises a heavy chain immunoglobulin variable region comprising a complementarity determining region 1 (CDR1) comprising the sequence of GFTFDDYA (SEQ ID NO:1996); a CDR2 comprising the sequence of ISWNSGSI (SEQ ID NO:1998); and a CDR3 comprising the sequence of AKRDNSNSFDYW (SEQ ID NO:253), AKRGNENSFDYW (SEQ ID NO:284), or AKRGNSNSQDYW (SEQ ID NO: 134).

In some instances, the disclosed antibodies or fragments thereof further comprise a tag sequence.

Disclosed are nucleic acid sequences that encode the disclosed antibodies or fragments thereof. For example, disclosed are nucleic acid sequences comprising

(SEQ ID NO: 2288)
CAGGTGCAGCTGGTGGAGTCTGGGGGAGGCTTGGTACAGCCTGGCAGGT
CCCTGAGACTCTCCTGTGCAGCCTCTGGATTCACCTTTGATGATTATGC
CATGCACTGGGTCCGGCAAGCTCCAGGGAAGGGCCTGGAGTGGGTCTCA
GGTATTAGTTGGAATAGTGGTAGCATAGGCTATGCGGACTCCGCGAAGG
GCCGGTTCACCATCTCCAGAGACAATTCCAAGAACACGCTGTATCTGCA
AATGAACAGCCTGAGAGCCGAGGACACGGCCGTCTATTACTGTGCGAAA
AGGGGGAACTCCAACTCTCAAGACTACTGGGGCCAGGGAACCCTGGTCA
CCGTCTCCTCACTCGAGGGTGGAGGCGGTTCAGGCGGAGGTGGCTCTGG
CGGTGGCGCTAGCGACATCCAGATGACCCAGTCTCCATCTTCCGTGTCT
GCATCTGTAGGAGACAGAGTCACCATCACTTGCCGGGCAAGTCAGAGCA
TTAGCAGCTATTTAAATTGGTATCAGCAGAAACCAGGGAAAGCCCCTAA
GCTCCTGATCTATGCTGCATCCAGTTTGCAAAGTGGGGTCCCATCAAGG
TTCAGTGGCAGTGGATCTGGGACAGATTTCACTCTCACCATCAGCAGTC
TGCAACCTGAAGATTTTGCAACTTACTACTGTCAACAGAGTTACAGTAC
CCCCTGGACGTTCGGCCAAGGGACCAAGCTGGAGATCAAACGT.

In some aspects, the nucleic acid sequence that encodes the CD229 antigen binding domain is

(SEQ ID NO: 2289)
CAGGTGCAGCTGGTGGAGTCTGGGGGAGGCTTGGTACAGCCTGGCAGGT
CCCTGAGACTCTCCTGTGCAGCCTCTGGATTCACCTTTGATGATTATGC
CATGCACTGGGTCCGGCAAGCTCCAGGGAAGGGCCTGGAGTGGGTCTCA
GGTATTAGTTGGAATAGTGGTAGCATAGGCTATGCGGACTCCGCGAAGG
GCCGGTTCACCATCTCCAGAGACAATTCCAAGAACACGCTGTATCTGCA
AATGAACAGCCTGAGAGCCGAGGACACGGCCGTCTATTACTGTGCGAAA
AGGGATAACTCCAACTCTTTTGACTACTGGGGCCAGGGAACCCTGGTCA
CCGTCTCCTCACTCGAGGGTGGAGGCGGTTCAGGCGGAGGTGGCTCTGG
CGGTGGCGCTAGCGACATCCAGATGACCCAGTCTCCATCTTCCGTGTCT
GCATCTGTAGGAGACAGAGTCACCATCACTTGCCGGGCAAGTCAGAGCA
TTAGCAGCTATTTAAATTGGTATCAGCAGAAACCAGGGAAAGCCCCTAA
GCTCCTGATCTATGCTGCATCCAGTTTGCAAAGTGGGGTCCCATCAAGG
TTCAGTGGCAGTGGATCTGGGACAGATTTCACTCTCACCATCAGCAGTC
TGCAACCTGAAGATTTTGCAACTTACTACTGTCAACAGAGTTACAGTAC
CCCCTGGACGTTCGGCCAAGGGACCAAGCTGGAGATCAAACGT.

In some aspects, the nucleic acid sequence that encodes the CD229 antigen binding domain is

(SEQ ID NO: 2290)
CAGGTGCAGCTGGTGGAGTCTGGGGGAGGCTTGGTACAGCCTGGCAGGT
CCCTGAGACTCTCCTGTGCAGCCTCTGGATTCACCTTTGATGATTATGC
CATGCACTGGGTCCGGCAAGCTCCAGGGAAGGGCCTGGAGTGGGTCTCA
GGTATTAGTTGGAATAGTGGTAGCATAGGCTATGCGGACTCCGCGAAGG
GCCGGTTCACCATCTCCAGAGACAATTCCAAGAACACGCTGTATCTGCA
AATGAACAGCCTGAGAGCCGAGGACACGGCCGTCTATTACTGTGCGAAA
AGGGGGAACGAAAACTCTTTTGACTACTGGGGCCAGGGAACCCTGGTCA
CCGTCTCCTCACTCGAGGGTGGAGGCGGTTCAGGCGGAGGTGGCTCTGG
CGGTGGCGCTAGCGACATCCAGATGACCCAGTCTCCATCTTCCGTGTCT
GCATCTGTAGGAGACAGAGTCACCATCACTTGCCGGGCAAGTCAGAGCA
TTAGCAGCTATTTAAATTGGTATCAGCAGAAACCAGGGAAAGCCCCTAA
GCTCCTGATCTATGCTGCATCCAGTTTGCAAAGTGGGGTCCCATCAAGG
TTCAGTGGCAGTGGATCTGGGACAGATTTCACTCTCACCATCAGCAGTC
TGCAACCTGAAGATTTTGCAACTTACTACTGTCAACAGAGTTACAGTAC
CCCCTGGACGTTCGGCCAAGGGACCAAGCTGGAGATCAAACGT.

In some instances, the disclosed antibodies or fragments thereof can be bispecific. For example, the antibody or fragment thereof can comprise a first Fab region comprising the heavy and light chain of one of SEQ ID NO: 134, SEQ ID NO:53, or SEQ ID NO:84 and a second Fab region comprising the heavy and light chain of one of SEQ ID NO:134, SEQ ID NO:53, or SEQ ID NO:84, wherein the first and second Fab regions are different.

In some instances, the bispecific antibodies can be trifunctional.

In some instances, the disclosed antibodies or fragments thereof can be mouse, human, humanized, chimeric, or a combination thereof.

In some instances, the disclosed antibodies or fragments thereof are monoclonal.

### G. Phage Display Library

Disclosed are phage display libraries comprising immunoglobulin genes. In some instances, the library displays scFv domains comprising both heavy and light chain variables of the sequences disclosed herein. In some instances, the library displays one or more of the antibodies disclosed herein.

### H. Compositions

Disclosed herein are compositions comprising any of the disclosed polypeptides, nucleic acids, vectors or cells. For example, disclosed are compositions comprising a CAR polypeptide comprising a CD229 antigen binding domain, a transmembrane domain, and an intracellular signaling domain, wherein the CD229 binding domain is a variant CD229 antigen binding domain. In some aspects, a CD229 antigen binding domain can comprise the sequence of SEQ ID NO: 134, SEQ ID NO:53, or SEQ ID NO:84.

Also disclosed herein are pharmaceutical compositions comprising the disclosed polypeptides, nucleic acids, vectors, or cells. Disclosed are pharmaceutical compositions comprising a CAR polypeptide comprising a CD229 antigen binding domain, a transmembrane domain, and an intracellular signaling domain, wherein the CD229 binding domain is a variant CD229 antigen binding domain. The disclosed compositions can further comprise a pharmaceutically acceptable carrier.

### 1. Delivery of Compositions

In the methods described herein, delivery (or administration) of the disclosed polypeptides, compositions, nucleic acids, cells or vectors disclosed herein to cells or a subject can be via a variety of mechanisms. The disclosed compositions can also include a carrier such as a pharmaceutically acceptable carrier. For example, disclosed are pharmaceutical compositions, comprising a CAR polypeptide comprising a CD229 antigen binding domain, a transmembrane domain, and an intracellular signaling domain, wherein the CD229 binding domain is a variant CD229 antigen binding domain as disclosed herein and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl choline (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically acceptable salt is used in the formulation to render the formulation isotonic. Other examples of the pharmaceutically acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed delivery techniques can be used not only for the disclosed compositions but also the disclosed polypeptides, nucleic acids, vectors or cells.

In some aspects, the disclosed compositions, polypeptides, nucleic acids, vectors, or cells are administered in combination with one or more additional agents. In some aspects, the additional agent can be, but is not limited to, a traditional therapeutic for the disease or disorder being treated. For example, a traditional therapeutic can be, but is not limited to, a therapeutic that treat cancer.

### I. Methods of Treating

### 1. Multiple Myeloma

Disclosed are methods of treating multiple myeloma comprising administering an effective amount of a T cell genetically modified to express one or more of the disclosed CAR polypeptides to a subject in need thereof. For example, disclosed are methods of treating multiple myeloma comprising administering an effective amount of a T cell genetically modified to express a CAR polypeptide comprising a CD229 antigen binding domain, a hinge and transmembrane domain, and an intracellular signaling domain, wherein the CD229 binding domain is a variant of SEQ ID NO:1.

Disclosed are methods of treating multiple myeloma comprising administering an effective amount of at least one of the disclosed antibodies or antibody fragments thereof to a subject in need thereof.

Disclosed are methods of treating multiple myeloma comprising administering an effective amount of at least one of the disclosed vectors to a subject in need thereof. For example, disclosed are methods of treating multiple myeloma comprising administering an effective amount of a vector comprising the nucleic acid sequence capable of encoding a disclosed CAR polypeptide to a subject in need thereof. In some instances, the vectors can comprise targeting moieties. In some instances, the targeting moieties target T cells.

Disclosed are methods of treating multiple myeloma comprising administering an effective amount of a composition comprising one or more of the disclosed antibodies or fragments thereof. For example, disclosed are methods of treating multiple myeloma comprising administering an effective amount of a composition comprising an antibody or fragment thereof comprising a CD229 antigen binding domain having at least 90% identity to the sequence set forth in SEQ ID NOs: 53, 84, or 134, wherein the CD229 antigen binding domain comprises 100% identity to SEQ ID NOs: 53, 84, or 134 at the HCDR3. Disclosed are methods of treating multiple myeloma comprising administering an effective amount of a composition comprising an antibody or fragment thereof comprising a CD229 antigen binding domain having at least 90% identity to the sequence set forth in SEQ ID NOs: 53, 84, or 134, wherein the CD229 antigen binding domain comprises 100% identity to SEQ ID NOs: 53, 84, or 134 at the substitution from wild type SEQ ID NO: 1.

In some instances, the disclosed methods of treating multiple myeloma further comprise administering a therapeutic agent. In some instances, the therapeutic agent can be, but is not limited to, conventional chemotherapy including but not limited to alkylating agents, antimetabolites, anti-microtubule agents, topoisomerase inhibitors, and cytotoxic antibiotics; high-dose chemotherapy including but not limited to high-dose Melphalan chemotherapy with or without stem cell transplant; proteasome inhibitors such as, but not limited to, bortezomib, ixazomib, and carfilzomib; immunomodulatory agents (IMiDS) such as, but not limited to, thalidomide, lenalidomide, and pomalidomide; histone deacetylase (HDAC) inhibitors such as, but not limited to panobinostat; monoclonal antibodies such as, but not limited to, daratumumab or elotuzumab; bispecific antibodies; and immune checkpoint inhibitors such as, but not limited to, ipilimumab, nivolumab, and pembrolizumab.

### 2. Lymphoma

Disclosed are methods of treating lymphoma comprising administering an effective amount of a T cell genetically modified to express one or more of the disclosed CAR polypeptides to a subject in need thereof. For example, disclosed are methods of treating lymphoma comprising administering an effective amount of a T cell genetically modified to express a CAR polypeptide comprising a CD229 antigen binding domain, a transmembrane domain, and an intracellular signaling domain, wherein the CD229 binding domain is a variant of SEQ ID NO:1.

Disclosed are methods of treating lymphoma comprising administering an effective amount of at least one of the disclosed vectors to a subject in need thereof. For example, disclosed are methods of treating lymphoma comprising administering an effective amount of a vector comprising the nucleic acid sequence capable of encoding a disclosed CAR polypeptide to a subject in need thereof. In some instances, the vectors can comprise targeting moieties. In some instances, the targeting moieties target T cells.

Disclosed are methods of treating lymphoma comprising administering an effective amount of at least one of the disclosed antibodies or antibody fragments to a subject in need thereof.

Disclosed are methods of treating lymphoma comprising administering an effective amount of a composition comprising one or more of the disclosed antibodies or fragments thereof. For example, disclosed are methods of treating lymphoma comprising administering an effective amount of a composition comprising an antibody or fragment thereof comprising a CD229 antigen binding domain having at least 90% identity to the sequence set forth in SEQ ID NOs:53, 84, or 134, wherein the CD229 antigen binding domain comprises 100% identity to SEQ ID NOs:53, 84, or 134 at the HCDR3. Disclosed are methods of treating multiple myeloma comprising administering an effective amount of a composition comprising an antibody or fragment thereof comprising a CD229 antigen binding domain having at least 90% identity to the sequence set forth in SEQ ID NOs:53, 84, or 134, wherein the CD229 antigen binding domain comprises 100% identity to SEQ ID NOs:53, 84, or 134 at the substitution from wild type SEQ ID NO:1.

In some instances, the disclosed methods of treating lymphoma further comprise administering a therapeutic agent. In some instances, the therapeutic agent can be, but is not limited to, conventional chemotherapy, vaccines, monoclonal antibodies, T cell immunotherapies, and other immunomodulatory agents.

A CAR-expressing cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

In one embodiment, a first CAR-expressing cell described herein, e.g., a CD229 CAR-expressing cell described herein, may be used in combination with a second CAR-expressing cell. In one embodiment, the second CAR-expressing cell expresses a CAR comprising a different anti-BMCA binding domain, e.g., an anti-CD229 binding domain described herein that differs from the anti-CD229 binding domain in the CAR expressed by the first CAR-expressing cell. In one embodiment, the second CAR-expressing cell expresses a CAR comprising an antigen-binding domain that targets an antigen other than CD229 (e.g., BCMA, CD19, CD20, CS-1, kappa light chain, CD139, Lewis Y antigen, or CD38). In one embodiment, a first CAR-expressing cell described herein, e.g., a CD229 CAR-expressing cell described herein, is used in combination with a second CAR-expressing cell comprising a CD19 CAR. In one embodiment, a CAR-expressing cell described herein is used in combination with a CD19 CAR-expressing cell to treat a BCMA-associated cancer described herein, e.g., multiple myeloma. In some embodiments, the multiple myeloma is CD19-negative, e.g., having a vast majority (e.g., at least 98%, 99%, 99.5%, 99.9%, or 99.95%) of the neoplastic plasma cells with a CD19-negative phenotype, e.g., as detected flow cytometry, RT-PCR, or both flow cytometry and RT-PCR.

In embodiments, a first CAR-expressing cell is administered to a subject, and a second CAR-expressing cell is administered to the subject. In embodiments, the first CAR-expressing cell comprises a CAR (e.g., CD229 CAR) comprising a CD27 costimulatory domain and a CD3zeta (mutant or wild type) primary signaling domain. In embodiments, the second CAR-expressing cell comprises a CAR (e.g., BCMA CAR) comprising a 4-1BB costimulatory domain and a CD3zeta (mutant or wild type) primary signaling domain. Without wishing to be bound by theory, in embodiments, the first CAR-expressing cell can be less toxic than the second CAR-expressing cell and be used to debulk a tumor.

In one embodiment, a CAR-expressing cell described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), innotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with the compounds of the present invention include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N2-[1,4-dioxo-[[4-(4-oxo-8-phenyl-4H-1-benzopyran-2-yl)morpholinium-4-yl]methoxy]butyl]-L-arginylglycyl-L-α-aspartylL-serine-(SEQ ID NO: 383), inner salt (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

In embodiments, a CAR-expressing cell described herein is administered to a subject in combination with fludarabine, cyclophosphamide, and/or rituximab. In embodiments, a CAR-expressing cell described herein is administered to a subject in combination with fludarabine, cyclophosphamide, and rituximab (FCR). In embodiments, the subject has CLL. For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgVH) gene. In other embodiments, the subject does not comprise a leukemic cell comprising a mutation in the immunoglobulin heavy-chain variable-region (IgVH) gene. In embodiments, the fludarabine is administered at a dosage of about 10-50 mg/m2 (e.g., about 10-15, 15-20, 20-25, 25-30, 30-35, 35-40, 40-45, or 45-50 mg/m2), e.g., intravenously. In embodiments, the cyclophosphamide is administered at a dosage of about 200-300 mg/m2 (e.g., about 200-225, 225-250, 250-275, or 275-300 mg/m2), e.g., intravenously. In embodiments, the rituximab is administered at a dosage of about 400-600 mg/m2 (e.g., 400-450, 450-500, 500-550, or 550-600 mg/m2), e.g., intravenously.

In embodiments, a CAR-expressing cell described herein is administered to a subject in combination with bendamustine and rituximab. In embodiments, the subject has CLL. For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgVH) gene. In other embodiments, the subject does not comprise a leukemic cell comprising a mutation in the immunoglobulin heavy-chain variable-region (IgVH) gene. In embodiments, the bendamustine is administered at a dosage of about 70-110 mg/m2 (e.g., 70-80, 80-90, 90-100, or 100-110 mg/m2), e.g., intravenously. In embodiments, the rituximab is administered at a dosage of about 400-600 mg/m2 (e.g., 400-450, 450-500, 500-550, or 550-600 mg/m2), e.g., intravenously.

In embodiments, a CAR-expressing cell described herein is administered to a subject in combination with rituximab, cyclophosphamide, doxorubicine, vincristine, and/or a corticosteroid (e.g., prednisone). In embodiments, a CAR-expressing cell described herein is administered to a subject in combination with rituximab, cyclophosphamide, doxorubicine, vincristine, and prednisone (R-CHOP). In embodiments, the subject has diffuse large B-cell lymphoma (DLBCL). In embodiments, the subject has nonbulky limited-stage DLBCL (e.g., comprises a tumor having a size/diameter of less than 7 cm). In embodiments, the subject is treated with radiation in combination with the R-CHOP. For example, the subject is administered R-CHOP (e.g., 1-6 cycles, e.g., 1, 2, 3, 4, 5, or 6 cycles of R-CHOP), followed by radiation. In some cases, the subject is administered R-CHOP (e.g., 1-6 cycles, e.g., 1, 2, 3, 4, 5, or 6 cycles of R-CHOP) following radiation.

In embodiments, a CAR-expressing cell described herein is administered to a subject in combination with etoposide, prednisone, vincristine, cyclophosphamide, doxorubicin, and/or rituximab. In embodiments, a CAR-expressing cell described herein is administered to a subject in combination with etoposide, prednisone, vincristine, cyclophosphamide, doxorubicin, and rituximab (EPOCH-R). In embodiments, a CAR-expressing cell described herein is administered to a subject in combination with dose-adjusted EPOCH-R (DA-EPOCH-R). In embodiments, the subject has a B cell lymphoma, e.g., a Myc-rearranged aggressive B cell lymphoma.

In embodiments, a CAR-expressing cell described herein is administered to a subject in combination with rituximab and/or lenalidomide. Lenalidomide ((RS)-3-(4-Amino-1-oxo 1,3-dihydro-2H-isoindol-2-yl)piperidine-2,6-dione) is an immunomodulator. In embodiments, a CAR-expressing cell described herein is administered to a subject in combination with rituximab and lenalidomide. In embodiments, the subject has follicular lymphoma (FL) or mantle cell lymphoma (MCL). In embodiments, the subject has FL and has not previously been treated with a cancer therapy. In embodiments, lenalidomide is administered at a dosage of about 10-20 mg (e.g., 10-15 or 15-20 mg), e.g., daily. In embodiments, rituximab is administered at a dosage of about 350-550 mg/m2 (e.g., 350-375, 375-400, 400-425, 425-450, 450-475, or 475-500 mg/m2), e.g., intravenously.

In embodiments, a CAR-expressing cell described herein is administered to a subject in combination with brentuximab. Brentuximab is an antibody-drug conjugate of anti-CD30 antibody and monomethyl auristatin E. In embodiments, the subject has Hodgkin's lymphoma (HL), e.g., relapsed or refractory HL. In embodiments, the subject comprises CD30+HL. In embodiments, the subject has undergone an autologous stem cell transplant (ASCT). In embodiments, the subject has not undergone an ASCT. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In embodiments, a CAR-expressing cell described herein is administered to a subject in combination with brentuximab and dacarbazine or in combination with brentuximab and bendamustine. Dacarbazine is an alkylating agent with a chemical name of 5-(3,3-Dimethyl-1-triazenyl)imidazole-4-carboxamide. Bendamustine is an alkylating agent with a chemical name of 4-[5-[Bis(2-chloroethyl)amino]-1-methylbenzimidazol-2-yl]butanoic acid. In embodiments, the subject has Hodgkin's lymphoma (HL). In embodiments, the subject has not previously been treated with a cancer therapy. In embodiments, the subject is at least 60 years of age, e.g., 60, 65, 70, 75, 80, 85, or older. In embodiments, dacarbazine is administered at a dosage of about 300-450 mg/m2 (e.g., about 300-325, 325-350, 350-375, 375-400, 400-425, or 425-450 mg/m2), e.g., intravenously. In embodiments, bendamustine is administered at a dosage of about 75-125 mg/m2 (e.g., 75-100 or 100-125 mg/m2, e.g., about 90 mg/m2), e.g., intravenously. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In some embodiments, a CAR-expressing cell described herein is administered to a subject in combination with a CD20 inhibitor, e.g., an anti-CD20 antibody (e.g., an anti-CD20 mono- or bispecific antibody) or a fragment thereof. Exemplary anti-CD20 antibodies include but are not limited to rituximab, ofatumumab, ocrelizumab, veltuzumab, obinutuzumab, TRU-015 (Trubion Pharmaceuticals), ocaratuzumab, and Pro131921 (Genentech). See, e.g., Lim et al. Haematologica. 95.1(2010):135-43.

In some embodiments, the anti-CD20 antibody comprises rituximab. Rituximab is a chimeric mouse/human monoclonal antibody IgG1 kappa that binds to CD20 and causes cytolysis of a CD20 expressing cell, e.g., as described in www.accessdata.fda.gov/drugsatfda_docs/label/2010/103705s53111bl.pdf. In embodiments, a CAR-expressing cell described herein is administered to a subject in combination with rituximab. In embodiments, the subject has CLL or SLL.

In some embodiments, rituximab is administered intravenously, e.g., as an intravenous infusion. For example, each infusion provides about 500-2000 mg (e.g., about 500-550, 550-600, 600-650, 650-700, 700-750, 750-800, 800-850, 850-900, 900-950, 950-1000, 1000-1100, 1100-1200, 1200-1300, 1300-1400, 1400-1500, 1500-1600, 1600-1700, 1700-1800, 1800-1900, or 1900-2000 mg) of rituximab. In some embodiments, rituximab is administered at a dose of 150 mg/m2 to 750 mg/m2, e.g., about 150-175 mg/m2, 175-200 mg/m2, 200-225 mg/m2, 225-250 mg/m2, 250-300 mg/m2, 300-325 mg/m2, 325-350 mg/m2, 350-375 mg/m2, 375-400 mg/m2, 400-425 mg/m2, 425-450 mg/m2, 450-475 mg/m2, 475-500 mg/m2, 500-525 mg/m2, 525-550 mg/m2, 550-575 mg/m2, 575-600 mg/m2, 600-625 mg/m2, 625-650 mg/m2, 650-675 mg/m2, or 675-700 mg/m2, where m2 indicates the body surface area of the subject. In some embodiments, rituximab is administered at a dosing interval of at least 4 days, e.g., 4, 7, 14, 21, 28, 35 days, or more. For example, rituximab is administered at a dosing interval of at least 0.5 weeks, e.g., 0.5, 1, 2, 3, 4, 5, 6, 7, 8 weeks, or more. In some embodiments, rituximab is administered at a dose and dosing interval described herein for a period of time, e.g., at least 2 weeks, e.g., at least 2, 3, 4, 5, 6, 7, 8, 9, 10, 11, 12, 13, 14, 15, 16, 17, 18, 19, 20 weeks, or greater. For example, rituximab is administered at a dose and dosing interval described herein for a total of at least 4 doses per treatment cycle (e.g., at least 4, 5, 6, 7, 8, 9, 10, 11, 12, 13, 14, 15, 16, or more doses per treatment cycle).

In some embodiments, the anti-CD20 antibody comprises ofatumumab. Ofatumumab is an anti-CD20 IgG1κ human monoclonal antibody with a molecular weight of approximately 149 kDa. For example, ofatumumab is generated using transgenic mouse and hybridoma technology and is expressed and purified from a recombinant murine cell line (NSO). See, e.g., www.accessdata.fda.gov/drugsatfda_docs/label/2009/1253261bl.pdf, and Clinical Trial Identifier number NCT01363128, NCT01515176, NCT01626352, and NCT01397591. In embodiments, a CAR-expressing cell described herein is administered to a subject in combination with ofatumumab. In embodiments, the subject has CLL or SLL.

In some embodiments, ofatumumab is administered as an intravenous infusion. For example, each infusion provides about 150-3000 mg (e.g., about 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1200, 1200-1400, 1400-1600, 1600-1800, 1800-2000, 2000-2200, 2200-2400, 2400-2600, 2600-2800, or 2800-3000 mg) of ofatumumab. In embodiments, ofatumumab is administered at a starting dosage of about 300 mg, followed by 2000 mg, e.g., for about 11 doses, e.g., for 24 weeks. In some embodiments, ofatumumab is administered at a dosing interval of at least 4 days, e.g., 4, 7, 14, 21, 28, 35 days, or more. For example, ofatumumab is administered at a dosing interval of at least 1 week, e.g., 1, 2, 3, 4, 5, 6, 7, 8, 9, 10, 11, 12, 24, 26, 28, 20, 22, 24, 26, 28, 30 weeks, or more. In some embodiments, ofatumumab is administered at a dose and dosing interval described herein for a period of time, e.g., at least 1 week, e.g., 1, 2, 3, 4, 5, 6, 7, 8, 9, 10, 11, 12, 13, 14, 15, 16, 17, 18, 19, 20, 22, 24, 26, 28, 30, 40, 50, 60 weeks or greater, or 1, 2, 3, 4, 5, 6, 7, 8, 9, 10, 11, 12 months or greater, or 1, 2, 3, 4, 5 years or greater. For example, ofatumumab is administered at a dose and dosing interval described herein for a total of at least 2 doses per treatment cycle (e.g., at least 2, 3, 4, 5, 6, 7, 8, 9, 10, 11, 12, 13, 14, 15, 16, 18, 20, or more doses per treatment cycle).

In some cases, the anti-CD20 antibody comprises ocrelizumab. Ocrelizumab is a humanized anti-CD20 monoclonal antibody, e.g., as described in Clinical Trials Identifier Nos. NCT00077870, NCT01412333, NCT00779220, NCT00673920, NCT01194570, and Kappos et al. Lancet. 19.378(2011):1779-87.

In some cases, the anti-CD20 antibody comprises veltuzumab. Veltuzumab is a humanized monoclonal antibody against CD20. See, e.g., Clinical Trial Identifier No. NCT00547066, NCT00546793, NCT01101581, and Goldenberg et al. Leuk Lymphoma. 51(5)(2010):747-55.

In some cases, the anti-CD20 antibody comprises GA101. GA101 (also called obinutuzumab or R05072759) is a humanized and glyco-engineered anti-CD20 monoclonal antibody. See, e.g., Robak. Curr. Opin. Investig. Drugs. 10.6(2009):588-96; Clinical Trial Identifier Numbers: NCT01995669, NCT01889797, NCT02229422, and NCT01414205; and www.accessdatafda.gov/drugsatfda_docs/label/2013/125486s000lbl.pdf.

In some cases, the anti-CD20 antibody comprises AME-133v. AME-133v (also called LY2469298 or ocaratuzumab) is a humanized IgG1 monoclonal antibody against CD20 with increased affinity for the FcγRIIIa receptor and an enhanced antibody dependent cellular cytotoxicity (ADCC) activity compared with rituximab. See, e.g., Robak et al. BioDrugs 25.1(2011):13-25; and Forero-Torres et al. Clin Cancer Res. 18.5(2012):1395-403.

In some cases, the anti-CD20 antibody comprises PRO131921. PRO131921 is a humanized anti-CD20 monoclonal antibody engineered to have better binding to FcγRIIIa and enhanced ADCC compared with rituximab. See, e.g., Robak et al. BioDrugs 25.1(2011):13-25; and Casulo et al. Clin Immunol. 154.1(2014):37-46; and Clinical Trial Identifier No. NCT00452127.

In some cases, the anti-CD20 antibody comprises TRU-015. TRU-015 is an anti-CD20 fusion protein derived from domains of an antibody against CD20. TRU-015 is smaller than monoclonal antibodies, but retains Fc-mediated effector functions. See, e.g., Robak et al. BioDrugs 25.1(2011):13-25. TRU-015 contains an anti-CD20 single-chain variable fragment (scFv) linked to human IgG1 hinge, CH2, and CH3 domains but lacks CH1 and CL domains.

In some embodiments, an anti-CD20 antibody described herein is conjugated or otherwise bound to a therapeutic agent, e.g., a chemotherapeutic agent (e.g., cytoxan, fludarabine, histone deacetylase inhibitor, demethylating agent, peptide vaccine, anti-tumor antibiotic, tyrosine kinase inhibitor, alkylating agent, anti-microtubule or anti-mitotic agent), anti-allergic agent, anti-nausea agent (or anti-emetic), pain reliever, or cytoprotective agent described herein.

In embodiments, a CAR-expressing cell described herein is administered to a subject in combination with a B-cell lymphoma 2 (BCL-2) inhibitor (e.g., venetoclax, also called ABT-199 or GDC-0199;) and/or rituximab. In embodiments, a CAR-expressing cell described herein is administered to a subject in combination with venetoclax and rituximab. Venetoclax is a small molecule that inhibits the anti-apoptotic protein, BCL-2. The structure of venetoclax (4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide) is shown below.

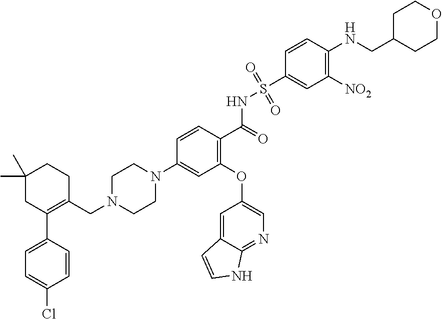 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

In embodiments, the subject has CLL. In embodiments, the subject has relapsed CLL, e.g., the subject has previously been administered a cancer therapy. In embodiments, venetoclax is administered at a dosage of about 15-600 mg (e.g., 15-20, 20-50, 50-75, 75-100, 100-200, 200-300, 300-400, 400-500, or 500-600 mg), e.g., daily. In embodiments, rituximab is administered at a dosage of about 350-550 mg/m2 (e.g., 350-375, 375-400, 400-425, 425-450, 450-475, or 475-500 mg/m2), e.g., intravenously, e.g., monthly.

Without being bound by theory, it is believed that in some cancers, B cells (e.g., B regulatory cells) can suppress T cells. Further, it is believed that a combination of oxiplatin and the B cell depleting agent may reduce tumor size and/or eliminate tumors in a subject. In some embodiments, a CAR-expressing cell described herein (e.g., BCMA CAR) is administered in combination with a B cell depleting agent (e.g., a CD19 CAR-expressing cell, a CD20 CAR-expressing cell, rituximab, ocrelizumab, epratuzumab, or belimumab) and oxiplatin. In embodiments, the cancer cell can be CD19 negative or CD19 positive; or BCMA negative or BMCA positive. In embodiments, a CAR-expressing cell described herein (e.g., BCMA CAR) is administered in combination with a B cell depleting agent and oxiplatin to treat a cancer, e.g., a cancer described herein, e.g., solid cancer, e.g., prostate cancer, pancreatic cancer, or lung cancer.

In embodiments, a CAR-expressing cell described herein (e.g., BCMA CAR) may deplete B cells (e.g., B cells having a plasma cell-like phenotype, e.g., that express BCMA, CD19, and/or CD20) in a subject. In embodiments, the B cell can be CD19 negative or CD19 positive; or BCMA negative or BMCA positive. In some embodiments, a CAR-expressing cell described herein (e.g., BCMA CAR) is administered in combination with oxiplatin. In embodiments, a CAR-expressing cell described herein is administered in combination with oxiplatin is used to treat a cancer, e.g., solid cancer, e.g., prostate cancer, pancreatic cancer, or lung cancer. In some embodiments, a CAR-expressing cell described herein is administered in combination with an oncolytic virus. In embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

In some embodiments, the oncolytic virus is a virus, e.g., recombinant oncolytic virus, described in US2010/0178684 A1, which is incorporated herein by reference in its entirety. In some embodiments, a recombinant oncolytic virus comprises a nucleic acid sequence (e.g., heterologous nucleic acid sequence) encoding an inhibitor of an immune or inflammatory response, e.g., as described in US2010/0178684 A1, incorporated herein by reference in its entirety. In embodiments, the recombinant oncolytic virus, e.g., oncolytic NDV, comprises a pro-apoptotic protein (e.g., apoptin), a cytokine (e.g., GM-CSF, interferon-gamma, interleukin-2 (IL-2), tumor necrosis factor-alpha), an immunoglobulin (e.g., an antibody against ED-B firbonectin), tumor associated antigen, a bispecific adapter protein (e.g., bispecific antibody or antibody fragment directed against NDV HN protein and a T cell co-stimulatory receptor, such as CD3 or CD28; or fusion protein between human IL-2 and single chain antibody directed against NDV HN protein). See, e.g., Zamarin et al. Future Microbiol. 7.3(2012):347-67, incorporated herein by reference in its entirety. In some embodiments, the oncolytic virus is a chimeric oncolytic NDV described in U.S. Pat. No. 8,591,881 B2, US 2012/0122185 A1, or US 2014/0271677 A1, each of which is incorporated herein by reference in their entireties.

In some embodiments, the oncolytic virus comprises a conditionally replicative adenovirus (CRAd), which is designed to replicate exclusively in cancer cells. See, e.g., Alemany et al. Nature Biotechnol. 18(2000):723-27. In some embodiments, an oncolytic adenovirus comprises one described in Table 1 on page 725 of Alemany et al., incorporated herein by reference in its entirety.

Exemplary oncolytic viruses include but are not limited to the following: Group B Oncolytic Adenovirus (ColoAd1) (PsiOxus Therapeutics Ltd.) (see, e.g., Clinical Trial Identifier: NCT02053220); ONCOS-102 (previously called CGTG-102), which is an adenovirus comprising granulocyte-macrophage colony stimulating factor (GM-CSF) (Oncos Therapeutics) (see, e.g., Clinical Trial Identifier: NCT01598129); VCN-01, which is a genetically modified oncolytic human adenovirus encoding human PH20 hyaluronidase (VCN Biosciences, S.L.) (see, e.g., Clinical Trial Identifiers: NCT02045602 and NCT02045589); Conditionally Replicative Adenovirus ICOVIR-5, which is a virus derived from wild-type human adenovirus serotype 5 (Had5) that has been modified to selectively replicate in cancer cells with a deregulated retinoblastoma/E2F pathway (Institut Catali d'Oncologia) (see, e.g., Clinical Trial Identifier: NCT01864759); Celyvir, which comprises bone marrow-derived autologous mesenchymal stem cells (MSCs) infected with ICOVIR5, an oncolytic adenovirus (Hospital Infantil Universitario Niño Jesús, Madrid, Spain/Ramon Alemany) (see, e.g., Clinical Trial Identifier: NCT01844661); CG0070, which is a conditionally replicating oncolytic serotype 5 adenovirus (Ad5) in which human E2F-1 promoter drives expression of the essential Ela viral genes, thereby restricting viral replication and cytotoxicity to Rb pathway-defective tumor cells (Cold Genesys, Inc.) (see, e.g., Clinical Trial Identifier: NCT02143804); orDNX-2401 (formerly named Delta-24-RGD), which is an adenovirus that has been engineered to replicate selectively in retinoblastoma (Rb)-pathway deficient cells and to infect cells that express certain RGD-binding integrins more efficiently (Clinica Universidad de Navarra, Universidad de Navarra/DNAtrix, Inc.) (see, e.g., Clinical Trial Identifier: NCT01956734).

In some embodiments, an oncolytic virus described herein is administering by injection, e.g., subcutaneous, intra-arterial, intravenous, intramuscular, intrathecal, or intraperitoneal injection. In embodiments, an oncolytic virus described herein is administered intratumorally, transdermally, transmuco sally, orally, intranasally, or via pulmonary administration.

In an embodiment, cells expressing a CAR described herein can be administered to a subject in combination with a molecule that decreases the Treg cell population. Methods that decrease the number of (e.g., deplete) Treg cells are known in the art and include, e.g., CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse. In one embodiment, a CAR expressing cell described herein is administered to a subject in combination with a a molecule targeting GITR and/or modulating GITR functions, such as a GITR agonist and/or a GITR antibody that depletes regulatory T cells (Tregs). In embodiments, cells expressing a CAR described herein are administered to a subject in combination with cyclophosphamide. In one embodiment, the GITR binding molecules and/or molecules modulating GITR functions (e.g., GITR agonist and/or Treg depleting GITR antibodies) are administered prior to administration of the CAR-expressing cell. For example, in one embodiment, the GITR agonist can be administered prior to apheresis of the cells. In embodiments, cyclophosphamide is administered to the subject prior to administration (e.g., infusion or re-infusion) of the CAR-expressing cell or prior to aphersis of the cells. In embodiments, cyclophosphamide and an anti-GITR antibody are administered to the subject prior to administration (e.g., infusion or re-infusion) of the CAR-expressing cell or prior to apheresis of the cells. In one embodiment, the subject has cancer (e.g., a solid cancer or a hematological cancer such as multiple myeloma, ALL or CLL). In an embodiment, the subject has CLL. In embodiments, the subject has multiple myeloma. In embodiments, the subject has a solid cancer, e.g., a solid cancer described herein. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, a CAR expressing cell described herein is administered to a subject in combination with an mTOR inhibitor, e.g., an mTOR inhibitor described herein, e.g., a rapalog such as everolimus. In one embodiment, the mTOR inhibitor is administered prior to the CAR-expressing cell. For example, in one embodiment, the mTOR inhibitor can be administered prior to apheresis of the cells.

In one embodiment, a CAR expressing cell described herein is administered to a subject in combination with a GITR agonist, e.g., a GITR agonist described herein. In one embodiment, the GITR agonist is administered prior to the CAR-expressing cell. For example, in one embodiment, the GITR agonist can be administered prior to apheresis of the cells.

In one embodiment, a CAR expressing cell described herein is administered to a subject in combination with a protein tyrosine phosphatase inhibitor, e.g., a protein tyrosine phosphatase inhibitor described herein. In one embodiment, the protein tyrosine phosphatase inhibitor is an SHP-1 inhibitor, e.g., an SHP-1 inhibitor described herein, such as, e.g., sodium stibogluconate. In one embodiment, the protein tyrosine phosphatase inhibitor is an SHP-2 inhibitor.

In one embodiment, a CAR-expressing cell described herein can be used in combination with a kinase inhibitor. In one embodiment, the kinase inhibitor is a CDK4 inhibitor, e.g., a CDK4 inhibitor described herein, e.g., a CDK4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In one embodiment, the kinase inhibitor is a BTK inhibitor, e.g., a BTK inhibitor described herein, such as, e.g., ibrutinib. In one embodiment, the kinase inhibitor is an mTOR inhibitor, e.g., an mTOR inhibitor described herein, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor described herein. In one embodiment, the kinase inhibitor is a MNK inhibitor, e.g., a MNK inhibitor described herein, such as, e.g., 4-amino-5-(4-fluoroanilino)-pyrazolo[3,4-d]pyrimidine. The MNK inhibitor can be, e.g., a MNKla, MNKlb, MNK2a and/or MNK2b inhibitor. In one embodiment, the kinase inhibitor is a dual PI3K/mTOR inhibitor described herein, such as, e.g., PF-04695102. In one embodiment, the kinase inhibitor is a DGK inhibitor, e.g., a DGK inhibitor described herein, such as, e.g., DGKinh1 (D5919) or DGKinh2 (D5794).

In one embodiment, the kinase inhibitor is a CDK4 inhibitor selected from aloisine A; flavopiridol or HMR-1275, 2-(2-chlorophenyl)-5,7-dihydroxy-8-[(3S,4R)-3-hydroxy-1-methyl-4-piperidinyl]-4-chromenone; crizotinib (PF-02341066; 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265); indisulam (E7070); roscovitine (CYC202); palbociclib (PD0332991); dinaciclib (SCH727965); N-[5-[[(5-tert-butyloxazol-2-yl)methyl]thio]thiazol-2-yl]piperidine-4-carboxamide (BMS 387032); 4-[[9-chloro-7-(2,6-difluorophenyl)-5H-pyrimido[5,4-d][2]benzazepin-2-yl]amino]-benzoic acid (MLN8054); 5-[3-(4,6-difluoro-1H-benzimidazol-2-yl)-1H-indazol-5-yl]-N-ethyl-4-methyl-3-pyridinemethanamine (AG-024322); 4-(2,6-dichlorobenzoylamino)-1H-pyrazole-3-carboxylic acid N-(piperidin-4-yl)amide (AT7519); 4-[2-methyl-1-(1-methylethyl)-1H-imidazol-5-yl]-N-[4-(methylsulfonyl)phenyl]-2-pyrimidinamine (AZD5438); and XL281 (BMS908662).

In one embodiment, the kinase inhibitor is a CDK4 inhibitor, e.g., palbociclib (PD0332991), and the palbociclib is administered at a dose of about 50 mg, 60 mg, 70 mg, 75 mg, 80 mg, 90 mg, 100 mg, 105 mg, 110 mg, 115 mg, 120 mg, 125 mg, 130 mg, 135 mg (e.g., 75 mg, 100 mg or 125 mg) daily for a period of time, e.g., daily for 14-21 days of a 28 day cycle, or daily for 7-12 days of a 21 day cycle. In one embodiment, 1, 2, 3, 4, 5, 6, 7, 8, 9, 10, 11, 12 or more cycles of palbociclib are administered.

In embodiments, a CAR-expressing cell described herein is administered to a subject in combination with a cyclin-dependent kinase (CDK) 4 or 6 inhibitor, e.g., a CDK4 inhibitor or a CDK6 inhibitor described herein. In embodiments, a CAR-expressing cell described herein is administered to a subject in combination with a CDK4/6 inhibitor (e.g., an inhibitor that targets both CDK4 and CDK6), e.g., a CDK4/6 inhibitor described herein. In an embodiment, the subject has MCL. MCL is an aggressive cancer that is poorly responsive to currently available therapies, i.e., essentially incurable. In many cases of MCL, cyclin D1 (a regulator of CDK4/6) is expressed (e.g., due to chromosomal translocation involving immunoglobulin and Cyclin D1 genes) in MCL cells. Thus, without being bound by theory, it is thought that MCL cells are highly sensitive to CDK4/6 inhibition with high specificity (i.e., minimal effect on normal immune cells). CDK4/6 inhibitors alone have had some efficacy in treating MCL, but have only achieved partial remission with a high relapse rate. An exemplary CDK4/6 inhibitor is LEE011 (also called ribociclib), the structure of which is shown below.

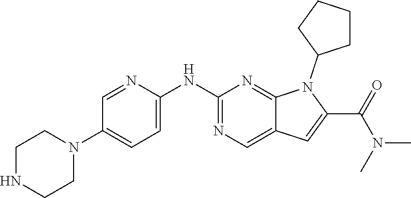 [SMILES: CN(C)C(=O)c1cc2cnc(Nc3ccc(N4CCNCC4)cn3)nc2n1C1CCCC1]

Without being bound by theory, it is believed that administration of a CAR-expressing cell described herein with a CDK4/6 inhibitor (e.g., LEE011 or other CDK4/6 inhibitor described herein) can achieve higher responsiveness, e.g., with higher remission rates and/or lower relapse rates, e.g., compared to a CDK4/6 inhibitor alone.

In one embodiment, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In a preferred embodiment, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In one embodiment, the kinase inhibitor is a BTK inhibitor, e.g., ibrutinib (PCI-32765). In embodiments, a CAR-expressing cell described herein is administered to a subject in combination with a BTK inhibitor (e.g., ibrutinib). In embodiments, a CAR-expressing cell described herein is administered to a subject in combination with ibrutinib (also called PCI-32765). The structure of ibrutinib (1-[(3R)-3-[4-Amino-3-(4-phenoxyphenyl)-1H-pyrazolo[3,4-d]pyrimidin-1-yl]piperidin-1-yl]prop-2-en-1-one) is shown below.

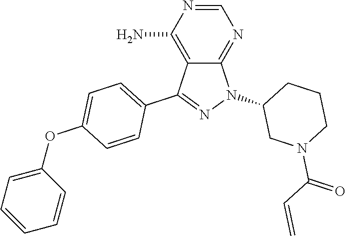

In embodiments, the subject has CLL, mantle cell lymphoma (MCL), or small lymphocytic lymphoma (SLL). For example, the subject has a deletion in the short arm of chromosome 17 (del(17p), e.g., in a leukemic cell). In other examples, the subject does not have a del(17p). In embodiments, the subject has relapsed CLL or SLL, e.g., the subject has previously been administered a cancer therapy (e.g., previously been administered one, two, three, or four prior cancer therapies). In embodiments, the subject has refractory CLL or SLL. In other embodiments, the subject has follicular lymphoma, e.g., relapse or refractory follicular lymphoma. In some embodiments, ibrutinib is administered at a dosage of about 300-600 mg/day (e.g., about 300-350, 350-400, 400-450, 450-500, 500-550, or 550-600 mg/day, e.g., about 420 mg/day or about 560 mg/day), e.g., orally. In embodiments, the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle cycle, or daily for 28 day cycle. In one embodiment, 1, 2, 3, 4, 5, 6, 7, 8, 9, 10, 11, 12 or more cycles of ibrutinib are administered. In some embodiments, ibrutinib is administered in combination with rituximab. See, e.g., Burger et al. (2013) Ibrutinib In Combination With Rituximab (iR) Is Well Tolerated and Induces a High Rate Of Durable Remissions In Patients With High-Risk Chronic Lymphocytic Leukemia (CLL): New, Updated Results Of a Phase II Trial In 40 Patients, Abstract 675 presented at 55th ASH Annual Meeting and Exposition, New Orleans, La. 7-10 December Without being bound by theory, it is thought that the addition of ibrutinib enhances the T cell proliferative response and may shift T cells from a T-helper-2 (Th2) to T-helper-1 (Th1) phenotype. Th1 and Th2 are phenotypes of helper T cells, with Th1 versus Th2 directing different immune response pathways. A Th1 phenotype is associated with proinflammatory responses, e.g., for killing cells, such as intracellular pathogens/viruses or cancerous cells, or perpetuating autoimmune responses. A Th2 phenotype is associated with eosinophil accumulation and anti-inflammatory responses.

In some embodiments of the methods, uses, and compositions herein, the BTK inhibitor is a BTK inhibitor described in International Application WO/2015/079417, which is herein incorporated by reference in its entirety. For instance, in some embodiments, the BTK inhibitor is a compound of formula (I) or a pharmaceutically acceptable salt thereof;

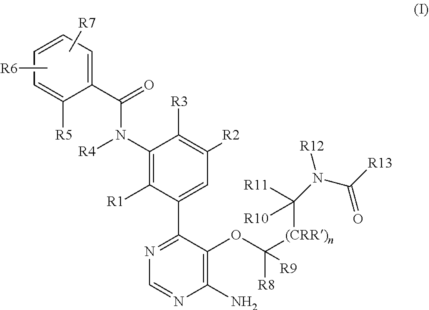

wherein,
R1 is hydrogen, C1-C6 alkyl optionally substituted by hydroxy;
R2 is hydrogen or halogen;
R3 is hydrogen or halogen;
R4 is hydrogen;
R5 is hydrogen or halogen;
or R4 and R5 are attached to each other and stand for a bond, —CH2-, —CH2-CH2-, —CH═CH—, —CH═CH—CH2-; —CH2-CH═CH—; or —CH2-CH2-CH2-; R6 and R7 stand independently from each other for H, C1-C6 alkyl optionally substituted by hydroxyl, C3-C6 cycloalkyl optionally substituted by halogen or hydroxy, or halogen; R8, R9, R, R′, R10 and R11 independently from each other stand for H, or C1-C6 alkyl optionally substituted by C1-C6 alkoxy; or any two of R8, R9, R, R′, R10 and R11 together with the carbon atom to which they are bound may form a 3-6 membered saturated carbocyclic ring; R12 is hydrogen or C1-C6 alkyl optionally substituted by halogen or C1-C6 alkoxy; or R12 and any one of R8, R9, R, R′, R10 or R11 together with the atoms to which they are bound may form a 4, 5, 6 or 7 membered azacyclic ring, which ring may optionally be substituted by halogen, cyano, hydroxyl, C1-C6 alkyl or C1-C6 alkoxy;
n is 0 or 1; and
R13 is C2-C6 alkenyl optionally substituted by C1-C6 alkyl, C1-C6 alkoxy orN,N-di-C1-C6 alkyl amino; C2-C6 alkynyl optionally substituted by C1-C6 alkyl or C1-C6 alkoxy; or C2-C6 alkylenyl oxide optionally substituted by C1-C6 alkyl.

In some embodiments, the BTK inhibitor of Formula I is chosen from: N-(3-(5-((1-Acryloylazetidin-3-yl)oxy)-6-aminopyrimidin-4-yl)-5-fluoro-2-methylphenyl)-4-cyclopropyl-2-fluorobenzamide; (E)-N-(3-(6-Amino-5-((1-(but-2-enoyl)azetidin-3-yl)oxy)pyrimidin-4-yl)-5-fluoro-2-methylphenyl)-4-cyclopropyl-2-fluorobenzamide; N-(3-(6-Amino-5-((1-propioloylazetidin-3-yl)oxy)pyrimidin-4-yl)-5-fluoro-2-methylphenyl)-4-cyclopropyl-2-fluorobenzamide; N-(3-(6-Amino-5-((1-(but-2-ynoyl)azetidin-3-yl)oxy)pyrimidin-4-yl)-5-fluoro-2-methylphenyl)-4-cyclopropyl-2-fluorobenzamide; N-(3-(5-((1-Acryloylpiperidin-4-yl)oxy)-6-aminopyrimidin-4-yl)-5-fluoro-2-methylphenyl)-4-cyclopropyl-2-fluorobenzamide; N-(3-(6-Amino-5-(2-(N-methylacrylamido)ethoxy)pyrimidin-4-yl)-5-fluoro-2-methylphenyl)-4-cyclopropyl-2-fluorobenzamide; (E)-N-(3-(6-Amino-5-(2-(N-methylbut-2-enamido)ethoxy)pyrimidin-4-yl)-5-fluoro-2-methylphenyl)-4-cyclopropyl-2-fluorobenzamide; N-(3-(6-Amino-5-(2-(N-methylpropiolamido)ethoxy)pyrimidin-4-yl)-5-fluoro-2-methylphenyl)-4-cyclopropyl-2-fluorobenzamide; (E)-N-(3-(6-Amino-5-(2-(4-methoxy-N-methylbut-2-enamido)ethoxy)pyrimidin-4-yl)-5-fluoro-2-methylphenyl)-4-cyclopropyl-2-fluorobenzamide; N-(3-(6-Amino-5-(2-(N-methylbut-2-ynamido)ethoxy)pyrimidin-4-yl)-5-fluoro-2-methylphenyl)-4-cyclopropyl-2-fluorobenzamide; N-(2-((4-Amino-6-(3-(4-cyclopropyl-2-fluorobenzamido)-5-fluoro-2-methylphenyl)pyrimidin-5-yl)oxy)ethyl)-N-methyloxirane-2-carboxamide; N-(2-((4-Amino-6-(3-(6-cyclopropyl-8-fluoro-1-oxoisoquinolin-2(1H)-yl)phenyl)pyrimidin-5-yl)oxy)ethyl)-N-methylacrylamide; N-(3-(5-(2-Acrylamidoethoxy)-6-aminopyrimidin-4-yl)-5-fluoro-2-methylphenyl)-4-cyclopropyl-2-fluorobenzamide; N-(3-(6-Amino-5-(2-(N-ethylacrylamido)ethoxy)pyrimidin-4-yl)-5-fluoro-2-methylphenyl)-4-cyclopropyl-2-fluorobenzamide; N-(3-(6-Amino-5-(2-(N-(2-fluoroethyl)acrylamido)ethoxy)pyrimidin-4-yl)-5-fluoro-2-methylphenyl)-4-cyclopropyl-2-fluorobenzamide; N-(3-(5-((1-Acrylamidocyclopropyl)methoxy)-6-aminopyrimidin-4-yl)-5-fluoro-2-methylphenyl)-4-cyclopropyl-2-fluorobenzamide; (S)—N-(3-(5-(2-Acrylamidopropoxy)-6-aminopyrimidin-4-yl)-5-fluoro-2-methylphenyl)-4-cyclopropyl-2-fluorobenzamide; (S)—N-(3-(6-Amino-5-(2-(but-2-ynamido)propoxy)pyrimidin-4-yl)-5-fluoro-2-methylphenyl)-4-cyclopropyl-2-fluorobenzamide; (S)—N-(3-(6-Amino-5-(2-(N-methylacrylamido)propoxy)pyrimidin-4-yl)-5-fluoro-2-methylphenyl)-4-cyclopropyl-2-fluorobenzamide; (S)—N-(3-(6-Amino-5-(2-(N-methylbut-2-ynamido)propoxy)pyrimidin-4-yl)-5-fluoro-2-methylphenyl)-4-cyclopropyl-2-fluorobenzamide; N-(3-(6-Amino-5-(3-(N-methylacrylamido)propoxy)pyrimidin-4-yl)-5-fluoro-2-methylphenyl)-4-cyclopropyl-2-fluorobenzamide; (S)—N-(3-(5-((1-Acryloylpyrrolidin-2-yl)methoxy)-6-aminopyrimidin-4-yl)-5-fluoro-2-methylphenyl)-4-cyclopropyl-2-fluorobenzamide; (S)—N-(3-(6-Amino-5-((1-(but-2-ynoyl)pyrrolidin-2-yl)methoxy)pyrimidin-4-yl)-5-fluoro-2-methylphenyl)-4-cyclopropyl-2-fluorobenzamide; (S)-2-(3-(5-((1-Acryloylpyrrolidin-2-yl)methoxy)-6-aminopyrimidin-4-yl)-5-fluoro-2-(hydroxymethyl)phenyl)-6-cyclopropyl-3,4-dihydroisoquinolin-1(2H)-one; N-(2-((4-Amino-6-(3-(6-cyclopropyl-1-oxo-3,4-dihydroisoquinolin-2(1H)-yl)-5-fluoro-2-(hydroxymethyl)phenyl)pyrimidin-5-yl)oxy)ethyl)-N-methylacrylamide; N-(3-(5-(((2S,4R)-1-Acryloyl-4-methoxypyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4R)-1-(but-2-ynoyl)-4-methoxypyrrolidin-2-yl)methoxy)pyrimidin-4-yl)-5-fluoro-2-methylphenyl)-4-cyclopropyl-2-fluorobenzamide; 2-(3-(5-(((2S,4R)-1-Acryloyl-4-methoxypyrrolidin-2-yl)methoxy)-6-aminopyrimidin-4-yl)-5-fluoro-2-(hydroxymethyl)phenyl)-6-cyclopropyl-3,4-dihydroisoquinolin-1(2H)-one; N-(3-(5-(((2S,4S)-1-Acryloyl-4-methoxypyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4S)-1-(but-2-ynoyl)-4-methoxypyrrolidin-2-yl)methoxy)pyrimidin-4-yl)-5-fluoro-2-methylphenyl)-4-cyclopropyl-2-fluorobenzamide; N-(3-(5-(((2S,4R)-1-Acryloyl-4-fluoropyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4R)-1-(but-2-ynoyl)-4-fluoropyrrolidin-2-yl)methoxy)pyrimidin-4-yl)-5-fluoro-2-methylphenyl)-4-cyclopropyl-2-fluorobenzamide; (S)—N-(3-(5-((1-Acryloylazetidin-2-yl)methoxy)-6-aminopyrimidin-4-yl)-5-fluoro-2-methylphenyl)-4-cyclopropyl-2-fluorobenzamide; (S)—N-(3-(6-Amino-5-((1-propioloylazetidin-2-yl)methoxy)pyrimidin-4-yl)-5-fluoro-2-methylphenyl)-4-cyclopropyl-2-fluorobenzamide; (S)-2-(3-(5-((1-Acryloylazetidin-2-yl)methoxy)-6-aminopyrimidin-4-yl)-5-fluoro-2-(hydroxymethyl)phenyl)-6-cyclopropyl-3,4-dihydroisoquinolin-1(2H)-one; (R)—N-(3-(5-((1-Acryloylazetidin-2-yl)methoxy)-6-aminopyrimidin-4-yl)-5-fluoro-2-methylphenyl)-4-cyclopropyl-2-fluorobenzamide; (R)—N-(3-(5-((1-Acryloylpiperidin-3-yl)methoxy)-6-aminopyrimidin-4-yl)-5-fluoro-2-methylphenyl)-4-cyclopropyl-2-fluorobenzamide; N-(3-(5-(((2R,3S)-1-Acryloyl-3-methoxypyrrolidin-2-yl)methoxy)-6-aminopyrimidin-4-yl)-5-fluoro-2-methylphenyl)-4-cyclopropyl-2-fluorobenzamide; N-(3-(5-(((2S,4R)-1-Acryloyl-4-cyanopyrrolidin-2-yl)methoxy)-6-aminopyrimidin-4-yl)-5-fluoro-2-methylphenyl)-4-cyclopropyl-2-fluorobenzamide; or N-(3-(5-(((2S,4S)-1-Acryloyl-4-cyanopyrrolidin-2-yl)methoxy)-6-aminopyrimidin-4-yl)-5-fluoro-2-methylphenyl)-4-cyclopropyl-2-fluorobenzamide.

Unless otherwise provided, the chemical terms used above in describing the BTK inhibitor of Formula I are used according to their meanings as set out in International Application WO/2015/079417, which is herein incorporated by reference in its entirety.

In one embodiment, the kinase inhibitor is an mTOR inhibitor selected from temsirolimus; ridaforolimus (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as AP23573 and MK8669; everolimus (RAD001); rapamycin (AY22989); simapimod; (5-{2,4-bis [(3S)-3-methylmorpholin-4-yl]pyrido[2,3-d]pyrimidin-7-yl}-2-methoxyphenyl)methanol (AZD8055); 2-amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502); and N2-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-(SEQ ID NO: 383), inner salt (SF1126); and XL765.

In one embodiment, the kinase inhibitor is an mTOR inhibitor, e.g., rapamycin, and the rapamycin is administered at a dose of about 3 mg, 4 mg, 5 mg, 6 mg, 7 mg, 8 mg, 9 mg, 10 mg (e.g., 6 mg) daily for a period of time, e.g., daily for 21 day cycle cycle, or daily for 28 day cycle. In one embodiment, 1, 2, 3, 4, 5, 6, 7, 8, 9, 10, 11, 12 or more cycles of rapamycin are administered. In one embodiment, the kinase inhibitor is an mTOR inhibitor, e.g., everolimus and the everolimus is administered at a dose of about 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg (e.g., 10 mg) daily for a period of time, e.g., daily for 28 day cycle. In one embodiment, 1, 2, 3, 4, 5, 6, 7, 8, 9, 10, 11, 12 or more cycles of everolimus are administered.

In one embodiment, the kinase inhibitor is an MNK inhibitor selected from CGP052088; 4-amino-3-(p-fluorophenylamino)-pyrazolo[3,4-d]pyrimidine (CGP57380); cercosporamide; ETC-1780445-2; and 4-amino-5-(4-fluoroanilino)-pyrazolo[3,4-d]pyrimidine.

In an embodiment, a CAR-expressing cell described herein is administered to a subject in combination with a phosphoinositide 3-kinase (PI3K) inhibitor (e.g., a PI3K inhibitor described herein, e.g., idelalisib or duvelisib) and/or rituximab. In embodiments, a CAR-expressing cell described herein is administered to a subject in combination with idelalisib and rituximab. In embodiments, a CAR-expressing cell described herein is administered to a subject in combination with duvelisib and rituximab. Idelalisib (also called GS-1101 or CAL-101; Gilead) is a small molecule that blocks the delta isoform of PI3K. The structure of idelalisib (5-Fluoro-3-phenyl-2-[(1S)-1-(7H-purin-6-ylamino)propyl]-4(3H)-quinazolinone) is shown below.

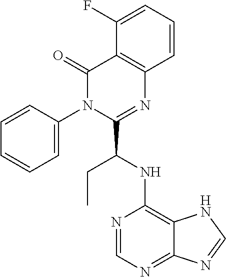 [SMILES: CC[C@H](Nc1ncnc2nc[nH]c12)c1nc2cccc(F)c2c(=O)n1-c1ccccc1]

Duvelisib (also called IPI-145; Infinity Pharmaceuticals and Abbvie) is a small molecule that blocks PI3K-δ,γ. The structure of duvelisib (8-Chloro-2-phenyl-3-[(1S)-1-(9H-purin-6-ylamino)ethyl]-1(2H)-isoquinolinone) is shown below.

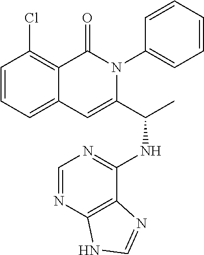 [SMILES: C[C@H](Nc1ncnc2[nH]cnc12)c1cc2cccc(Cl)c2c(=O)n1-c1ccccc1]

In embodiments, the subject has CLL. In embodiments, the subject has relapsed CLL, e.g., the subject has previously been administered a cancer therapy (e.g., previously been administered an anti-CD20 antibody or previously been administered ibrutinib). For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgVH) gene. In other embodiments, the subject does not comprise a leukemic cell comprising a mutation in the immunoglobulin heavy-chain variable-region (IgVH) gene. In embodiments, the subject has a deletion in the long arm of chromosome 11 (del(11 q)). In other embodiments, the subject does not have a del(11q). In embodiments, idelalisib is administered at a dosage of about 100-400 mg (e.g., 100-125, 125-150, 150-175, 175-200, 200-225, 225-250, 250-275, 275-300, 325-350, 350-375, or 375-400 mg), e.g., BID. In embodiments, duvelisib is administered at a dosage of about 15-100 mg (e.g., about 15-25, 25-50, 50-75, or 75-100 mg), e.g., twice a day. In embodiments, rituximab is administered at a dosage of about 350-550 mg/m2 (e.g., 350-375, 375-400, 400-425, 425-450, 450-475, or 475-500 mg/m2), e.g., intravenously.

In embodiments, a CAR-expressing cell described herein is administered to a subject in combination with an anaplastic lymphoma kinase (ALK) inhibitor. Exemplary ALK kinases include but are not limited to crizotinib (Pfizer), ceritinib (Novartis), alectinib (Chugai), brigatinib (also called AP26113; Ariad), entrectinib (Ignyta), PF-06463922 (Pfizer), TSR-011 (Tesaro) (see, e.g., Clinical Trial Identifier No. NCT02048488), CEP-37440 (Teva), and X-396 (Xcovery). In some embodiments, the subject has a solid cancer, e.g., a solid cancer described herein, e.g., lung cancer.

The chemical name of crizotinib is 3-[(1R)-1-(2,6-dichloro-3-fluorophenyl)ethoxy]-5-(1-piperidin-4-ylpyrazol-4-yl)pyridin-2-amine. The chemical name of ceritinib is 5-Chloro-N2-[2-isopropoxy-5-methyl-4-(4-piperidinyl)phenyl]-N4-[2-(isopropylsulfonyl)phenyl]-2,4-pyrimidinediamine. The chemical name of alectinib is 9-ethyl-6,6-dimethyl-8-(4-morpholinopiperidin-1-yl)-11-oxo-6,11-dihydro-5H-benzo[b]carbazole-3-carbonitrile. The chemical name of brigatinib is 5-Chloro-N2-{4-[4-(dimethylamino)-1-piperidinyl]-2-methoxyphenyl}-N4-[2-(dimethylphosphoryl)phenyl]-2,4-pyrimidinediamine. The chemical name of entrectinib is N-(5-(3,5-difluorobenzyl)-1H-indazol-3-yl)-4-(4-methylpiperazin-1-yl)-2-((tetrahydro-2H-pyran-4-yl)amino)benzamide. The chemical name of PF-06463922 is (10R)-7-Amino-12-fluoro-2,10,16-trimethyl-15-oxo-10,15,16,17-tetrahydro-2H-8,4-(metheno)pyrazolo[4,3-h][2,5,11]-benzoxadiazacyclotetradecine-3-carbonitrile. The chemical structure of CEP-37440 is (S)-2-((5-chloro-2-((6-(4-(2-hydroxyethyl)piperazin-1-yl)-1-methoxy-6,7,8,9-tetrahydro-5H-benzo[7]annulen-2-yl)amino)pyrimidin-4-yl)amino)-N-methylbenzamide. The chemical name of X-396 is (R)-6-amino-5-(1-(2,6-dichloro-3-fluorophenyl)ethoxy)-N-(4-(4-methylpiperazine-1-carbonyl)phenyl)pyridazine-3-carboxamide.

In one embodiment, the kinase inhibitor is a dual phosphatidylinositol 3-kinase (P13K) and mTOR inhibitor selected from 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF-04691502); N-[4-[[4-(Dimethylamino)-1-piperidinyl]carbonyl]phenyl]-N-[4-(4,6-di-4-morpholinyl-1,3,5-triazin-2-yl)phenyl]urea (PF-05212384, PKI-587); 2-Methyl-2-{4-[3-methyl-2-oxo-8-(quinolin-3-yl)-2,3-dihydro-1H-imidazo[4,5-c]quinolin-1-yl]phenyl}propanenitrile (BEZ-235); apitolisib (GDC-0980, RG7422); 2,4-Difluoro-N-{2-(methyloxy)-5-[4-(4-pyridazinyl)-6-quinolinyl]-3-pyridinyl}benzenesulfonamide (GSK2126458); 8-(6-methoxypyridin-3-yl)-3-methyl-1-(4-(piperazin-1-yl)-3-(trifluoromethyl)phenyl)-1H-imidazo[4,5-c]quinolin-2(3H)-one Maleic acid (NVP-BGT226); 3-[4-(4-Morpholinylpyrido[3′,2′:4,5]furo[3,2-d]pyrimidin-2-yl]phenol (PI-103); 5-(9-isopropyl-8-methyl-2-morpholino-9H-purin-6-yl)pyrimidin-2-amine (VS-5584, SB2343); and N-[2-[(3,5-Dimethoxyphenyl)amino]quinoxalin-3-yl]-4-[(4-methyl-3-methoxyphenyl)carbonyl]aminophenylsulfonamide (XL765).

Drugs that inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993) can also be used. In a further aspect, the cell compositions of the present invention may be administered to a patient in conjunction with (e.g., before, simultaneously or following) bone marrow transplantation, T cell ablative therapy using chemotherapy agents such as, fludarabine, external-beam radiation therapy (XRT), cyclophosphamide, and/or antibodies such as OKT3 or CAMPATH. In one aspect, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

In one embodiment, a CAR expressing cell described herein is administered to a subject in combination with a biphosphonate, e.g., Pamidronate (Aredia®); Zoledronic acid or Zoledronate (Zometa®, Zomera®, Aclasta®, or Reclast®); Alendronate (Fosamax®); Risedronate (Actonel®); Ibandronate (Boniva®); Clondronate (Bonefos®); Etidronate (Didronel®); Tiludronate (Skelid®); Pamidronate (Aredia@); Neridronate (Nerixia®); Strontiun ranelate (Protelos®, or Protos®); and Teriparatide (Forteo®).

In one embodiment, a CAR expressing cell described herein is administered to a subject in combination with a corticosteroid, e.g., dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate@ and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

In one embodiment, a CAR expressing cell described herein is administered to a subject in combination with an immunomodulator, e.g., Afutuzumab (available from Roche®); Pegfilgrastim (Neulasta®); Lenalidomide (CC-5013, Revlimid®); Thalidomide (Thalomid®), Actimid (CC4047); and IRX-2 (mixture of human cytokines including interleukin 1, interleukin 2, and interferon γ, CAS 951209-71-5, available from IRX Therapeutics.

In one embodiment, a CAR expressing cell described herein is administered to a subject in combination with a proteasome inhibitor, e.g., Bortezomib (Velcade®); Ixazomib citrate (MLN9708, CAS 1201902-80-8); Danoprevir (RG7227, CAS 850876-88-9); Ixazomib (MLN2238, CAS 1072833-77-2); and (S)—N-[(phenylmethoxy)carbonyl]-L-leucyl-N-(1-formyl-3-methylbutyl)-L-Leucinamide (MG-132, CAS 133407-82-6).

In one embodiment, a CAR expressing cell described herein is administered to a subject in combination with a vascular endothelial growth factor (VEGF) receptor, e.g., Bevacizumab (Avastin®), axitinib (Inlyta®); Brivanib alaninate (BMS-582664, (S)-((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®).

In one embodiment, a CAR expressing cell described herein is administered to a subject in combination with a CD20 antibody or a conjugate thereof, e.g., Rituximab (Riuxan® and MabThera®); and Tositumomab (Bexxar®); and Ofatumumab (Arzerra®), Ibritumomab tiuxetan (Zevalin®); and Tositumomab,

In one embodiment, a CAR expressing cell described herein is administered to a subject in combination with an anticonvulsant, e.g., Anticonvulsants (antiepileptic or antiseizure drugs): aldehydes, e.g., paraldehyde; aromatic allylic alcohols, e.g., stiripentol (Diacomit®); barbiturates, e.g., phenobarbital (Luminal®), methylphenobarbital (Mebaral®), barbexaclone (Maliasin®), benzodiazepines, e.g., clobazam (Onfi®), clonazepam (Klonopin®), clorazepate (Tranxene® and Novo-Clopate®), diazepam (Valium®, Lembrol®, Diastat®), midazolam (Versed®), lorazepam (Ativan® and Orfidal®), nitrazepam (Alodorm®, Arem®, Insoma®), temazepam (Restoril®, Normison®), nimetzepam (Erimin®), bromides, e.g., potassium bromide; carbamates, e.g., felbamate (Felbatol®); carboxamides, e.g., carbamazepine (Tegretol®, Equetro®), oxcarbazepine (Trileptal®, Oxcarb@), eslicarbazepine acetate (Aptiom®); fatty acids, e.g., valproates (valproic acid, sodium valproate, divalproex sodium), vigabatrin (Sabril®), progabide (Gabren®), tiagabine (Gabitril®); fructose derivatives, e.g., topiramate (Topamax®); GABA analogs, e.g., gabapentin (Neurontin®), pregabalin (Lyrica®); hydantoins, e.g., ethotoin (Peganone®), phenytoin (Dilantin®), mephenytoin (Mesantoin®), fosphenytoin (Cerebyx®, Prodilantin®); oxazolidinediones, e.g., paramethadione (Paradione®), trimethadione (Tridione®); propionates, e.g., beclamide (Choracon®, Hibicon®, Posedrine®); pyrimidinediones, e.g., primidone (Mysoline®); pyrrolidines, e.g., brivaracetam, levetiracetam, seletracetam (Keppra®); succinimides, e.g., ethosuximide (Zarontin®), phensuximide (Milontin®), mesuximide (Celontin®, Petinutin®); sulfonamides, e.g., acetazolamide (Diamox®), sultiame (Ospolot®), methazolamide (Neptazane®), zonisamide (Zonegran®); triazines, e.g., lamotrigine (Lamictal®); ureas, e.g., pheneturide, phenacemide (Phenurone®); valproylamides (amide derivaties of valproate), e.g., valpromide (Depamide®), valnoctamide; AMPA receptor antagonist, e.g., perampanel (Fycompa®).

In embodiments, a CAR-expressing cell described herein is administered to a subject in combination with an indoleamine 2,3-dioxygenase (IDO) inhibitor. IDO is an enzyme that catalyzes the degradation of the amino acid, L-tryptophan, to kynurenine. Many cancers overexpress IDO, e.g., prostatic, colorectal, pancreatic, cervical, gastric, ovarian, head, and lung cancer. pDCs, macrophages, and dendritic cells (DCs) can express IDO. Without being bound by theory, it is thought that a decrease in L-tryptophan (e.g., catalyzed by IDO) results in an immunosuppressive milieu by inducing T-cell anergy and apoptosis. Thus, without being bound by theory, it is thought that an IDO inhibitor can enhance the efficacy of a CAR-expressing cell described herein, e.g., by decreasing the suppression or death of a CAR-expressing immune cell. In embodiments, the subject has a solid tumor, e.g., a solid tumor described herein, e.g., prostatic, colorectal, pancreatic, cervical, gastric, ovarian, head, or lung cancer. Exemplary inhibitors of IDO include but are not limited to 1-methyl-tryptophan, indoximod (NewLink Genetics) (see, e.g., Clinical Trial Identifier Nos. NCT01191216; NCT01792050), and INCB024360 (Incyte Corp.) (see, e.g., Clinical Trial Identifier Nos. NCT01604889; NCT01685255)

In embodiments, a CAR-expressing cell described herein is administered to a subject in combination with a modulator of myeloid-derived suppressor cells (MDSCs). MDSCs accumulate in the periphery and at the tumor site of many solid tumors. These cells suppress T cell responses, thereby hindering the efficacy of CAR-expressing cell therapy. Without being bound by theory, it is thought that administration of a MDSC modulator enhances the efficacy of a CAR-expressing cell described herein. In an embodiment, the subject has a solid tumor, e.g., a solid tumor described herein, e.g., glioblastoma. Exemplary modulators of MDSCs include but are not limited to MCS 110 and BLZ945. MCS110 is a monoclonal antibody (mAb) against macrophage colony-stimulating factor (M-CSF). See, e.g., Clinical Trial Identifier No. NCT00757757. BLZ945 is a small molecule inhibitor of colony stimulating factor 1 receptor (CSF1R). See, e.g., Pyonteck et al. Nat. Med. 19(2013):1264-72. The structure of BLZ945 is shown below.

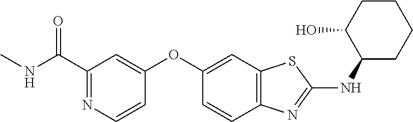 [SMILES: CNC(=O)c1cc(Oc2ccc3nc(N[C@@H]4CCCC[C@H]4O)sc3c2)ccn1]

In embodiments, a CAR-expressing cell described herein is administered to a subject in combination with a CD19 CART cell (e.g., CTL019, e.g., as described in WO2012/079000, incorporated herein by reference). In embodiments, the subject has acute myeloid leukemia (AML), e.g., a CD19 positive AML or a CD19 negative AML. In embodiments, the subject has a CD19+ lymphoma, e.g., a CD19+Non-Hodgkin's Lymphoma (NHL), a CD19+FL, or a CD19+DLBCL. In embodiments, the subject has a relapsed or refractory CD19+ lymphoma. In embodiments, a lymphodepleting chemotherapy is administered to the subject prior to, concurrently with, or after administration (e.g., infusion) of CD19 CART cells. In an example, the lymphodepleting chemotherapy is administered to the subject prior to administration of CD19 CART cells. For example, the lymphodepleting chemotherapy ends 1-4 days (e.g., 1, 2, 3, or 4 days) prior to CD19 CART cell infusion. In embodiments, multiple doses of CD19 CART cells are administered, e.g., as described herein. For example, a single dose comprises about 5×108 CD19 CART cells. In embodiments, a lymphodepleting chemotherapy is administered to the subject prior to, concurrently with, or after administration (e.g., infusion) of a CAR-expressing cell described herein, e.g., a non-CD19 CAR-expressing cell. In embodiments, a CD19 CART is administered to the subject prior to, concurrently with, or after administration (e.g., infusion) of a non-CD19 CAR-expressing cell, e.g., a non-CD19 CAR-expressing cell described herein.

In some embodiments, a CAR-expressing cell described herein is administered to a subject in combination with a CD19 CAR-expressing cell, e.g., CTL019, e.g., as described in WO2012/079000, incorporated herein by reference, for treatment of a disease associated with the expression of BCMA, e.g., a cancer described herein. Without being bound by theory, it is believed that administering a CD19 CAR-expressing cell in combination with a CAR-expressing cell improves the efficacy of a CAR-expressing cell described herein by targeting early lineage cancer cells, e.g., cancer stem cells, modulating the immune response, depleting regulatory B cells, and/or improving the tumor microenvironment. For example, a CD19 CAR-expressing cell targets cancer cells that express early lineage markers, e.g., cancer stem cells and CD19-expressing cells, while the CAR-expressing cell described herein targets cancer cells that express later lineage markers, e.g., BCMA. This preconditioning approach can improve the efficacy of the CAR-expressing cell described herein. In such embodiments, the CD19 CAR-expressing cell is administered prior to, concurrently with, or after administration (e.g., infusion) of a CAR-expressing cell described herein.

In embodiments, a CAR-expressing cell described herein also expresses a CAR targeting CD19, e.g., a CD19 CAR. In an embodiment, the cell expressing a CAR described herein and a CD19 CAR is administered to a subject for treatment of a cancer described herein, e.g., AML. In an embodiment, the configurations of one or both of the CAR molecules comprise a primary intracellular signaling domain and a costimulatory signaling domain. In another embodiment, the configurations of one or both of the CAR molecules comprise a primary intracellular signaling domain and two or more, e.g., 2, 3, 4, or 5 or more, costimulatory signaling domains. In such embodiments, the CAR molecule described herein and the CD19 CAR may have the same or a different primary intracellular signaling domain, the same or different costimulatory signaling domains, or the same number or a different number of costimulatory signaling domains. Alternatively, the CAR described herein and the CD19 CAR are configured as a split CAR, in which one of the CAR molecules comprises an antigen binding domain and a costimulatory domain (e.g., 4-1BB), while the other CAR molecule comprises an antigen binding domain and a primary intracellular signaling domain (e.g., CD3 zeta).

In some embodiments, a CAR-expressing cell described herein is administered to a subject in combination with a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15 (Admune Therapeutics, LLC). hetIL-15 is a heterodimeric non-covalent complex of IL-15 and IL-15Ra. hetIL-15 is described in, e.g., U.S. Pat. No. 8,124,084, U.S. 2012/0177598, U.S. 2009/0082299, U.S. 2012/0141413, and U.S. 2011/0081311, incorporated herein by reference. In embodiments, het-IL-15 is administered subcutaneously. In embodiments, the subject has a cancer, e.g., solid cancer, e.g., melanoma or colon cancer. In embodiments, the subject has a metastatic cancer.

In one embodiment, the subject can be administered an agent which reduces or ameliorates a side effect associated with the administration of a CAR-expressing cell. Side effects associated with the administration of a CAR-expressing cell include, but are not limited to CRS, and hemophagocytic lymphohistiocytosis (HLH), also termed Macrophage Activation Syndrome (MAS). Symptoms of CRS include high fevers, nausea, transient hypotension, hypoxia, and the like. CRS may include clinical constitutional signs and symptoms such as fever, fatigue, anorexia, myalgias, arthalgias, nausea, vomiting, and headache. CRS may include clinical skin signs and symptoms such as rash. CRS may include clinical gastrointestinal signs and symsptoms such as nausea, vomiting and diarrhea. CRS may include clinical respiratory signs and symptoms such as tachypnea and hypoxemia. CRS may include clinical cardiovascular signs and symptoms such as tachycardia, widened pulse pressure, hypotension, increased cardiac output (early) and potentially diminished cardiac output (late). CRS may include clinical coagulation signs and symptoms such as elevated d-dimer, hypofibrinogenemia with or without bleeding. CRS may include clinical renal signs and symptoms such as azotemia. CRS may include clinical hepatic signs and symptoms such as transaminitis and hyperbilirubinemia. CRS may include clinical neurologic signs and symptoms such as headache, mental status changes, confusion, delirium, word finding difficulty or frank aphasia, hallucinations, tremor, dymetria, altered gait, and seizures.

Accordingly, the methods described herein can comprise administering a CAR-expressing cell described herein to a subject and further administering one or more agents to manage elevated levels of a soluble factor resulting from treatment with a CAR-expressing cell. In one embodiment, the soluble factor elevated in the subject is one or more of IFN-γ, TNFα, IL-2 and IL-6. In an embodiment, the factor elevated in the subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antibody fragment. Examples of such agents include, but are not limited to a steroid (e.g., corticosteroid), an inhibitor of TNFα, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitors of TNFα include, but are not limited to, xanthine derivatives (e.g. pentoxifylline) and bupropion. An example of an IL-6 inhibitor is an anti-IL-6 antibody molecule such as tocilizumab (toc), sarilumab, elsilimomab, CNTO 328, ALD518/BMS-945429, CNTO 136, CPSI-2364, CDP6038, VX30, ARGX-109, FE301, and FM101. In one embodiment, the anti-IL-6 antibody molecule is tocilizumab. An example of an IL-1R based inhibitor is anakinra.

In some embodiment, the subject is administered a corticosteroid, such as, e.g., methylprednisolone, hydrocortisone, among others.

In some embodiments, the subject is administered a vasopressor, such as, e.g., norepinephrine, dopamine, phenylephrine, epinephrine, vasopressin, or a combination thereof.

In an embodiment, the subject can be administered an antipyretic agent. In an embodiment, the subject can be administered an analgesic agent.

In one embodiment, the subject can be administered an agent which enhances the activity of a CAR-expressing cell. For example, in one embodiment, the agent can be an agent which inhibits an inhibitory molecule, e.g., the agent is a checkpoint inhibitor. Inhibitory molecules, e.g., Programmed Death 1 (PD1), can, in some embodiments, decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGFR beta. Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used to inhibit expression of an inhibitory molecule in the CAR-expressing cell. In an embodiment the inhibitor is an shRNA. In an embodiment, the inhibitory molecule is inhibited within a CAR-expressing cell. In these embodiments, a dsRNA molecule that inhibits expression of the inhibitory molecule is linked to the nucleic acid that encodes a component, e.g., all of the components, of the CAR. In embodiments, a CAR-expressing cell described herein is administered in combination with an inhibitor of an inhibitory molecule, e.g., in combination with a checkpoint inhibitor, e.g., in combination with an inhibitor of PD1 and/or PD-L1. In embodiments, a CAR-expressing cell described herein is administered in combination with an inhibitor of PD1. In embodiments, a CAR-expressing cell described herein is administered in combination with an inhibitor of PD-L1.

In an embodiment, a nucleic acid molecule that encodes a dsRNA molecule that inhibits expression of the molecule that modulates or regulates, e.g., inhibits, T-cell function is operably linked to a promoter, e.g., a H1- or a U6-derived promoter such that the dsRNA molecule that inhibits expression of the molecule that modulates or regulates, e.g., inhibits, T-cell function is expressed, e.g., is expressed within a CAR-expressing cell. See e.g., Tiscornia G., “Development of Lentiviral Vectors Expressing siRNA,” Chapter 3, in Gene Transfer: Delivery and Expression of DNA and RNA (eds. Friedmann and Rossi). Cold Spring Harbor Laboratory Press, Cold Spring Harbor, N.Y., USA, 2007; Brummelkamp T R, et al. (2002) Science 296: 550-553; Miyagishi M, et al. (2002) Nat. Biotechnol. 19: 497-500. In an embodiment the nucleic acid molecule that encodes a dsRNA molecule that inhibits expression of the molecule that modulates or regulates, e.g., inhibits, T-cell function is present on the same vector, e.g., a lentiviral vector, that comprises a nucleic acid molecule that encodes a component, e.g., all of the components, of the CAR. In such an embodiment, the nucleic acid molecule that encodes a dsRNA molecule that inhibits expression of the molecule that modulates or regulates, e.g., inhibits, T-cell function is located on the vector, e.g., the lentiviral vector, 5′- or 3′- to the nucleic acid that encodes a component, e.g., all of the components, of the CAR. The nucleic acid molecule that encodes a dsRNA molecule that inhibits expression of the molecule that modulates or regulates, e.g., inhibits, T-cell function can be transcribed in the same or different direction as the nucleic acid that encodes a component, e.g., all of the components, of the CAR. In an embodiment the nucleic acid molecule that encodes a dsRNA molecule that inhibits expression of the molecule that modulates or regulates, e.g., inhibits, T-cell function is present on a vector other than the vector that comprises a nucleic acid molecule that encodes a component, e.g., all of the components, of the CAR. In an embodiment, the nucleic acid molecule that encodes a dsRNA molecule that inhibits expression of the molecule that modulates or regulates, e.g., inhibits, T-cell function it transiently expressed within a CAR-expressing cell. In an embodiment, the nucleic acid molecule that encodes a dsRNA molecule that inhibits expression of the molecule that modulates or regulates, e.g., inhibits, T-cell function is stably integrated into the genome of a CAR-expressing cell. FIGS. 41A-41E depicts examples of vectors for expressing a component, e.g., all of the components, of the CAR with a dsRNA molecule that inhibits expression of the molecule that modulates or regulates, e.g., inhibits, T-cell function.

dsRNA molecules can also be useful in the disclosed methods for inhibiting expression of a molecule that modulates or regulates, e.g., inhibits, T-cell function, wherein the molecule that modulates or regulates, e.g., inhibits, T-cell function is PD-1.

In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206).). In an embodiment, the agent is an antibody or antibody fragment that binds to TIM3. In an embodiment, the agent is an antibody or antibody fragment that binds to LAG3. In embodiments, the agent that enhances the activity of a CAR-expressing cell, e.g., inhibitor of an inhibitory molecule, is administered in combination with an allogeneic CAR, e.g., an allogeneic CAR described herein (e.g., described in the Allogeneic CAR section herein).

PD-1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD-1, PD-L1 and PD-L2 have been shown to downregulate T cell activation upon binding to PD-1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD-1 with PD-L1. Antibodies, antibody fragments, and other inhibitors of PD-1, PD-L1 and PD-L2 are available in the art and may be used combination with a cars of the present invention described herein. For example, nivolumab (also referred to as BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are disclosed in WO2009/101611. Pembrolizumab (formerly known as lambrolizumab, and also referred to as MK03475; Merck) is a humanized IgG4 monoclonal antibody that binds to PD-1. Pembrolizumab and other humanized anti-PD-1 antibodies are disclosed in U.S. Pat. No. 8,354,509 and WO2009/114335. MEDI4736 (Medimmune) is a human monoclonal antibody that binds to PDL1, and inhibits interaction of the ligand with PD1. MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. Other anti-PD-L1 binding agents include YW243.55.570 (heavy and light chain variable regions are shown in SEQ ID NOs 20 and 21 in WO2010/077634) and MDX-1 105 (also referred to as BMS-936559, and, e.g., anti-PD-L1 binding agents disclosed in WO2007/005874). AMP-224 (B7-DCIg; Amplimmune; e.g., disclosed in WO2010/027827 and WO2011/066342), is a PD-L2 Fc fusion soluble receptor that blocks the interaction between PD-1 and B7-H1. Other anti-PD-1 antibodies include AMP 514 (Amplimmune), among others, e.g., anti-PD-1 antibodies disclosed in U.S. Pat. No. 8,609,089, US 2010028330, and/or US 20120114649.

TIM3 (T cell immunoglobulin-3) also negatively regulates T cell function, particularly in IFN-g-secreting CD4+ T helper 1 and CD8+ T cytotoxic 1 cells, and plays a critical role in T cell exhaustion. Inhibition of the interaction between TIM3 and its ligands, e.g., galectin-9 (Gal9), phosphotidylserine (PS), and HMGB1, can increase immune response. Antibodies, antibody fragments, and other inhibitors of TIM3 and its ligands are available in the art and may be used combination with a CD19 or BCMA CAR described herein. For example, antibodies, antibody fragments, small molecules, or peptide inhibitors that target TIM3 binds to the IgV domain of TIM3 to inhibit interaction with its ligands. Antibodies and peptides that inhibit TIM3 are disclosed in WO2013/006490 and US20100247521. Other anti-TIM3 antibodies include humanized versions of RMT3-23 (disclosed in Ngiow et al., 2011, Cancer Res, 71:3540-3551), and clone 8B.2C12 (disclosed in Monney et al., 2002, Nature, 415:536-541). Bi-specific antibodies that inhibit TIM3 and PD-1 are disclosed in US20130156774.

In other embodiments, the agent which enhances the activity of a CAR-expressing cell is a CEACAM inhibitor (e.g., CEACAM-1, CEACAM-3, and/or CEACAM-5 inhibitor). In one embodiment, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI: 10:1371/journal.pone.0021146), or crossreacts with CEACAM-1 and CEACAM-5 as described in, e.g., WO 2013/054331 and US 2014/0271618.

Without wishing to be bound by theory, carcinoembryonic antigen cell adhesion molecules (CEACAM), such as CEACAM-1 and CEACAM-5, are believed to mediate, at least in part, inhibition of an anti-tumor immune response (see e.g., Markel et al. J Immunol. 2002 Mar. 15; 168(6):2803-10; Markel et al. J Immunol. 2006 Nov. 1; 177(9):6062-71; Markel et al. Immunology. 2009 February; 126(2):186-200; Markel et al. Cancer Immunol Immunother. 2010 February; 59(2):215-30; Ortenberg et al. Mol Cancer Ther. 2012 June; 11(6):1300-10; Stem et al. J Immunol. 2005 Jun. 1; 174(11):6692-701; Zheng et al. PLoS One. 2010 Sep. 2; 5(9). pii: e12529). For example, CEACAM-1 has been described as a heterophilic ligand for TIM-3 and as playing a role in TIM-3-mediated T cell tolerance and exhaustion (see e.g., WO 2014/022332; Huang, et al. (2014) Nature doi:10.1038/nature13848). In embodiments, co-blockade of CEACAM-1 and TIM-3 has been shown to enhance an anti-tumor immune response in xenograft colorectal cancer models (see e.g., WO 2014/022332; Huang, et al. (2014), supra). In other embodiments, co-blockade of CEACAM-1 and PD-1 reduce T cell tolerance as described, e.g., in WO 2014/059251. Thus, CEACAM inhibitors can be used with the other immunomodulators described herein (e.g., anti-PD-1 and/or anti-TIM-3 inhibitors) to enhance an immune response against a cancer, e.g., a melanoma, a lung cancer (e.g., NSCLC), a bladder cancer, a colon cancer an ovarian cancer, and other cancers as described herein.

LAG3 (lymphocyte activation gene-3 or CD223) is a cell surface molecule expressed on activated T cells and B cells that has been shown to play a role in CD8+ T cell exhaustion. Antibodies, antibody fragments, and other inhibitors of LAG3 and its ligands are available in the art and may be used combination with a CD19 or BCMA CAR described herein. For example, BMS-986016 (Bristol-Myers Squib) is a monoclonal antibody that targets LAW. IMP701 (Immutep) is an antagonist LAG3 antibody and IMP731 (Immutep and GlaxoSmithKline) is a depleting LAG3 antibody. Other LAG3 inhibitors include IMP321 (Immutep), which is a recombinant fusion protein of a soluble portion of LAG3 and Ig that binds to MHC class II molecules and activates antigen presenting cells (APC). Other antibodies are disclosed, e.g., in WO2010/019570.

In some embodiments, the agent which enhances the activity of a CAR-expressing cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an antracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the CAR. In another embodiment, the fusion protein is expressed by a cell, e.g., a T cell or NK cell that does not express an anti-BCMA CAR.

In one embodiment, the agent which enhances activity of a CAR-expressing cell described herein is miR-17-92.

In one embodiment, the agent which enhances activity of a CAR-described herein is a cytokine. Cytokines have important functions related to T cell expansion, differentiation, survival, and homeostatis. Cytokines that can be administered to the subject receiving a CAR-expressing cell described herein include: IL-2, IL-4, IL-7, IL-9, IL-15, IL-18, and IL-21, or a combination thereof. In preferred embodiments, the cytokine administered is IL-7, IL-15, or IL-21, or a combination thereof. The cytokine can be administered once a day or more than once a day, e.g., twice a day, three times a day, or four times a day. The cytokine can be administered for more than one day, e.g. the cytokine is administered for 2 days, 3 days, 4 days, 5 days, 6 days, 1 week, 2 weeks, 3 weeks, or 4 weeks. For example, the cytokine is administered once a day for 7 days.

In embodiments, the cytokine is administered in combination with CAR-expressing T cells. The cytokine can be administered simultaneously or concurrently with the CAR-expressing T cells, e.g., administered on the same day. The cytokine may be prepared in the same pharmaceutical composition as the CAR-expressing T cells, or may be prepared in a separate pharmaceutical composition. Alternatively, the cytokine can be administered shortly after administration of the CAR-expressing T cells, e.g., 1 day, 2 days, 3 days, 4 days, 5 days, 6 days, or 7 days after administration of the CAR-expressing T cells. In embodiments where the cytokine is administered in a dosing regimen that occurs over more than one day, the first day of the cytokine dosing regimen can be on the same day as administration with the CAR-expressing T cells, or the first day of the cytokine dosing regimen can be 1 day, 2 days, 3 days, 4 days, 5 days, 6 days, or 7 days after administration of the CAR-expressing T cells. In one embodiment, on the first day, the CAR-expressing T cells are administered to the subject, and on the second day, a cytokine is administered once a day for the next 7 days. In a preferred embodiment, the cytokine to be administered in combination with CAR-expressing T cells is IL-7, IL-15, or IL-21.

In other embodiments, the cytokine is administered a period of time after administration of CAR-expressing cells, e.g., at least 2 weeks, 3 weeks, 4 weeks, 6 weeks, 8 weeks, 10 weeks, 12 weeks, 4 months, 5 months, 6 months, 7 months, 8 months, 9 months, 10 months, 11 months, or 1 year or more after administration of CAR-expressing cells. In one embodiment, the cytokine is administered after assessment of the subject's response to the CAR-expressing cells. For example, the subject is administered CAR-expressing cells according to the dosage and regimens described herein. The response of the subject to CAR-expressing cell therapy is assessed at 2 weeks, 3 weeks, 4 weeks, 6 weeks, 8 weeks, 10 weeks, 12 weeks, 4 months, 5 months, 6 months, 7 months, 8 months, 9 months, 10 months, 11 months, or 1 year or more after administration of CAR-expressing cells, using any of the methods described herein, including inhibition of tumor growth, reduction of circulating tumor cells, or tumor regression. Subjects that do not exhibit a sufficient response to CAR-expressing cell therapy can be administered a cytokine. Administration of the cytokine to the subject that has sub-optimal response to the CAR-expressing cell therapy improves CAR-expressing cell efficacy or anti-cancer activity. In a preferred embodiment, the cytokine administered after administration of CAR-expressing cells is IL-7.

In some embodiments, the methods disclosed herein can use low, immune enhancing, doses of mTOR inhibitors, e.g., allosteric mTOR inhibitors, including rapalogs such as RAD001. Administration of a low, immune enhancing, dose of an mTOR inhibitor (e.g., a dose that is insufficient to completely suppress the immune system, but sufficient to improve immune function) can optimize the performance of immune effector cells, e.g., T cells or CAR-expressing cells, in the subject. Methods for measuring mTOR inhibition, dosages, treatment regimens, and suitable pharmaceutical compositions are described in U.S. Patent Application No. 2015/01240036, hereby incorporated by reference.

In an embodiment, administration of a low, immune enhancing, dose of an mTOR inhibitor results in one or more of the following: i) a decrease in the number of PD-1 positive immune effector cells; ii) an increase in the number of PD-1 negative immune effector cells; iii) an increase in the ratio of PD-1 negative immune effector cells/PD-1 positive immune effector cells; iv) an increase in the number of naive T cells; v) an increase in the expression of one or more of the following markers: CD62Lhigh, CD127high, CD27+, and BCL2, e.g., on memory T cells, e.g., memory T cell precursors; vi) a decrease in the expression of KLRG1, e.g., on memory T cells, e.g., memory T cell precursors; or vii) an increase in the number of memory T cell precursors, e.g., cells with any one or combination of the following characteristics: increased CD62Lhigh increased CD127high increased CD27+, decreased KLRG1, and increased BCL2; and wherein any of the foregoing, e.g., i), ii), iii), iv), v), vi), or vii), occurs e.g., at least transiently, e.g., as compared to a non-treated subject

In another embodiment, administration of a low, immune enhancing, dose of an mTOR inhibitor results in increased or prolonged proliferation or persistence of CAR-expressing cells, e.g., in culture or in a subject, e.g., as compared to non-treated CAR-expressing cells or a non-treated subject. In embodiments, increased proliferation or persistence is associated with in an increase in the number of CAR-expressing cells. Methods for measuring increased or prolonged proliferation are described in Examples 15 and 16. In another embodiment, administration of a low, immune enhancing, dose of an mTOR inhibitor results in increased killing of cancer cells by CAR-expressing cells, e.g., in culture or in a subject, e.g., as compared to non-treated CAR-expressing cells or a non-treated subject. In embodiments, increased killing of cancer cells is associated with in a decrease in tumor volume. Methods for measuring increased killing of cancer cells are described herein, e.g., in Examples 2, 5-6, 8, and 13. In one embodiment, the cells expressing a CAR molecule, e.g., a CAR molecule described herein, are administered in combination with a low, immune enhancing dose of an mTOR inhibitor, e.g., an allosteric mTOR inhibitor, e.g., RAD001, or a catalytic mTOR inhibitor. For example, administration of the low, immune enhancing, dose of the mTOR inhibitor can be initiated prior to administration of a CAR-expressing cell described herein; completed prior to administration of a CAR-expressing cell described herein; initiated at the same time as administration of a CAR-expressing cell described herein; overlapping with administration of a CAR-expressing cell described herein; or continuing after administration of a CAR-expressing cell described herein.

Alternatively or in addition, administration of a low, immune enhancing, dose of an mTOR inhibitor can optimize immune effector cells to be engineered to express a CAR molecule described herein. In such embodiments, administration of a low, immune enhancing, dose of an mTOR inhibitor, e.g., an allosteric inhibitor, e.g., RAD001, or a catalytic inhibitor, is initiated or completed prior to harvest of immune effector cells, e.g., T cells or NK cells, to be engineered to express a CAR molecule described herein, from a subject.

In another embodiment, immune effector cells, e.g., T cells or NK cells, to be engineered to express a CAR molecule described herein, e.g., after harvest from a subject, or CAR-expressing immune effector cells, e.g., T cells or NK cells, e.g., prior to administration to a subject, can be cultured in the presence of a low, immune enhancing, dose of an mTOR inhibitor.

In an embodiment, administering to the subject a low, immune enhancing, dose of an mTOR inhibitor comprises administering, e.g., once per week, e.g., in an immediate release dosage form, 0.1 to 20, 0.5 to 10, 2.5 to 7.5, 3 to 6, or about 5, mgs of RAD001, or a bioequivalent dose thereof. In an embodiment, administering to the subject a low, immune enhancing, dose of an mTOR inhibitor comprises administering, e.g., once per week, e.g., in a sustained release dosage form, 0.3 to 60, 1.5 to 30, 7.5 to 22.5, 9 to 18, or about 15 mgs of RAD001, or a bioequivalent dose thereof.

In an embodiment, a dose of an mTOR inhibitor is associated with, or provides, mTOR inhibition of at least 5 but no more than 90%, at least 10 but no more than 90%, at least 15, but no more than 90%, at least 20 but no more than 90%, at least 30 but no more than 90%, at least 40 but no more than 90%, at least 50 but no more than 90%, at least 60 but no more than 90%, at least 70 but no more than 90%, at least 5 but no more than 80%, at least 10 but no more than 80%, at least 15, but no more than 80%, at least 20 but no more than 80%, at least 30 but no more than 80%, at least 40 but no more than 80%, at least 50 but no more than 80%, at least 60 but no more than 80%, at least 5 but no more than 70%, at least 10 but no more than 70%, at least 15, but no more than 70%, at least 20 but no more than 70%, at least 30 but no more than 70%, at least 40 but no more than 70%, at least 50 but no more than 70%, at least 5 but no more than 60%, at least 10 but no more than 60%, at least 15, but no more than 60%, at least 20 but no more than 60%, at least 30 but no more than 60%, at least 40 but no more than 60%, at least 5 but no more than 50%, at least 10 but no more than 50%, at least 15, but no more than 50%, at least 20 but no more than 50%, at least 30 but no more than 50%, at least 40 but no more than 50%, at least 5 but no more than 40%, at least 10 but no more than 40%, at least 15, but no more than 40%, at least 20 but no more than 40%, at least 30 but no more than 40%, at least 35 but no more than 40%, at least 5 but no more than 30%, at least 10 but no more than 30%, at least 15, but no more than 30%, at least 20 but no more than 30%, or at least 25 but no more than 30%.

In an embodiment, administering to the subject a low, immune enhancing, dose of an mTOR inhibitor comprises administering, e.g., once per week, e.g., in an immediate release dosage form, 0.1 to 20, 0.5 to 10, 2.5 to 7.5, 3 to 6, or about 5, mgs of RAD001, or a bioequivalent dose thereof. In an embodiment, administering to the subject a low, immune enhancing, dose of an mTOR inhibitor comprises administering, e.g., once per week, e.g., in a sustained release dosage form, 0.3 to 60, 1.5 to 30, 7.5 to 22.5, 9 to 18, or about 15 mgs of RAD001, or a bioequivalent dose thereof.

The extent of mTOR inhibition can be conveyed as, or corresponds to, the extent of P70 S6 kinase inhibition, e.g., the extent of mTOR inhibition can be determined by the level of decrease in P70 S6 kinase activity, e.g., by the decrease in phosphorylation of a P70 S6 kinase substrate. The level of mTOR inhibition can be evaluated by various methods, such as measuring P70 S6 kinase activity by the Boulay assay, as described in U.S. Patent Application No. 2015/01240036, hereby incorporated by reference, or as described in U.S. Pat. No. 7,727,950, hereby incorporated by reference; measuring the level of phosphorylated S6 by western blot; or evaluating a change in the ratio of PD1 negative immune effector cells to PD1 positive immune effector cells.

As used herein, the term “mTOR inhibitor” refers to a compound or ligand, or a pharmaceutically acceptable salt thereof, which inhibits the mTOR kinase in a cell. In an embodiment, an mTOR inhibitor is an allosteric inhibitor. Allosteric mTOR inhibitors include the neutral tricyclic compound rapamycin (sirolimus), rapamycin-related compounds, that is compounds having structural and functional similarity to rapamycin including, e.g., rapamycin derivatives, rapamycin analogs (also referred to as rapalogs) and other macrolide compounds that inhibit mTOR activity. In an embodiment, an mTOR inhibitor is a catalytic inhibitor.

Rapamycin is a known macrolide antibiotic produced by Streptomyces hygroscopicus having the structure shown in Formula A.

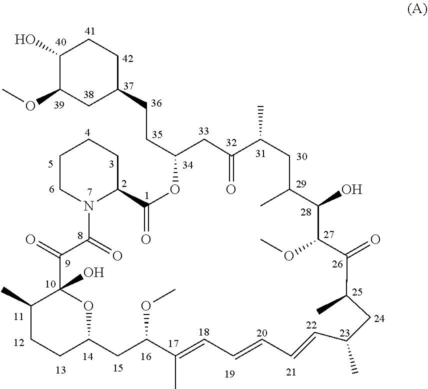 [SMILES: CO[C@H]1C[C@@H]2CC[C@@H](C)[C@@](O)(O2)C(=O)C(=O)N2CCCC[C@H]2C(=O)O[C@H](CC[C@@H]2CC[C@@H](O)[C@H](OC)C2)CC(=O)[C@H](C)CC(C)[C@@H](O)[C@@H](OC)C(=O)[C@H](C)C[C@H](C)/C=C/C=C/C=C/1C]

See, e.g., McAlpine, J. B., et al., J. Antibiotics (1991) 44: 688; Schreiber, S. L., et al., J. Am. Chem. Soc. (1991) 113: 7433; U.S. Pat. No. 3,929,992. There are various numbering schemes proposed for rapamycin. To avoid confusion, when specific rapamycin analogs are named herein, the names are given with reference to rapamycin using the numbering scheme of formula A.

Rapamycin analogs useful in the invention are, for example, 0-substituted analogs in which the hydroxyl group on the cyclohexyl ring of rapamycin is replaced by OR1 in which R1 is hydroxyalkyl, hydroxyalkoxyalkyl, acylaminoalkyl, or aminoalkyl; e.g. RAD001, also known as everolimus, as described in U.S. Pat. No. 5,665,772 and WO94/09010, the contents of each are incorporated by reference.

Other suitable rapamycin analogs include those substituted at the 26- or 28-position. The rapamycin analog may be an epimer of an analog mentioned above, particularly an epimer of an analog substituted in position 40, 28 or 26, and may optionally be further hydrogenated, e.g. as described in U.S. Pat. No. 6,015,815, WO95/14023 and WO99/15530 the contents of which are incorporated by reference, e.g. ABT578 also known as zotarolimus or a rapamycin analog described in U.S. Pat. No. 7,091,213, WO98/02441 and WO01/14387 the contents of which are incorporated by reference, e.g. AP23573 also known as ridaforolimus.

Examples of rapamycin analogs suitable for use in the present invention from U.S. Pat. No. 5,665,772 include, but are not limited to, 40-O-benzyl-rapamycin, 40-O-(4′-hydroxymethyl)benzyl-rapamycin, 40-O-[4′-(1,2-dihydroxyethyl)]benzyl-rapamycin, 40-O-allyl-rapamycin, 40-O-[3′-(2,2-dimethyl-1,3-dioxolan-4(S)-yl)-prop-2′-en-1′-yl]-rapamycin, (2′E,4'S)-40-O-(4′,5′-dihydroxypent-2′-en-1′-yl)-rapamycin, 40-O-(2-hydroxy)ethoxycarbonylmethyl-rapamycin, 40-O-(2-hydroxy)ethyl-rapamycin, 40-O-(3-hydroxy)propyl-rapamycin, 40-O-(6-hydroxy)hexyl-rapamycin, 40-O-[2-(2-hydroxy)ethoxy]ethyl-rapamycin, 40-O-[(35)-2,2-dimethyldioxolan-3-yl]methyl-rapamycin, 40-0-[(2S)-2,3-dihydroxyprop-1-yl]-rapamycin, 40-O-(2-acetoxy)ethyl-rapamycin, 40-O-(2-nicotinoyloxy)ethyl-rapamycin, 40-O-[2-(N-morpholino)acetoxy]ethyl-rapamycin, 40-O-(2-N-imidazolylacetoxy)ethyl-rapamycin, 40-O-[2-(N-methyl-N′-piperazinyl)acetoxy]ethyl-rapamycin, 39-O-desmethyl-39,40-O,O-ethylene-rapamycin, (26R)-26-dihydro-40-O-(2-hydroxy)ethyl-rapamycin, 40-O-(2-aminoethyl)-rapamycin, 40-O-(2-acetaminoethyl)-rapamycin, 40-O-(2-nicotinamidoethyl)-rapamycin, 40-O-(2-(N-methyl-imidazo-2′-ylcarbethoxamido)ethyl)-rapamycin, 40-O-(2-ethoxycarbonylaminoethyl)-rapamycin, 40-O-(2-tolylsulfonamidoethyl)-rapamycin and 40-O-[2-(4′,5′-dicarboethoxy-1′,2′,3′-triazol-1′-yl)-ethyl]-rapamycin.

Other rapamycin analogs useful in the present invention are analogs where the hydroxyl group on the cyclohexyl ring of rapamycin and/or the hydroxy group at the 28 position is replaced with an hydroxyester group are known, for example, rapamycin analogs found in U.S. RE44,768, e.g. temsirolimus.

Other rapamycin analogs useful in the preset invention include those wherein the methoxy group at the 16 position is replaced with another substituent, preferably (optionally hydroxy-substituted) alkynyloxy, benzyl, orthomethoxybenzyl or chlorobenzyl and/or wherein the mexthoxy group at the 39 position is deleted together with the 39 carbon so that the cyclohexyl ring of rapamycin becomes a cyclopentyl ring lacking the 39 position methyoxy group; e.g. as described in WO95/16691 and WO96/41807, the contents of which are incorporated by reference. The analogs can be further modified such that the hydroxy at the 40-position of rapamycin is alkylated and/or the 32-carbonyl is reduced.

Rapamycin analogs from WO95/16691 include, but are not limited to, 16-demthoxy-16-(pent-2-ynyl)oxy-rapamycin, 16-demthoxy-16-(but-2-ynyl)oxy-rapamycin, 16-demthoxy-16-(propargyl)oxy-rapamycin, 16-demethoxy-16-(4-hydroxy-but-2-ynyl)oxy-rapamycin, 16-demthoxy-16-benzyloxy-40-O-(2-hydroxyethyl)-rapamycin, 16-demthoxy-16-benzyloxy-rapamycin, 16-demethoxy-16-ortho-methoxybenzyl-rapamycin, 16-demethoxy-40-O-(2-methoxyethyl)-16-pent-2-ynyl)oxy-rapamycin, 39-demethoxy-40-desoxy-39-formyl-42-nor-rapamycin, 39-demethoxy-40-desoxy-39-hydroxymethyl-42-nor-rapamycin, 39-demethoxy-40-desoxy-39-carboxy-42-nor-rapamycin, 39-demethoxy-40-desoxy-39-(4-methyl-piperazin-1-yl)carbonyl-42-nor-rapamycin, 39-demethoxy-40-desoxy-39-(morpholin-4-yl)carbonyl-42-nor-rapamycin, 39-demethoxy-40-desoxy-39-[N-methyl, N-(2-pyridin-2-yl-ethyl)]carbamoyl-42-nor-rapamycin and 39-demethoxy-40-desoxy-39-(p-toluenesulfonylhydrazonomethyl)-42-nor-rapamycin.

Rapamycin analogs from WO96/41807 include, but are not limited to, 32-deoxo-rapamycin, 16-O-pent-2-ynyl-32-deoxo-rapamycin, 16-O-pent-2-ynyl-32-deoxo-40-O-(2-hydroxy-ethyl)-rapamycin, 16-O-pent-2-ynyl-32-(S)-dihydro-40-O-(2-hydroxyethyl)-rapamycin, 32(S)-dihydro-40-O-(2-methoxy)ethyl-rapamycin and 32(S)-dihydro-40-O-(2-hydroxyethyl)-rapamycin.

Another suitable rapamycin analog is umirolimus as described in US2005/0101624 the contents of which are incorporated by reference.

RAD001, otherwise known as everolimus (Afinitor®), has the chemical name (1R,9S,12S,15R,16E,18R,19R,21R,23S,24E,26E,28E,30S,32S,35R)-1,18-dihydroxy-12-{(1R)-2-[(1S,3R,4R)-4-(2-hydroxyethoxy)-3-methoxycyclohexyl]-1-methylethyl}-19,30-dimethoxy-15,17,21,23,29,35-hexamethyl-11,36-dioxa-4-aza-tricyclo[30.3.1.04,9]hexatriaconta-16,24,26,28-tetraene-2,3,10,14,20-pentaone, as described in U.S. Pat. No. 5,665,772 and WO94/09010, the contents of each are incorporated by reference.

Further examples of allosteric mTOR inhibitors include sirolimus (rapamycin, AY-22989), 40-[3-hydroxy-2-(hydroxymethyl)-2-methylpropanoate]-rapamycin (also called temsirolimus or CCI-779) and ridaforolimus (AP-23573/MK-8669). Other examples of allosteric mTor inhibitors include zotarolimus (ABT578) and umirolimus.

Alternatively or additionally, catalytic, ATP-competitive mTOR inhibitors have been found to target the mTOR kinase domain directly and target both mTORC1 and mTORC2. These are also more effective inhibitors of mTORC1 than such allosteric mTOR inhibitors as rapamycin, because they modulate rapamycin-resistant mTORC1 outputs such as 4EBP1-T37/46 phosphorylation and cap-dependent translation.

Catalytic inhibitors include: BEZ235 or 2-methyl-2-[4-(3-methyl-2-oxo-8-quinolin-3-yl-2,3-dihydro-imidazo[4,5-c]quinolin-1-yl)-phenyl]-propionitrile, or the monotosylate salt form (the synthesis of BEZ235 is described in WO2006/122806); CCG168 (otherwise known as AZD-8055, Chresta, C. M., et al., Cancer Res, 2010, 70(1), 288-298) which has the chemical name {5-[2,4-bis-((S)-3-methyl-morpholin-4-yl)-pyrido[2,3d]pyrimidin-7-yl]-2-methoxyphenyl}-methanol; 3-[2,4-bis[(35)-3-methylmorpholin-4-yl]pyrido[2,3-d]pyrimidin-7-yl]-N-methylbenzamide (WO09104019); 3-(2-aminobenzo[d]oxazol-5-yl)-1-isopropyl-1H-pyrazolo[3,4-d]pyrimidin-4-amine (WO10051043 and WO2013023184); A N-(3-(N-(3-((3,5-dimethoxyphenyl)amino)quinoxaline-2-yl)sulfamoyl)phenyl)-3-methoxy-4-methylbenzamide (WO07044729 and WO12006552); PKI-587 (Venkatesan, A. M., J. Med. Chem., 2010, 53, 2636-2645) which has the chemical name 1-[4-[4-(dimethylamino)piperidine-1-carbonyl]phenyl]-3-[4-(4,6-dimorpholino-1,3,5-triazin-2-yl)phenyl]urea; GSK-2126458 (ACS Med. Chem. Lett., 2010, 1, 39-43) which has the chemical name 2,4-difluoro-N-{2-methoxy-5-[4-(4-pyridazinyl)-6-quinolinyl]-3-pyridinyl}benzenesulfonamide; 5-(9-isopropyl-8-methyl-2-morpholino-9H-purin-6-yl)pyrimidin-2-amine (WO10114484); and (E)-N-(8-(6-amino-5-(trifluoromethyl)pyridin-3-yl)-1-(6-(2-cyanopropan-2-yl)pyridin-3-yl)-3-methyl-1H-imidazo[4,5-c]quinolin-2(3H)-ylidene) cyanamide (WO12007926).

Further examples of catalytic mTOR inhibitors include 8-(6-methoxy-pyridin-3-yl)-3-methyl-1-(4-piperazin-1-yl-3-trifluoromethyl-phenyl)-1,3-dihydro-imidazo[4,5-c]quinolin-2-one (WO2006/122806) and Ku-0063794 (Garcia-Martinez J M, et al., Biochem J., 2009, 421(1), 29-42. Ku-0063794 is a specific inhibitor of the mammalian target of rapamycin (mTOR).) WYE-354 is another example of a catalytic mTOR inhibitor (Yu K, et al. (2009). Biochemical, Cellular, and In vivo Activity of Novel ATP-Competitive and Selective Inhibitors of the Mammalian Target of Rapamycin. Cancer Res. 69(15): 6232-6240).

mTOR inhibitors useful according to the present invention also include prodrugs, derivatives, pharmaceutically acceptable salts, or analogs thereof of any of the foregoing.

mTOR inhibitors, such as RAD001, may be formulated for delivery based on well-established methods in the art based on the particular dosages described herein. In particular, U.S. Pat. No. 6,004,973 (incorporated herein by reference) provides examples of formulations useable with the mTOR inhibitors described herein.

### Methods and Biomarkers for Evaluating CAR-Effectiveness or Sample Suitability

In another aspect, the invention features a method of evaluating or monitoring the effectiveness of a CAR-expressing cell therapy (e.g., a BCMACAR therapy), in a subject (e.g., a subject having a cancer, e.g., a hematological cancer), or the suitability of a sample (e.g., an apheresis sample) for a CAR therapy (e.g., a BCMACAR therapy). The method includes acquiring a value of effectiveness to the CAR therapy, or sample suitability, wherein said value is indicative of the effectiveness or suitability of the CAR-expressing cell therapy.

In embodiments, the value of effectiveness to the CAR therapy, or sample suitability, comprises a measure of one, two, three, four, five, six or more (all) of the following: (i) the level or activity of one, two, three, or more (e.g., all) of resting TEFF cells, resting TREG cells, younger T cells (e.g., younger CD4 or CD8 cells, or gamma/delta T cells), or early memory T cells, or a combination thereof, in a sample (e.g., an apheresis sample or a manufactured CAR-expressing cell product sample); (ii) the level or activity of one, two, three, or more (e.g., all) of activated TEFF cells, activated TREG cells, older T cells (e.g., older CD4 or CD8 cells), or late memory T cells, or a combination thereof, in a sample (e.g., an apheresis sample or a manufactured CAR-expressing cell product sample); (iii) the level or activity of an immune cell exhaustion marker, e.g., one, two or more immune checkpoint inhibitors (e.g., PD-1, PD-L1, TIM-3 and/or LAG-3) in a sample (e.g., an apheresis sample or a manufactured CAR-expressing cell product sample). In one embodiment, an immune cell has an exhausted phenotype, e.g., co-expresses at least two exhaustion markers, e.g., co-expresses PD-1 and TIM-3. In other embodiments, an immune cell has an exhausted phenotype, e.g., co-expresses at least two exhaustion markers, e.g., co-expresses PD-1 and LAG-3; (iv) the level or activity of CD27 and/or CD45RO− (e.g., CD27+CD45RO−) immune effector cells, e.g., in a CD4+ or a CD8+ T cell population, in a sample (e.g., an apheresis sample or a manufactured CAR-expressing cell product sample); (v) the level or activity of one, two, three, four, five, ten, twenty or more of the biomarkers chosen from CCL20, IL-17a and/or IL-6, PD-1, PD-L1, LAG-3, TIM-3, CD57, CD27, CD122, CD62L, KLRG1; (vi) a cytokine level or activity (e.g., quality of cytokine reportoire) in a CAR-expressing cell product sample, e.g., BCMA-expressing cell product sample; or (vii) a transduction efficiency of a CAR-expressing cell in a manufactured CAR-expressing cell product sample.

In some embodiments of any of the methods disclosed herein, the CAR-expressing cell therapy comprises a plurality (e.g., a population) of CAR-expressing immune effector cells, e.g., a plurality (e.g., a population) of T cells or NK cells, or a combination thereof. In one embodiment, the CAR-expressing cell therapy is a BCMACAR therapy.

In some embodiments of any of the methods disclosed herein, the measure of one or more of (i)-(vii) is obtained from an apheresis sample acquired from the subject. The apheresis sample can be evaluated prior to infusion or re-infusion.

In some embodiments of any of the methods disclosed herein, the measure of one or more of (i)-(vii) is obtained from a manufactured CAR-expressing cell product sample, e.g., BCMACAR-expressing cell product sample. The manufactured CAR-expressing cell product can be evaluated prior to infusion or re-infusion.

In some embodiments of any of the methods disclosed herein, the subject is evaluated prior to receiving, during, or after receiving, the CAR-expressing cell therapy.

In some embodiments of any of the methods disclosed herein, the measure of one or more of (i)-(vii) evaluates a profile for one or more of gene expression, flow cytometry or protein expression.

In some embodiments of any of the methods disclosed herein, the method further comprises identifying the subject as a responder, a non-responder, a relapser or a non-relapser, based on a measure of one or more of (i)-(vii).

In some embodiments of any of the methods disclosed herein, a responder (e.g., a complete responder) has, or is identified as having, a greater level or activity of one, two, or more (all) of GZMK, PPF1BP2, or naive T cells as compared to a non-responder.

In some embodiments of any of the methods disclosed herein, a non-responder has, or is identified as having, a greater level or activity of one, two, three, four, five, six, seven, or more (e.g., all) of IL22, IL-2RA, IL-21, IRF8, IL8, CCL17, CCL22, effector T cells, or regulatory T cells, as compared to a responder.

In an embodiment, a relapser is a patient having, or who is identified as having, an increased level of expression of one or more of (e.g., 2, 3, 4, or all of) the following genes, compared to non relapsers: MIR199A1, MIR1203, uc021ovp, ITM2C, and HLA-DQB1 and/or a decreased levels of expression of one or more of (e.g., 2, 3, 4, 5, 6, 7, 8, 9, 10, 11, or all of) the following genes, compared to non relapsers: PPIAL4D, TTTY10, TXLNG2P, MIR4650-1, KDM5D, USP9Y, PRKY, RPS4Y2, RPS4Y1, NCRNA00185, SULTIEl, and EIF1AY.

In some embodiments of any of the methods disclosed herein, a complete responder has, or is identified as having, a greater, e.g., a statistically significant greater, percentage of CD8+ T cells compared to a reference value, e.g., a non-responder percentage of CD8+ T cells.

In some embodiments of any of the methods disclosed herein, a complete responder has, or is identified as having, a greater percentage of CD27+CD45RO− immune effector cells, e.g., in the CD8+ population, compared to a reference value, e.g., a non-responder number of CD27+CD45RO− immune effector cells.

In some embodiments of any of the methods disclosed herein, a complete responder or a partial responder has, or is identified as having, a greater, e.g., a statistically significant greater, percentage of CD4+ T cells compared to a reference value, e.g., a non-responder percentage of CD4+ T cells.

In some embodiments of any of the methods disclosed herein, a complete responder has, or is identified as having, a greater percentage of one, two, three, or more (e.g., all) of resting TEFF cells, resting TREG cells, younger T cells (e.g., younger CD4 or CD8 cells, or gamma/delta T cells), or early memory T cells, or a combination thereof, compared to a reference value, e.g., a non-responder number of resting TEFF cells, resting TREG cells, younger T cells (e.g., younger CD4 or CD8 cells), or early memory T cells.

In some embodiments of any of the methods disclosed herein, a non-responder has, or is identified as having, a greater percentage of one, two, three, or more (e.g., all) of activated TEFF cells, activated TREG cells, older T cells (e.g., older CD4 or CD8 cells), or late memory T cells, or a combination thereof, compared to a reference value, e.g., a responder number of activated TEFF cells, activated TREG cells, older T cells (e.g., older CD4 or CD8 cells), or late memory T cells.

In some embodiments of any of the methods disclosed herein, a non-responder has, or is identified as having, a greater percentage of an immune cell exhaustion marker, e.g., one, two or more immune checkpoint inhibitors (e.g., PD-1, PD-L1, TIM-3 and/or LAG-3). In one embodiment, a non-responder has, or is identified as having, a greater percentage of PD-1, PD-L1, or LAG-3 expressing immune effector cells (e.g., CD4+ T cells and/or CD8+ T cells) (e.g., CAR-expressing CD4+ cells and/or CD8+ T cells) compared to the percentage of PD-1 or LAG-3 expressing immune effector cells from a responder.

In one embodiment, a non-responder has, or is identified as having, a greater percentage of immune cells having an exhausted phenotype, e.g., immune cells that co-express at least two exhaustion markers, e.g., co-expresses PD-1, PD-L1 and/or TIM-3. In other embodiments, a non-responder has, or is identified as having, a greater percentage of immune cells having an exhausted phenotype, e.g., immune cells that co-express at least two exhaustion markers, e.g., co-expresses PD-1 and LAG-3.

In some embodiments of any of the methods disclosed herein, a non-responder has, or is identified as having, a greater percentage of PD-1/PD-L1+/LAG-3+ cells in the CAR-expressing cell population (e.g., a BCMACAR+ cell population) compared to a responder (e.g., a complete responder) to the CAR-expressing cell therapy.

In some embodiments of any of the methods disclosed herein, a partial responder has, or is identified as having, a higher percentages of PD-1/PD-L1+/LAG-3+ cells, than a responder, in the CAR-expressing cell population (e.g., a BCMACAR+ cell population).

In some embodiments of any of the methods disclosed herein, a non-responder has, or is identified as having, an exhausted phenotype of PD1/PD-L1+CAR+ and co-expression of LAG3 in the CAR-expressing cell population (e.g., a BCMACAR+ cell population).

In some embodiments of any of the methods disclosed herein, a non-responder has, or is identified as having, a greater percentage of PD-1/PD-L1+/TIM-3+ cells in the CAR-expressing cell population (e.g., a BCMACAR+ cell population) compared to the responder (e.g., a complete responder).

In some embodiments of any of the methods disclosed herein, a partial responders has, or is identified as having, a higher percentage of PD-1/PD-L1+/TIM-3+ cells, than responders, in the CAR-expressing cell population (e.g., a BCMACAR+ cell population).

In some embodiments of any of the methods disclosed herein, the presence of CD8+CD27+CD45RO− T cells in an apheresis sample is a positive predictor of the subject response to a CAR-expressing cell therapy (e.g., a BCMACAR therapy).

In some embodiments of any of the methods disclosed herein, a high percentage of PD1+CAR+ and LAG3+ or TIM3+ T cells in an apheresis sample is a poor prognostic predictor of the subject response to a CAR-expressing cell therapy (e.g., a BCMACAR therapy).

In some embodiments of any of the methods disclosed herein, the responder (e.g., the complete or partial responder) has one, two, three or more (or all) of the following profile: (i) has a greater number of CD27+ immune effector cells compared to a reference value, e.g., a non-responder number of CD27+ immune effector cells; (ii) (i) has a greater number of CD8+ T cells compared to a reference value, e.g., a non-responder number of CD8+ T cells; (iii) has a lower number of immune cells expressing one or more checkpoint inhibitors, e.g., a checkpoint inhibitor chosen from PD-1, PD-L1, LAG-3, TIM-3, or KLRG-1, or a combination, compared to a reference value, e.g., a non-responder number of cells expressing one or more checkpoint inhibitors; or (iv) has a greater number of one, two, three, four or more (all) of resting TEFF cells, resting TREG cells, naïve CD4 cells, unstimulated memory cells or early memory T cells, or a combination thereof, compared to a reference value, e.g., a non-responder number of resting TEFF cells, resting TREG cells, naïve CD4 cells, unstimulated memory cells or early memory T cells.

In some embodiments of any of the methods disclosed herein, the cytokine level or activity of (vi) is chosen from one, two, three, four, five, six, seven, eight, or more (or all) of cytokine CCL20/MIP3a, IL17A, IL6, GM-CSF, IFNγ, IL10, IL13, IL2, IL21, IL4, IL5, IL9 or TNFα, or a combination thereof. The cytokine can be chosen from one, two, three, four or more (all) of IL-17a, CCL20, IL2, IL6, or TNFα. In one embodiment, an increased level or activity of a cytokine is chosen from one or both of IL-17a and CCL20, is indicative of increased responsiveness or decreased relapse.

In some embodiments of any of the methods disclosed herein, a transduction efficiency of 15% or higher in (vii) is indicative of increased responsiveness or decreased relapse.

In some embodiments of any of the methods disclosed herein, a transduction efficiency of less than 15% in (vii) is indicative of decreased responsiveness or increased relapse.

In embodiments, the responder, a non-responder, a relapser or a non-relapser identified by the methods herein can be further evaluated according to clinical criteria. For example, a complete responder has, or is identified as, a subject having a disease, e.g., a cancer, who exhibits a complete response, e.g., a complete remission, to a treatment. A complete response may be identified, e.g., using the NCCN Guidelines®, or Cheson et al, J Clin Oncol 17:1244 (1999) and Cheson et al., “Revised Response Criteria for Malignant Lymphoma”, J Clin Oncol 25:579-586 (2007) (both of which are incorporated by reference herein in their entireties), as described herein. A partial responder has, or is identified as, a subject having a disease, e.g., a cancer, who exhibits a partial response, e.g., a partial remission, to a treatment. A partial response may be identified, e.g., using the NCCN Guidelines®, or Cheson criteria as described herein. A non-responder has, or is identified as, a subject having a disease, e.g., a cancer, who does not exhibit a response to a treatment, e.g., the patient has stable disease or progressive disease. A non-responder may be identified, e.g., using the NCCN Guidelines®, or Cheson criteria as described herein.

Alternatively, or in combination with the methods disclosed herein, responsive to said value, performing one, two, three four or more of: administering e.g., to a responder or a non-relapser, a CAR-expressing cell therapy; administered an altered dosing of a CAR-expressing cell therapy; altering the schedule or time course of a CAR-expressing cell therapy; administering, e.g., to a non-responder or a partial responder, an additional agent in combination with a CAR-expressing cell therapy, e.g., a checkpoint inhibitor, e.g., a checkpoint inhibitor described herein; administering to a non-responder or partial responder a therapy that increases the number of younger T cells in the subject prior to treatment with a CAR-expressing cell therapy; modifying a manufacturing process of a CAR-expressing cell therapy, e.g., enriching for younger T cells prior to introducing a nucleic acid encoding a CAR, or increasing the transduction efficiency, e.g., for a subject identified as a non-responder or a partial responder; administering an alternative therapy, e.g., for a non-responder or partial responder or relapser; or if the subject is, or is identified as, a non-responder or a relapser, decreasing the TREG cell population and/or TREG gene signature, e.g., by one or more of CD25 depletion, administration of cyclophosphamide, anti-GITR antibody, or a combination thereof.

In certain embodiments, the subject is pre-treated with an anti-GITR antibody. In certain embodiment, the subject is treated with an anti-GITR antibody prior to infusion or re-infusion.

### Biopolymer Delivery Methods

In some embodiments, one or more CAR-expressing cells as disclosed herein can be administered or delivered to the subject via a biopolymer scaffold, e.g., a biopolymer implant.

Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic.

Examples of suitable biopolymers include, but are not limited to, agar, agarose, alginate, alginate/calcium phosphate cement (CPC), beta-galactosidase (β-GAL), (1,2,3,4,6-pentaacetyl a-D-galactose), cellulose, chitin, chitosan, collagen, elastin, gelatin, hyaluronic acid collagen, hydroxyapatite, poly(3-hydroxybutyrate-co-3-hydroxy-hexanoate) (PHBHHx), poly(lactide), poly(caprolactone) (PCL), poly(lactide-co-glycolide) (PLG), polyethylene oxide (PEO), poly(lactic-co-glycolic acid) (PLGA), polypropylene oxide (PPO), polyvinyl alcohol) (PVA), silk, soy protein, and soy protein isolate, alone or in combination with any other polymer composition, in any concentration and in any ratio. The biopolymer can be augmented or modified with adhesion- or migration-promoting molecules, e.g., collagen-mimetic peptides that bind to the collagen receptor of lymphocytes, and/or stimulatory molecules to enhance the delivery, expansion, or function, e.g., anti-cancer activity, of the cells to be delivered. The biopolymer scaffold can be an injectable, e.g., a gel or a semi-solid, or a solid composition.

In some embodiments, CAR-expressing cells described herein are seeded onto the biopolymer scaffold prior to delivery to the subject. In embodiments, the biopolymer scaffold further comprises one or more additional therapeutic agents described herein (e.g., another CAR-expressing cell, an antibody, or a small molecule) or agents that enhance the activity of a CAR-expressing cell, e.g., incorporated or conjugated to the biopolymers of the scaffold. In embodiments, the biopolymer scaffold is injected, e.g., intratumorally, or surgically implanted at the tumor or within a proximity of the tumor sufficient to mediate an anti-tumor effect. Additional examples of biopolymer compositions and methods for their delivery are described in Stephan et al., Nature Biotechnology, 2015, 33:97-101; and WO2014/110591.

### J. Methods of Detecting

Disclosed are methods of detecting CD229 on a cell comprising administering a composition comprising one or more of the disclosed antibodies or fragments thereof to a sample and detecting the binding of the antibody or fragment thereof to CD229. For example, the antibody or fragment thereof can comprise a CD229 antigen binding domain comprising SEQ ID NO: 134, SEQ ID NO:53, or SEQ ID NO:84.

In some instances, detecting the binding of the antibody or fragment thereof to CD229 comprises immunostaining.

### K. Methods of Killing CD229 Cells

Disclosed are methods of killing CD229 positive cells comprising administering an effective amount of a cell genetically modified to express one or more of the disclosed CAR polypeptides to a sample comprising CD229 positive cells. Cells genetically modified to express one or more of the disclosed CAR polypeptides can be, but are not limited to, T cells or NK cells. In some instances, the T cell can be a γδ T cell or an αβ T cell.

Disclosed are methods of killing CD229 positive cells comprising administering an effective amount of a T cell genetically modified to express one or more of the disclosed CAR polypeptides to a sample comprising CD229 positive cells. For example, disclosed are methods of killing CD229 positive cells comprising administering an effective amount of a T cell genetically modified to express a CAR polypeptide comprising a CD229 antigen binding domain, a transmembrane domain, and an intracellular signaling domain.

In some aspects, the methods of killing CD229 positive cells only occurs in cancer cells, such as multiple myeloma cells. Healthy cells expressing CD229 are not killed. Thus, disclosed are methods of killing CD229 positive cells comprising administering an effective amount of a T cell genetically modified to express a CAR polypeptide to a sample comprising CD229 positive cells, wherein the CAR polypeptide comprises a CD229 antigen binding domain, a transmembrane domain, and an intracellular signaling domain, wherein the CD229 binding domain is a variant CD229 antigen binding domain, particularly a variant of SEQ ID NO:1. In some aspects, the CD229 antigen binding domain can comprise the sequence of SEQ ID NO: 134, SEQ ID NO:53, or SEQ ID NO:84.

Disclosed are methods of killing CD229 positive cells comprising administering an effective amount of one or more of the disclosed antibodies or antibody fragments thereof to a sample comprising CD229 positive cells

### L. Methods of Preferentially Targeting Cancer Cells

Disclosed are methods of preferentially targeting cancer cells comprising administering a composition comprising one or more of the disclosed CAR polypeptides, antibodies or fragments thereof to a sample and detecting the binding of the antibody or fragment thereof to CD229. For example, the antibody or fragment thereof can comprise a CD229 antigen binding domain comprising SEQ ID NO: 134, SEQ ID NO:53, or SEQ ID NO:84.

In some aspects, preferentially targeting cancer cells means healthy cells are not targeted. For example, in some embodiments, multiple myeloma cells are preferentially targeted with the disclosed polypeptides while healthy cells are not targeted.

### M. Methods of Making Cells

Disclosed are methods of making a cell comprising transducing a cell with one or more of the disclosed vectors. In some instances, the cell can be, but is not limited to, T cells or NK cells. In some instances, the T cell can be a γδ T cell or an αβ T cell.

Disclosed are methods of making a cell comprising transducing a T cell with one or more of the disclosed vectors. For example, disclosed are methods of making a cell comprising transducing a T cell with a vector comprising the nucleic acid sequence capable of encoding a disclosed CAR polypeptide to a subject in need thereof.

### N. Methods of Activating T Cells

Disclosed are methods of activating a T cell expressing one of the CAR polypeptides disclosed herein comprising culturing the T cell with a cell expressing CD229 and detecting the presence or absence of IFN-γ after culturing, wherein the presence of IFN-γ indicates the activation of the T cell.

### O. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the antibodies or fragments thereof disclosed herein.

Also disclosed are kits comprising one or more of the vectors disclosed herein.

### EXAMPLES

### A. Example 1: Systematic CAR T Cell Optimization Based on Universal Affinity-Tuning Platform Retains Anti-Tumor Activity while Eliminating On-Target Off-Tumor Toxicity

T cells expressing chimeric antigen receptors (CAR) using single-chain variable fragments (scFv) to target cancer-associated surface antigens are highly effective against several hematologic malignancies, including B cell lymphoma(1) and multiple myeloma(2, 3). However, their extraordinary cytotoxic activity poses new challenges, such as the unintended killing of healthy tissues expressing the targeted antigen, despite often at substantially lower levels(4). In the case of the widely used CD19 CAR T cells, this on-target off-tumor toxicity results in the elimination of healthy B cells(5, 6) and various other CAR T cell approaches have resulted in life-threatening toxicities and even patient deaths due to the targeting of healthy tissues(7-9). It has been shown previously that CAR T cells exert potent anti-tumor activity across a wide range of affinities(10-12) and many CAR T cell strategies currently in clinical use likely exceed the required affinity threshold. Consequently, low affinity antibodies have been developed for several cancer targets to increase cancer selectivity as well as CAR T cell persistence and function(13-16). However, none of these binding domains were derived from existing and extensively tested high-affinity antibodies already in clinical use and had to, again, undergo rigorous preclinical evaluation with the risk for substantial liabilities, such as off-target reactivity and unstable epitopes.

A universal affinity-tuning platform has been developed for the generation of low-affinity antibody variants derived from existing high-affinity antibodies. CAR T cells based on antibody variants developed using this approach show increased selectivity for tumor cells, increased expansion, maintained anti-tumor activity in vitro and in vivo, and reduced trogocytosis, the stripping of target antigen from tumor cells by CAR T cells, potentially augmenting their persistence in vivo. The approach of the systematic optimization of antibody affinity of existing CAR binding domains by way of modifying parental high-affinity antibodies can be an important tool in the development of more effective and safer CAR T cell approaches.

### 1. Results

i. Generation of CD229 Antibody Variants to Increase CAR T Cell Selectivity

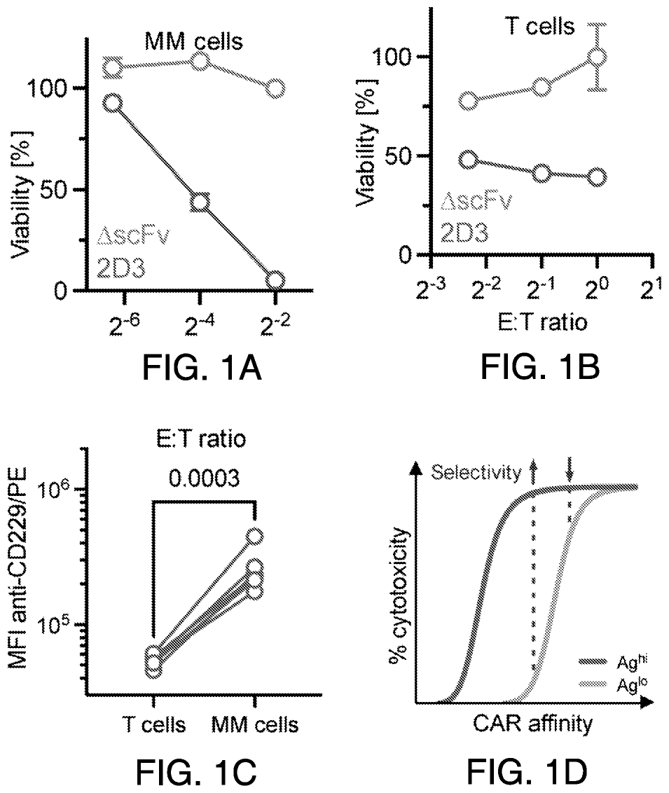

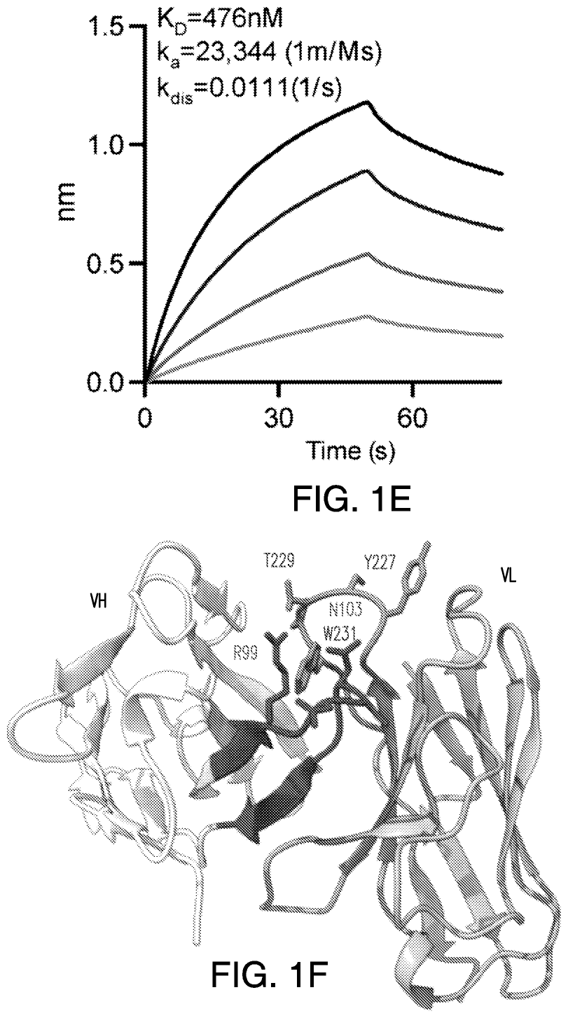

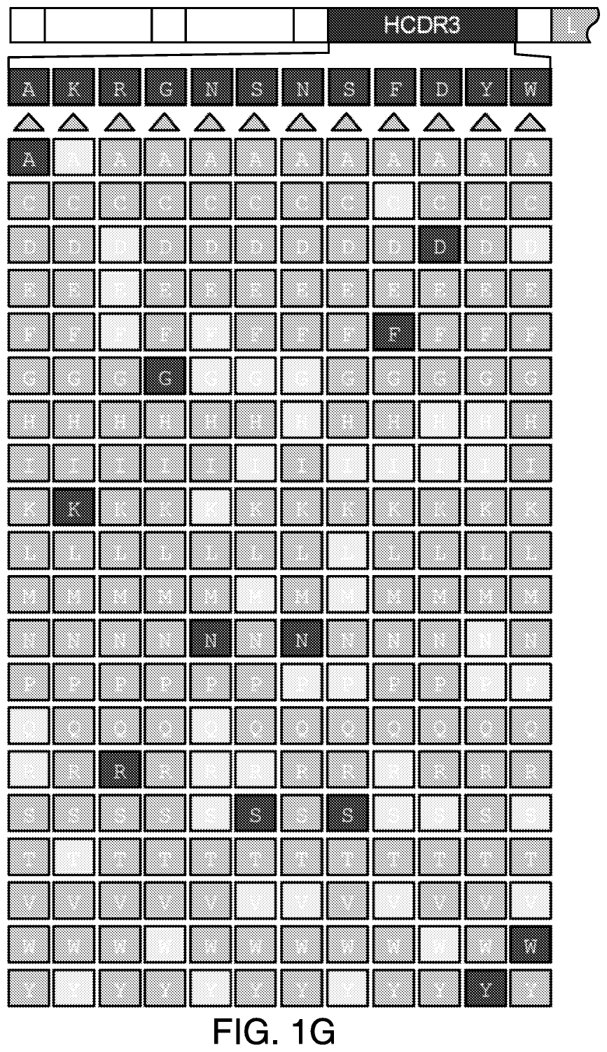

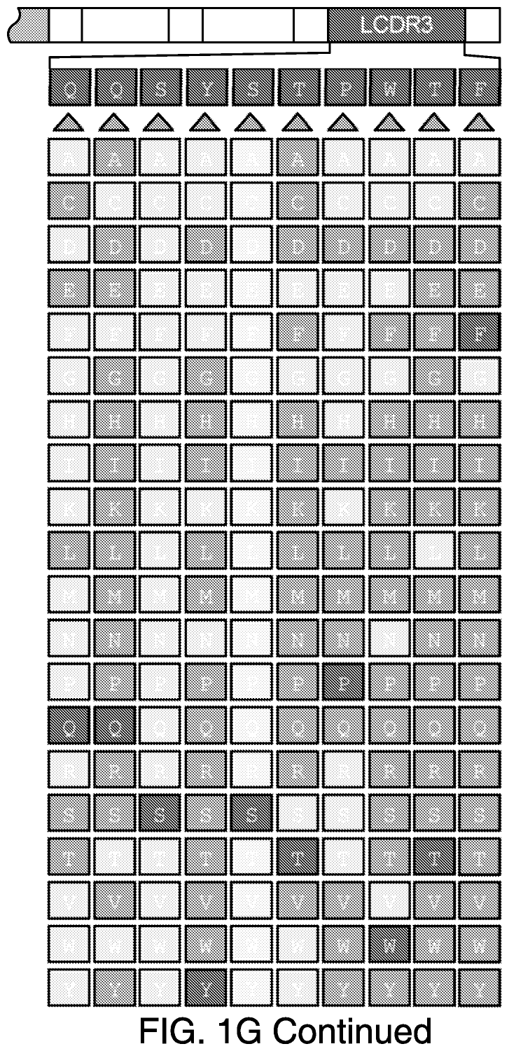

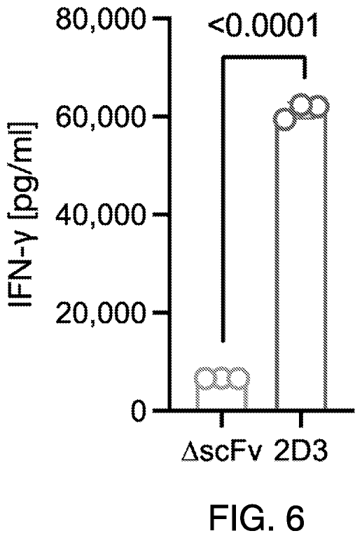

CAR T cells targeting B cell maturation antigen (BCMA), an antigen otherwise exclusively expressed on plasma cells, have been FDA approved for the treatment of multiple myeloma (MM)(17, 18). However, most patients relapse within the first year(2) potentially due to incomplete targeting of MM-propagating cells in the memory B cell pool(19, 20). Recently, an alternative CAR T cell approach based on the phage-display derived, fully human anti-CD229 antibody 2D3 was developed showing targeting of both terminally differentiated MM plasma cells and MM-propagating cells (21). While CD229 CAR T cells indeed show efficient targeting of MM cells (FIG. 1A, FIG. 6), they also target healthy T cells (FIG. 1B), indicating the potential for substantial toxicities. However, analyzing expression of CD229 on MM cells and normal T cells from patients with relapsed-refractory MM using flow cytometry, it was found that T cells express significantly lower levels of CD229 than MM cells (FIG. 1C).

Affinity-tuning of CAR binding domains has previously been shown to reduce targeting of cells expressing lower levels of the targeted antigen (FIG. 1D). Compared to other commonly used CAR binding domains, such as the CD19-specific antibody FMC63(22), the affinity of wildtype 2D3 is already relatively low at 476 nM (FIG. 1E). Considering the high specificity and extensive preclinical characterization of 2D3, as well as the established anti-tumor activity and functionality of 2D3-based CAR T cells(21), an affinity-tuning platform was developed to generate low-affinity single-chain variable fragment (scFv) variants of 2D3. Affinity-tuning was carried out by comprehensively mutating heavy and light CDR3 regions (FIG. 1F) in combination with high-throughput screening and antibody characterization assays.

305 single amino acid substitution variants of 2D3 (FIG. 1G) were generated with the goal of substantially reducing 2D3 affinity while only minimally disrupting the CD229 epitope, 2D3 specificity, CAR T cell expansion, and anti-tumor activity.

ii. Single Amino Acid Substitutions Result in Substantially Reduced CD229 Binding

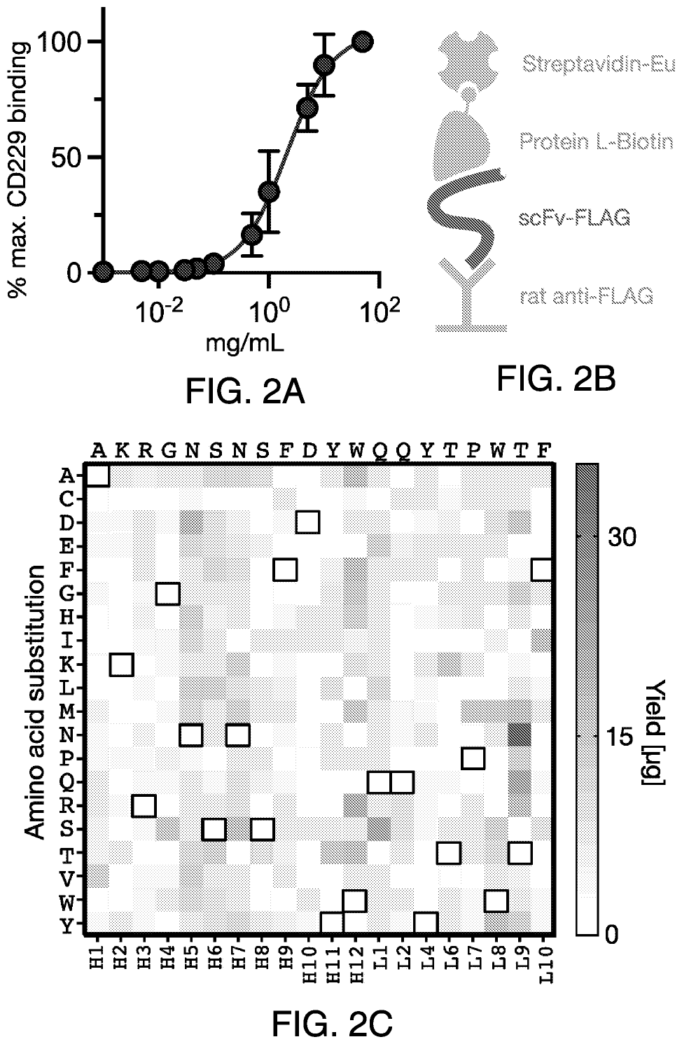

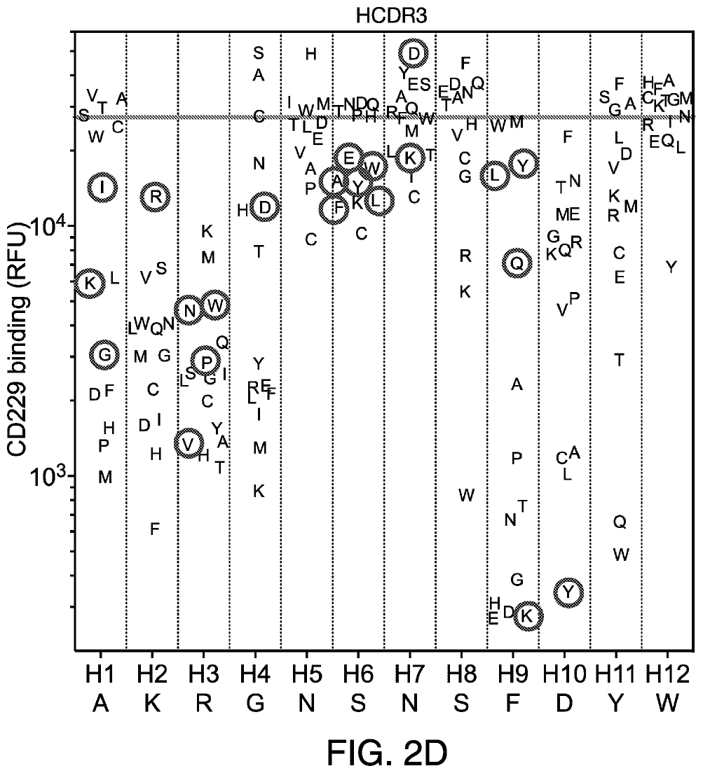

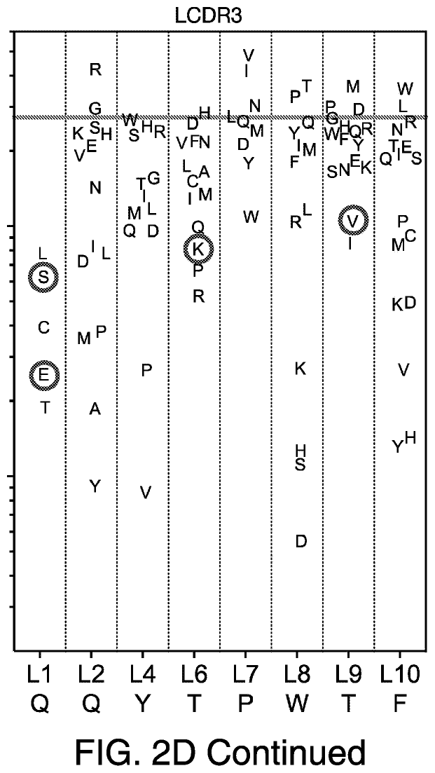

Identification of antibodies with reduced affinity represents a relatively uncommon objective in antibody discovery and poses unique challenges when developing appropriate screening approaches. Most common primary antibody screening assays, such as standard solid-phase binding assays using large sets of non-purified antibodies, are unable to differentiate between expression and affinity. As expected, this was also the case for 2D3, which showed a clear dependence of CD229 binding on antibody concentration, especially in concentration ranges commonly observed in standard high-throughput expression cultures (FIG. 2A). When screening for high-affinity antibodies this may be acceptable as the highest assay signals are likely the result of a combination of high affinity and high expression, both being desirable properties. In the case of low-affinity antibody screening, however, the potential conflation of low-expressing/high-affinity antibodies with high-expressing/low-affinity antibodies would render such data relatively meaningless. A high-throughput scFv quantification assay was developed relying on the binding of Protein L to the κ light chain in 2D3 (FIG. 2B) and total scFv yields were determined of all generated variants using a time-resolved fluorescence (TRF) assay (FIG. 2C). As expected, substitutions to cysteine and proline generally resulted in poor antibody expression, while alanine and threonine appeared to be tolerated well in various positions (FIG. 2C). In addition, it was found that some positions were able to accommodate almost any amino acid. This included some unexpected positions, such as asparagine in position H5, an exposed residue in the center of the groove formed by both CDR3 loops, as well as tryptophan in H12 and threonine in L9 (FIG. 2C). Importantly, several substitutions resulted in substantially improved expression, in line with established approaches to improve antibody stability via mutation of non-contact residues(23, 24). Following normalization of antibody concentrations, binding to recombinant CD229 in a standard solid-phase binding assay was determined (FIG. 2D). While most mutations did not reduce 2D3 variant binding to CD229, and some, as expected, increased binding, the comprehensive mutagenesis approach taken enabled the identification of a large set of scFvs showing various levels of reduced binding. Of the variant scFvs that showed reduced binding, scFvs with mutations in the outer residues of both CDR3s showed drastically reduced binding. Importantly, alanine scanning(25), one of the most widely used approaches to reduce protein-protein binding would not have resulted in a similarly comprehensive set of variants as alanine substitutions in many cases did not alter binding compared to parental 2D3 when other non-alanine substitutions substantially affected binding. In addition, alanine substitutions never represented the variants with the lowest binding signal in any position.

Taken together, the data indicate that comprehensive single amino acid substitution can be preferable to conventional mutagenesis strategies and is able to generate large sets of antibodies showing reduced antigen binding even in already relatively low-affinity binders. Based on scFv expression and CD229 binding data, 26 2D3 scFv variants were then selected for downstream analyses.

iii. Affinity Tuning Approach Results in Predominantly Off-Rate Driven Affinity Reductions

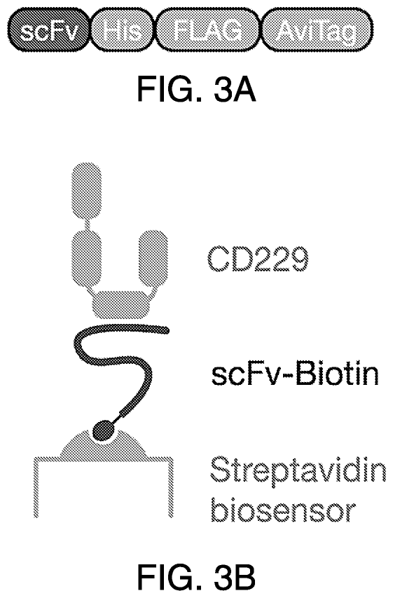

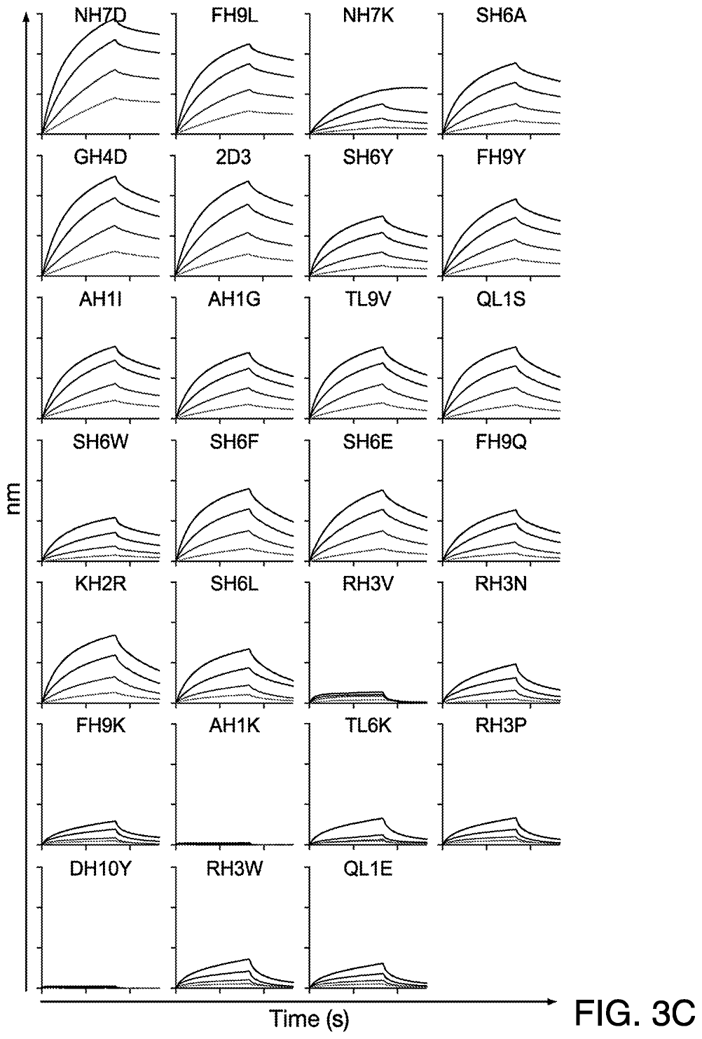

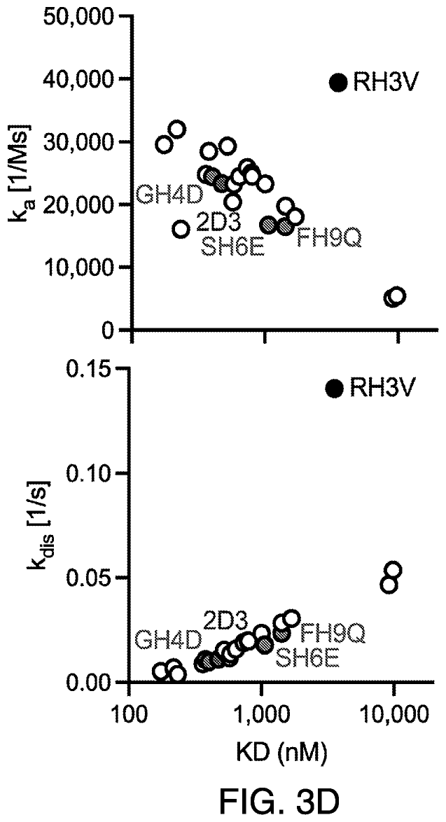

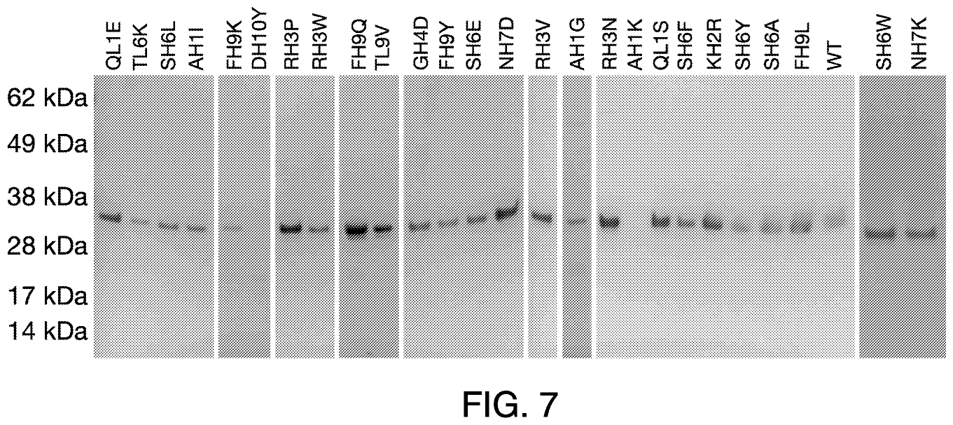

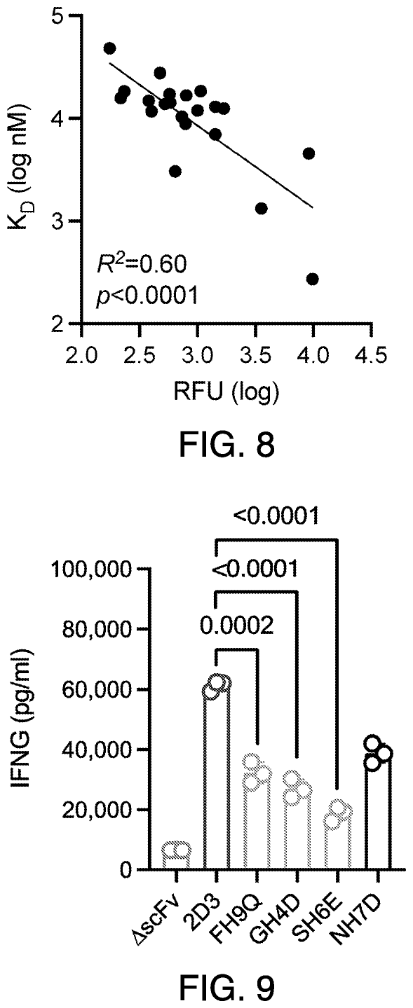

To confirm that the single amino acid substitution mutagenesis in fact resulted in relevantly altered affinities and rate constants, in vivo biotinylated 2D3 variant scFvs (FIG. 3A) were purified (FIG. 7). These antibodies were subjected to bio-layer interferometry (BLI) using streptavidin biosensors and recombinant CD229 (FIG. 3B), allowing the use of relatively high analyte concentrations due to non-destructive sampling(26). This process facilitated the characterization even of very weak binders (FIG. 3C). Affinities of 2D3 variants with single amino acid substitutions ranged from 175 nM to >10,000 nM (Table 4) and a very close correlation was observed between antibody affinity (K_{D}) and TRF binding (FIG. 8). Differences in affinity were generally driven by faster off-rates (Table 4). This result is likely related to the use of a solid-phase binding assay for primary variant screening, which can have biased clone selection towards variants with faster off-rates. In contrast to this finding, a dramatic reduction was observed in the on-rate of variants in which arginine in H3 was replaced (FIG. 3C, Table 4). A noted exception to this finding is RH3V, which in fact showed a faster on-rate but also a much faster off-rate (FIG. 3D). Combined, these data indicate a key role of the H3 arginine in the orchestration of the 2D3 epitope and among other possibilities might point to a model of 2D3 binding to CD229 in which RH3 binding facilitates interactions by other residues.

Single amino acid substitution together with BLI resulted in the identification of a set of clones with a wide range of mostly off-rate driven differences in affinity and provided initial data regarding the mode of 2D3-CD229 binding, which may aide further lead optimization.

iv. CD229 CAR T Cells Based on Variant Antibodies can be Manufactured, Show Efficient CAR Surface Expression, and Allow Identification of Clones with Increased Selectivity.

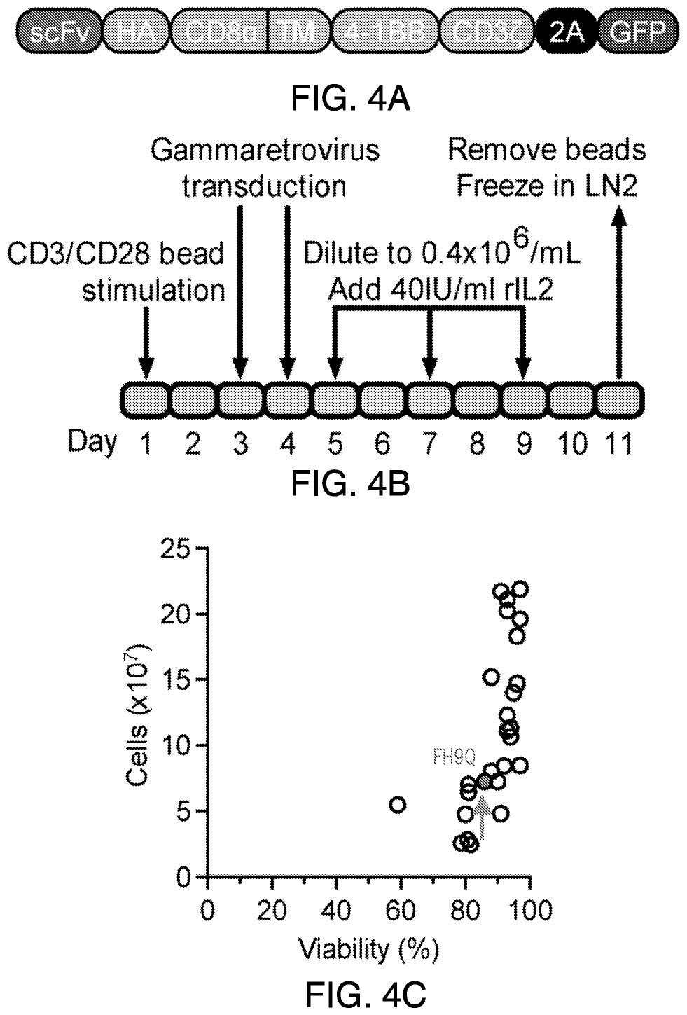

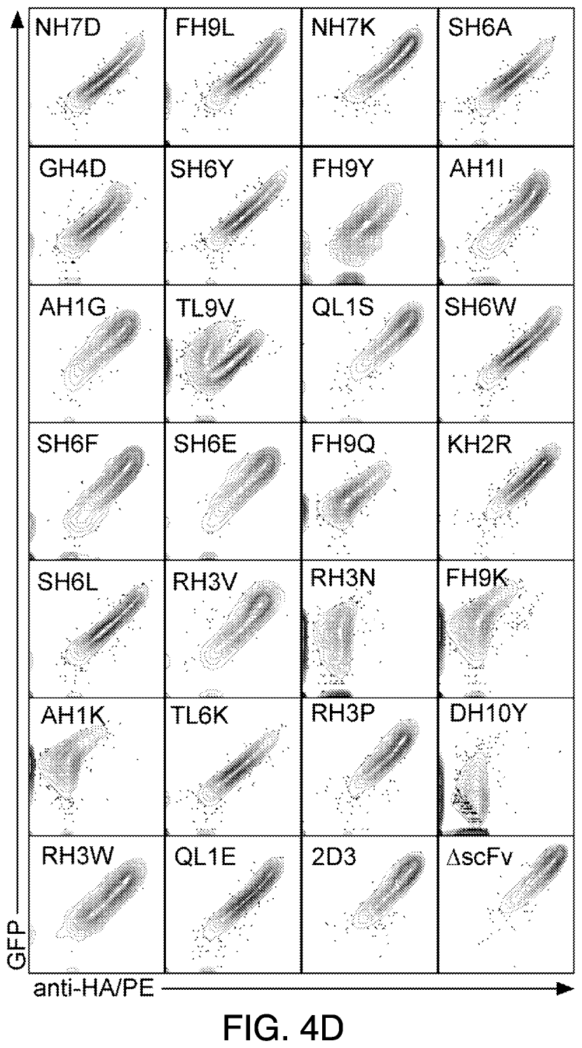

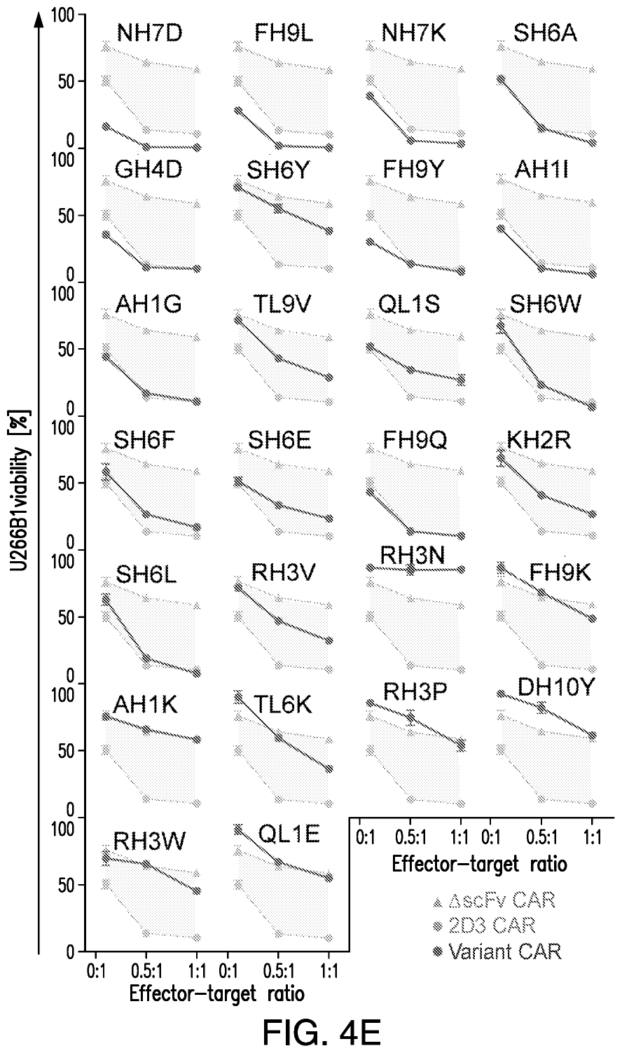

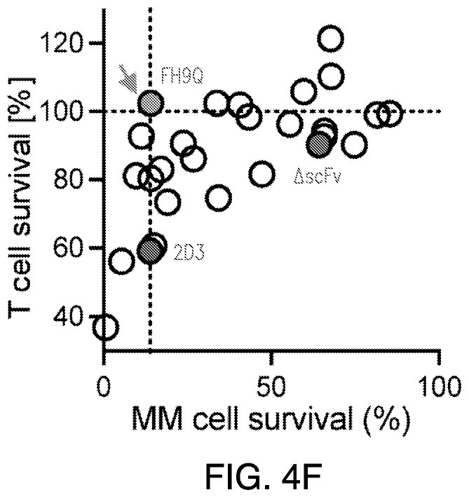

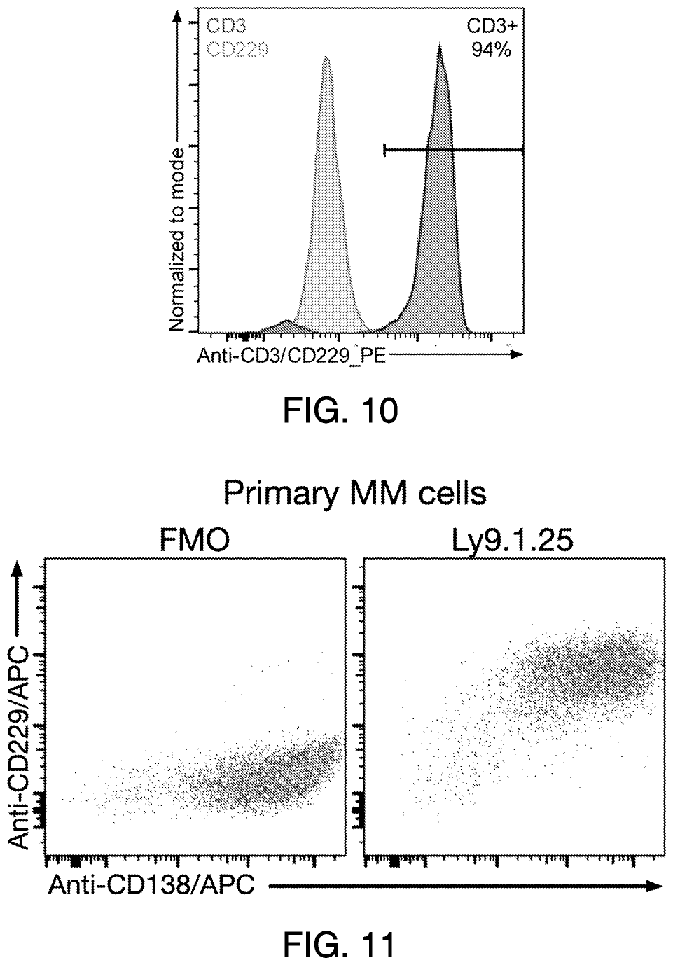

Optimal CAR affinity remains an active area of research but likely depends on various parameters, such as need for selectivity, epitope, as well as antigen- and CAR-density. Ideally, CAR affinity for a given target antigen will be chosen empirically, thus requiring a sufficiently large set of binders with different affinities available for CAR construction. To test the ability of 2D3 variants to confer cytotoxicity as CAR binding domains, all 26 scFvs were converted into CAR constructs (FIG. 4A) and produced primary human CAR T cells using a standard manufacturing process (FIG. 4B). It has previously been shown that even parental 2D3-based CAR T cells can be manufactured without the CAR T cells targeting each other because CD229 is downregulated upon CD3/CD28-bead activation at the start of manufacturing(21). We determined the viability and total CAR T cell yields on day 7 of manufacturing and found that yields varied substantially between constructs (FIG. 4C), possibly indicating increased tonic signaling (27, 28). Considering the substantial differences in soluble scFv expression levels between variants (FIG. 2C), CAR surface expression was determined of wildtype 2D3 and all variant CAR constructs, as well as T cells expressing a CAR without an scFv binding domain (AscFv). While all variant constructs expressed similar levels of the linked GFP reporter, two constructs, FH10K and AH1K, showed relatively low CAR surface expression and two other constructs, DH10Y and RH3N, did not show any CAR surface expression at all (FIG. 4D). One construct, TL9V, showed a bimodal distribution, potentially indicating recombination during retroviral packaging. Whether mutagenesis had resulted in altered anti-tumor activity by determining killing of MM cells by all variant CAR T cells at multiple effector-target ratios was determined. It was found that while several constructs indeed showed reduced tumor cell killing, likely due to the substantially reduced affinity of those variants, several constructs, including SH6A, GH4D, FH9Y, AH1I, AH1G, and FH9Q showed either equal or enhanced anti-tumor activity (FIG. 4E, FIG. 9). Next, determining whether reducing CAR affinity resulted in increased selectivity by measuring killing of purified T cells was performed (FIG. 10). At an effector-target ratio of 0.5:1, it was found that, as expected, numerous constructs that had shown reduced cytotoxic activity against tumor cells, now also showed reduced killing of T cells (FIG. 4F). However, among the variants showing comparable or increased tumor cell killing several constructs showed reduced T cell killing in this screening assay. For downstream analyses one construct was selected, FH9Q, which showed dramatically reduced killing of T cells (FIG. 4F).

v. FH9Q CAR T Cells Maintain Anti-Tumor Activity In Vitro and In Vivo, do not Target Healthy T Cells, and Evidence Reduced Trogocytosis

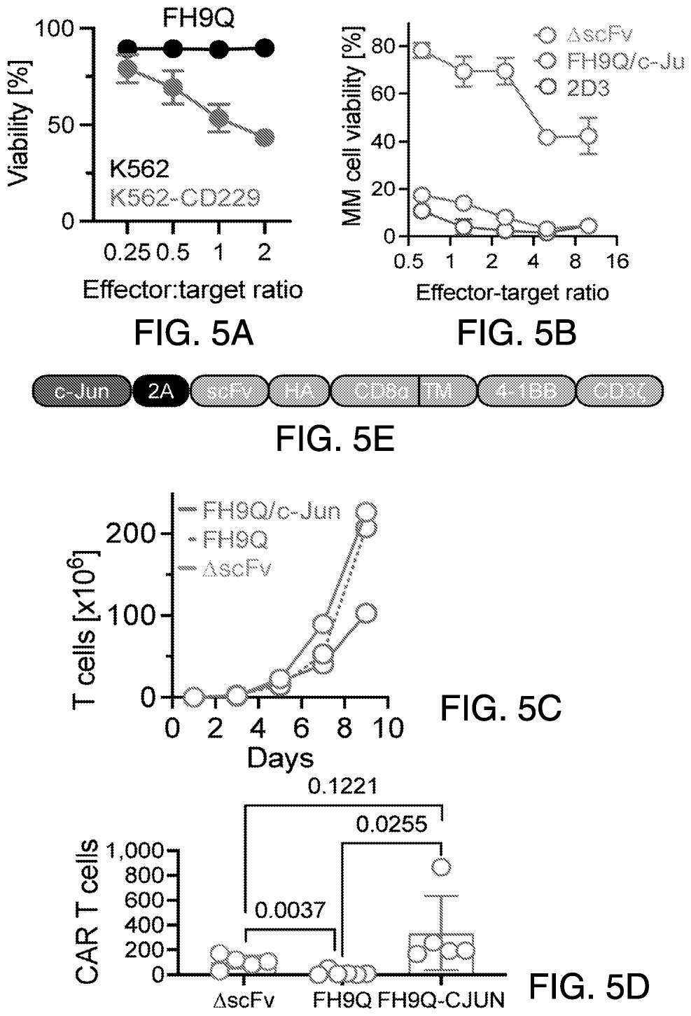

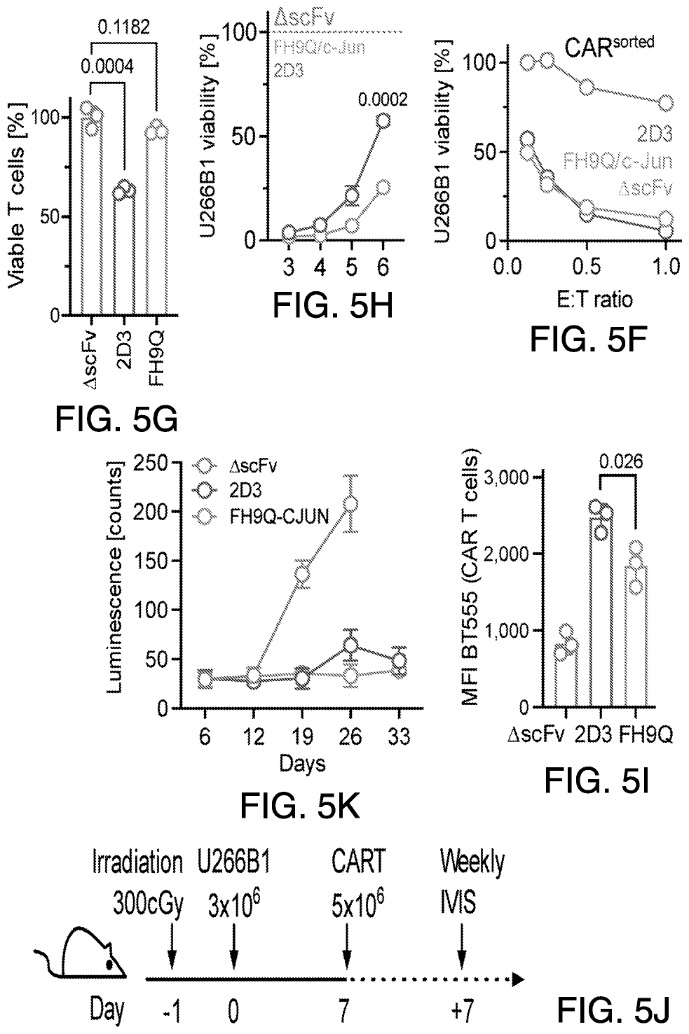

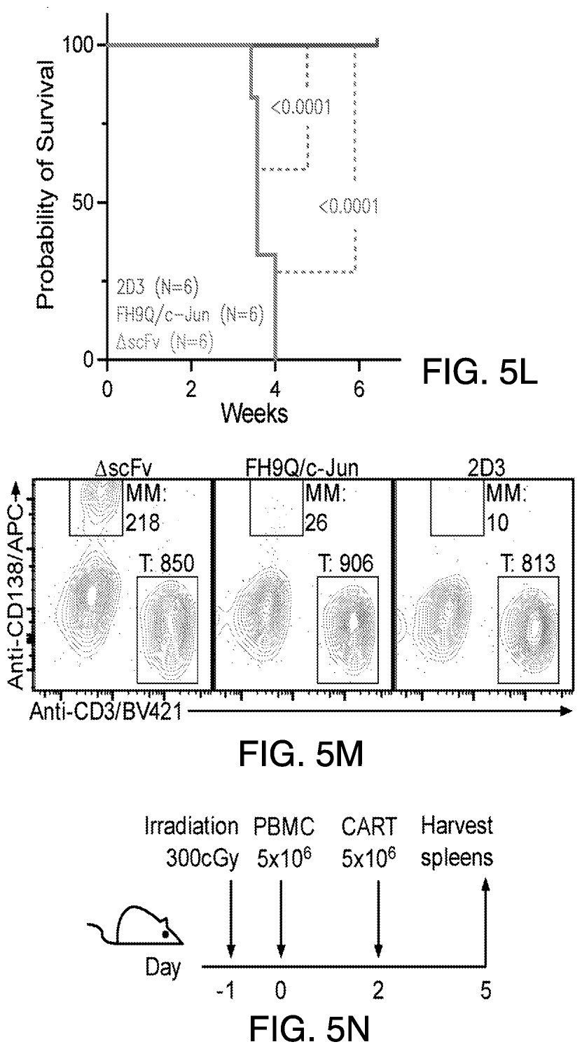

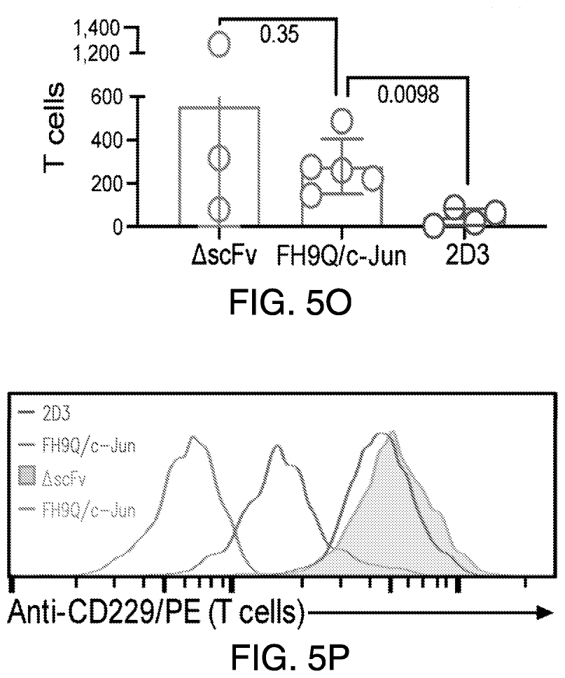

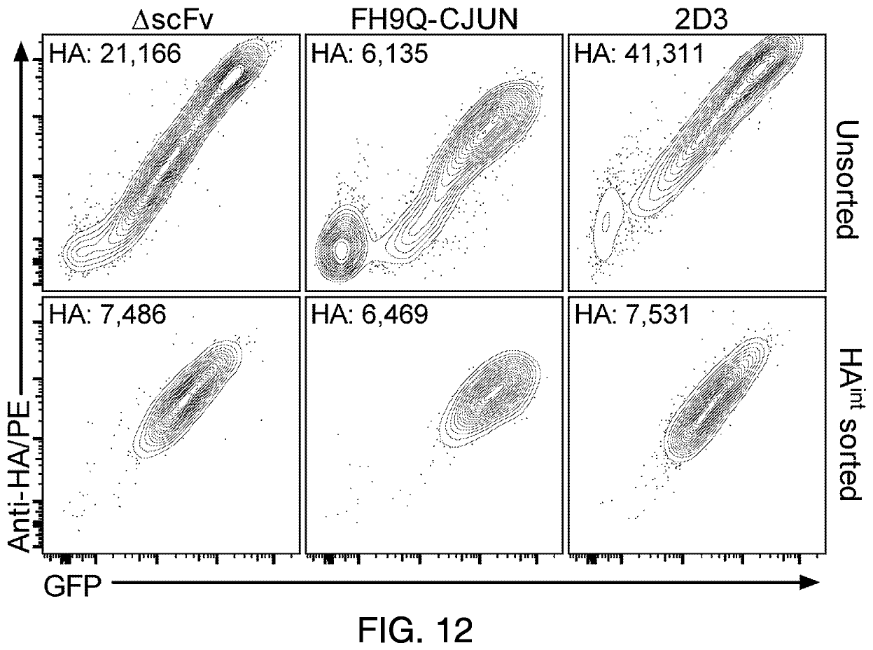

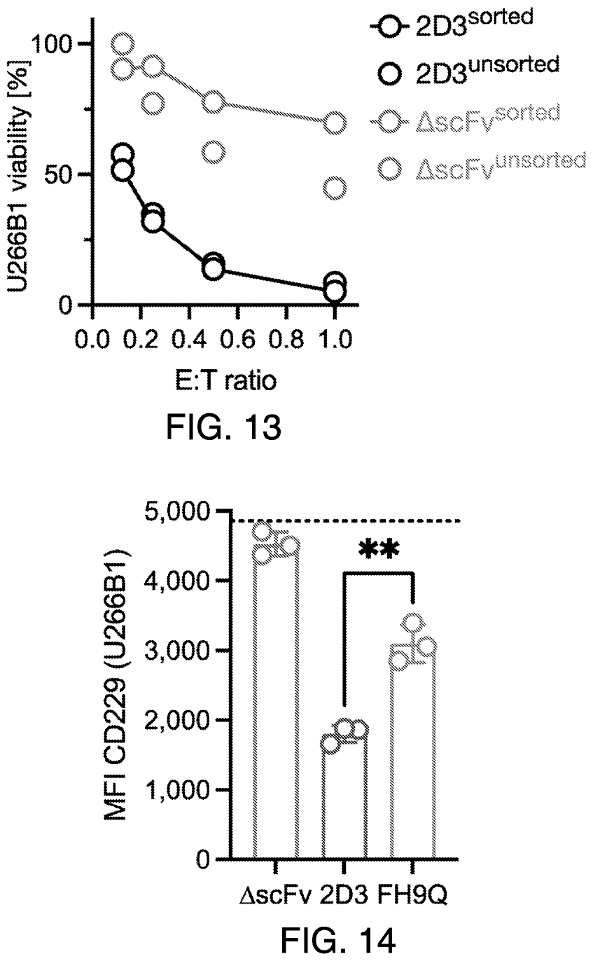

Although it was hypothesized that a single amino acid substitution would be unlikely to substantially alter the specificity of the binding domain as a whole, it was next determined whether FH9Q-based CAR T cells would still specifically target cells expressing CD229. It was indeed found that FH9Q CAR T cells did not kill a CD229-negative cell line, K562, but showed substantial killing of K562 cells engineered to express CD229 (K562-CD229, FIG. 5A), as well as primary CD229-expressing (FIG. 11) human MM cells (FIG. 5B). When scaling up FH9Q CAR T cell production, FH9Q CAR T cells, like many of the other variants (FIG. 4C) showed reduced expansion compared to ΔscFv CAR T cells in vitro (FIG. 5C) and in vivo (FIG. 5D). This is in direct contrast to the parental 2D3-based CAR T cells which expanded comparably to ΔscFv CAR T cells(21). These results are consistent with a previous report describing reduced in vitro and in vivo expansion of CAR T cells evidencing tonic signaling (27)—spontaneous T cell activation by the CAR in the absence of antigen(27). Tonic signaling is thought to be a consequence of receptor aggregation mediated by the CAR binding domain. As a result, the persistent signaling has been shown to lead to the rapid depletion of c-Jun, a component of the T cell activating AP-1 transcription factor. Overexpression of c-Jun was shown to efficiently rescue function and expansion of CAR T cells evidencing tonic signaling (28). An FH9Q CAR construct was generated to simultaneously overexpress c-Jun (FIG. 5E) and it was found that c-Jun overexpression efficiently restored FH9Q CAR T cell expansion in vitro (FIG. 5C) and in vivo (FIG. 5D). The possibility that the FH9Q CAR can be prone to aggregation is further substantiated by its reduced surface expression (FIG. 4D), as it had previously been shown that membrane-bound antibodies with higher levels of aggregation exhibit reduced surface expression (29). This difference in CAR surface expression between the FH9Q and 2D3 CARs can contribute to the FH9Q CAR T cells' increased selectivity. To determine the influence of surface expression levels on selectivity, 2D3, ΔscFv, and FH9Q CAR T cells were sorted to normalize for CAR surface expression (FIG. 12). Comparing sorted and unsorted 2D3 CAR T cells, no differences were observed in their ability to kill MM cells (FIG. 13). The ability of FH9Q and 2D3 CAR T cells sorted were compared for comparable CAR surface expression to kill MM cells and found that killing by these cells remained identical as well (FIG. 5F). Importantly, in a direct co-culture of CAR T cells with healthy T cells, significantly reduced killing of healthy T cells by FH9Q CAR T cells was observed compared to 2D3 CAR T cells even following normalization of CAR surface expression (FIG. 5G). These data indicate that the increased selectivity of FH9Q CAR T cells is likely the result of their reduced affinity and not due to altered CAR surface expression levels.

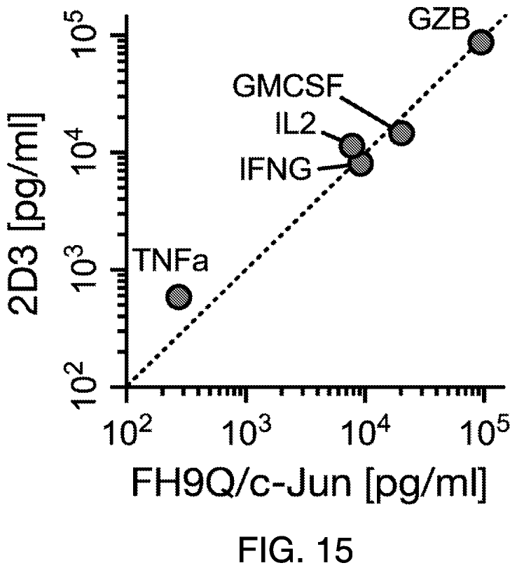

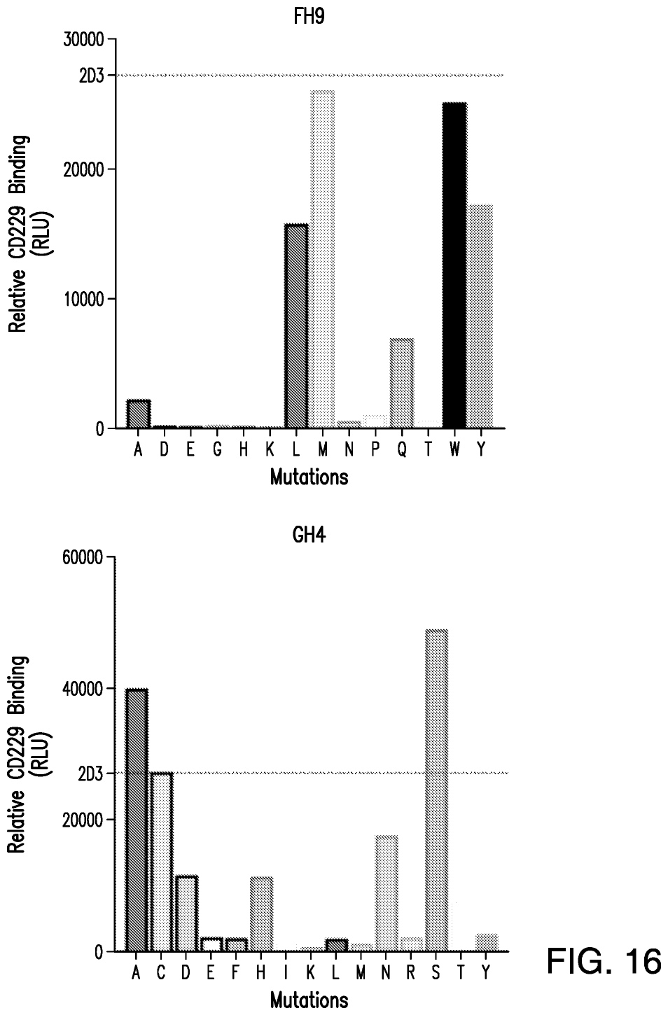

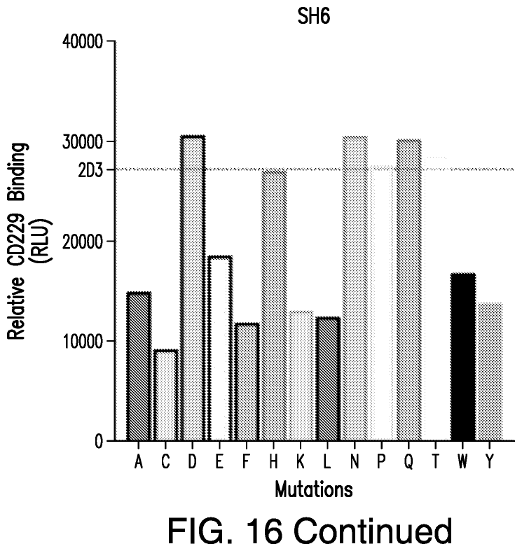

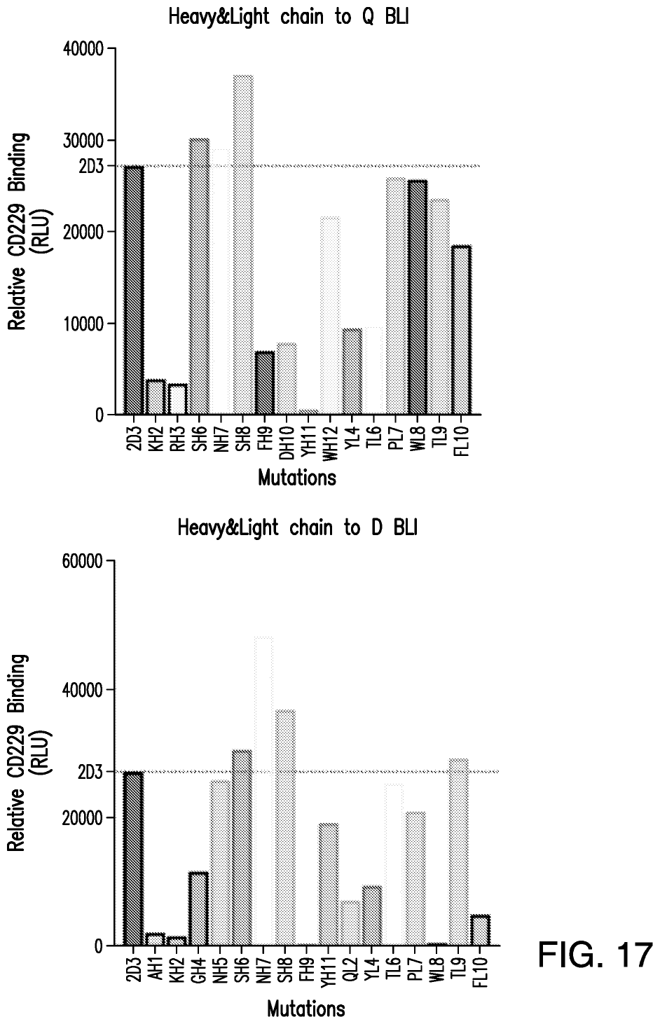

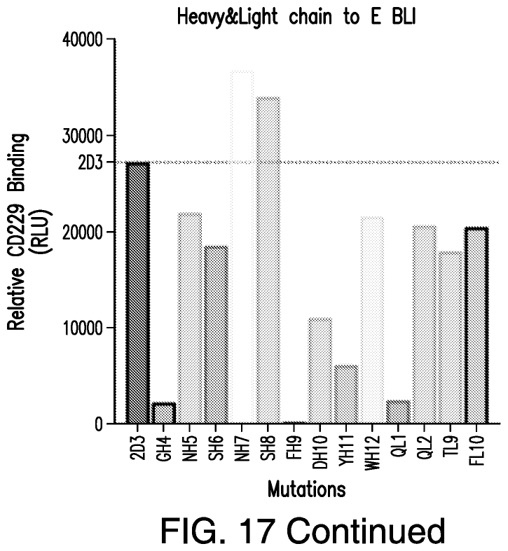

The next question that was asked was whether the substantial reduction in CAR affinity would result in incomplete tumor cell killing or suboptimal CAR T cell stimulation leading to reduced long-term disease control by FH9Q CAR T cells. To answer this question, an in vitro re-challenge assay was performed and FH9Q CAR T cells were observed to in fact show significantly increased long-term disease control compared to 2D3 CAR T cells (FIG. 5H). This finding can be related to the overexpression of c-Jun together with FH9Q or more physiological T cell stimulation by low affinity CAR constructs(13), but could also be the result of reduced trogocytosis by low affinity CAR T cells. Trogocytosis is the stripping of target antigen together with target cell membrane and their incorporation into the CAR T cell membrane, resulting in antigen-negative tumor cells and antigen-positive CAR T cells(30, 31). This phenomenon can lead to fratricide—the killing of antigen-positive CAR T cells by other CAR T cells and reduced trogocytosis can enhance CAR T cell persistence(31). To determine how much FH9Q CAR T cells confer trogocytosis compared to 2D3 CAR T cells, the amount of target antigen and membrane transferred from MM cells to CAR T cells was determined using flow cytometry. It was found that FH9Q CAR T cells had transferred significantly less tumor cell membrane (FIG. 5I) and target antigen (FIG. 14) than 2D3 CAR T cells, which can in turn have contributed to improved CAR T cell persistence and long-term disease control. In vivo experiments were conducted in NOD.Cg-Rag1^{tm1Mom} Il2rg^{tm1WjI}/SzJ (NRG) mice with luciferase-expressing human MM cells (FIG. 5J). Both 2D3 and FH9Q CAR T cells significantly reduced tumor burden (FIG. 5K) and prolonged survival compared to ΔscFv CAR T cells (FIG. 5L), indicating that FH9Q CAR T cells exhibit long-term anti-tumor activity comparable to 2D3 CAR T cells. In addition, comparable levels of major effector cytokines secreted by 2D3 and FH9Q CAR T cells were observed when co-cultured with MM cells, indicating comparable levels of T cell activation by FH9Q CAR T cells (FIG. 15).

As killing of healthy T cells represents the main liability of CD229 CAR T cells, the apparent lack of targeting of healthy T cells by FH9Q CAR T cells was explored in this screening assay (FIG. 4F). While significant killing of healthy T cells was observed in a direct-co-culture by 2D3 CAR T cells (FIG. 5G), only minor T cell killing by 2D3 CAR T cells was found in a mixed co-culture containing both MM and healthy T cells as targets (FIG. 5M). These results indicate that 2D3 CAR T cells already provide a degree of selectivity in the presence of tumor cells. However, in the absence of MM cells, not only was substantial killing in vitro observed (FIG. 5G) but also in an in vivo cytotoxicity assay (FIGS. 5N and 5O) with 2D3 CAR T cells. Importantly, in contrast to 2D3 CAR T cells, FH9Q CAR T cells did not show killing of healthy T cells in any of our in vitro and in vivo assays. Previously, it was observed that exposure of healthy T cells to 2D3 CAR T cells results in the selection of a population of CD229^{low} healthy T cells in vitro and in vivo (21). Analyzing CD229 expression on healthy T cells following co-culture with 2D3 or FH9Q CAR T cells, emergence of a CD229^{low} T cell population was observed when treated with 2D3 CAR T cells, but no selection of a CD229^{low}population was observed when treated with FH9Q CAR T cells. This result further substantiates the lack of targeting of healthy T cells by FH9Q CAR T cells (FIG. 5P). Overall, the data demonstrate that FH9Q CAR T cells maintain the anti-tumor activity of 2D3 CAR T cells in vitro and in vivo but lack their cytotoxic activity against healthy T cells.

### 2. Discussion

CAR T cells have revolutionized cancer immunotherapy but there is a critical need for the development of safer and more effective CAR T cell approaches to achieve more widespread adoption of CAR T cell treatment and increased patient benefit. A key challenge remains on-target off-tumor toxicity, the CAR T cell-mediated killing of healthy cells showing expression of the target antigen albeit generally at substantially lower levels. This not only leads to clinically relevant toxicities of existing CAR T cell approaches but also currently prevents the use of otherwise viable target antigens due to their shared expression on healthy tissues. Different combinatorial logic-gate approaches have been developed to address this issue, including AND-gates(32-35) (requiring recognition of multiple antigens) and NOT-gates(36) (requiring recognition of a single antigen in the absence of another) but have not yet been tested in the clinic. An alternative to these strategies is CAR affinity-tuning, which has come into focus for its effect on tumor selectivity and CAR T cell function(13-16) and does not require the identification and validation of an appropriate second antigen like in the above-mentioned logic-gate approaches. In this study, a systematic approach is provided for the development of minimally modified low-affinity antibody variants based on established and extensively tested antibodies that does not require detailed structural information including the antibody's exact epitope. The variants produced using this approach show significantly increased selectivity and improved CAR T cell function, while maintaining the original epitope and specificity. The majority of variants generated using this approach show a predominantly off-rate based reduction in affinity, likely as a consequence of using a solid-phase binding assay for primary variant screening. While data regarding the relative contribution of on-rate and off-rate on CAR T cell signaling remain scarce, prior approaches to modulate CAR affinity have also predominantly focused on changes in off-rates(37-39). This includes one of the most advanced approaches, which is already demonstrating the clinical benefits of the use of low affinity CAR constructs targeting CD19 (13, 38).

One potential liability of the approach is the development of tonic signaling in variant CAR T cells despite only minor changes to the binding domain. As shown here, overexpression of c-Jun in these CAR T cells can overcome this problem by rendering the cells resistant to the downstream effects of tonic signaling, however, it is likely that the success of this solution depends on the degree of tonic signaling. An alternative strategy to address the problem of tonic signaling and to improve CAR T cell function in general is an elegant approach using protease-sensitive CAR constructs (40). In this system, small molecule-mediated protease inhibition allows tight control over CAR surface expression levels, preventing early exhaustion resulting from tonic signaling, maintenance of a stem-like phenotype, and dramatically increased anti-tumor activity even when using aggregation-prone CAR constructs.

### 3. Methods

i. Cell Lines and Primary Human Cells

U266B1, K562, and Phoenix-Ampho cells were purchased from ATCC and cultured according to ATCC instructions. Lenti-X 293T cells were purchased from Takara and cultured according to the manufacturer's instructions. Cell lines were authenticated by their respective supplier. Healthy donor buffy coats were obtained from the Blood Centers of America and the New York Blood Center and peripheral blood mononuclear cells were isolated from buffy coats by density gradient using FicollPaque (GE) as previously described(21).

ii. Flow Cytometry Expression Analysis

Flow cytometry staining and analyses were performed as previously described(21). CD229 surface expression was determined using a mouse monoclonal anti-CD229 antibody (clone: HLy9.1.25). Other antibodies used for flow cytometry analyses are listed in Table 5. Commercially available antibodies were used at dilutions recommended by the respective manufacturer. For the analysis of CD229 expression on tumor cells and T cells from MM patients, data was acquired on a CytoFLEX LX (Beckman Coulter) and analyzed using Kaluza 2.1 (BC). All other flow cytometry data was acquired on an LSR Fortessa or LSR II flow cytometer (BD) and analyzed using FlowJo 10 (BD).

TABLE 5
Table of monoclonal antibodies and viability
dyes used for flow cytometry analyses.

| Target | Clone | Fluorophore | Supplier | Target cells |
|---|---|---|---|---|
| huCD229 | HLy9.1.25 | PE | Biolegend/ | Multiple |
| | | | R&D Systems | |
| huCD3 | UCHT1 | BUV496 | Biolegend | Pan-T |
| huCD138 | MI15 | BV510 | Biolegend | MM/Plasma |
| icVS38c | vs38c | FITC | Dako/Agilent | Primary MM |
| CD56 | N901 | ECD | Beckman | Primary MM |
| icKappa | TB28-2 | APC | Biolegend | Primary MM |
| icLambda | MHL-38 | APC-A750 | Biolegend | Primary MM |
| CD38 | HB7 | BUV395 | Fisher | Primary MM |
| CD19 | HIB19 | BUV737 | BD | Primary MM |
| CD45 | HI30 | AF790 | Thermo | Primary MM |
| Hemagglutinin | 6E2 | PE/APC | Cell Signaling | CAR T |
| | | | Technology | |
| LIVE/DEAD | L34957 | N/A | Life | Live/dead |
| Aqua | | | Technologies | |
| Propidium | 421301 | N/A | Biolegend | Live/dead |
| iodide | | | | |
| DAPI | D1306 | N/A | Life | Live/dead |
| | | | Technologies | |
| DYKDDDDK | M2 | PE | Sigma-Aldrich | Multiple |
| DYKDDDDK | L2 | PE/APC | Biolegend | Multiple |

iii. Single Amino Acid Substitution Library Production

Parental 2D3 was cloned into the pSANG10 bacterial expression plasmid(41) and the single amino acid substitution library produced by was generated using high-throughput gene synthesis by Twist Bioscience. Individual mutations were confirmed by Sanger sequencing. To produce single-site biotinylated scFvs, a C-terminal AviTag was added to the pSANG10 expression constructs (pSANG10-Avi) and scFv constructs in the bacterial expression plasmids were transformed into BL21[DE3] cells (Lucigen) containing the pBirAcm plasmid (Avidity).

iv. scFv Expression and Purification

Monoclonal scFvs were expressed overnight in 25 mL MagicMedia E. coli autoexpression medium (Thermo-Fisher). Periplasmic extracts were generated from autoinduction cultures using standard procedures. For some experiments, scFvs were purified by immobilized metal affinity chromatography using NiNTA resin (Thermo). Concentrations of purified scFvs were determined by bicinchoninic assay (Thermo). pSANG10-Avi clones were expressed in the same way in the presence of 50 μM free D-biotin (Sigma).

v. Interferon-Gamma (IFN-γ) Enzyme-Linked Immunosorbent Assay (ELISA)

Supernatants were harvested from 96-well overnight co-cultures and immediately frozen at −80° C. IFN-γ concentrations were determined via standard curve using a commercial ELISA kit according to the manufacturer's instructions (Biolegend). Absorbance was measured on a multi-mode plate reader (Tecan).

vi. CAR T Cell Production

Selected scFvs were cloned into a previously described second generation 4-1BB-based CAR construct(21) in the gammaretroviral SFG backbone. Amphotropic gammaretrovirus was generated by transfection of Phoenix-Ampho cells (ATCC #CRL-3213) using the SFG-based transfer plasmids using Lipofectamine 2000 according to the manufacturer's instructions. Virus-containing supernatants were concentrated with Retro-X concentrator (Takara). PBMCs were stimulated for 2 days with CD3/CD28 T cell activation beads (Thermo #11131D) in the presence of 40 IU/mL IL2 (R&D Systems #202-IL-010) in AIM V media (Thermo) supplemented with 5% human serum (Sigma #H3667) and incubated at 37° C./5% CO₂. Bead-stimulated cells were transferred to Retronectin-coated (Takara) virus-containing plates and incubated overnight. Transduction was repeated the next day before counting and diluting cells to 0.4×10⁶ cells/ml. After the second transduction cells were grown for an additional 7 days before removing beads using a DynaMag-15 magnet (Thermo). IL-2 was replenished every 2 days to 40 IU/mL. Cells were frozen in 90% FCS/10% DMSO and stored in liquid nitrogen.

vii. Trogocytosis Assay

CAR T cells were co-cultured with target cells at the specified effector-target ratio. Target cells were first labelled with BioTracker 555 (Sigma #SCT107) according to the manufacturer's instructions. After the specified amount of time, cells were stained with antibodies and 500 ng/mL 4′,6-diamidine-2′-phenylindole dihydrochloride (DAPI, Invitrogen #D1306). Samples were analyzed on an LSR II flow cytometer (BD).

viii. Flow Cytometry-Based Cytotoxicity Assay.

A flow cytometry-based cytotoxicity assay was used to determine CAR T cell cytotoxicity against healthy T cells from the same healthy donor as well as primary MM cells. T cells were collected using negative selection (Stemcell Technologies EasySep Human T Cell Isolation Kit) from autologous healthy donor PBMCs. MM cells and T cells were stained with Cell Trace Far Red dye (CTD, Invitrogen) according to the manufacturer's instructions. Target cells at 5×10⁴ cells/well were co-cultured with different amounts of CAR T cells overnight in a round bottom 96 well plate at 37° C./5% CO₂. Following co-culture, Accucheck counting beads (Life Technologies) and 500 ng/mL DAPI were added to the cells. DAPI⁻CTD⁺ T Cells were immediately quantified on an LSR II flow cytometer (BD).

ix. Luciferase-Based Cytotoxicity Assay

To determine the cytotoxicity of variant CD229 CAR T cells against the multiple myeloma cell line U266B1 and chronic lymphocytic leukemia cell line K562, cell lines were transduced with pHIV-Luc-ZsGreen lentivirus and sorted on a FACSaria flow cytometer (BD) for GFP expression. CD229-negative K562 cells were also transduced with a CD229 expression construct as previously described(21). As with the flow cytometry-based cytotoxicity assay, 5×10⁴ target cells were seeded in each well of a round bottom 96-well plate. Various ratios of CAR T cells were co-cultured with target cells overnight at 37° C./5% CO₂. After the co-culture, cells were suspended by gentle pipetting and 100 uL were moved to a 96-well black flat bottom plate. D-luciferin at 150 μg/ml (Gold Biotechnology #LUCNA-2G) was added to the cells and incubated for 5 mins at 37° C. Luminescence was determined on a multi-mode plate reader (Tecan Spark). For the re-challenge assay, luminescence was determined daily before adding 5×10⁴ target cells to each well.

x. Time-Resolved Fluorescence (TRF) Assays

Two different TRF assays were used. To determine the concentration of scFv in the PPEs, 250 ng of rat-anti-FLAG (clone: L5, Biolegend) in 50 μL PBS was immobilized on a black 96-well plate (Greiner Bio-One) overnight at 4° C. Plates were washed using an automated plate washer (Tecan HydroFlex) twice with PBS containing 0.1% Tween-20, and twice with PBS, in between each incubation. After immobilization, all other incubations were performed at room temperature at 400 rpm. Following immobilization, plates were blocked in 3% non-fat milk in PBS (M-PBS). Then PPEs or purified 2D3 were added to plates in 3% M-PBS and incubated for 1 h. Next, plates were incubated with 250 ng biotinylated protein L (Thermo Scientific) in 3% M-PBS for 1-hour. Finally, plates were incubated with streptavidin-Europium (PerkinElmer) in PBS for 30 minutes. After a final wash, plates were incubated with DELFIA Enhancement solution (PerkinElmer) for 10 minutes. TRF was determined on a multi-mode plate reader (Tecan Spark). A purified parental 2D3 standard was used to calculate the scFv concentration in each PPE.

To determine the relative binding of variant scFvs to CD229, 5 μg/ml recombinant human CD229 (R&D Systems) was immobilized on a black 96-well plate (Greiner Bio-One) overnight at 4° C. After immobilization, plates were washed between each step and incubated at room temperature at 400 rpm as described above. Following immobilization, plates were blocked in 3% M-PBS. Then, plates were incubated with 100 ng of each scFv in 3% M-PBS. Next, plates were incubated with anti-FLAG M2 (Sigma-Aldrich) in 3% M-PBS. Finally, plates were incubated with anti-mouse IgG-Europium antibody (PerkinElmer) for 1 hour. After a final wash, plates were incubated with DELFIA Enhancement solution (PerkinElmer) for 10 minutes. TRF was determined on a multi-mode plate reader (Tecan Spark).

xi. Bio-Layer Interferometry (BLI)

Streptavidin (SA) biosensensors (ForteBio) were hydrated in 1× Octet® Kinetics Buffer (Sartorius) for at least 10 minutes. SA biosensors were loaded using the Octet K2 (Sartorius). A baseline in Octet Kinetics Buffer was collected for 1 minute. Then the sensors were loaded with variant scFvs using a threshold of 2 nm and subsequently blocked in Biocytin. Once loaded, sensors were placed back into the sensor tray and kept hydrated in kinetics buffer. Once sensors were loaded, a kinetic run was performed. SA biosensors loaded with 2 nm of biotinylated scFv and blocked with biocytin went through a 60-second baseline read in kinetics buffer, 50-second association in 2, 1, 0.5, and 0.25 μM recombinant human CD229 (R&D Systems) in kinetics buffer, and finally 60-second dissociation in kinetics buffer. BLI was run at 30° C. and 10,000 rpm. Data was analyzed using Octet® K2 System Data Analysis 9.0 software.

### xii. 2D3 Structure Prediction

The structure of the wildtype 2D3 scFv was generated using AlphaFold2 with default parameters (42). Structures were visualized using UCSF ChimeraX.

xiii. Multiple Myeloma Xenograft Mouse Model

Six- to 8-week-old male NOD.Cg-Rag1^{tmlMom} Il12rg^{tmlWjl}/SzJ (NRG, The Jackson Laboratory (Cat #005557)) mice were irradiated with a sublethal dose of 300 cGy (Rad-Source RS-2000) and injected on the next day via the lateral tail vein with the indicated numbers of U266B1 cells stably expressing luciferase. On day 7 after tumor cell injection, the indicated numbers of CD229 CAR T cells or CAR T cells lacking a binding domain (AscFv) were injected into the tail vein. Animals were weighed twice weekly and monitored for signs of distress in accordance with institutional regulations. For in vivo imagining, mice received an intraperitoneal injection of 3.3 mg D-luciferin. Photographic and luminescent images were acquired starting 10 minutes after the D-luciferin injection, both in prone and supine position using a IVIS imaging system. Myeloma progression was monitored every 7 days until the study endpoint. Average radiance (p/s/cm²/sr) was quantified for individual animals using Living Image software (PerkinElmer).

xiv. In Vivo Cytotoxicity Assay Using Human PBMCs

Eight-week-old male NRG mice were irradiated with a sublethal dose of 300 cGy (Rad-Source RS-2000) and on the following day injected with 5×10⁶ PBMCs isolated from healthy donors. On day 2 after PBMC injection, mice were injected with 5×10⁶ CD229 CAR T cells via tail vein. On day 5 after PBMC injection, animals were sacrificed and spleens were collected for flow cytometry analysis. After a 5-minute incubation in red blood cell lysis buffer (Biolegend), cells were washed twice in PBS, incubated with human and mouse FcR blocking reagents (Miltenyi Biotec) for 15 minutes, and then stained with population-specific antibodies and DAPI for 30 minutes. Stained samples were analyzed on an LSR II flow cytometer (BD) and cell numbers normalized using counting beads (Thermo).

xv. CodePlex Secretome Assay

Supernatants were harvested from overnight co-cultures and stored at −80° C. until further use. On the day of the assay, samples were thawed and 11 μl of supernatant per sample was added to CodePlex Human Adaptive Immune secretome chips (Isoplexis). Chips were loaded into the Isolight reader and cytokines measured using default settings. Automated analysis of raw data was performed using IsoSpeak (Isoplexis).

xvi. Statistical Analysis

Significance of differences in cell numbers, cytokine levels, and mean fluorescence intensity levels were calculated by Student's t-test. All statistical tests were performed using Prism 9 (GraphPad Software). Results were considered significant when p<0.05.

### B. Example 2: Comparison Data

FIG. 16 shows the binding comparison of the different substitutions in the positions that are changed in the 3 candidate clones (FH9, GH4, SH6). FIG. 17 shows the substitutions to the same new amino acids as in the candidates (Q, D, E) but in other positions. FIG. 16 shows that in those three positions the original amino acid could not have been replaced with any other amino acid to substantially reduce the level of binding; it had to be one of very few (in the case of FH9, it can really only be Q) to achieve the desired effect. FIG. 17 shows that changing any other residue to the new amino acid in the respective candidate clone would not have resulted in the same reduction in binding. These are similar data to those shown in FIG. 2D and a similar method was used.

These findings demonstrate that only a small subset of mutations in specific positions maintained the ability of the resulting clones to bind to CD229 while substantially reducing the level of binding. These data further confirm that screening of antibody variants obtained by site-saturation mutagenesis is superior to conventional methods such as alanine scanning. Importantly, in many cases, the identified mutations using our approach were not obvious candidates for substitution based on structural or biochemical similarity of the parental and the new residue.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. M. Sadelain, CD19 CAR T Cells. Cell 171, 1471 (2017).
2. N. Raje et al., Anti-BCMA CAR T-Cell Therapy bb2121 in Relapsed or Refractory Multiple Myeloma. The New England journal of medicine 380, 1726-1737 (2019).
3. A. Mullard, FDA approves 100th monoclonal antibody product. Nat Rev Drug Discov, (2021).
4. M. A. Cheever et al., The prioritization of cancer antigens: a national cancer institute pilot project for the acceleration of translational research. Clin Cancer Res 15, 5323-5337 (2009).
5. S. Sun, H. Hao, G. Yang, Y. Zhang, Y. Fu, Immunotherapy with CAR-Modified T Cells: Toxicities and Overcoming Strategies. Journal of immunology research 2018, 2386187-2386187 (2018).
6. A. V. Hirayama, C. J. Turtle, Toxicities of CD19 CAR-T cell immunotherapy. American Journal of Hematology 94, S42-S49 (2019).
7. M. Mamonkin, R. H. Rouce, H. Tashiro, M. K. Brenner, A T-cell-directed chimeric antigen receptor for the selective treatment of T-cell malignancies. Blood 126, 983-992 (2015).
8. X. Liu, N. Zhang, H. Shi, Driving better and safer HER2-specific CARs for cancer therapy. Oncotarget 8, 62730-62741 (2017).
9. R. A. Morgan et al., Case Report of a Serious Adverse Event Following the Administration of T Cells Transduced With a Chimeric Antigen Receptor Recognizing ERBB2. Molecular Therapy 18, 843-851 (2010).
10. M. Chmielewski, A. Hombach, C. Heuser, G. P. Adams, H. Abken, T Cell Activation by Antibody-Like Immunoreceptors: Increase in Affinity of the Single-Chain Fragment Domain above Threshold Does Not Increase T Cell Activation against Antigen-Positive Target Cells but Decreases Selectivity. The Journal of Immunology 173, 7647 (2004).
11. X. Liu et al., Affinity-Tuned ErbB2 or EGFR Chimeric Antigen Receptor T Cells Exhibit an Increased Therapeutic Index against Tumors in Mice. Cancer Res 75, 3596-3607 (2015).
12. F. Turatti et al., Redirected activity of human antitumor chimeric immune receptors is governed by antigen and receptor expression levels and affinity of interaction. J Immunother 30, 684-693 (2007).
13. S. Ghorashian et al., Enhanced CAR T cell expansion and prolonged persistence in pediatric patients with ALL treated with a low-affinity CD19 CAR. Nature Medicine 25, 1408-1414 (2019).
14. T. Heitner et al., Selection of cell binding and internalizing epidermal growth factor receptor antibodies from a phage display library. J Immunol Methods 248, 17-30 (2001).
15. J. Yang et al., Therapeutic potential and challenges of targeting receptor tyrosine kinase ROR1 with monoclonal antibodies in B-cell malignancies. PloS one 6, e21018-e21018 (2011).
16. M. Hudecek et al., Receptor Affinity and Extracellular Domain Modifications Affect Tumor Recognition by ROR1-Specific Chimeric Antigen Receptor T Cells. Clinical Cancer Research 19, 3153 (2013).
17. S. A. Ali et al., T cells expressing an anti-B-cell maturation antigen chimeric antigen receptor cause remissions of multiple myeloma. Blood 128, 1688-1700 (2016).
18. K. M. Friedman et al., Effective Targeting of Multiple B-Cell Maturation Antigen-Expressing Hematological Malignances by Anti-B-Cell Maturation Antigen Chimeric Antigen Receptor T Cells. Hum Gene Ther 29, 585-601 (2018).
19. A. L. Garfall et al., Chimeric Antigen Receptor T Cells against CD19 for Multiple Myeloma. The New England journal of medicine 373, 1040-1047 (2015).
20. K. Boucher et al., Sternness of B-cell progenitors in multiple myeloma bone marrow. Clin Cancer Res 18, 6155-6168 (2012).
21. S. V. Radhakrishnan et al., CD229 CAR T cells eliminate multiple myeloma and tumor propagating cells without fratricide. Nat Commun 11, 798 (2020).
22. I. C. Nicholson et al., Construction and characterisation of a functional CD19 specific single chain Fv fragment for immunotherapy of B lineage leukaemia and lymphoma. Mol Immunol 34, 1157-1165 (1997).
23. T. Tiller et al., A fully synthetic human Fab antibody library based on fixed VH/VL framework pairings with favorable biophysical properties. MAbs 5, 445-470 (2013).
24. S. J. Wu et al., Structure-based engineering of a monoclonal antibody for improved solubility. Protein Eng Des Sel 23, 643-651 (2010).
25. G. A. Weiss, C. K. Watanabe, A. Zhong, A. Goddard, S. S. Sidhu, Rapid mapping of protein functional epitopes by combinatorial alanine scanning. Proc Natl Acad Sci USA 97, 8950-8954 (2000).
26. R. L. Petersen, Strategies Using Bio-Layer Interferometry Biosensor Technology for Vaccine Research and Development. Biosensors (Basel) 7, (2017).
27. A. H. Long et al., 4-1BB costimulation ameliorates T cell exhaustion induced by tonic signaling of chimeric antigen receptors. Nat Med 21, 581-590 (2015).
28. R. C. Lynn et al., c-Jun overexpression in CAR T cells induces exhaustion resistance. Nature 576, 293-300 (2019).
29. M. R. Dyson et al., Beyond affinity: selection of antibody variants with optimal biophysical properties and reduced immunogenicity from mammalian display libraries. MAbs 12, 1829335 (2020).
30. M. Hamieh et al., CAR T cell trogocytosis and cooperative killing regulate tumour antigen escape. Nature 568, 112-116 (2019).
31. M. L. Olson et al., Low-affinity CAR T cells exhibit reduced trogocytosis, preventing rapid antigen loss, and increasing CAR T cell expansion. Leukemia, (2022).
32. C. C. Kloss, M. Condomines, M. Cartellieri, M. Bachmann, M. Sadelain, Combinatorial antigen recognition with balanced signaling promotes selective tumor eradication by engineered T cells. Nat Biotechnol 31, 71-75 (2013).
33. E. Lanitis et al., Chimeric antigen receptor T Cells with dissociated signaling domains exhibit focused antitumor activity with reduced potential for toxicity in vivo. Cancer immunology research 1, 43-53 (2013).
34. K. T. Roybal et al., Engineering T Cells with Customized Therapeutic Response Programs Using Synthetic Notch Receptors. Cell 167, 419-432 e416 (2016).
35. A. M. Tousley et al., Coopting T cell proximal signaling molecules enables Boolean logic-gated CAR T cell control. bioRxiv, 2022.2006.2017.496457 (2022).
36. V. D. Fedorov, M. Themeli, M. Sadelain, PD-1- and CTLA-4-based inhibitory chimeric antigen receptors (iCARs) divert off-target immunotherapy responses. Sci Transl Med 5, 215ra172 (2013).
37. S. Arcangeli et al., Balance of Anti-CD123 Chimeric Antigen Receptor Binding Affinity and Density for the Targeting of Acute Myeloid Leukemia. Mol Ther 25, 1933-1945 (2017).
38. C. Roddie et al., Durable Responses and Low Toxicity After Fast Off-Rate CD19 Chimeric Antigen Receptor-T Therapy in Adults With Relapsed or Refractory B-Cell Acute Lymphoblastic Leukemia. J Clin Oncol, JCO2100917 (2021).
39. E. Drent et al., A Rational Strategy for Reducing On-Target Off-Tumor Effects of CD38-Chimeric Antigen Receptors by Affinity Optimization. Mol Ther 25, 1946-1958 (2017).
40. L. Labanieh et al., Enhanced safety and efficacy of protease-regulated CAR-T cell receptors. Cell 185, 1745-1763 e1722 (2022).
41. D. J. Schofield et al., Application of phage display to high throughput antibody generation and characterization. Genome biology 8, R254 (2007).
42. J. Jumper et al., Highly accurate protein structure prediction with AlphaFold. Nature 596, 583-589 (2021).

We claim:

1. A chimeric antigen receptor (CAR) polypeptide, comprising a CD229 antigen binding domain, a transmembrane domain, and an intracellular signaling domain, wherein the CD229 binding domain is a variant of SEQ ID NO:1.

2. The CAR polypeptide of claim 1, wherein the CD229 antigen binding domain comprises the sequence of
QVQLVESGGGLVQPGRSLRLSCAASGFTFDDYAMHWVRQAPGKGLEWVS
GISWNSGSIGYADSAKGRFTISRDNSKNTLYLQMNSLRAEDTAVYYCAK
RGNSNSQDYWGQGTLVTVSSLEGGGGSGGGGSGGGASDIQMTQSPSSVS
ASVGDRVTITCRASQSISSYLNWYQQKPGKAPKLLIYAASSLQSGVPSR
FSGSGSGTDFTLTISSLQPEDFATYYCQQSYSTPWTFGQGTKLEIKR.

3. The CAR polypeptide of claim 1, wherein the CD229 antigen binding domain comprises the sequence of
QVQLVESGGGLVQPGRSLRLSCAASGFTFDDYAMHWVRQAPGKGLEWVS
GISWNSGSIGYADSAKGRFTISRDNSKNTLYLQMNSLRAEDTAVYYCAK
RDNSNSFDYWGQGTLVTVSSLEGGGGSGGGGSGGGASDIQMTQSPSSVS
ASVGDRVTITCRASQSISSYLNWYQQKPGKAPKLLIYAASSLQSGVPSR
FSGSGSGTDFTLTISSLQPEDFATYYCQQSYSTPWTFGQGTKLEIKR.

4. The CAR polypeptide of claim 1, wherein the CD229 antigen binding domain comprises one or more of the amino acid substitutions illustrated in FIG. 2.

5. The CAR polypeptide of any of the preceding claims, wherein the CD229 antigen binding domain is an antibody fragment or an antigen-binding fragment that specifically binds to CD229.

6. The CAR polypeptide of any of the preceding claims, wherein the CD229 antigen binding domain is a Fab or a single-chain variable fragment (scFv) of an antibody that specifically binds CD229.

7. The CAR polypeptide of any of the preceding claims, wherein the CD229 antigen binding domain comprises one or more amino acid substitutions in the HCDR3 of SEQ ID NO:1.

8. The CAR polypeptide of any of the preceding claims, wherein the CD229 antigen binding domain comprises a HCDR3 comprising the sequence of AKRDNSNSFDYW, AKRGNENSFDYW, or AKRGNSNSQDYW.

9. The CAR polypeptide of any one of the preceding claims, wherein the variant CD229 antigen binding domain comprises one or more of the amino acid substitutions in the LCDR3 of the 2D3 scFv.

10. The CAR polypeptide of any of the preceding claims, wherein the CD229 antigen binding domain comprises a LCDR3 comprising the sequence of any of those in Table 2.

11. The CAR polypeptide of any one of the preceding claims, wherein the CD229 antigen binding domain comprises a sequence having at least 90% identity to the sequence set forth in SEQ ID NOs:53, 84, or 134, wherein the CD229 antigen binding domain comprises 100% identity to SEQ ID NOs:53, 84, or 134 at the HCDR3.

12. The CAR polypeptide of any of the preceding claims, wherein the CD229 antigen binding domain comprises a heavy chain immunoglobulin variable region comprising:
a. a complementarity determining region 1 (CDR1) comprising the sequence of GFTFDDYA;
b. a CDR2 comprising the sequence of ISWNSGSI; and
c. a CDR3 comprising the sequence of AKRDNSNSFDYW, AKRGNENSFDYW, or AKRGNSNSQDYW.

13. The CAR polypeptide of any of the preceding claims, wherein the variant CD229 antigen binding domain comprises a light chain immunoglobulin variable region comprising any of those of Table 2.

14. The CAR polypeptide of any of the preceding claims, wherein the CD229 antigen binding domain comprises an altered affinity for CD229.

15. The CAR polypeptide of claim 14, wherein the altered affinity is a lower affinity.

16. The CAR polypeptide of claim 14, wherein the altered affinity is a high affinity.

17. The CAR polypeptide of claim 16, wherein the CD229 antigen binding domain comprises the sequence of
QVQLVESGGGLVQPGRSLRLSCAASGFTFDDYAMHWVRQAPGKGLEWVS
GISWNSGSIGYADSAKGRFTISRDNSKNTLYLQMNSLRAEDTAVYYCAK
RGNSDSFDYWGQGTLVTVSSLEGGGGSGGGGSGGGASDIQMTQSPSSVS
ASVGDRVTITCRASQSISSYLNWYQQKPGKAPKLLIYAASSLQSGVPSR
FSGSGSGTDFTLTISSLQPEDFATYYCQQSYSTPWTFGQGTKLEIK.

18. The CAR polypeptide of any of the preceding claims, wherein the intracellular signaling domain comprises a co-stimulatory signaling region.

19. The CAR polypeptide of any of the preceding claims, wherein the co-stimulatory signaling region comprises the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

20. The CAR polypeptide of any of the preceding claims, wherein the intracellular signaling domain is a T cell signaling domain.

21. The CAR polypeptide of any of the preceding claims, wherein the intracellular signaling domain comprises a CD3 zeta (CD3ζ) signaling domain.

22. The CAR polypeptide of any of the preceding claims, wherein the intracellular signaling domain comprises a CD3ζ signaling domain and a co-stimulatory signaling region, wherein the co-stimulatory signaling region comprises the cytoplasmic domain of CD28 or 4-1BB.

23. The CAR polypeptide of any of the preceding claims, wherein the transmembrane domain comprises an immunoglobulin Fc domain.

24. The CAR polypeptide of claim 20 wherein the immunoglobulin Fc domain is an immunoglobulin G Fc domain.

25. The CAR polypeptide of any of the preceding claims, wherein the transmembrane domain comprises a CD8α domain, CD3ζ, FcεR1γ, CD4, CD7, CD28, OX40, or H2-Kb.

26. The CAR polypeptide of any of the preceding claims, wherein the transmembrane domain is located between the CD229 antigen binding domain and the intracellular signaling domain.

27. The CAR polypeptide of any of the preceding claims, further comprising a tag sequence.

28. The CAR polypeptide of claim 269, wherein the tag sequence is located between the CD229 antigen binding domain and the transmembrane domain.

29. The CAR polypeptide of any one of claims 24-25, wherein the tag sequence is a hemagglutinin tag.

30. The CAR polypeptide of any of the preceding claims further comprising a hinge region.

31. The CAR polypeptide of claim 27, wherein the hinge region is located between the CD229 antigen binding domain and the transmembrane domain.

32. A nucleic acid sequence capable of encoding the CAR polypeptide of any of the preceding claims.

33. A vector comprising the nucleic acid sequence of claim 32.

34. The vector of claim 33, wherein the vector is selected from the group consisting of a DNA, a RNA, a plasmid, and a viral vector.

35. The vector of any of claims 33-34, wherein the vector comprises a promoter.

36. A cell comprising the CAR polypeptide of any one of claims 1-31, the nucleic acid of claim 32, or the vector of any one of claims 33-35.

37. The cell of claim 3636, wherein the cell is a T cell.

38. The cell of claim 37, wherein the T cell is a CD8+ T cell.

39. The cell of any of claims 36-38, wherein the cell is a human cell.

40. A T cell expressing the CAR polypeptide of any one of claims 1-31.

41. A T cell expressing a CAR polypeptide of any one of claims 1-31 that binds human CD229, wherein the T cell has increased specificity to multiple myeloma cells.

42. An antibody or fragment thereof that binds to human CD229, wherein said antibody comprises a heavy chain immunoglobulin variable region comprising:
a. a complementarity determining region 1 (CDR1) comprising the sequence of GFTFDDYA;
b. a CDR2 comprising the sequence of ISWNSGSI; and
c. a CDR3 comprising the sequence of AKRDNSNSFDYW, AKRGNENSFDYW, or AKRGNSNSQDYW.

43. An antibody or fragment thereof that binds to human CD229, wherein said antibody comprises a light chain immunoglobulin variable region comprising one or more of the light chain sequences of Table 2.

44. The antibody or fragment thereof of any of claims 42-43, wherein the antibody or fragment thereof comprises one or more of the amino acid substitutions illustrated in FIG. 2.

45. The antibody or fragment thereof of any one of claims 42-44, further comprising a tag sequence.

46. A nucleic acid sequence capable of encoding the antibody or fragment thereof of any one of claims 42-45.

47. A method of treating multiple myeloma comprising administering an effective amount of a T cell genetically modified to express the CAR polypeptide of any one of claims 1-31 to a subject in need thereof.

48. A method of treating multiple myeloma comprising administering an effective amount of a composition comprising the antibody or fragment thereof of any one of claims 42-45 to a subject in need thereof45.

49. The method of any one of claims 47-48 further comprising administering a therapeutic agent.

50. The method of claim 49, wherein the therapeutic agent is chemotherapy, proteasome inhibitors, immunomodulatory agents, histone deacetylase inhibitors, monoclonal antibodies, bispecific antibodies, or immune checkpoint inhibitors.

51. The method of any one of claims 47-50, wherein trogocytosis is reduced.

52. A method of detecting CD229 on a cell comprising administering a composition comprising the antibody or fragment thereof of any one of claims 42-45 to a sample and detecting the binding of the antibody or fragment thereof to CD229.

53. The method of claim 52, wherein detecting the binding of the antibody or fragment thereof to CD229 comprises immunostaining.

54. A method of killing CD229 positive cells comprising administering an effective amount of a T cell genetically modified to express the CAR polypeptide of any one of claims 1-31 to a sample comprising CD229 positive cells.

55. A method of making a cell comprising transducing a T cell with the vector of any of claims 32-35.

56. A method of activating a T cell of any one of claims 37-39 comprising culturing the T cell with a cell expressing CD229 and detecting the presence or absence of IFN-γ after culturing, wherein the presence of IFN-γ indicates the activation of the T cell.

57. A method of increasing specificity of CD229 CAR T cells to multiple myeloma cells comprising administering an effective amount of a T cell genetically modified to express the CAR polypeptide of any one of claims 1-31 to a subject in need thereof.

58. The method of claim 57, wherein the multiple myeloma cells are targeted with a higher affinity than healthy T cells.

59. A composition comprising any one of the CAR polypeptides of claims 1-31, the nucleic acids of claims 32 or 46, the vectors of claims 33-35, the cells of claims 36-41, or antibody or fragments thereof of claims 42-45.

60. A kit comprising any of the compositions of claim 59.
